(19) United States
(12) Patent Application Publication
(10) Pub. No.: US 2026/0116963 A1
(43) Pub. Date: Apr. 30, 2026

(54) ANTI-IL-13 ANTIBODIES AND METHODS OF USE THEREOF

(71) Applicant: Generate Biomedicines, Inc., Somerville, MA (US)

(72) Inventors: Adam ROOT, Somerville, MA (US); Jaileene HERNANDEZ ESCALANTE, Somerville, MA (US); Nadine SHABAN, Somerville, MA (US); Monica MENZENSKI, Somerville, MA (US); Kristen JOHNSON, Somerville, MA (US); Brinda MONIAN, Somerville, MA (US); Todd ASHWORTH, Somerville, MA (US); Kapil MAYAWALA, Somerville, MA (US); Heather VAN EPPS, Somerville, MA (US); Tanvi Krishnakumar GAWDE, Somerville, MA (US); Vikram SADINENI, Somerville, MA (US)

(21) Appl. No.: 19/009,049

(22) Filed: Jan. 3, 2025

Related U.S. Application Data
(60) Provisional application No. 63/618,322, filed on Jan. 6, 2024.
(60) Provisional application No. 63/626,442, filed on Jan. 29, 2024.

(51) Int. Cl. C07K 16/24; G16B 15/30

(52) U.S. Cl. CPC C07K 16/244; G16B 15/30; C07K 2317/21; C07K 2317/31; C07K 2317/565; C07K 2317/622

(57) ABSTRACT

The present application provides anti-IL-13 antibodies and antigen binding fragments thereof. Such antibodies and antigen binding fragments thereof may be used in methods for the treatment and/or prevention of an immunological disease or disorder.

### CROSS-REFERENCE TO RELATED APPLICATIONS

The present application claims the benefit of and priority to U.S. Provisional Application No. 63/618,322, filed on Jan. 6, 2024, and U.S. Provisional Application No. 63/626,442, filed on Jan. 29, 2024, both of which are incorporated herein by reference in their entirety.

### FIELD

Provided herein are antibodies and antigen binding fragments thereof that bind to interleukin-13 (IL-13).

### SEQUENCE LISTING

This application contains a sequence listing, which has been submitted in XML format via Patent Center. The contents of the XML copy named “134524-5008_Sequence_Listing_v5,” which was created on Dec. 23, 2024, and is 295,766 bytes in size, are incorporated herein by reference in their entirety.

### INCORPORATION BY REFERENCE OF MATERIAL IN AN ASCII FILE

Without limiting the foregoing, this application incorporates by reference the code of respective Appendices A and B. This application incorporates by reference the Computer Program Listing contained in the following ASCII file being submitted concurrently herewith:
a) File name: score.py; uploaded Dec. 23, 2024 and available at https://zenodo.org/records/14549062 or at https://doi.org/10.5281/zenodo. 14549062 and selecting “score.py”, 3,975 Bytes in size. The entire teachings of the above file are incorporated herein by reference in their entirety.
b) File name: model.etab; uploaded Dec. 23, 2024 and available at https://zenodo.org/records/14549062 or at https://doi.org/10.5281/zenodo. 14549062 and selecting “model.etab”, 3,500,928 Bytes in size. The entire teachings of the above file are incorporated herein by reference in their entirety.

The entire teachings of the above files are incorporated herein by reference in their entirety. The script in score.py is intended to be run in a Python environment in the same folder as model.etab to use the data of model.etab.

### BACKGROUND

IL-13 (also known as Interleukin-13 or NC30) is a cytokine protein which is produced by immune cells such as T helper type 2 (Th2) cells (see, e.g., Rael and Lockey, World Allergy Organ J. 4:54-64; 2011). It is largely found in the extracellular matrix and regulates physiological cellular changes related to allergic inflammation across many tissues (see, e.g., Minty et al., Nature. 362:248-250; 1993).

IL-13 plays many immunological roles in the cell. For example, it stimulates B cell proliferation and is involved in the activation of other immune cells including eosinophils, basophils, and mast cells (see, e.g., Defrance et al., J Exp Med. 179:135-143; 1994; Luttmann et al., J Immunol. 157:1678-1683; 1996). Importantly, IL-13 contributes to IgE synthesis from activated B cells, wherein high levels of IgE antibodies are characteristic of parasitic infection or an allergic response (see, e.g., Punnonen et al., J Allergy Clin Immunol. 100(6):792-801; 1997).

IL-13 is considered to be a key driver of inflammation in conditions such as atopic dermatitis where it is found to be notably overexpressed in skin lesions. Despite development of treatments for IL-13 mediated diseases and disorders such treatments are inefficacious due to requirement for repeated dosing every 2-4 weeks resulting in lack of patient compliance to course of maintenance therapy. Accordingly, a need exists for additional therapeutics that offer extended dosing frequency as a means to improve patient compliance and response to course of treatment.

### SUMMARY

Described herein, inter alia, are binding molecules (e.g., proteins), such as antibodies or antigen binding fragments thereof that bind to IL-13; nucleic acids encoding such binding molecules; methods for using and preparing such binding molecules; pharmaceutical compositions that comprise such binding molecules, for prophylactic, therapeutic or diagnostic purposes; and methods of treating disease or disorders that comprise administering the binding molecules to a subject in need thereof.

In some aspects, described herein are anti-IL-13 antibodies or antigen binding fragments thereof comprising: i) a light chain variable region comprising SEQ ID NO: 1 (LCDR1), SEQ ID NO: 2 (LCDR2), and SEQ ID NO: 3 (LCDR3), and a heavy chain variable region comprising SEQ ID NO: 4 (HCDR1), SEQ ID NO: 5 (HCDR2), and SEQ ID NO: 6 (HCDR3); ii) a light chain variable region comprising SEQ ID NO: 7 (LCDR1), SEQ ID NO: 8 (LCDR2), and SEQ ID NO: 9 (LCDR3), and a heavy chain variable region comprising SEQ ID NO: 10 (HCDR1), SEQ ID NO: 11 (HCDR2), and SEQ ID NO: 12 (HCDR3); iii) a light chain variable region comprising SEQ ID NO: 13 (LCDR1), SEQ ID NO: 14 (LCDR2), and SEQ ID NO: 15 (LCDR3), and a heavy chain variable region comprising SEQ ID NO: 16 (HCDR1), SEQ ID NO: 17 (HCDR2), and SEQ ID NO: 18 (HCDR3); iv) a light chain variable region comprising SEQ ID NO: 19 (LCDR1), SEQ ID NO: 20 (LCDR2), and SEQ ID NO: 21 (LCDR3), and a heavy chain variable region comprising SEQ ID NO: 22 (HCDR1), SEQ ID NO: 23 (HCDR2), and SEQ ID NO: 24 (HCDR3); v) a light chain variable region comprising SEQ ID NO: 25 (LCDR1), SEQ ID NO: 26 (LCDR2), and SEQ ID NO: 27 (LCDR3), and a heavy chain variable region comprising SEQ ID NO: 28 (HCDR1), SEQ ID NO: 29 (HCDR2), and SEQ ID NO: 30 (HCDR3); vi) a light chain variable region comprising SEQ ID NO: 31 (LCDR1), SEQ ID NO: 32 (LCDR2), and SEQ ID NO: 33 (LCDR3), and a heavy chain variable region comprising SEQ ID NO: 34 (HCDR1), SEQ ID NO: 35 (HCDR2), and SEQ ID NO: 36 (HCDR3); vii) a light chain variable region comprising SEQ ID NO: 37 (LCDR1), SEQ ID NO: 38 (LCDR2), and SEQ ID NO: 39 (LCDR3), and a heavy chain variable region comprising SEQ ID NO: 40 (HCDR1), SEQ ID NO: 41 (HCDR2), and SEQ ID NO: 42 (HCDR3); viii) a light chain variable region comprising SEQ ID NO: 43 (LCDR1), SEQ ID NO: 44 (LCDR2), and SEQ ID NO: 45 (LCDR3), and a heavy chain variable region comprising SEQ ID NO: 46 (HCDR1), SEQ ID NO: 47 (HCDR2), and SEQ ID NO: 48 (HCDR3); ix) a light chain variable region comprising SEQ ID NO: 49 (LCDR1), SEQ ID NO: 50 (LCDR2), and SEQ ID NO: 51 (LCDR3), and a heavy chain variable region comprising SEQ ID NO: 52 (HCDR1), SEQ ID NO: 53 (HCDR2), and SEQ ID NO: 54 (HCDR3); x) a light chain variable region comprising SEQ ID NO: 55 (LCDR1), SEQ ID NO: 56 (LCDR2), and SEQ ID NO: 57 (LCDR3), and a heavy chain variable region comprising SEQ ID NO: 58 (HCDR1), SEQ ID NO: 59 (HCDR2), and SEQ ID NO: 60 (HCDR3); xi) a light chain variable region comprising SEQ ID NO: 61 (LCDR1), SEQ ID NO: 62 (LCDR2), and SEQ ID NO: 63 (LCDR3), and a heavy chain variable region comprising SEQ ID NO: 64 (HCDR1), SEQ ID NO: 65 (HCDR2), and SEQ ID NO: 66 (HCDR3); xii) a light chain variable region comprising SEQ ID NO: 67 (LCDR1), SEQ ID NO: 68 (LCDR2), and SEQ ID NO: 69 (LCDR3), and a heavy chain variable region comprising SEQ ID NO: 70 (HCDR1), SEQ ID NO: 71 (HCDR2), and SEQ ID NO: 72 (HCDR3); xiii) a light chain variable region comprising SEQ ID NO: 73 (LCDR1), SEQ ID NO: 74 (LCDR2), and SEQ ID NO: 75 (LCDR3), and a heavy chain variable region comprising SEQ ID NO: 76 (HCDR1), SEQ ID NO: 77 (HCDR2), and SEQ ID NO: 78 (HCDR3); xiv) a light chain variable region comprising SEQ ID NO: 79 (LCDR1), SEQ ID NO: 80 (LCDR2), and SEQ ID NO: 81 (LCDR3), and a heavy chain variable region comprising SEQ ID NO: 82 (HCDR1), SEQ ID NO: 83 (HCDR2), and SEQ ID NO: 84 (HCDR3); xv) a light chain variable region comprising SEQ ID NO: 85 (LCDR1), SEQ ID NO: 86 (LCDR2), and SEQ ID NO: 87 (LCDR3), and a heavy chain variable region comprising SEQ ID NO: 88 (HCDR1), SEQ ID NO: 89 (HCDR2), and SEQ ID NO: 90 (HCDR3); or xvi) a light chain variable region comprising SEQ ID NO: 91 (LCDR1), SEQ ID NO: 92 (LCDR2), and SEQ ID NO: 93 (LCDR3), and a heavy chain variable region comprising SEQ ID NO: 94 (HCDR1), SEQ ID NO: 95 (HCDR2), and SEQ ID NO: 96 (HCDR3).

In some embodiments, the anti-IL-13 antibodies or antigen binding fragments thereof comprise a light chain variable region comprising an amino acid sequence that is at least about 90%, at least about 95%, at least about 96%, at least about 97%, at least about 98%, or at least about 99% identical to SEQ ID NO: 97, 99, 101, 103, 105, 107, 109, 111, 113, 115, 117, 119, 121, 123, 125, or 127; and/or a heavy chain variable region comprising an amino acid sequence that is at least about 90%, at least about 95%, at least about 96%, at least about 97%, at least about 98%, or at least about 99% identical to SEQ ID NO: 98, 100, 102, 104, 106, 108, 110, 112, 114, 116, 118, 120, 122, 124, 126, or 128.

In some embodiments, the anti-IL-13 antibodies or antigen binding fragments thereof comprise a light chain variable region comprising an amino acid sequence that is about 90%, about 95%, about 96%, about 97%, about 98%, or about 99% identical to SEQ ID NO: 97, 99, 101, 103, 105, 107, 109, 111, 113, 115, 117, 119, 121, 123, 125, or 127; and/or a heavy chain variable region comprising an amino acid sequence that is about 90%, about 95%, about 96%, about 97%, about 98%, or about 99% identical to SEQ ID NO: 98, 100, 102, 104, 106, 108, 110, 112, 114, 116, 118, 120, 122, 124, 126, or 128.

In some embodiments, the anti-IL-13 antibodies or antigen binding fragments thereof comprise a light chain variable region comprising an amino acid sequence as set forth in SEQ ID NO: 97, 99, 101, 103, 105, 107, 109, 111, 113, 115, 117, 119, 121, 123, 125, or 127; and/or a heavy chain variable region comprising an amino acid sequence as set forth in SEQ ID NO: 98, 100, 102, 104, 106, 108, 110, 112, 114, 116, 118, 120, 122, 124, 126, or 128.

In some embodiments, the anti-IL-13 antibodies or antigen binding fragments thereof comprise: i) a light chain variable region comprising an amino acid sequence as set forth in in SEQ ID NO: 97, and a heavy chain variable region comprising an amino acid sequence as set forth in in SEQ ID NO: 98; ii) a light chain variable region comprising an amino acid sequence as set forth in in SEQ ID NO: 99, and a heavy chain variable region comprising an amino acid sequence as set forth in in SEQ ID NO: 100; iii) a light chain variable region comprising an amino acid sequence as set forth in in SEQ ID NO: 101, and a heavy chain variable region comprising an amino acid sequence as set forth in in SEQ ID NO: 102; vi) a light chain variable region comprising an amino acid sequence as set forth in in SEQ ID NO: 103, and a heavy chain variable region comprising an amino acid sequence as set forth in in SEQ ID NO: 104; v) a light chain variable region comprising an amino acid sequence as set forth in in SEQ ID NO: 105, and a heavy chain variable region comprising an amino acid sequence as set forth in in SEQ ID NO: 106; vi) a light chain variable region comprising an amino acid sequence as set forth in in SEQ ID NO: 107, and a heavy chain variable region comprising an amino acid sequence as set forth in in SEQ ID NO: 108; vii) a light chain variable region comprising an amino acid sequence as set forth in in SEQ ID NO: 109, and a heavy chain variable region comprising an amino acid sequence as set forth in in SEQ ID NO: 110; vii) a light chain variable region comprising an amino acid sequence as set forth in in SEQ ID NO: 111, and a heavy chain variable region comprising an amino acid sequence as set forth in in SEQ ID NO: 112; ix) a light chain variable region comprising an amino acid sequence as set forth in in SEQ ID NO: 113, and a heavy chain variable region comprising an amino acid sequence as set forth in in SEQ ID NO: 114; x) a light chain variable region comprising an amino acid sequence as set forth in in SEQ ID NO: 115, and a heavy chain variable region comprising an amino acid sequence as set forth in in SEQ ID NO: 116; xi) a light chain variable region comprising an amino acid sequence as set forth in in SEQ ID NO: 117, and a heavy chain variable region comprising an amino acid sequence as set forth in in SEQ ID NO: 118: xii) a light chain variable region comprising an amino acid sequence as set forth in in SEQ ID NO: 119, and a heavy chain variable region comprising an amino acid sequence as set forth in in SEQ ID NO: 120; xiii) a light chain variable region comprising an amino acid sequence as set forth in in SEQ ID NO: 121, and a heavy chain variable region comprising an amino acid sequence as set forth in in SEQ ID NO: 122; xiv) a light chain variable region comprising an amino acid sequence as set forth in in SEQ ID NO: 123, and a heavy chain variable region comprising an amino acid sequence as set forth in in SEQ ID NO: 124; xv) a light chain variable region comprising an amino acid sequence as set forth in in SEQ ID NO: 125, and a heavy chain variable region comprising an amino acid sequence as set forth in in SEQ ID NO: 126; or xvi) a light chain variable region comprising an amino acid sequence as set forth in in SEQ ID NO: 127, and a heavy chain variable region comprising an amino acid sequence as set forth in in SEQ ID NO: 128.

In some embodiments, the anti-IL-13 antibodies or antigen binding fragments thereof comprise a light chain comprising an amino acid sequence that is at least about 90%, at least about 95%, at least about 96%, at least about 97%, at least about 98%, or at least about 99% identical to SEQ ID NO: 129, 131, 133, 135, 137, 139, 141, 143, 145, 147, 149, 151, 153, 155, 157, or 159; and/or a heavy chain comprising an amino acid sequence that is at least about 90%, at least about 95%, at least about 96%, at least about 97%, at least about 98%, or at least about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the anti-IL-13 antibodies or antigen binding fragments thereof comprise a light chain comprising an amino acid sequence that is about 90%, about 95%, about 96%, about 97%, about 98%, or about 99% identical to SEQ ID NO: 129, 131, 133, 135, 137, 139, 141, 143, 145, 147, 149, 151, 153, 155, 157, or 159; and/or a heavy chain comprising an amino acid sequence that is about 90%, about 95%, about 96%, about 97%, about 98%, or about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the anti-IL-13 antibodies or antigen binding fragments thereof comprises a light chain comprising an amino acid sequence as set forth in SEQ ID NO: 129, 131, 133, 135, 137, 139, 141, 143, 145, 147, 149, 151, 153, 155, 157, or 159; and/or a heavy chain comprising an amino acid sequence as set forth in SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the anti-IL-13 antibodies or antigen binding fragments thereof comprise: i) a light chain comprising an amino acid sequence as set forth in SEQ ID NO: 129, and a heavy chain comprising an amino acid sequence as set forth in SEQ ID NO: 130; ii) a light chain comprising an amino acid sequence as set forth in SEQ ID NO: 131, and a heavy chain comprising an amino acid sequence as set forth in SEQ ID NO: 132; iii) a light chain comprising an amino acid sequence as set forth in SEQ ID NO: 133, and a heavy chain comprising an amino acid sequence as set forth in SEQ ID NO: 134; iv) a light chain comprising an amino acid sequence as set forth in SEQ ID NO: 135, and a heavy chain comprising an amino acid sequence as set forth in SEQ ID NO: 136; v) a light chain comprising an amino acid sequence as set forth in SEQ ID NO: 137, and a heavy chain comprising an amino acid sequence as set forth in SEQ ID NO: 138; vi) a light chain comprising an amino acid sequence as set forth in SEQ ID NO: 139, and a heavy chain comprising an amino acid sequence as set forth in SEQ ID NO: 140; vii) a light chain comprising an amino acid sequence as set forth in SEQ ID NO: 141, and a heavy chain comprising an amino acid sequence as set forth in SEQ ID NO: 142; viii) a light chain comprising an amino acid sequence as set forth in SEQ ID NO: 143, and a heavy chain comprising an amino acid sequence as set forth in SEQ ID NO: 144; ix) a light chain comprising an amino acid sequence as set forth in SEQ ID NO: 145, and a heavy chain comprising an amino acid sequence as set forth in SEQ ID NO: 146; x) a light chain comprising an amino acid sequence as set forth in SEQ ID NO: 147, and a heavy chain comprising an amino acid sequence as set forth in SEQ ID NO: 148; xi) a light chain comprising an amino acid sequence as set forth in SEQ ID NO: 149, and a heavy chain comprising an amino acid sequence as set forth in SEQ ID NO: 150; xii) a light chain comprising an amino acid sequence as set forth in SEQ ID NO: 151, and a heavy chain comprising an amino acid sequence as set forth in SEQ ID NO: 152; xiii) a light chain comprising an amino acid sequence as set forth in SEQ ID NO: 153, and a heavy chain comprising an amino acid sequence as set forth in SEQ ID NO: 154; xiv) a light chain comprising an amino acid sequence as set forth in SEQ ID NO: 155, and a heavy chain comprising an amino acid sequence as set forth in SEQ ID NO: 156; xv) a light chain comprising an amino acid sequence as set forth in SEQ ID NO: 157, and a heavy chain comprising an amino acid sequence as set forth in SEQ ID NO: 158; xvi) a light chain comprising an amino acid sequence as set forth in SEQ ID NO: 159, and a heavy chain comprising an amino acid sequence as set forth in SEQ ID NO: 160; xvii) a light chain comprising an amino acid sequence as set forth in SEQ ID NO: 153, and a heavy chain comprising an amino acid sequence as set forth in SEQ ID NO: 182; xviii) a light chain comprising an amino acid sequence as set forth in SEQ ID NO: 147, and a heavy chain comprising an amino acid sequence as set forth in SEQ ID NO: 183; xix) a light chain comprising an amino acid sequence as set forth in SEQ ID NO: 141, and a heavy chain comprising an amino acid sequence as set forth in SEQ ID NO: 184; xx) a light chain comprising an amino acid sequence as set forth in SEQ ID NO: 153, and a heavy chain comprising an amino acid sequence as set forth in SEQ ID NO: 185; xxi) a light chain comprising an amino acid sequence as set forth in SEQ ID NO: 147, and a heavy chain comprising an amino acid sequence as set forth in SEQ ID NO: 186; xxii) a light chain comprising an amino acid sequence as set forth in SEQ ID NO: 141, and a heavy chain comprising an amino acid sequence as set forth in SEQ ID NO: 187; xxiii) a light chain comprising an amino acid sequence as set forth in SEQ ID NO: 153, and a heavy chain comprising an amino acid sequence as set forth in SEQ ID NO: 188; xxiv) a light chain comprising an amino acid sequence as set forth in SEQ ID NO: 147, and a heavy chain comprising an amino acid sequence as set forth in SEQ ID NO: 189; xxv) a light chain comprising an amino acid sequence as set forth in SEQ ID NO: 141, and a heavy chain comprising an amino acid sequence as set forth in SEQ ID NO: 190; xxvi) a light chain comprising an amino acid sequence as set forth in SEQ ID NO: 153, and a heavy chain comprising an amino acid sequence as set forth in SEQ ID NO: 191; xxvii) a light chain comprising an amino acid sequence as set forth in SEQ ID NO: 147, and a heavy chain comprising an amino acid sequence as set forth in SEQ ID NO: 192; xxviii) a light chain comprising an amino acid sequence as set forth in SEQ ID NO: 141, and a heavy chain comprising an amino acid sequence as set forth in SEQ ID NO: 193; xxix) a light chain comprising an amino acid sequence as set forth in SEQ ID NO: 153, and a heavy chain comprising an amino acid sequence as set forth in SEQ ID NO: 194; xxx) a light chain comprising an amino acid sequence as set forth in SEQ ID NO: 147, and a heavy chain comprising an amino acid sequence as set forth in SEQ ID NO: 195; xxxi) a light chain comprising an amino acid sequence as set forth in SEQ ID NO: 141, and a heavy chain comprising an amino acid sequence as set forth in SEQ ID NO: 196; xxxii) a light chain comprising an amino acid sequence as set forth in SEQ ID NO: 153, and a heavy chain comprising an amino acid sequence as set forth in SEQ ID NO: 197; xxxiii) a light chain comprising an amino acid sequence as set forth in SEQ ID NO: 147, and a heavy chain comprising an amino acid sequence as set forth in SEQ ID NO: 198; xxxiv) a light chain comprising an amino acid sequence as set forth in SEQ ID NO: 141, and a heavy chain comprising an amino acid sequence as set forth in SEQ ID NO: 199; xxxv) a light chain comprising an amino acid sequence as set forth in SEQ ID NO: 153, and a heavy chain comprising an amino acid sequence as set forth in SEQ ID NO: 200; xxxvi) a light chain comprising an amino acid sequence as set forth in SEQ ID NO: 147, and a heavy chain comprising an amino acid sequence as set forth in SEQ ID NO: 201; xxxvii) a light chain comprising an amino acid sequence as set forth in SEQ ID NO: 141, and a heavy chain comprising an amino acid sequence as set forth in SEQ ID NO: 202; xxxviii) a light chain comprising an amino acid sequence as set forth in SEQ ID NO: 153, and a heavy chain comprising an amino acid sequence as set forth in SEQ ID NO: 203; xxxix) a light chain comprising an amino acid sequence as set forth in SEQ ID NO: 147, and a heavy chain comprising an amino acid sequence as set forth in SEQ ID NO: 204; xl) a light chain comprising an amino acid sequence as set forth in SEQ ID NO: 141, and a heavy chain comprising an amino acid sequence as set forth in SEQ ID NO: 205; xli) a light chain comprising an amino acid sequence as set forth in SEQ ID NO: 153, and a heavy chain comprising an amino acid sequence as set forth in SEQ ID NO: 206; xlii) a light chain comprising an amino acid sequence as set forth in SEQ ID NO: 147, and a heavy chain comprising an amino acid sequence as set forth in SEQ ID NO: 207; xliii) a light chain comprising an amino acid sequence as set forth in SEQ ID NO: 141, and a heavy chain comprising an amino acid sequence as set forth in SEQ ID NO: 208; xliv) a light chain comprising an amino acid sequence as set forth in SEQ ID NO: 153, and a heavy chain comprising an amino acid sequence as set forth in SEQ ID NO: 209; xlv) a light chain comprising an amino acid sequence as set forth in SEQ ID NO: 147, and a heavy chain comprising an amino acid sequence as set forth in SEQ ID NO: 210; or xlvi) a light chain comprising an amino acid sequence as set forth in SEQ ID NO: 141, and a heavy chain comprising an amino acid sequence as set forth in SEQ ID NO: 211.

In some embodiments, the anti-IL-13 antibodies or antigen binding fragments thereof are monoclonal antibodies.

In some embodiments, the anti-IL-13 antibodies or antigen binding fragments thereof are human antibodies.

In some embodiments, the anti-IL-13 antibodies or antigen binding fragments thereof are bispecific antibodies.

In some embodiments, the antigen binding fragment thereof is selected from the group consisting of: Fab, Fab′, F(ab′)₂, Fd, Fv, scFv, a single-chain antibody, a minibody, and a diabody.

In some embodiments, the antigen binding fragments thereof is an scFv.

In some aspects, provided herein are anti-IL-13 antibodies or antigen binding fragments thereof used as a medicament.

In some aspects, provided herein is a pharmaceutical composition comprising anti-IL-13 antibodies or antigen binding fragments thereof and a pharmaceutically acceptable carrier.

In some aspects, provided herein is a nucleic acid composition comprising one or more nucleic acids encoding anti-IL-13 antibodies or antigen binding fragments thereof.

In some aspects, provided herein is an expression vector composition comprising one or more expression vectors comprising a nucleic acid composition as provided herein.

In some aspects, provided herein is a host cell comprising a nucleic acid composition or an expression vector composition as provided herein.

In some aspects, provided herein is a method for making anti-IL-13 antibodies or antigen binding fragments thereof comprising culturing the host cell as described herein under conditions where the anti-IL-13 antibodies or antigen binding fragments thereof are expressed and recovering the anti-IL-13 antibodies or binding fragments thereof.

In some aspects, also provided herein are anti-IL-13 antibodies or antigen binding fragments thereof comprising: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence at least about 90%, at least about 95%, or at least about 99% identical to SEQ ID NO: 97, 99, 101, 103, 105, 107, 109, 111, 113, 115, 117, 119, 121, 123, 125, or 127; and b2) a heavy chain variable region comprising an amino acid sequence at least about 90%, at least about 95%, or at least about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some aspects, also provided herein are anti-IL-13 antibodies or antigen binding fragments thereof comprising: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence about 90%, about 95%, or about 99% identical to SEQ ID NO: 97, 99, 101, 103, 105, 107, 109, 111, 113, 115, 117, 119, 121, 123, 125, or 127; and b2) a heavy chain variable region comprising an amino acid sequence about 90%, about 95%, or about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some embodiments, the anti-IL-13 antibodies or antigen binding fragments thereof comprise a LCDR1 comprising a serine (S) at amino acid position 27D and an arginine (R) at amino acid position 30; a LCDR1 comprising a tyrosine (Y) at amino acid position 27D and a histidine (H) at amino acid position 30; a LCDR1 comprising a tyrosine (Y) at amino acid position 27D and an asparagine (N) at amino acid position 30; or a LCDR1 comprises a serine (S) at amino acid position 27D and a tyrosine (Y) at amino acid position 32, wherein the amino acid positions are based on Kabat numbering.

In some aspects, also provided herein are anti-IL-13 antibodies or antigen binding fragments thereof comprising a light chain variable region and a heavy chain variable region, wherein the light chain variable region is at least about 95%, at least about 96%, at least about 97%, at least about 98%, or at least about 99% identical to SEQ ID NO: 97, 99, 101, and 103; wherein the heavy chain variable region is at least about 95%, at least about 96%, at least about 97%, at least about 98%, or at least about 99% identical to SEQ ID NO: 98, 100, 102, and 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, and wherein the amino acid positions are based on Kabat numbering.

In some aspects, also provided herein are anti-IL-13 antibodies or antigen binding fragments thereof comprising a light chain variable region and a heavy chain variable region, wherein the light chain variable region is about 95%, about 96%, about 97%, about 98%, or about 99% identical to SEQ ID NO: 97, 99, 101, and 103; wherein the heavy chain variable region is about 95%, about 96%, about 97%, about 98%, or about 99% identical to SEQ ID NO: 98, 100, 102, and 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, and wherein the amino acid positions are based on Kabat numbering.

In some embodiments, the anti-IL-13 antibodies or antigen binding fragments thereof are monoclonal antibodies.

In some embodiments, the anti-IL-13 antibodies or antigen binding fragments thereof are human antibodies.

In some embodiments, the anti-IL-13 antibodies or antigen binding fragments thereof are bispecific antibodies.

In some embodiments, the antigen binding fragment thereof is selected from the group consisting of: Fab, Fab′, F(ab′)₂, Fd, Fv, scFv, a single-chain antibody, a minibody, and a diabody.

In some embodiments, the antigen binding fragment thereof is an scFv.

In some aspects, provided herein are anti-IL-13 antibodies or antigen binding fragments thereof used as a medicament.

In some aspects, provided herein is a pharmaceutical composition comprising anti-IL-13 antibodies or an antigen binding fragments thereof and a pharmaceutically acceptable carrier.

In some aspects, provided herein is a nucleic acid composition comprising one or more nucleic acids encoding anti-IL-13 antibodies or antigen binding fragments thereof.

In some aspects, provided herein is an expression vector composition comprising one or more expression vectors comprising a nucleic acid composition as provided herein.

In some aspects, provided herein is a host cell comprising a nucleic acid composition or an expression vector composition as provided herein.

In some aspects, provided herein is a method for making anti-IL-13 antibodies or antigen binding fragments thereof comprising culturing the host cell as described herein under conditions where the anti-IL-13 antibodies or binding fragments thereof are expressed and recovering the anti-IL-13 antibodies or binding fragments thereof.

In some aspects, described herein is a method of treating a disease or disorder in a subject in need thereof, the method comprising administering to the subject a therapeutically effective dose of anti-IL-13 antibodies or antigen binding fragments thereof, wherein the anti-IL-13 antibodies or antigen binding fragments thereof comprise: i) a light chain variable region comprising SEQ ID NO: 1 (LCDR1), SEQ ID NO: 2 (LCDR2), and SEQ ID NO: 3 (LCDR3), and a heavy chain variable region comprising SEQ ID NO: 4 (HCDR1), SEQ ID NO: 5 (HCDR2), and SEQ ID NO: 6 (HCDR3); ii) a light chain variable region comprising SEQ ID NO: 7 (LCDR1). SEQ ID NO: 8 (LCDR2), and SEQ ID NO: 9 (LCDR3), and a heavy chain variable region comprising SEQ ID NO: 10 (HCDR1), SEQ ID NO: 11 (HCDR2), and SEQ ID NO: 12 (HCDR3); iii) a light chain variable region comprising SEQ ID NO: 13 (LCDR1), SEQ ID NO: 14 (LCDR2), and SEQ ID NO: 15 (LCDR3), and a heavy chain variable region comprising SEQ ID NO: 16 (HCDR1), SEQ ID NO: 17 (HCDR2), and SEQ ID NO: 18 (HCDR3); iv) a light chain variable region comprising SEQ ID NO: 19 (LCDR1), SEQ ID NO: 20 (LCDR2), and SEQ ID NO: 21 (LCDR3), and a heavy chain variable region comprising SEQ ID NO: 22 (HCDR1), SEQ ID NO: 23 (HCDR2), and SEQ ID NO: 24 (HCDR3); v) a light chain variable region comprising SEQ ID NO: 25 (LCDR1), SEQ ID NO: 26 (LCDR2), and SEQ ID NO: 27 (LCDR3), and a heavy chain variable region comprising SEQ ID NO: 28 (HCDR1), SEQ ID NO: 29 (HCDR2), and SEQ ID NO: 30 (HCDR3); vi) a light chain variable region comprising SEQ ID NO: 31 (LCDR1), SEQ ID NO: 32 (LCDR2), and SEQ ID NO: 33 (LCDR3), and a heavy chain variable region comprising SEQ ID NO: 34 (HCDR1), SEQ ID NO: 35 (HCDR2), and SEQ ID NO: 36 (HCDR3); vii) a light chain variable region comprising SEQ ID NO: 37 (LCDR1), SEQ ID NO: 38 (LCDR2), and SEQ ID NO: 39 (LCDR3), and a heavy chain variable region comprising SEQ ID NO: 40 (HCDR1), SEQ ID NO: 41 (HCDR2), and SEQ ID NO: 42 (HCDR3); viii) a light chain variable region comprising SEQ ID NO: 43 (LCDR1), SEQ ID NO: 44 (LCDR2), and SEQ ID NO: 45 (LCDR3), and a heavy chain variable region comprising SEQ ID NO: 46 (HCDR1), SEQ ID NO: 47 (HCDR2), and SEQ ID NO: 48 (HCDR3); ix) a light chain variable region comprising SEQ ID NO: 49 (LCDR1), SEQ ID NO: 50 (LCDR2), and SEQ ID NO: 51 (LCDR3), and a heavy chain variable region comprising SEQ ID NO: 52 (HCDR1), SEQ ID NO: 53 (HCDR2), and SEQ ID NO: 54 (HCDR3); x) a light chain variable region comprising SEQ ID NO: 55 (LCDR1), SEQ ID NO: 56 (LCDR2), and SEQ ID NO: 57 (LCDR3), and a heavy chain variable region comprising SEQ ID NO: 58 (HCDR1), SEQ ID NO: 59 (HCDR2), and SEQ ID NO: 60 (HCDR3); xi) a light chain variable region comprising SEQ ID NO: 61 (LCDR1), SEQ ID NO: 62 (LCDR2), and SEQ ID NO: 63 (LCDR3), and a heavy chain variable region comprising SEQ ID NO: 64 (HCDR1), SEQ ID NO: 65 (HCDR2), and SEQ ID NO: 66 (HCDR3); xii) a light chain variable region comprising SEQ ID NO: 67 (LCDR1), SEQ ID NO: 68 (LCDR2), and SEQ ID NO: 69 (LCDR3), and a heavy chain variable region comprising SEQ ID NO: 70 (HCDR1), SEQ ID NO: 71 (HCDR2), and SEQ ID NO: 72 (HCDR3); xiii) a light chain variable region comprising SEQ ID NO: 73 (LCDR1), SEQ ID NO: 74 (LCDR2), and SEQ ID NO: 75 (LCDR3), and a heavy chain variable region comprising SEQ ID NO: 76 (HCDR1), SEQ ID NO: 77 (HCDR2), and SEQ ID NO: 78 (HCDR3); xiv) a light chain variable region comprising SEQ ID NO: 79 (LCDR1), SEQ ID NO: 80 (LCDR2), and SEQ ID NO: 81 (LCDR3), and a heavy chain variable region comprising SEQ ID NO: 82 (HCDR1), SEQ ID NO: 83 (HCDR2), and SEQ ID NO: 84 (HCDR3): xv) a light chain variable region comprising SEQ ID NO: 85 (LCDR1), SEQ ID NO: 86 (LCDR2), and SEQ ID NO: 87 (LCDR3), and a heavy chain variable region comprising SEQ ID NO: 88 (HCDR1), SEQ ID NO: 89 (HCDR2), and SEQ ID NO: 90 (HCDR3); or xvi) a light chain variable region comprising SEQ ID NO: 91 (LCDR1), SEQ ID NO: 92 (LCDR2), and SEQ ID NO: 93 (LCDR3), and a heavy chain variable region comprising SEQ ID NO: 94 (HCDR1), SEQ ID NO: 95 (HCDR2), and SEQ ID NO: 96 (HCDR3).

In some embodiments, the disease or disorder is selected from the group consisting of: asthma (including, but not limited to, mild asthma, mild to moderate asthma, moderate asthma, moderate to severe asthma, and/or severe asthma), allergic asthma, allergen-induced airway obstruction due to asthma and/or allergic asthma, atopic dermatitis (including, but not limited to, mild atopic dermatitis, mild to moderate atopic dermatitis, moderate atopic dermatitis, moderate-to-severe, and/or severe atopic dermatitis), eczema, acute urticaria, idiopathic pulmonary fibrosis (IPF), chronic obstructive pulmonary disease (COPD), chronic obstructive airway disease, emphysema, chronic bronchitis, pulmonary fibrosis, systemic sclerosis, scleroderma, cryptogenic fibrosing alveolitis, usual interstitial pneumonitis, idiopathic interstitial pneumonitis, wound, systemic lupus erythematosus (SLE), rheumatoid arthritis (RA), systemic sclerosis (SS), ulcerative colitis (UC), type 1 diabetes (T1D), sjogren's syndrome (SS), prurigo nodularis, nasal polyposis, recurrent urticaria, aspergillosis, bullous pemphigoid, chronic sinusitis, alopecia areata, hay fever, pemphigus, allergic rhinitis, psoriasis, rosacea, allergic drug reactions, allergic reactions, anaphylaxis, acne, food allergies, inflammatory bowel disease (IBS), Crohn's disease, ulcerative colitis, chronic rhinosinusitis with nasal polyps (CRSwNP), eosinophilic esophagitis, eosinophilic colitis, eosinophilic gastritis, eosinophilic cystitis, eosinophilic fasciitis, eosinophilic pustular folliculitis, eosinophilic granulomatosis, eosinophilic polyangiitis, Churg-Strauss syndrome, biliary cancer, brain cancer, breast cancer, colorectal cancer, genitourinary cancer, head and neck cancer, liver cancer, Hodgkin's lymphoma, esophageal cancer, pancreatic cancer, prostate cancer, renal cancer, Kaposi's sarcoma, sinusitis, or chronic urticaria.

In some embodiments, the light chain variable region comprises an amino acid sequence that is at least about 90%, at least about 95%, or at least about 99% identical to SEQ ID NO: 97, 99, 101, 103, 105, 107, 109, 111, 113, 115, 117, 119, 121, 123, 125, or 127; and the heavy chain variable region comprises an amino acid sequence that is at least about 90%, at least about 95%, or at least about 99% identical to SEQ ID NO: 98, 100, 102, 104, 106, 108, 110, 112, 114, 116, 118, 120, 122, 124, 126, or 128.

In some embodiments, the light chain variable region comprises an amino acid sequence that is about 90%, about 95%, or about 99% identical to SEQ ID NO: 97, 99, 101, 103, 105, 107, 109, 111, 113, 115, 117, 119, 121, 123, 125, or 127; and the heavy chain variable region comprises an amino acid sequence that is about 90%, about 95%, or about 99% identical to SEQ ID NO: 98, 100, 102, 104, 106, 108, 110, 112, 114, 116, 118, 120, 122, 124, 126, or 128.

In some embodiments, the light chain variable region comprises an amino acid sequence as set forth in SEQ ID NO: 97, 99, 101, 103, 105, 107, 109, 111, 113, 115, 117, 119, 121, 123, 125, or 127; and/or the heavy chain variable region comprises an amino acid sequence as set forth in SEQ ID NO: 98, 100, 102, 104, 106, 108, 110, 112, 114, 116, 118, 120, 122, 124, 126, or 128.

In some embodiments, the light chain variable region comprises an amino acid sequence as set forth in SEQ ID NO: 97, and the heavy chain variable region comprises an amino acid sequence as set forth in SEQ ID NO: 98; the light chain variable region comprises an amino acid sequence as set forth in SEQ ID NO: 99, and the heavy chain variable region comprises an amino acid sequence as set forth in SEQ ID NO: 100; the light chain variable region comprises an amino acid sequence as set forth in SEQ ID NO: 101, and the heavy chain variable region comprises an amino acid sequence as set forth in SEQ ID NO: 102; the light chain variable region comprises an amino acid sequence as set forth in SEQ ID NO: 103, and the heavy chain variable region comprises an amino acid sequence as set forth in SEQ ID NO: 104; the light chain variable region comprises an amino acid sequence as set forth in SEQ ID NO: 105, and the heavy chain variable region comprises an amino acid sequence as set forth in SEQ ID NO: 106; the light chain variable region comprises an amino acid sequence as set forth in SEQ ID NO: 107, and the heavy chain variable region comprises an amino acid sequence as set forth in SEQ ID NO: 108; the light chain variable region comprises an amino acid sequence as set forth in SEQ ID NO: 109, and the heavy chain variable region comprises an amino acid sequence as set forth in SEQ ID NO: 110; the light chain variable region comprises an amino acid sequence as set forth in SEQ ID NO: 111, and the heavy chain variable region comprises an amino acid sequence as set forth in SEQ ID NO: 112; the light chain variable region comprises an amino acid sequence as set forth in SEQ ID NO: 113, and the heavy chain variable region comprises an amino acid sequence as set forth in SEQ ID NO: 114; the light chain variable region comprises an amino acid sequence as set forth in SEQ ID NO: 115, and the heavy chain variable region comprises an amino acid sequence as set forth in SEQ ID NO: 116; the light chain variable region comprises an amino acid sequence as set forth in SEQ ID NO: 117, and the heavy chain variable region comprises an amino acid sequence as set forth in SEQ ID NO: 118; the light chain variable region comprises an amino acid sequence as set forth in SEQ ID NO: 119, and the heavy chain variable region comprises an amino acid sequence as set forth in SEQ ID NO: 120; the light chain variable region comprises an amino acid sequence as set forth in SEQ ID NO: 121, and the heavy chain variable region comprises an amino acid sequence as set forth in SEQ ID NO: 122; the light chain variable region comprises an amino acid sequence as set forth in SEQ ID NO: 123, and the heavy chain variable region comprises an amino acid sequence as set forth in SEQ ID NO: 124; the light chain variable region comprises an amino acid sequence as set forth in SEQ ID NO: 125, and the heavy chain variable region comprises an amino acid sequence as set forth in SEQ ID NO: 126; or the light chain variable region comprises an amino acid sequence as set forth in SEQ ID NO: 127, and the heavy chain variable region comprises an amino acid sequence as set forth in SEQ ID NO: 128.

In some embodiments, the anti-IL-13 antibodies or antigen binding fragments thereof comprise a light chain comprising an amino acid sequence that is at least about 90%, at least about 95%, or at least about 99% identical to SEQ ID NO: 129, 131, 133, 135, 137, 139, 141, 143, 145, 147, 149, 151, 153, 155, 157, or 159; and/or a heavy chain comprising an amino acid sequence that is at least about 90%, at least about 95%, or at least about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the anti-IL-13 antibodies or antigen binding fragments thereof comprise a light chain comprising an amino acid sequence that is about 90%, about 95%, or about 99% identical to SEQ ID NO: 129, 131, 133, 135, 137, 139, 141, 143, 145, 147, 149, 151, 153, 155, 157, or 159; and/or a heavy chain comprising an amino acid sequence that is about 90%, about 95%, or about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the light chain comprises an amino acid sequence as set forth in SEQ ID NO: 129, 131, 133, 135, 137, 139, 141, 143, 145, 147, 149, 151, 153, 155, 157, or 159; and/or the heavy chain comprises an amino acid sequence as set forth in SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the light chain comprises an amino acid sequence as set forth in SEQ ID NO: 129, and the heavy chain comprises an amino acid sequence as set forth in SEQ ID NO: 130; the light chain comprises an amino acid sequence as set forth in SEQ ID NO: 131, and the heavy chain comprises an amino acid sequence as set forth in SEQ ID NO: 132; the light chain comprises an amino acid sequence as set forth in SEQ ID NO: 133, and the heavy chain comprises an amino acid sequence as set forth in SEQ ID NO: 134; the light chain comprises an amino acid sequence as set forth in SEQ ID NO: 135, and the heavy chain comprises an amino acid sequence as set forth in SEQ ID NO: 136; the light chain comprises an amino acid sequence as set forth in SEQ ID NO: 137, and the heavy chain comprises an amino acid sequence as set forth in SEQ ID NO: 138; the light chain comprises an amino acid sequence as set forth in SEQ ID NO: 139, and the heavy chain comprises an amino acid sequence as set forth in SEQ ID NO: 140; the light chain comprises an amino acid sequence as set forth in SEQ ID NO: 141, and the heavy chain comprises an amino acid sequence as set forth in SEQ ID NO: 142; the light chain comprises an amino acid sequence as set forth in SEQ ID NO: 143, and the heavy chain comprises an amino acid sequence as set forth in SEQ ID NO: 144; the light chain comprises an amino acid sequence as set forth in SEQ ID NO: 145, and the heavy chain comprises an amino acid sequence as set forth in SEQ ID NO: 146; the light chain comprises an amino acid sequence as set forth in SEQ ID NO: 147, and the heavy chain comprises an amino acid sequence as set forth in SEQ ID NO: 148; the light chain comprises an amino acid sequence as set forth in SEQ ID NO: 149, and the heavy chain comprises an amino acid sequence as set forth in SEQ ID NO: 150; the light chain comprises an amino acid sequence as set forth in SEQ ID NO: 151, and the heavy chain comprises an amino acid sequence as set forth in SEQ ID NO: 152; the light chain comprises an amino acid sequence as set forth in SEQ ID NO: 153, and the heavy chain comprises an amino acid sequence as set forth in SEQ ID NO: 154; the light chain comprises an amino acid sequence as set forth in SEQ ID NO: 155, and the heavy chain comprises an amino acid sequence as set forth in SEQ ID NO: 156; the light chain comprises an amino acid sequence as set forth in SEQ ID NO: 157, and the heavy chain comprises an amino acid sequence as set forth in SEQ ID NO: 158; the light chain comprises an amino acid sequence as set forth in SEQ ID NO: 159, and the heavy chain comprises an amino acid sequence as set forth in SEQ ID NO: 160; the light chain comprises an amino acid sequence as set forth in SEQ ID NO: 153, and the heavy chain comprises an amino acid sequence as set forth in SEQ ID NO: 182; the light chain comprises an amino acid sequence as set forth in SEQ ID NO: 147, and the heavy chain comprises an amino acid sequence as set forth in SEQ ID NO: 183; the light chain comprises an amino acid sequence as set forth in SEQ ID NO: 141, and the heavy chain comprises an amino acid sequence as set forth in SEQ ID NO: 184; the light chain comprises an amino acid sequence as set forth in SEQ ID NO: 153, and the heavy chain comprises an amino acid sequence as set forth in SEQ ID NO: 185; the light chain comprises an amino acid sequence as set forth in SEQ ID NO: 147, and the heavy chain comprises an amino acid sequence as set forth in SEQ ID NO: 186; the light chain comprises an amino acid sequence as set forth in SEQ ID NO: 141, and the heavy chain comprises an amino acid sequence as set forth in SEQ ID NO: 187; the light chain comprises an amino acid sequence as set forth in SEQ ID NO: 153, and the heavy chain comprises an amino acid sequence as set forth in SEQ ID NO: 188; the light chain comprises an amino acid sequence as set forth in SEQ ID NO: 147, and the heavy chain comprises an amino acid sequence as set forth in SEQ ID NO: 189; the light chain comprises an amino acid sequence as set forth in SEQ ID NO: 141, and the heavy chain comprises an amino acid sequence as set forth in SEQ ID NO: 190; the light chain comprises an amino acid sequence as set forth in SEQ ID NO: 153, and the heavy chain comprises an amino acid sequence as set forth in SEQ ID NO: 191; the light chain comprises an amino acid sequence as set forth in SEQ ID NO: 147, and the heavy chain comprises an amino acid sequence as set forth in SEQ ID NO: 192; the light chain comprises an amino acid sequence as set forth in SEQ ID NO: 141, and the heavy chain comprises an amino acid sequence as set forth in SEQ ID NO: 193; the light chain comprises an amino acid sequence as set forth in SEQ ID NO: 153, and the heavy chain comprises an amino acid sequence as set forth in SEQ ID NO: 194; the light chain comprises an amino acid sequence as set forth in SEQ ID NO: 147, and the heavy chain comprises an amino acid sequence as set forth in SEQ ID NO: 195; the light chain comprises an amino acid sequence as set forth in SEQ ID NO: 141, and the heavy chain comprises an amino acid sequence as set forth in SEQ ID NO: 196; the light chain comprises an amino acid sequence as set forth in SEQ ID NO: 153, and the heavy chain comprises an amino acid sequence as set forth in SEQ ID NO: 197; the light chain comprises an amino acid sequence as set forth in SEQ ID NO: 147, and the heavy chain comprises an amino acid sequence as set forth in SEQ ID NO: 198; the light chain comprises an amino acid sequence as set forth in SEQ ID NO: 141, and the heavy chain comprises an amino acid sequence as set forth in SEQ ID NO: 199; the light chain comprises an amino acid sequence as set forth in SEQ ID NO: 153, and the heavy chain comprises an amino acid sequence as set forth in SEQ ID NO: 200; the light chain comprises an amino acid sequence as set forth in SEQ ID NO: 147, and the heavy chain comprises an amino acid sequence as set forth in SEQ ID NO: 201; the light chain comprises an amino acid sequence as set forth in SEQ ID NO: 141, and the heavy chain comprises an amino acid sequence as set forth in SEQ ID NO: 202; the light chain comprises an amino acid sequence as set forth in SEQ ID NO: 153, and the heavy chain comprises an amino acid sequence as set forth in SEQ ID NO: 203; the light chain comprises an amino acid sequence as set forth in SEQ ID NO: 147, and the heavy chain comprises an amino acid sequence as set forth in SEQ ID NO: 204; the light chain comprises an amino acid sequence as set forth in SEQ ID NO: 141, and the heavy chain comprises an amino acid sequence as set forth in SEQ ID NO: 205; the light chain comprises an amino acid sequence as set forth in SEQ ID NO: 153, and the heavy chain comprises an amino acid sequence as set forth in SEQ ID NO: 206; the light chain comprises an amino acid sequence as set forth in SEQ ID NO: 147, and the heavy chain comprises an amino acid sequence as set forth in SEQ ID NO: 207; the light chain comprises an amino acid sequence as set forth in SEQ ID NO: 141, and the heavy chain comprises an amino acid sequence as set forth in SEQ ID NO: 208; the light chain comprises an amino acid sequence as set forth in SEQ ID NO: 153, and the heavy chain comprises an amino acid sequence as set forth in SEQ ID NO: 209; the light chain comprises an amino acid sequence as set forth in SEQ ID NO: 147, and the heavy chain comprises an amino acid sequence as set forth in SEQ ID NO: 210; or the light chain comprises an amino acid sequence as set forth in SEQ ID NO: 141, and the heavy chain comprises an amino acid sequence as set forth in SEQ ID NO: 211.

In some embodiments, the anti-IL-13 antibodies or antigen binding fragments thereof are monoclonal antibodies.

In some embodiments, the anti-IL-13 antibodies or antigen binding fragments thereof are human antibodies.

In some embodiments, the anti-IL-13 antibodies or antigen binding fragments thereof are bispecific antibodies.

In some embodiments, the antigen binding fragment thereof is selected from the group consisting of Fab, Fab′, F(ab′)₂, Fd, Fv, scFv, a single-chain antibody, a minibody, and a diabody. In some embodiments, the antigen binding fragment thereof is an scFv.

In some aspects, described herein are antibodies or antigen binding fragments thereof comprising: i) a light chain variable region comprising SEQ ID NO: 1 (LCDR1), SEQ ID NO: 2 (LCDR2), and SEQ ID NO: 3 (LCDR3), and a heavy chain variable region comprising SEQ ID NO: 4 (HCDR1), SEQ ID NO: 5 (HCDR2), and SEQ ID NO: 6 (HCDR3); ii) a light chain variable region comprising SEQ ID NO: 7 (LCDR1), SEQ ID NO: 8 (LCDR2), and SEQ ID NO: 9 (LCDR3), and a heavy chain variable region comprising SEQ ID NO: 10 (HCDR1), SEQ ID NO: 11 (HCDR2), and SEQ ID NO: 12 (HCDR3); iii) a light chain variable region comprising SEQ ID NO: 13 (LCDR1), SEQ ID NO: 14 (LCDR2), and SEQ ID NO: 15 (LCDR3), and a heavy chain variable region comprising SEQ ID NO: 16 (HCDR1), SEQ ID NO: 17 (HCDR2), and SEQ ID NO: 18 (HCDR3); iv) a light chain variable region comprising SEQ ID NO: 19 (LCDR1), SEQ ID NO: 20 (LCDR2), and SEQ ID NO: 21 (LCDR3), and a heavy chain variable region comprising SEQ ID NO: 22 (HCDR1). SEQ ID NO: 23 (HCDR2), and SEQ ID NO: 24 (HCDR3); v) a light chain variable region comprising SEQ ID NO: 25 (LCDR1), SEQ ID NO: 26 (LCDR2), and SEQ ID NO: 27 (LCDR3), and a heavy chain variable region comprising SEQ ID NO: 28 (HCDR1), SEQ ID NO: 29 (HCDR2), and SEQ ID NO: 30 (HCDR3); vi) a light chain variable region comprising SEQ ID NO: 31 (LCDR1), SEQ ID NO: 32 (LCDR2), and SEQ ID NO: 33 (LCDR3), and a heavy chain variable region comprising SEQ ID NO: 34 (HCDR1), SEQ ID NO: 35 (HCDR2), and SEQ ID NO: 36 (HCDR3); vii) a light chain variable region comprising SEQ ID NO: 37 (LCDR1), SEQ ID NO: 38 (LCDR2), and SEQ ID NO: 39 (LCDR3), and a heavy chain variable region comprising SEQ ID NO: 40 (HCDR1), SEQ ID NO: 41 (HCDR2), and SEQ ID NO: 42 (HCDR3); viii) a light chain variable region comprising SEQ ID NO: 43 (LCDR1), SEQ ID NO: 44 (LCDR2), and SEQ ID NO: 45 (LCDR3), and a heavy chain variable region comprising SEQ ID NO: 46 (HCDR1), SEQ ID NO: 47 (HCDR2), and SEQ ID NO: 48 (HCDR3); ix) a light chain variable region comprising SEQ ID NO: 49 (LCDR1), SEQ ID NO: 50 (LCDR2), and SEQ ID NO: 51 (LCDR3), and a heavy chain variable region comprising SEQ ID NO: 52 (HCDR1), SEQ ID NO: 53 (HCDR2), and SEQ ID NO: 54 (HCDR3); x) a light chain variable region comprising SEQ ID NO: 55 (LCDR1), SEQ ID NO: 56 (LCDR2), and SEQ ID NO: 57 (LCDR3), and a heavy chain variable region comprising SEQ ID NO: 58 (HCDR1). SEQ ID NO: 59 (HCDR2), and SEQ ID NO: 60 (HCDR3); xi) a light chain variable region comprising SEQ ID NO: 61 (LCDR1), SEQ ID NO: 62 (LCDR2), and SEQ ID NO: 63 (LCDR3), and a heavy chain variable region comprising SEQ ID NO: 64 (HCDR1), SEQ ID NO: 65 (HCDR2), and SEQ ID NO: 66 (HCDR3); xii) a light chain variable region comprising SEQ ID NO: 67 (LCDR1), SEQ ID NO: 68 (LCDR2), and SEQ ID NO: 69 (LCDR3), and a heavy chain variable region comprising SEQ ID NO: 70 (HCDR1), SEQ ID NO: 71 (HCDR2), and SEQ ID NO: 72 (HCDR3); xiii) a light chain variable region comprising SEQ ID NO: 73 (LCDR1), SEQ ID NO: 74 (LCDR2), and SEQ ID NO: 75 (LCDR3), and a heavy chain variable region comprising SEQ ID NO: 76 (HCDR1), SEQ ID NO: 77 (HCDR2), and SEQ ID NO: 78 (HCDR3); xiv) a light chain variable region comprising SEQ ID NO: 79 (LCDR1), SEQ ID NO: 80 (LCDR2), and SEQ ID NO: 81 (LCDR3), and a heavy chain variable region comprising SEQ ID NO: 82 (HCDR1), SEQ ID NO: 83 (HCDR2), and SEQ ID NO: 84 (HCDR3); xv) a light chain variable region comprising SEQ ID NO: 85 (LCDR1), SEQ ID NO: 86 (LCDR2), and SEQ ID NO: 87 (LCDR3), and a heavy chain variable region comprising SEQ ID NO: 88 (HCDR1), SEQ ID NO: 89 (HCDR2), and SEQ ID NO: 90 (HCDR3); or xvi) a light chain variable region comprising SEQ ID NO: 91 (LCDR1), SEQ ID NO: 92 (LCDR2), and SEQ ID NO: 93 (LCDR3), and a heavy chain variable region comprising SEQ ID NO: 94 (HCDR1), SEQ ID NO: 95 (HCDR2), and SEQ ID NO: 96 (HCDR3).

In some embodiments, the antibodies or antigen binding fragments thereof comprise a light chain variable region comprising an amino acid sequence that is at least about 90%, at least about 95%, at least about 96%, at least about 97%, at least about 98%, or at least about 99% identical to SEQ ID NO: 97, 99, 101, 103, 105, 107, 109, 111, 113, 115, 117, 119, 121, 123, 125, or 127; and/or a heavy chain variable region comprising an amino acid sequence that is at least about 90%, at least about 95%, at least about 96%, at least about 97%, at least about 98%, or at least about 99% identical to SEQ ID NO: 98, 100, 102, 104, 106, 108, 110, 112, 114, 116, 118, 120, 122, 124, 126, or 128.

In some embodiments, the antibodies or antigen binding fragments thereof comprise a light chain variable region comprising an amino acid sequence that is about 90%, about 95%, about 96%, about 97%, about 98%, or about 99% identical to SEQ ID NO: 97, 99, 101, 103, 105, 107, 109, 111, 113, 115, 117, 119, 121, 123, 125, or 127; and/or a heavy chain variable region comprising an amino acid sequence that is about 90%, about 95%, about 96%, about 97%, about 98%, or about 99% identical to SEQ ID NO: 98, 100, 102, 104, 106, 108, 110, 112, 114, 116, 118, 120, 122, 124, 126, or 128.

In some embodiments, the antibodies or antigen binding fragments thereof comprise a light chain variable region comprising an amino acid sequence as set forth in SEQ ID NO: 97, 99, 101, 103, 105, 107, 109, 111, 113, 115, 117, 119, 121, 123, 125, or 127; and/or a heavy chain variable region comprising an amino acid sequence as set forth in SEQ ID NO: 98, 100, 102, 104, 106, 108, 110, 112, 114, 116, 118, 120, 122, 124, 126, or 128.

In some embodiments, the antibodies or antigen binding fragments thereof comprise: i) a light chain variable region comprising an amino acid sequence as set forth in in SEQ ID NO: 97, and a heavy chain variable region comprising an amino acid sequence as set forth in in SEQ ID NO: 98; ii) a light chain variable region comprising an amino acid sequence as set forth in in SEQ ID NO: 99, and a heavy chain variable region comprising an amino acid sequence as set forth in in SEQ ID NO: 100; iii) a light chain variable region comprising an amino acid sequence as set forth in in SEQ ID NO: 101, and a heavy chain variable region comprising an amino acid sequence as set forth in in SEQ ID NO: 102; vi) a light chain variable region comprising an amino acid sequence as set forth in in SEQ ID NO: 103, and a heavy chain variable region comprising an amino acid sequence as set forth in in SEQ ID NO: 104; v) a light chain variable region comprising an amino acid sequence as set forth in in SEQ ID NO: 105, and a heavy chain variable region comprising an amino acid sequence as set forth in in SEQ ID NO: 106; vi) a light chain variable region comprising an amino acid sequence as set forth in in SEQ ID NO: 107, and a heavy chain variable region comprising an amino acid sequence as set forth in in SEQ ID NO: 108; vii) a light chain variable region comprising an amino acid sequence as set forth in in SEQ ID NO: 109, and a heavy chain variable region comprising an amino acid sequence as set forth in in SEQ ID NO: 110; vii) a light chain variable region comprising an amino acid sequence as set forth in in SEQ ID NO: 111, and a heavy chain variable region comprising an amino acid sequence as set forth in in SEQ ID NO: 112; ix) a light chain variable region comprising an amino acid sequence as set forth in in SEQ ID NO: 113, and a heavy chain variable region comprising an amino acid sequence as set forth in in SEQ ID NO: 114; x) a light chain variable region comprising an amino acid sequence as set forth in in SEQ ID NO: 115, and a heavy chain variable region comprising an amino acid sequence as set forth in in SEQ ID NO: 116; xi) a light chain variable region comprising an amino acid sequence as set forth in in SEQ ID NO: 117, and a heavy chain variable region comprising an amino acid sequence as set forth in in SEQ ID NO: 118; xii) a light chain variable region comprising an amino acid sequence as set forth in in SEQ ID NO: 119, and a heavy chain variable region comprising an amino acid sequence as set forth in in SEQ ID NO: 120; xiii) a light chain variable region comprising an amino acid sequence as set forth in in SEQ ID NO: 121, and a heavy chain variable region comprising an amino acid sequence as set forth in in SEQ ID NO: 122; xiv) a light chain variable region comprising an amino acid sequence as set forth in in SEQ ID NO: 123, and a heavy chain variable region comprising an amino acid sequence as set forth in in SEQ ID NO: 124; xv) a light chain variable region comprising an amino acid sequence as set forth in in SEQ ID NO: 125, and a heavy chain variable region comprising an amino acid sequence as set forth in in SEQ ID NO: 126; or xvi) a light chain variable region comprising an amino acid sequence as set forth in in SEQ ID NO: 127, and a heavy chain variable region comprising an amino acid sequence as set forth in in SEQ ID NO: 128.

In some embodiments, the antibodies or antigen binding fragments thereof comprise a light chain comprising an amino acid sequence that is at least about 90%, at least about 95%, at least about 96%, at least about 97%, at least about 98%, or at least about 99% identical to SEQ ID NO: 129, 131, 133, 135, 137, 139, 141, 143, 145, 147, 149, 151, 153, 155, 157, or 159; and/or a heavy chain comprising an amino acid sequence that is at least about 90%, at least about 95%, at least about 96%, at least about 97%, at least about 98%, or at least about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof comprise a light chain comprising an amino acid sequence that is about 90%, about 95%, about 96%, about 97%, about 98%, or about 99% identical to SEQ ID NO: 129, 131, 133, 135, 137, 139, 141, 143, 145, 147, 149, 151, 153, 155, 157, or 159; and/or a heavy chain comprising an amino acid sequence that is about 90%, about 95%, about 96%, about 97%, about 98%, or about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof comprises a light chain comprising an amino acid sequence as set forth in SEQ ID NO: 129, 131, 133, 135, 137, 139, 141, 143, 145, 147, 149, 151, 153, 155, 157, or 159; and/or a heavy chain comprising an amino acid sequence as set forth in SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof comprise: i) a light chain comprising an amino acid sequence as set forth in SEQ ID NO: 129, and a heavy chain comprising an amino acid sequence as set forth in SEQ ID NO: 130; ii) a light chain comprising an amino acid sequence as set forth in SEQ ID NO: 131, and a heavy chain comprising an amino acid sequence as set forth in SEQ ID NO: 132; iii) a light chain comprising an amino acid sequence as set forth in SEQ ID NO: 133, and a heavy chain comprising an amino acid sequence as set forth in SEQ ID NO: 134; iv) a light chain comprising an amino acid sequence as set forth in SEQ ID NO: 135, and a heavy chain comprising an amino acid sequence as set forth in SEQ ID NO: 136; v) a light chain comprising an amino acid sequence as set forth in SEQ ID NO: 137, and a heavy chain comprising an amino acid sequence as set forth in SEQ ID NO: 138; vi) a light chain comprising an amino acid sequence as set forth in SEQ ID NO: 139, and a heavy chain comprising an amino acid sequence as set forth in SEQ ID NO: 140; vii) a light chain comprising an amino acid sequence as set forth in SEQ ID NO: 141, and a heavy chain comprising an amino acid sequence as set forth in SEQ ID NO: 142; viii) a light chain comprising an amino acid sequence as set forth in SEQ ID NO: 143, and a heavy chain comprising an amino acid sequence as set forth in SEQ ID NO: 144; ix) a light chain comprising an amino acid sequence as set forth in SEQ ID NO: 145, and a heavy chain comprising an amino acid sequence as set forth in SEQ ID NO: 146; x) a light chain comprising an amino acid sequence as set forth in SEQ ID NO: 147, and a heavy chain comprising an amino acid sequence as set forth in SEQ ID NO: 148; xi) a light chain comprising an amino acid sequence as set forth in SEQ ID NO: 149, and a heavy chain comprising an amino acid sequence as set forth in SEQ ID NO: 150; xii) a light chain comprising an amino acid sequence as set forth in SEQ ID NO: 151, and a heavy chain comprising an amino acid sequence as set forth in SEQ ID NO: 152; xiii) a light chain comprising an amino acid sequence as set forth in SEQ ID NO: 153, and a heavy chain comprising an amino acid sequence as set forth in SEQ ID NO: 154; xiv) a light chain comprising an amino acid sequence as set forth in SEQ ID NO: 155, and a heavy chain comprising an amino acid sequence as set forth in SEQ ID NO: 156; xv) a light chain comprising an amino acid sequence as set forth in SEQ ID NO: 157, and a heavy chain comprising an amino acid sequence as set forth in SEQ ID NO: 158; xvi) a light chain comprising an amino acid sequence as set forth in SEQ ID NO: 159, and a heavy chain comprising an amino acid sequence as set forth in SEQ ID NO: 160; xvii) a light chain comprising an amino acid sequence as set forth in SEQ ID NO: 153, and a heavy chain comprising an amino acid sequence as set forth in SEQ ID NO: 182; xviii) a light chain comprising an amino acid sequence as set forth in SEQ ID NO: 147, and a heavy chain comprising an amino acid sequence as set forth in SEQ ID NO: 183; xix) a light chain comprising an amino acid sequence as set forth in SEQ ID NO: 141, and a heavy chain comprising an amino acid sequence as set forth in SEQ ID NO: 184; xx) a light chain comprising an amino acid sequence as set forth in SEQ ID NO: 153, and a heavy chain comprising an amino acid sequence as set forth in SEQ ID NO: 185; xxi) a light chain comprising an amino acid sequence as set forth in SEQ ID NO: 147, and a heavy chain comprising an amino acid sequence as set forth in SEQ ID NO: 186; xxii) a light chain comprising an amino acid sequence as set forth in SEQ ID NO: 141, and a heavy chain comprising an amino acid sequence as set forth in SEQ ID NO: 187; xxiii) a light chain comprising an amino acid sequence as set forth in SEQ ID NO: 153, and a heavy chain comprising an amino acid sequence as set forth in SEQ ID NO: 188; xxiv) a light chain comprising an amino acid sequence as set forth in SEQ ID NO: 147, and a heavy chain comprising an amino acid sequence as set forth in SEQ ID NO: 189; xxv) a light chain comprising an amino acid sequence as set forth in SEQ ID NO: 141, and a heavy chain comprising an amino acid sequence as set forth in SEQ ID NO: 190; xxvi) a light chain comprising an amino acid sequence as set forth in SEQ ID NO: 153, and a heavy chain comprising an amino acid sequence as set forth in SEQ ID NO: 191; xxvii) a light chain comprising an amino acid sequence as set forth in SEQ ID NO: 147, and a heavy chain comprising an amino acid sequence as set forth in SEQ ID NO: 192; xxviii) a light chain comprising an amino acid sequence as set forth in SEQ ID NO: 141, and a heavy chain comprising an amino acid sequence as set forth in SEQ ID NO: 193; xxix) a light chain comprising an amino acid sequence as set forth in SEQ ID NO: 153, and a heavy chain comprising an amino acid sequence as set forth in SEQ ID NO: 194; xxx) a light chain comprising an amino acid sequence as set forth in SEQ ID NO: 147, and a heavy chain comprising an amino acid sequence as set forth in SEQ ID NO: 195; xxxi) a light chain comprising an amino acid sequence as set forth in SEQ ID NO: 141, and a heavy chain comprising an amino acid sequence as set forth in SEQ ID NO: 196; xxxii) a light chain comprising an amino acid sequence as set forth in SEQ ID NO: 153, and a heavy chain comprising an amino acid sequence as set forth in SEQ ID NO: 197; xxxiii) a light chain comprising an amino acid sequence as set forth in SEQ ID NO: 147, and a heavy chain comprising an amino acid sequence as set forth in SEQ ID NO: 198; xxxiv) a light chain comprising an amino acid sequence as set forth in SEQ ID NO: 141, and a heavy chain comprising an amino acid sequence as set forth in SEQ ID NO: 199; xxxv) a light chain comprising an amino acid sequence as set forth in SEQ ID NO: 153, and a heavy chain comprising an amino acid sequence as set forth in SEQ ID NO: 200; xxxvi) a light chain comprising an amino acid sequence as set forth in SEQ ID NO: 147, and a heavy chain comprising an amino acid sequence as set forth in SEQ ID NO: 201; xxxvii) a light chain comprising an amino acid sequence as set forth in SEQ ID NO: 141, and a heavy chain comprising an amino acid sequence as set forth in SEQ ID NO: 202; xxxviii) a light chain comprising an amino acid sequence as set forth in SEQ ID NO: 153, and a heavy chain comprising an amino acid sequence as set forth in SEQ ID NO: 203; xxxix) a light chain comprising an amino acid sequence as set forth in SEQ ID NO: 147, and a heavy chain comprising an amino acid sequence as set forth in SEQ ID NO: 204; xl) a light chain comprising an amino acid sequence as set forth in SEQ ID NO: 141, and a heavy chain comprising an amino acid sequence as set forth in SEQ ID NO: 205; xli) a light chain comprising an amino acid sequence as set forth in SEQ ID NO: 153, and a heavy chain comprising an amino acid sequence as set forth in SEQ ID NO: 206; xlii) a light chain comprising an amino acid sequence as set forth in SEQ ID NO: 147, and a heavy chain comprising an amino acid sequence as set forth in SEQ ID NO: 207; xliii) a light chain comprising an amino acid sequence as set forth in SEQ ID NO: 141, and a heavy chain comprising an amino acid sequence as set forth in SEQ ID NO: 208; xliv) a light chain comprising an amino acid sequence as set forth in SEQ ID NO: 153, and a heavy chain comprising an amino acid sequence as set forth in SEQ ID NO: 209; xlv) a light chain comprising an amino acid sequence as set forth in SEQ ID NO: 147, and a heavy chain comprising an amino acid sequence as set forth in SEQ ID NO: 210; or xlvi) a light chain comprising an amino acid sequence as set forth in SEQ ID NO: 141, and a heavy chain comprising an amino acid sequence as set forth in SEQ ID NO: 211.

In some embodiments, the antibodies or antigen binding fragments thereof are monoclonal antibodies.

In some embodiments, the antibodies or antigen binding fragments thereof are human antibodies.

In some embodiments, the antibodies or antigen binding fragments thereof are bispecific antibodies.

In some embodiments, the antigen binding fragment thereof is selected from the group consisting of: Fab, Fab′, F(ab′)₂, Fd, Fv, scFv, a single-chain antibody, a minibody, and a diabody.

In some embodiments, the antigen binding fragments thereof is an scFv.

In some aspects, provided herein are antibodies or antigen binding fragments thereof used as a medicament.

In some aspects, provided herein is a pharmaceutical composition comprising antibodies or antigen binding fragments thereof and a pharmaceutically acceptable carrier.

In some aspects, provided herein is a nucleic acid composition comprising one or more nucleic acids encoding antibodies or antigen binding fragments thereof.

In some aspects, provided herein is an expression vector composition comprising one or more expression vectors comprising a nucleic acid composition as provided herein.

In some aspects, provided herein is a host cell comprising a nucleic acid composition or an expression vector composition as provided herein.

In some aspects, provided herein is a method for making anti-IL-13 antibodies or antigen binding fragments thereof comprising culturing the host cell as described herein under conditions where the anti-IL-13 antibodies or antigen binding fragments thereof are expressed and recovering the anti-IL-13 antibodies or binding fragments thereof.

In some aspects, also provided herein are antibodies or antigen binding fragments thereof comprising: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence at least about 90%, at least about 95%, or at least about 99% identical to SEQ ID NO: 97, 99, 101, 103, 105, 107, 109, 111, 113, 115, 117, 119, 121, 123, 125, or 127; and b2) a heavy chain variable region comprising an amino acid sequence at least about 90%, at least about 95%, or at least about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some aspects, also provided herein are antibodies or antigen binding fragments thereof comprising: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence about 90%, about 95%, or about 99% identical to SEQ ID NO: 97, 99, 101, 103, 105, 107, 109, 111, 113, 115, 117, 119, 121, 123, 125, or 127; and b2) a heavy chain variable region comprising an amino acid sequence about 90%, about 95%, or about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some embodiments, the antibodies or antigen binding fragments thereof comprise a LCDR1 comprising a serine (S) at amino acid position 27D and an arginine (R) at amino acid position 30; a LCDR1 comprising a tyrosine (Y) at amino acid position 27D and a histidine (H) at amino acid position 30; a LCDR1 comprising a tyrosine (Y) at amino acid position 27D and an asparagine (N) at amino acid position 30; or a LCDR1 comprises a serine (S) at amino acid position 27D and a tyrosine (Y) at amino acid position 32, wherein the amino acid positions are based on Kabat numbering.

In some aspects, also provided herein are antibodies or antigen binding fragments thereof comprising a light chain variable region and a heavy chain variable region, wherein the light chain variable region is at least about 95%, at least about 96%, at least about 97%, at least about 98%, or at least about 99% identical to SEQ ID NO: 97, 99, 101, and 103; wherein the heavy chain variable region is at least about 95%, at least about 96%, at least about 97%, at least about 98%, or at least about 99% identical to SEQ ID NO: 98, 100, 102, and 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, and wherein the amino acid positions are based on Kabat numbering.

In some aspects, also provided herein are antibodies or antigen binding fragments thereof comprising a light chain variable region and a heavy chain variable region, wherein the light chain variable region is about 95%, about 96%, about 97%, about 98%, or about 99% identical to SEQ ID NO: 97, 99, 101, and 103; wherein the heavy chain variable region is about 95%, about 96%, about 97%, about 98%, or about 99% identical to SEQ ID NO: 98, 100, 102, and 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, and wherein the amino acid positions are based on Kabat numbering.

In some embodiments, the antibodies or antigen binding fragments thereof are monoclonal antibodies.

In some embodiments, the antibodies or antigen binding fragments thereof are human antibodies.

In some embodiments, the antibodies or antigen binding fragments thereof are bispecific antibodies.

In some embodiments, the antigen binding fragment thereof is selected from the group consisting of: Fab, Fab′, F(ab′)₂, Fd, Fv, scFv, a single-chain antibody, a minibody, and a diabody.

In some embodiments, the antigen binding fragment thereof is an scFv.

In some aspects, provided herein are antibodies or antigen binding fragments thereof used as a medicament.

In some aspects, provided herein is a pharmaceutical composition comprising antibodies or an antigen binding fragments thereof and a pharmaceutically acceptable carrier.

In some aspects, provided herein is a nucleic acid composition comprising one or more nucleic acids encoding antibodies or antigen binding fragments thereof.

In some aspects, provided herein is an expression vector composition comprising one or more expression vectors comprising a nucleic acid composition as provided herein.

In some aspects, provided herein is a host cell comprising a nucleic acid composition or an expression vector composition as provided herein.

In some aspects, provided herein is a method for making anti-IL-13 antibodies or antigen binding fragments thereof comprising culturing the host cell as described herein under conditions where the anti-IL-13 antibodies or antigen binding fragments thereof are expressed and recovering the anti-IL-13 antibodies or binding fragments thereof.

In some aspects, described herein is a method of treating a disease or disorder in a subject in need thereof, the method comprising administering to the subject a therapeutically effective dose of antibodies or antigen binding fragments thereof, wherein the antibodies or antigen binding fragments thereof comprise: i) a light chain variable region comprising SEQ ID NO: 1 (LCDR1), SEQ ID NO: 2 (LCDR2), and SEQ ID NO: 3 (LCDR3), and a heavy chain variable region comprising SEQ ID NO: 4 (HCDR1), SEQ ID NO: 5 (HCDR2), and SEQ ID NO: 6 (HCDR3); ii) a light chain variable region comprising SEQ ID NO: 7 (LCDR1), SEQ ID NO: 8 (LCDR2), and SEQ ID NO: 9 (LCDR3), and a heavy chain variable region comprising SEQ ID NO: 10 (HCDR1), SEQ ID NO: 11 (HCDR2), and SEQ ID NO: 12 (HCDR3); iii) a light chain variable region comprising SEQ ID NO: 13 (LCDR1), SEQ ID NO: 14 (LCDR2), and SEQ ID NO: 15 (LCDR3), and a heavy chain variable region comprising SEQ ID NO: 16 (HCDR1), SEQ ID NO: 17 (HCDR2), and SEQ ID NO: 18 (HCDR3); iv) a light chain variable region comprising SEQ ID NO: 19 (LCDR1), SEQ ID NO: 20 (LCDR2), and SEQ ID NO: 21 (LCDR3), and a heavy chain variable region comprising SEQ ID NO: 22 (HCDR1), SEQ ID NO: 23 (HCDR2), and SEQ ID NO: 24 (HCDR3): v) a light chain variable region comprising SEQ ID NO: 25 (LCDR1), SEQ ID NO: 26 (LCDR2), and SEQ ID NO: 27 (LCDR3), and a heavy chain variable region comprising SEQ ID NO: 28 (HCDR1), SEQ ID NO: 29 (HCDR2), and SEQ ID NO: 30 (HCDR3); vi) a light chain variable region comprising SEQ ID NO: 31 (LCDR1), SEQ ID NO: 32 (LCDR2), and SEQ ID NO: 33 (LCDR3), and a heavy chain variable region comprising SEQ ID NO: 34 (HCDR1), SEQ ID NO: 35 (HCDR2), and SEQ ID NO: 36 (HCDR3); vii) a light chain variable region comprising SEQ ID NO: 37 (LCDR1), SEQ ID NO: 38 (LCDR2), and SEQ ID NO: 39 (LCDR3), and a heavy chain variable region comprising SEQ ID NO: 40 (HCDR1), SEQ ID NO: 41 (HCDR2), and SEQ ID NO: 42 (HCDR3): viii) a light chain variable region comprising SEQ ID NO: 43 (LCDR1), SEQ ID NO: 44 (LCDR2), and SEQ ID NO: 45 (LCDR3), and a heavy chain variable region comprising SEQ ID NO: 46 (HCDR1), SEQ ID NO: 47 (HCDR2), and SEQ ID NO: 48 (HCDR3); ix) a light chain variable region comprising SEQ ID NO: 49 (LCDR1), SEQ ID NO: 50 (LCDR2), and SEQ ID NO: 51 (LCDR3), and a heavy chain variable region comprising SEQ ID NO: 52 (HCDR1), SEQ ID NO: 53 (HCDR2), and SEQ ID NO: 54 (HCDR3); x) a light chain variable region comprising SEQ ID NO: 55 (LCDR1), SEQ ID NO: 56 (LCDR2), and SEQ ID NO: 57 (LCDR3), and a heavy chain variable region comprising SEQ ID NO: 58 (HCDR1), SEQ ID NO: 59 (HCDR2), and SEQ ID NO: 60 (HCDR3); xi) a light chain variable region comprising SEQ ID NO: 61 (LCDR1), SEQ ID NO: 62 (LCDR2), and SEQ ID NO: 63 (LCDR3), and a heavy chain variable region comprising SEQ ID NO: 64 (HCDR1), SEQ ID NO: 65 (HCDR2), and SEQ ID NO: 66 (HCDR3); xii) a light chain variable region comprising SEQ ID NO: 67 (LCDR1), SEQ ID NO: 68 (LCDR2), and SEQ ID NO: 69 (LCDR3), and a heavy chain variable region comprising SEQ ID NO: 70 (HCDR1), SEQ ID NO: 71 (HCDR2), and SEQ ID NO: 72 (HCDR3); xiii) a light chain variable region comprising SEQ ID NO: 73 (LCDR1), SEQ ID NO: 74 (LCDR2), and SEQ ID NO: 75 (LCDR3), and a heavy chain variable region comprising SEQ ID NO: 76 (HCDR1), SEQ ID NO: 77 (HCDR2), and SEQ ID NO: 78 (HCDR3); xiv) a light chain variable region comprising SEQ ID NO: 79 (LCDR1), SEQ ID NO: 80 (LCDR2), and SEQ ID NO: 81 (LCDR3), and a heavy chain variable region comprising SEQ ID NO: 82 (HCDR1), SEQ ID NO: 83 (HCDR2), and SEQ ID NO: 84 (HCDR3); xv) a light chain variable region comprising SEQ ID NO: 85 (LCDR1), SEQ ID NO: 86 (LCDR2), and SEQ ID NO: 87 (LCDR3), and a heavy chain variable region comprising SEQ ID NO: 88 (HCDR1), SEQ ID NO: 89 (HCDR2), and SEQ ID NO: 90 (HCDR3); or xvi) a light chain variable region comprising SEQ ID NO: 91 (LCDR1), SEQ ID NO: 92 (LCDR2), and SEQ ID NO: 93 (LCDR3), and a heavy chain variable region comprising SEQ ID NO: 94 (HCDR1), SEQ ID NO: 95 (HCDR2), and SEQ ID NO: 96 (HCDR3).

In some embodiments, the disease or disorder is selected from the group consisting of: asthma (including, but not limited to, mild asthma, mild to moderate asthma, moderate asthma, moderate to severe asthma, and/or severe asthma), allergic asthma, allergen-induced airway obstruction due to asthma and/or allergic asthma, atopic dermatitis (including, but not limited to, mild atopic dermatitis, mild to moderate atopic dermatitis, moderate atopic dermatitis, moderate-to-severe, and/or severe atopic dermatitis), eczema, acute urticaria, idiopathic pulmonary fibrosis (IPF), chronic obstructive pulmonary disease (COPD), chronic obstructive airway disease, emphysema, chronic bronchitis, pulmonary fibrosis, systemic sclerosis, scleroderma, cryptogenic fibrosing alveolitis, usual interstitial pneumonitis, idiopathic interstitial pneumonitis, wound, systemic lupus erythematosus (SLE), rheumatoid arthritis (RA), systemic sclerosis (SS), ulcerative colitis (UC), type 1 diabetes (T1D), sjogren's syndrome (SS), prurigo nodularis, nasal polyposis, recurrent urticaria, aspergillosis, bullous pemphigoid, chronic sinusitis, alopecia areata, hay fever, pemphigus, allergic rhinitis, psoriasis, rosacea, allergic drug reactions, allergic reactions, anaphylaxis, acne, food allergies, inflammatory bowel disease (IBS), Crohn's disease, ulcerative colitis, chronic rhinosinusitis with nasal polyps (CRSwNP), eosinophilic esophagitis, eosinophilic colitis, eosinophilic gastritis, eosinophilic cystitis, eosinophilic fasciitis, eosinophilic pustular folliculitis, eosinophilic granulomatosis, eosinophilic polyangiitis, Churg-Strauss syndrome, biliary cancer, brain cancer, breast cancer, colorectal cancer, genitourinary cancer, head and neck cancer, liver cancer, Hodgkin's lymphoma, esophageal cancer, pancreatic cancer, prostate cancer, renal cancer, Kaposi's sarcoma, sinusitis, or chronic urticaria.

In some embodiments, the light chain variable region comprises an amino acid sequence that is at least about 90%, at least about 95%, or at least about 99% identical to SEQ ID NO: 97, 99, 101, 103, 105, 107, 109, 111, 113, 115, 117, 119, 121, 123, 125, or 127; and the heavy chain variable region comprises an amino acid sequence that is at least about 90%, at least about 95%, or at least about 99% identical to SEQ ID NO: 98, 100, 102, 104, 106, 108, 110, 112, 114, 116, 118, 120, 122, 124, 126, or 128.

In some embodiments, the light chain variable region comprises an amino acid sequence that is about 90%, about 95%, or about 99% identical to SEQ ID NO: 97, 99, 101, 103, 105, 107, 109, 111, 113, 115, 117, 119, 121, 123, 125, or 127; and the heavy chain variable region comprises an amino acid sequence that is about 90%, about 95%, or about 99% identical to SEQ ID NO: 98, 100, 102, 104, 106, 108, 110, 112, 114, 116, 118, 120, 122, 124, 126, or 128.

In some embodiments, the light chain variable region comprises an amino acid sequence as set forth in SEQ ID NO: 97, 99, 101, 103, 105, 107, 109, 111, 113, 115, 117, 119, 121, 123, 125, or 127; and/or the heavy chain variable region comprises an amino acid sequence as set forth in SEQ ID NO: 98, 100, 102, 104, 106, 108, 110, 112, 114, 116, 118, 120, 122, 124, 126, or 128.

In some embodiments, the light chain variable region comprises an amino acid sequence as set forth in SEQ ID NO: 97, and the heavy chain variable region comprises an amino acid sequence as set forth in SEQ ID NO: 98; the light chain variable region comprises an amino acid sequence as set forth in SEQ ID NO: 99, and the heavy chain variable region comprises an amino acid sequence as set forth in SEQ ID NO: 100; the light chain variable region comprises an amino acid sequence as set forth in SEQ ID NO: 101, and the heavy chain variable region comprises an amino acid sequence as set forth in SEQ ID NO: 102; the light chain variable region comprises an amino acid sequence as set forth in SEQ ID NO: 103, and the heavy chain variable region comprises an amino acid sequence as set forth in SEQ ID NO: 104; the light chain variable region comprises an amino acid sequence as set forth in SEQ ID NO: 105, and the heavy chain variable region comprises an amino acid sequence as set forth in SEQ ID NO: 106; the light chain variable region comprises an amino acid sequence as set forth in SEQ ID NO: 107, and the heavy chain variable region comprises an amino acid sequence as set forth in SEQ ID NO: 108; the light chain variable region comprises an amino acid sequence as set forth in SEQ ID NO: 109, and the heavy chain variable region comprises an amino acid sequence as set forth in SEQ ID NO: 110; the light chain variable region comprises an amino acid sequence as set forth in SEQ ID NO: 111, and the heavy chain variable region comprises an amino acid sequence as set forth in SEQ ID NO: 112; the light chain variable region comprises an amino acid sequence as set forth in SEQ ID NO: 113, and the heavy chain variable region comprises an amino acid sequence as set forth in SEQ ID NO: 114; the light chain variable region comprises an amino acid sequence as set forth in SEQ ID NO: 115, and the heavy chain variable region comprises an amino acid sequence as set forth in SEQ ID NO: 116; the light chain variable region comprises an amino acid sequence as set forth in SEQ ID NO: 117, and the heavy chain variable region comprises an amino acid sequence as set forth in SEQ ID NO: 118; the light chain variable region comprises an amino acid sequence as set forth in SEQ ID NO: 119, and the heavy chain variable region comprises an amino acid sequence as set forth in SEQ ID NO: 120; the light chain variable region comprises an amino acid sequence as set forth in SEQ ID NO: 121, and the heavy chain variable region comprises an amino acid sequence as set forth in SEQ ID NO: 122; the light chain variable region comprises an amino acid sequence as set forth in SEQ ID NO: 123, and the heavy chain variable region comprises an amino acid sequence as set forth in SEQ ID NO: 124; the light chain variable region comprises an amino acid sequence as set forth in SEQ ID NO: 125, and the heavy chain variable region comprises an amino acid sequence as set forth in SEQ ID NO: 126; or the light chain variable region comprises an amino acid sequence as set forth in SEQ ID NO: 127, and the heavy chain variable region comprises an amino acid sequence as set forth in SEQ ID NO: 128.

In some embodiments, the antibodies or antigen binding fragments thereof comprise a light chain comprising an amino acid sequence that is at least about 90%, at least about 95%, or at least about 99% identical to SEQ ID NO: 129, 131, 133, 135, 137, 139, 141, 143, 145, 147, 149, 151, 153, 155, 157, or 159; and/or a heavy chain comprising an amino acid sequence that is at least about 90%, at least about 95%, or at least about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof comprise a light chain comprising an amino acid sequence that is about 90%, about 95%, or about 99% identical to SEQ ID NO: 129, 131, 133, 135, 137, 139, 141, 143, 145, 147, 149, 151, 153, 155, 157, or 159; and/or a heavy chain comprising an amino acid sequence that is about 90%, about 95%, or about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the light chain comprises an amino acid sequence as set forth in SEQ ID NO: 129, 131, 133, 135, 137, 139, 141, 143, 145, 147, 149, 151, 153, 155, 157, or 159; and/or the heavy chain comprises an amino acid sequence as set forth in SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the light chain comprises an amino acid sequence as set forth in SEQ ID NO: 129, and the heavy chain comprises an amino acid sequence as set forth in SEQ ID NO: 130; the light chain comprises an amino acid sequence as set forth in SEQ ID NO: 131, and the heavy chain comprises an amino acid sequence as set forth in SEQ ID NO: 132; the light chain comprises an amino acid sequence as set forth in SEQ ID NO: 133, and the heavy chain comprises an amino acid sequence as set forth in SEQ ID NO: 134; the light chain comprises an amino acid sequence as set forth in SEQ ID NO: 135, and the heavy chain comprises an amino acid sequence as set forth in SEQ ID NO: 136; the light chain comprises an amino acid sequence as set forth in SEQ ID NO: 137, and the heavy chain comprises an amino acid sequence as set forth in SEQ ID NO: 138; the light chain comprises an amino acid sequence as set forth in SEQ ID NO: 139, and the heavy chain comprises an amino acid sequence as set forth in SEQ ID NO: 140; the light chain comprises an amino acid sequence as set forth in SEQ ID NO: 141, and the heavy chain comprises an amino acid sequence as set forth in SEQ ID NO: 142; the light chain comprises an amino acid sequence as set forth in SEQ ID NO: 143, and the heavy chain comprises an amino acid sequence as set forth in SEQ ID NO: 144; the light chain comprises an amino acid sequence as set forth in SEQ ID NO: 145, and the heavy chain comprises an amino acid sequence as set forth in SEQ ID NO: 146; the light chain comprises an amino acid sequence as set forth in SEQ ID NO: 147, and the heavy chain comprises an amino acid sequence as set forth in SEQ ID NO: 148; the light chain comprises an amino acid sequence as set forth in SEQ ID NO: 149, and the heavy chain comprises an amino acid sequence as set forth in SEQ ID NO: 150; the light chain comprises an amino acid sequence as set forth in SEQ ID NO: 151, and the heavy chain comprises an amino acid sequence as set forth in SEQ ID NO: 152; the light chain comprises an amino acid sequence as set forth in SEQ ID NO: 153, and the heavy chain comprises an amino acid sequence as set forth in SEQ ID NO: 154; the light chain comprises an amino acid sequence as set forth in SEQ ID NO: 155, and the heavy chain comprises an amino acid sequence as set forth in SEQ ID NO: 156; the light chain comprises an amino acid sequence as set forth in SEQ ID NO: 157, and the heavy chain comprises an amino acid sequence as set forth in SEQ ID NO: 158; the light chain comprises an amino acid sequence as set forth in SEQ ID NO: 159, and the heavy chain comprises an amino acid sequence as set forth in SEQ ID NO: 160; the light chain comprises an amino acid sequence as set forth in SEQ ID NO: 153, and the heavy chain comprises an amino acid sequence as set forth in SEQ ID NO: 182; the light chain comprises an amino acid sequence as set forth in SEQ ID NO: 147, and the heavy chain comprises an amino acid sequence as set forth in SEQ ID NO: 183; the light chain comprises an amino acid sequence as set forth in SEQ ID NO: 141, and the heavy chain comprises an amino acid sequence as set forth in SEQ ID NO: 184; the light chain comprises an amino acid sequence as set forth in SEQ ID NO: 153, and the heavy chain comprises an amino acid sequence as set forth in SEQ ID NO: 185; the light chain comprises an amino acid sequence as set forth in SEQ ID NO: 147, and the heavy chain comprises an amino acid sequence as set forth in SEQ ID NO: 186; the light chain comprises an amino acid sequence as set forth in SEQ ID NO: 141, and the heavy chain comprises an amino acid sequence as set forth in SEQ ID NO: 187; the light chain comprises an amino acid sequence as set forth in SEQ ID NO: 153, and the heavy chain comprises an amino acid sequence as set forth in SEQ ID NO: 188; the light chain comprises an amino acid sequence as set forth in SEQ ID NO: 147, and the heavy chain comprises an amino acid sequence as set forth in SEQ ID NO: 189; the light chain comprises an amino acid sequence as set forth in SEQ ID NO: 141, and the heavy chain comprises an amino acid sequence as set forth in SEQ ID NO: 190; the light chain comprises an amino acid sequence as set forth in SEQ ID NO: 153, and the heavy chain comprises an amino acid sequence as set forth in SEQ ID NO: 191; the light chain comprises an amino acid sequence as set forth in SEQ ID NO: 147, and the heavy chain comprises an amino acid sequence as set forth in SEQ ID NO: 192; the light chain comprises an amino acid sequence as set forth in SEQ ID NO: 141, and the heavy chain comprises an amino acid sequence as set forth in SEQ ID NO: 193; the light chain comprises an amino acid sequence as set forth in SEQ ID NO: 153, and the heavy chain comprises an amino acid sequence as set forth in SEQ ID NO: 194; the light chain comprises an amino acid sequence as set forth in SEQ ID NO: 147, and the heavy chain comprises an amino acid sequence as set forth in SEQ ID NO: 195; the light chain comprises an amino acid sequence as set forth in SEQ ID NO: 141, and the heavy chain comprises an amino acid sequence as set forth in SEQ ID NO: 196; the light chain comprises an amino acid sequence as set forth in SEQ ID NO: 153, and the heavy chain comprises an amino acid sequence as set forth in SEQ ID NO: 197; the light chain comprises an amino acid sequence as set forth in SEQ ID NO: 147, and the heavy chain comprises an amino acid sequence as set forth in SEQ ID NO: 198; the light chain comprises an amino acid sequence as set forth in SEQ ID NO: 141, and the heavy chain comprises an amino acid sequence as set forth in SEQ ID NO: 199; the light chain comprises an amino acid sequence as set forth in SEQ ID NO: 153, and the heavy chain comprises an amino acid sequence as set forth in SEQ ID NO: 200; the light chain comprises an amino acid sequence as set forth in SEQ ID NO: 147, and the heavy chain comprises an amino acid sequence as set forth in SEQ ID NO: 201; the light chain comprises an amino acid sequence as set forth in SEQ ID NO: 141, and the heavy chain comprises an amino acid sequence as set forth in SEQ ID NO: 202; the light chain comprises an amino acid sequence as set forth in SEQ ID NO: 153, and the heavy chain comprises an amino acid sequence as set forth in SEQ ID NO: 203; the light chain comprises an amino acid sequence as set forth in SEQ ID NO: 147, and the heavy chain comprises an amino acid sequence as set forth in SEQ ID NO: 204; the light chain comprises an amino acid sequence as set forth in SEQ ID NO: 141, and the heavy chain comprises an amino acid sequence as set forth in SEQ ID NO: 205; the light chain comprises an amino acid sequence as set forth in SEQ ID NO: 153, and the heavy chain comprises an amino acid sequence as set forth in SEQ ID NO: 206; the light chain comprises an amino acid sequence as set forth in SEQ ID NO: 147, and the heavy chain comprises an amino acid sequence as set forth in SEQ ID NO: 207; the light chain comprises an amino acid sequence as set forth in SEQ ID NO: 141, and the heavy chain comprises an amino acid sequence as set forth in SEQ ID NO: 208; the light chain comprises an amino acid sequence as set forth in SEQ ID NO: 153, and the heavy chain comprises an amino acid sequence as set forth in SEQ ID NO: 209; the light chain comprises an amino acid sequence as set forth in SEQ ID NO: 147, and the heavy chain comprises an amino acid sequence as set forth in SEQ ID NO: 210; or the light chain comprises an amino acid sequence as set forth in SEQ ID NO: 141, and the heavy chain comprises an amino acid sequence as set forth in SEQ ID NO: 211.

In some embodiments, the antibodies or antigen binding fragments thereof are monoclonal antibodies.

In some embodiments, the antibodies or antigen binding fragments thereof are human antibodies.

In some embodiments, the antibodies or antigen binding fragments thereof are bispecific antibodies.

In some embodiments, the antigen binding fragment thereof is selected from the group consisting of Fab, Fab′, F(ab′)₂, Fd, Fv, scFv, a single-chain antibody, a minibody, and a diabody.

In some embodiments, the antigen binding fragment thereof is an scFv.

In some aspects, described herein are antibodies or antigen binding fragments thereof comprising: i) a light chain variable region comprising SEQ ID NO: 1 (LCDR1), SEQ ID NO: 2 (LCDR2), and SEQ ID NO: 3 (LCDR3), and a heavy chain variable region comprising SEQ ID NO: 4 (HCDR1), SEQ ID NO: 5 (HCDR2), and SEQ ID NO: 6 (HCDR3); ii) a light chain variable region comprising SEQ ID NO: 7 (LCDR1), SEQ ID NO: 8 (LCDR2), and SEQ ID NO: 9 (LCDR3), and a heavy chain variable region comprising SEQ ID NO: 10 (HCDR1), SEQ ID NO: 11 (HCDR2), and SEQ ID NO: 12 (HCDR3); iii) a light chain variable region comprising SEQ ID NO: 13 (LCDR1), SEQ ID NO: 14 (LCDR2), and SEQ ID NO: 15 (LCDR3), and a heavy chain variable region comprising SEQ ID NO: 16 (HCDR1), SEQ ID NO: 17 (HCDR2), and SEQ ID NO: 18 (HCDR3); iv) a light chain variable region comprising SEQ ID NO: 19 (LCDR1), SEQ ID NO: 20 (LCDR2), and SEQ ID NO: 21 (LCDR3), and a heavy chain variable region comprising SEQ ID NO: 22 (HCDR1), SEQ ID NO: 23 (HCDR2), and SEQ ID NO: 24 (HCDR3); v) a light chain variable region comprising SEQ ID NO: 25 (LCDR1), SEQ ID NO: 26 (LCDR2), and SEQ ID NO: 27 (LCDR3), and a heavy chain variable region comprising SEQ ID NO: 28 (HCDR1), SEQ ID NO: 29 (HCDR2), and SEQ ID NO: 30 (HCDR3); vi) a light chain variable region comprising SEQ ID NO: 31 (LCDR1), SEQ ID NO: 32 (LCDR2), and SEQ ID NO: 33 (LCDR3), and a heavy chain variable region comprising SEQ ID NO: 34 (HCDR1), SEQ ID NO: 35 (HCDR2), and SEQ ID NO: 36 (HCDR3); vii) a light chain variable region comprising SEQ ID NO: 37 (LCDR1), SEQ ID NO: 38 (LCDR2), and SEQ ID NO: 39 (LCDR3), and a heavy chain variable region comprising SEQ ID NO: 40 (HCDR1), SEQ ID NO: 41 (HCDR2), and SEQ ID NO: 42 (HCDR3); viii) a light chain variable region comprising SEQ ID NO: 43 (LCDR1), SEQ ID NO: 44 (LCDR2), and SEQ ID NO: 45 (LCDR3), and a heavy chain variable region comprising SEQ ID NO: 46 (HCDR1), SEQ ID NO: 47 (HCDR2), and SEQ ID NO: 48 (HCDR3); ix) a light chain variable region comprising SEQ ID NO: 49 (LCDR1), SEQ ID NO: 50 (LCDR2), and SEQ ID NO: 51 (LCDR3), and a heavy chain variable region comprising SEQ ID NO: 52 (HCDR1), SEQ ID NO: 53 (HCDR2), and SEQ ID NO: 54 (HCDR3); x) a light chain variable region comprising SEQ ID NO: 55 (LCDR1), SEQ ID NO: 56 (LCDR2), and SEQ ID NO: 57 (LCDR3), and a heavy chain variable region comprising SEQ ID NO: 58 (HCDR1), SEQ ID NO: 59 (HCDR2), and SEQ ID NO: 60 (HCDR3); xi) a light chain variable region comprising SEQ ID NO: 61 (LCDR1), SEQ ID NO: 62 (LCDR2), and SEQ ID NO: 63 (LCDR3), and a heavy chain variable region comprising SEQ ID NO: 64 (HCDR1), SEQ ID NO: 65 (HCDR2), and SEQ ID NO: 66 (HCDR3); xii) a light chain variable region comprising SEQ ID NO: 67 (LCDR1), SEQ ID NO: 68 (LCDR2), and SEQ ID NO: 69 (LCDR3), and a heavy chain variable region comprising SEQ ID NO: 70 (HCDR1), SEQ ID NO: 71 (HCDR2), and SEQ ID NO: 72 (HCDR3); xiii) a light chain variable region comprising SEQ ID NO: 73 (LCDR1), SEQ ID NO: 74 (LCDR2), and SEQ ID NO: 75 (LCDR3), and a heavy chain variable region comprising SEQ ID NO: 76 (HCDR1), SEQ ID NO: 77 (HCDR2), and SEQ ID NO: 78 (HCDR3); xiv) a light chain variable region comprising SEQ ID NO: 79 (LCDR1), SEQ ID NO: 80 (LCDR2), and SEQ ID NO: 81 (LCDR3), and a heavy chain variable region comprising SEQ ID NO: 82 (HCDR1), SEQ ID NO: 83 (HCDR2), and SEQ ID NO: 84 (HCDR3); xv) a light chain variable region comprising SEQ ID NO: 85 (LCDR1), SEQ ID NO: 86 (LCDR2), and SEQ ID NO: 87 (LCDR3), and a heavy chain variable region comprising SEQ ID NO: 88 (HCDR1), SEQ ID NO: 89 (HCDR2), and SEQ ID NO: 90 (HCDR3); or xvi) a light chain variable region comprising SEQ ID NO: 91 (LCDR1), SEQ ID NO: 92 (LCDR2), and SEQ ID NO: 93 (LCDR3), and a heavy chain variable region comprising SEQ ID NO: 94 (HCDR1), SEQ ID NO: 95 (HCDR2), and SEQ ID NO: 96 (HCDR3).

In some embodiments, the antibodies or antigen binding fragments thereof that bind IL-13 comprise a light chain variable region comprising an amino acid sequence that is at least about 90%, at least about 95%, at least about 96%, at least about 97%, at least about 98%, or at least about 99% identical to SEQ ID NO: 97, 99, 101, 103, 105, 107, 109, 111, 113, 115, 117, 119, 121, 123, 125, or 127; and/or a heavy chain variable region comprising an amino acid sequence that is at least about 90%, at least about 95%, at least about 96%, at least about 97%, at least about 98%, or at least about 99% identical to SEQ ID NO: 98, 100, 102, 104, 106, 108, 110, 112, 114, 116, 118, 120, 122, 124, 126, or 128.

In some embodiments, the antibodies or antigen binding fragments thereof that bind IL-13 comprise a light chain variable region comprising an amino acid sequence that is about 90%, about 95%, about 96%, about 97%, about 98%, or about 99% identical to SEQ ID NO: 97, 99, 101, 103, 105, 107, 109, 111, 113, 115, 117, 119, 121, 123, 125, or 127; and/or a heavy chain variable region comprising an amino acid sequence that is about 90%, about 95%, about 96%, about 97%, about 98%, or about 99% identical to SEQ ID NO: 98, 100, 102, 104, 106, 108, 110, 112, 114, 116, 118, 120, 122, 124, 126, or 128.

In some embodiments, the antibodies or antigen binding fragments thereof that bind IL-13 comprise a light chain variable region comprising an amino acid sequence as set forth in SEQ ID NO: 97, 99, 101, 103, 105, 107, 109, 111, 113, 115, 117, 119, 121, 123, 125, or 127; and/or a heavy chain variable region comprising an amino acid sequence as set forth in SEQ ID NO: 98, 100, 102, 104, 106, 108, 110, 112, 114, 116, 118, 120, 122, 124, 126, or 128.

In some embodiments, the antibodies or antigen binding fragments thereof that bind IL-13 comprise: i) a light chain variable region comprising an amino acid sequence as set forth in in SEQ ID NO: 97, and a heavy chain variable region comprising an amino acid sequence as set forth in in SEQ ID NO: 98; ii) a light chain variable region comprising an amino acid sequence as set forth in in SEQ ID NO: 99, and a heavy chain variable region comprising an amino acid sequence as set forth in in SEQ ID NO: 100; iii) a light chain variable region comprising an amino acid sequence as set forth in in SEQ ID NO: 101, and a heavy chain variable region comprising an amino acid sequence as set forth in in SEQ ID NO: 102; vi) a light chain variable region comprising an amino acid sequence as set forth in in SEQ ID NO: 103, and a heavy chain variable region comprising an amino acid sequence as set forth in in SEQ ID NO: 104; v) a light chain variable region comprising an amino acid sequence as set forth in in SEQ ID NO: 105, and a heavy chain variable region comprising an amino acid sequence as set forth in in SEQ ID NO: 106; vi) a light chain variable region comprising an amino acid sequence as set forth in in SEQ ID NO: 107, and a heavy chain variable region comprising an amino acid sequence as set forth in in SEQ ID NO: 108; vii) a light chain variable region comprising an amino acid sequence as set forth in in SEQ ID NO: 109, and a heavy chain variable region comprising an amino acid sequence as set forth in in SEQ ID NO: 110; vii) a light chain variable region comprising an amino acid sequence as set forth in in SEQ ID NO: 111, and a heavy chain variable region comprising an amino acid sequence as set forth in in SEQ ID NO: 112; ix) a light chain variable region comprising an amino acid sequence as set forth in in SEQ ID NO: 113, and a heavy chain variable region comprising an amino acid sequence as set forth in in SEQ ID NO: 114; x) a light chain variable region comprising an amino acid sequence as set forth in in SEQ ID NO: 115, and a heavy chain variable region comprising an amino acid sequence as set forth in in SEQ ID NO: 116; xi) a light chain variable region comprising an amino acid sequence as set forth in in SEQ ID NO: 117, and a heavy chain variable region comprising an amino acid sequence as set forth in in SEQ ID NO: 118; xii) a light chain variable region comprising an amino acid sequence as set forth in in SEQ ID NO: 119, and a heavy chain variable region comprising an amino acid sequence as set forth in in SEQ ID NO: 120; xiii) a light chain variable region comprising an amino acid sequence as set forth in in SEQ ID NO: 121, and a heavy chain variable region comprising an amino acid sequence as set forth in in SEQ ID NO: 122; xiv) a light chain variable region comprising an amino acid sequence as set forth in in SEQ ID NO: 123, and a heavy chain variable region comprising an amino acid sequence as set forth in in SEQ ID NO: 124; xv) a light chain variable region comprising an amino acid sequence as set forth in in SEQ ID NO: 125, and a heavy chain variable region comprising an amino acid sequence as set forth in in SEQ ID NO: 126; or xvi) a light chain variable region comprising an amino acid sequence as set forth in in SEQ ID NO: 127, and a heavy chain variable region comprising an amino acid sequence as set forth in in SEQ ID NO: 128.

In some embodiments, the antibodies or antigen binding fragments thereof that bind IL-13 comprise a light chain comprising an amino acid sequence that is at least about 90%, at least about 95%, at least about 96%, at least about 97%, at least about 98%, or at least about 99% identical to SEQ ID NO: 129, 131, 133, 135, 137, 139, 141, 143, 145, 147, 149, 151, 153, 155, 157, or 159; and/or a heavy chain comprising an amino acid sequence that is at least about 90%, at least about 95%, at least about 96%, at least about 97%, at least about 98%, or at least about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof that bind IL-13 comprise a light chain comprising an amino acid sequence that is about 90%, about 95%, about 96%, about 97%, about 98%, or about 99% identical to SEQ ID NO: 129, 131, 133, 135, 137, 139, 141, 143, 145, 147, 149, 151, 153, 155, 157, or 159; and/or a heavy chain comprising an amino acid sequence that is about 90%, about 95%, about 96%, about 97%, about 98%, or about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof that bind IL-13 comprises a light chain comprising an amino acid sequence as set forth in SEQ ID NO: 129, 131, 133, 135, 137, 139, 141, 143, 145, 147, 149, 151, 153, 155, 157, or 159; and/or a heavy chain comprising an amino acid sequence as set forth in SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof that bind IL-13 comprise: i) a light chain comprising an amino acid sequence as set forth in SEQ ID NO: 129, and a heavy chain comprising an amino acid sequence as set forth in SEQ ID NO: 130; ii) a light chain comprising an amino acid sequence as set forth in SEQ ID NO: 131, and a heavy chain comprising an amino acid sequence as set forth in SEQ ID NO: 132; iii) a light chain comprising an amino acid sequence as set forth in SEQ ID NO: 133, and a heavy chain comprising an amino acid sequence as set forth in SEQ ID NO: 134; iv) a light chain comprising an amino acid sequence as set forth in SEQ ID NO: 135, and a heavy chain comprising an amino acid sequence as set forth in SEQ ID NO: 136; v) a light chain comprising an amino acid sequence as set forth in SEQ ID NO: 137, and a heavy chain comprising an amino acid sequence as set forth in SEQ ID NO: 138; vi) a light chain comprising an amino acid sequence as set forth in SEQ ID NO: 139, and a heavy chain comprising an amino acid sequence as set forth in SEQ ID NO: 140; vii) a light chain comprising an amino acid sequence as set forth in SEQ ID NO: 141, and a heavy chain comprising an amino acid sequence as set forth in SEQ ID NO: 142; viii) a light chain comprising an amino acid sequence as set forth in SEQ ID NO: 143, and a heavy chain comprising an amino acid sequence as set forth in SEQ ID NO: 144; ix) a light chain comprising an amino acid sequence as set forth in SEQ ID NO: 145, and a heavy chain comprising an amino acid sequence as set forth in SEQ ID NO: 146; x) a light chain comprising an amino acid sequence as set forth in SEQ ID NO: 147, and a heavy chain comprising an amino acid sequence as set forth in SEQ ID NO: 148; xi) a light chain comprising an amino acid sequence as set forth in SEQ ID NO: 149, and a heavy chain comprising an amino acid sequence as set forth in SEQ ID NO: 150; xii) a light chain comprising an amino acid sequence as set forth in SEQ ID NO: 151, and a heavy chain comprising an amino acid sequence as set forth in SEQ ID NO: 152; xiii) a light chain comprising an amino acid sequence as set forth in SEQ ID NO: 153, and a heavy chain comprising an amino acid sequence as set forth in SEQ ID NO: 154; xiv) a light chain comprising an amino acid sequence as set forth in SEQ ID NO: 155, and a heavy chain comprising an amino acid sequence as set forth in SEQ ID NO: 156; xv) a light chain comprising an amino acid sequence as set forth in SEQ ID NO: 157, and a heavy chain comprising an amino acid sequence as set forth in SEQ ID NO: 158; xvi) a light chain comprising an amino acid sequence as set forth in SEQ ID NO: 159, and a heavy chain comprising an amino acid sequence as set forth in SEQ ID NO: 160; xvii) a light chain comprising an amino acid sequence as set forth in SEQ ID NO: 153, and a heavy chain comprising an amino acid sequence as set forth in SEQ ID NO: 182; xviii) a light chain comprising an amino acid sequence as set forth in SEQ ID NO: 147, and a heavy chain comprising an amino acid sequence as set forth in SEQ ID NO: 183; xix) a light chain comprising an amino acid sequence as set forth in SEQ ID NO: 141, and a heavy chain comprising an amino acid sequence as set forth in SEQ ID NO: 184; xx) a light chain comprising an amino acid sequence as set forth in SEQ ID NO: 153, and a heavy chain comprising an amino acid sequence as set forth in SEQ ID NO: 185; xxi) a light chain comprising an amino acid sequence as set forth in SEQ ID NO: 147, and a heavy chain comprising an amino acid sequence as set forth in SEQ ID NO: 186; xxii) a light chain comprising an amino acid sequence as set forth in SEQ ID NO: 141, and a heavy chain comprising an amino acid sequence as set forth in SEQ ID NO: 187; xxiii) a light chain comprising an amino acid sequence as set forth in SEQ ID NO: 153, and a heavy chain comprising an amino acid sequence as set forth in SEQ ID NO: 188; xxiv) a light chain comprising an amino acid sequence as set forth in SEQ ID NO: 147, and a heavy chain comprising an amino acid sequence as set forth in SEQ ID NO: 189; xxv) a light chain comprising an amino acid sequence as set forth in SEQ ID NO: 141, and a heavy chain comprising an amino acid sequence as set forth in SEQ ID NO: 190; xxvi) a light chain comprising an amino acid sequence as set forth in SEQ ID NO: 153, and a heavy chain comprising an amino acid sequence as set forth in SEQ ID NO: 191; xxvii) a light chain comprising an amino acid sequence as set forth in SEQ ID NO: 147, and a heavy chain comprising an amino acid sequence as set forth in SEQ ID NO: 192; xxviii) a light chain comprising an amino acid sequence as set forth in SEQ ID NO: 141, and a heavy chain comprising an amino acid sequence as set forth in SEQ ID NO: 193; xxix) a light chain comprising an amino acid sequence as set forth in SEQ ID NO: 153, and a heavy chain comprising an amino acid sequence as set forth in SEQ ID NO: 194; xxx) a light chain comprising an amino acid sequence as set forth in SEQ ID NO: 147, and a heavy chain comprising an amino acid sequence as set forth in SEQ ID NO: 195; xxxi) a light chain comprising an amino acid sequence as set forth in SEQ ID NO: 141, and a heavy chain comprising an amino acid sequence as set forth in SEQ ID NO: 196; xxxii) a light chain comprising an amino acid sequence as set forth in SEQ ID NO: 153, and a heavy chain comprising an amino acid sequence as set forth in SEQ ID NO: 197; xxxiii) a light chain comprising an amino acid sequence as set forth in SEQ ID NO: 147, and a heavy chain comprising an amino acid sequence as set forth in SEQ ID NO: 198; xxxiv) a light chain comprising an amino acid sequence as set forth in SEQ ID NO: 141, and a heavy chain comprising an amino acid sequence as set forth in SEQ ID NO: 199; xxxv) a light chain comprising an amino acid sequence as set forth in SEQ ID NO: 153, and a heavy chain comprising an amino acid sequence as set forth in SEQ ID NO: 200; xxxvi) a light chain comprising an amino acid sequence as set forth in SEQ ID NO: 147, and a heavy chain comprising an amino acid sequence as set forth in SEQ ID NO: 201; xxxvii) a light chain comprising an amino acid sequence as set forth in SEQ ID NO: 141, and a heavy chain comprising an amino acid sequence as set forth in SEQ ID NO: 202; xxxviii) a light chain comprising an amino acid sequence as set forth in SEQ ID NO: 153, and a heavy chain comprising an amino acid sequence as set forth in SEQ ID NO: 203; xxxix) a light chain comprising an amino acid sequence as set forth in SEQ ID NO: 147, and a heavy chain comprising an amino acid sequence as set forth in SEQ ID NO: 204; xl) a light chain comprising an amino acid sequence as set forth in SEQ ID NO: 141, and a heavy chain comprising an amino acid sequence as set forth in SEQ ID NO: 205; xli) a light chain comprising an amino acid sequence as set forth in SEQ ID NO: 153, and a heavy chain comprising an amino acid sequence as set forth in SEQ ID NO: 206; xlii) a light chain comprising an amino acid sequence as set forth in SEQ ID NO: 147, and a heavy chain comprising an amino acid sequence as set forth in SEQ ID NO: 207; xliii) a light chain comprising an amino acid sequence as set forth in SEQ ID NO: 141, and a heavy chain comprising an amino acid sequence as set forth in SEQ ID NO: 208; xliv) a light chain comprising an amino acid sequence as set forth in SEQ ID NO: 153, and a heavy chain comprising an amino acid sequence as set forth in SEQ ID NO: 209; xlv) a light chain comprising an amino acid sequence as set forth in SEQ ID NO: 147, and a heavy chain comprising an amino acid sequence as set forth in SEQ ID NO: 210; or xlvi) a light chain comprising an amino acid sequence as set forth in SEQ ID NO: 141, and a heavy chain comprising an amino acid sequence as set forth in SEQ ID NO: 211.

In some embodiments, the antibodies or antigen binding fragments thereof that bind IL-13 are monoclonal antibodies.

In some embodiments, the antibodies or antigen binding fragments thereof that bind IL-13 are human antibodies.

In some embodiments, the antibodies or antigen binding fragments thereof that bind IL-13 are bispecific antibodies.

In some embodiments, the antigen binding fragment thereof is selected from the group consisting of: Fab, Fab′, F(ab′)₂, Fd, Fv, scFv, a single-chain antibody, a minibody, and a diabody.

In some embodiments, the antigen binding fragments thereof is an scFv.

In some aspects, provided herein are antibodies or antigen binding fragments thereof that bind IL-13 used as a medicament.

In some aspects, provided herein is a pharmaceutical composition comprising antibodies or antigen binding fragments thereof that bind IL-13 and a pharmaceutically acceptable carrier.

In some aspects, provided herein is a nucleic acid composition comprising one or more nucleic acids encoding antibodies or antigen binding fragments thereof that bind IL-13.

In some aspects, provided herein is an expression vector composition comprising one or more expression vectors comprising a nucleic acid composition as provided herein.

In some aspects, provided herein is a host cell comprising a nucleic acid composition or an expression vector composition as provided herein.

In some aspects, provided herein is a method for making anti-IL-13 antibodies or antigen binding fragments thereof comprising culturing the host cell as described herein under conditions where the anti-IL-13 antibodies or antigen binding fragments thereof are expressed and recovering the anti-IL-13 antibodies or binding fragments thereof.

In some aspects, also provided herein are antibodies or antigen binding fragments thereof that bind IL-13 comprising: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence at least about 90%, at least about 95%, or at least about 99% identical to SEQ ID NO: 97, 99, 101, 103, 105, 107, 109, 111, 113, 115, 117, 119, 121, 123, 125, or 127; and b2) a heavy chain variable region comprising an amino acid sequence at least about 90%, at least about 95%, or at least about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some aspects, also provided herein are antibodies or antigen binding fragments thereof that bind IL-13 comprising: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence about 90%, about 95%, or about 99% identical to SEQ ID NO: 97, 99, 101, 103, 105, 107, 109, 111, 113, 115, 117, 119, 121, 123, 125, or 127; and b2) a heavy chain variable region comprising an amino acid sequence about 90%, about 95%, or about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some embodiments, the antibodies or antigen binding fragments thereof that bind IL-13 comprise a LCDR1 comprising a serine (S) at amino acid position 27D and an arginine (R) at amino acid position 30; a LCDR1 comprising a tyrosine (Y) at amino acid position 27D and a histidine (H) at amino acid position 30; a LCDR1 comprising a tyrosine (Y) at amino acid position 27D and an asparagine (N) at amino acid position 30; or a LCDR1 comprises a serine (S) at amino acid position 27D and a tyrosine (Y) at amino acid position 32, wherein the amino acid positions are based on Kabat numbering.

In some aspects, also provided herein are antibodies or antigen binding fragments thereof that bind IL-13 comprising a light chain variable region and a heavy chain variable region, wherein the light chain variable region is at least about 95%, at least about 96%, at least about 97%, at least about 98%, or at least about 99% identical to SEQ ID NO: 97, 99, 101, and 103; wherein the heavy chain variable region is at least about 95%, at least about 96%, at least about 97%, at least about 98%, or at least about 99% identical to SEQ ID NO: 98, 100, 102, and 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, and wherein the amino acid positions are based on Kabat numbering.

In some aspects, also provided herein are antibodies or antigen binding fragments thereof that bind IL-13 comprising a light chain variable region and a heavy chain variable region, wherein the light chain variable region is about 95%, about 96%, about 97%, about 98%, or about 99% identical to SEQ ID NO: 97, 99, 101, and 103; wherein the heavy chain variable region is about 95%, about 96%, about 97%, about 98%, or about 99% identical to SEQ ID NO: 98, 100, 102, and 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, and wherein the amino acid positions are based on Kabat numbering.

In some embodiments, the antibodies or antigen binding fragments thereof that bind IL-13 are monoclonal antibodies.

In some embodiments, the antibodies or antigen binding fragments thereof that bind IL-13 are human antibodies.

In some embodiments, the antibodies or antigen binding fragments thereof that bind IL-13 are bispecific antibodies.

In some embodiments, the antigen binding fragment thereof is selected from the group consisting of: Fab, Fab′, F(ab′)₂, Fd, Fv, scFv, a single-chain antibody, a minibody, and a diabody.

In some embodiments, the antigen binding fragment thereof is an scFv.

In some aspects, provided herein are antibodies or antigen binding fragments thereof that bind IL-13 used as a medicament.

In some aspects, provided herein is a pharmaceutical composition comprising antibodies or an antigen binding fragments thereof and a pharmaceutically acceptable carrier.

In some aspects, provided herein is a nucleic acid composition comprising one or more nucleic acids encoding antibodies or antigen binding fragments thereof that bind IL-13.

In some aspects, provided herein is an expression vector composition comprising one or more expression vectors comprising a nucleic acid composition as provided herein.

In some aspects, provided herein is a host cell comprising a nucleic acid composition or an expression vector composition as provided herein.

In some aspects, provided herein is a method for making anti-IL-13 antibodies or antigen binding fragments thereof comprising culturing the host cell as described herein under conditions where the anti-IL-13 antibodies or antigen binding fragments thereof are expressed and recovering the anti-IL-13 antibodies or binding fragments thereof.

In some aspects, described herein is a method of treating a disease or disorder in a subject in need thereof, the method comprising administering to the subject a therapeutically effective dose of antibodies or antigen binding fragments thereof that bind IL-13, wherein the antibodies or antigen binding fragments thereof that bind IL-13 comprise: i) a light chain variable region comprising SEQ ID NO: 1 (LCDR1), SEQ ID NO: 2 (LCDR2), and SEQ ID NO: 3 (LCDR3), and a heavy chain variable region comprising SEQ ID NO: 4 (HCDR1), SEQ ID NO: 5 (HCDR2), and SEQ ID NO: 6 (HCDR3): ii) a light chain variable region comprising SEQ ID NO: 7 (LCDR1). SEQ ID NO: 8 (LCDR2), and SEQ ID NO: 9 (LCDR3), and a heavy chain variable region comprising SEQ ID NO: 10 (HCDR1), SEQ ID NO: 11 (HCDR2), and SEQ ID NO: 12 (HCDR3); iii) a light chain variable region comprising SEQ ID NO: 13 (LCDR1), SEQ ID NO: 14 (LCDR2), and SEQ ID NO: 15 (LCDR3), and a heavy chain variable region comprising SEQ ID NO: 16 (HCDR1), SEQ ID NO: 17 (HCDR2), and SEQ ID NO: 18 (HCDR3); iv) a light chain variable region comprising SEQ ID NO: 19 (LCDR1), SEQ ID NO: 20 (LCDR2), and SEQ ID NO: 21 (LCDR3), and a heavy chain variable region comprising SEQ ID NO: 22 (HCDR1), SEQ ID NO: 23 (HCDR2), and SEQ ID NO: 24 (HCDR3); v) a light chain variable region comprising SEQ ID NO: 25 (LCDR1), SEQ ID NO: 26 (LCDR2), and SEQ ID NO: 27 (LCDR3), and a heavy chain variable region comprising SEQ ID NO: 28 (HCDR1), SEQ ID NO: 29 (HCDR2), and SEQ ID NO: 30 (HCDR3); vi) a light chain variable region comprising SEQ ID NO: 31 (LCDR1), SEQ ID NO: 32 (LCDR2), and SEQ ID NO: 33 (LCDR3), and a heavy chain variable region comprising SEQ ID NO: 34 (HCDR1), SEQ ID NO: 35 (HCDR2), and SEQ ID NO: 36 (HCDR3); vii) a light chain variable region comprising SEQ ID NO: 37 (LCDR1), SEQ ID NO: 38 (LCDR2), and SEQ ID NO: 39 (LCDR3), and a heavy chain variable region comprising SEQ ID NO: 40 (HCDR1), SEQ ID NO: 41 (HCDR2), and SEQ ID NO: 42 (HCDR3); viii) a light chain variable region comprising SEQ ID NO: 43 (LCDR1), SEQ ID NO: 44 (LCDR2), and SEQ ID NO: 45 (LCDR3), and a heavy chain variable region comprising SEQ ID NO: 46 (HCDR1), SEQ ID NO: 47 (HCDR2), and SEQ ID NO: 48 (HCDR3); ix) a light chain variable region comprising SEQ ID NO: 49 (LCDR1), SEQ ID NO: 50 (LCDR2), and SEQ ID NO: 51 (LCDR3), and a heavy chain variable region comprising SEQ ID NO: 52 (HCDR1), SEQ ID NO: 53 (HCDR2), and SEQ ID NO: 54 (HCDR3); x) a light chain variable region comprising SEQ ID NO: 55 (LCDR1), SEQ ID NO: 56 (LCDR2), and SEQ ID NO: 57 (LCDR3), and a heavy chain variable region comprising SEQ ID NO: 58 (HCDR1), SEQ ID NO: 59 (HCDR2), and SEQ ID NO: 60 (HCDR3); xi) a light chain variable region comprising SEQ ID NO: 61 (LCDR1), SEQ ID NO: 62 (LCDR2), and SEQ ID NO: 63 (LCDR3), and a heavy chain variable region comprising SEQ ID NO: 64 (HCDR1), SEQ ID NO: 65 (HCDR2), and SEQ ID NO: 66 (HCDR3): xii) a light chain variable region comprising SEQ ID NO: 67 (LCDR1), SEQ ID NO: 68 (LCDR2), and SEQ ID NO: 69 (LCDR3), and a heavy chain variable region comprising SEQ ID NO: 70 (HCDR1), SEQ ID NO: 71 (HCDR2), and SEQ ID NO: 72 (HCDR3); xiii) a light chain variable region comprising SEQ ID NO: 73 (LCDR1), SEQ ID NO: 74 (LCDR2), and SEQ ID NO: 75 (LCDR3), and a heavy chain variable region comprising SEQ ID NO: 76 (HCDR1), SEQ ID NO: 77 (HCDR2), and SEQ ID NO: 78 (HCDR3); xiv) a light chain variable region comprising SEQ ID NO: 79 (LCDR1), SEQ ID NO: 80 (LCDR2), and SEQ ID NO: 81 (LCDR3), and a heavy chain variable region comprising SEQ ID NO: 82 (HCDR1), SEQ ID NO: 83 (HCDR2), and SEQ ID NO: 84 (HCDR3): xv) a light chain variable region comprising SEQ ID NO: 85 (LCDR1), SEQ ID NO: 86 (LCDR2), and SEQ ID NO: 87 (LCDR3), and a heavy chain variable region comprising SEQ ID NO: 88 (HCDR1), SEQ ID NO: 89 (HCDR2), and SEQ ID NO: 90 (HCDR3); or xvi) a light chain variable region comprising SEQ ID NO: 91 (LCDR1), SEQ ID NO: 92 (LCDR2), and SEQ ID NO: 93 (LCDR3), and a heavy chain variable region comprising SEQ ID NO: 94 (HCDR1), SEQ ID NO: 95 (HCDR2), and SEQ ID NO: 96 (HCDR3).

In some embodiments, the disease or disorder is selected from the group consisting of: asthma (including, but not limited to, mild asthma, mild to moderate asthma, moderate asthma, moderate to severe asthma, and/or severe asthma), allergic asthma, allergen-induced airway obstruction due to asthma and/or allergic asthma, atopic dermatitis (including, but not limited to, mild atopic dermatitis, mild to moderate atopic dermatitis, moderate atopic dermatitis, moderate-to-severe, and/or severe atopic dermatitis), eczema, acute urticaria, idiopathic pulmonary fibrosis (IPF), chronic obstructive pulmonary disease (COPD), chronic obstructive airway disease, emphysema, chronic bronchitis, pulmonary fibrosis, systemic sclerosis, scleroderma, cryptogenic fibrosing alveolitis, usual interstitial pneumonitis, idiopathic interstitial pneumonitis, wound, systemic lupus erythematosus (SLE), rheumatoid arthritis (RA), systemic sclerosis (SS), ulcerative colitis (UC), type 1 diabetes (T1D), sjogren's syndrome (SS), prurigo nodularis, nasal polyposis, recurrent urticaria, aspergillosis, bullous pemphigoid, chronic sinusitis, alopecia areata, hay fever, pemphigus, allergic rhinitis, psoriasis, rosacea, allergic drug reactions, allergic reactions, anaphylaxis, acne, food allergies, inflammatory bowel disease (IBS), Crohn's disease, ulcerative colitis, chronic rhinosinusitis with nasal polyps (CRSwNP), eosinophilic esophagitis, eosinophilic colitis, eosinophilic gastritis, eosinophilic cystitis, eosinophilic fasciitis, eosinophilic pustular folliculitis, eosinophilic granulomatosis, eosinophilic polyangiitis, Churg-Strauss syndrome, biliary cancer, brain cancer, breast cancer, colorectal cancer, genitourinary cancer, head and neck cancer, liver cancer, Hodgkin's lymphoma, esophageal cancer, pancreatic cancer, prostate cancer, renal cancer, Kaposi's sarcoma, sinusitis, or chronic urticaria.

In some embodiments, the light chain variable region comprises an amino acid sequence that is at least about 90%, at least about 95%, or at least about 99% identical to SEQ ID NO: 97, 99, 101, 103, 105, 107, 109, 111, 113, 115, 117, 119, 121, 123, 125, or 127; and the heavy chain variable region comprises an amino acid sequence that is at least about 90%, at least about 95%, or at least about 99% identical to SEQ ID NO: 98, 100, 102, 104, 106, 108, 110, 112, 114, 116, 118, 120, 122, 124, 126, or 128.

In some embodiments, the light chain variable region comprises an amino acid sequence that is about 90%, about 95%, or about 99% identical to SEQ ID NO: 97, 99, 101, 103, 105, 107, 109, 111, 113, 115, 117, 119, 121, 123, 125, or 127; and the heavy chain variable region comprises an amino acid sequence that is about 90%, about 95%, or about 99% identical to SEQ ID NO: 98, 100, 102, 104, 106, 108, 110, 112, 114, 116, 118, 120, 122, 124, 126, or 128.

In some embodiments, the light chain variable region comprises an amino acid sequence as set forth in SEQ ID NO: 97, 99, 101, 103, 105, 107, 109, 111, 113, 115, 117, 119, 121, 123, 125, or 127; and/or the heavy chain variable region comprises an amino acid sequence as set forth in SEQ ID NO: 98, 100, 102, 104, 106, 108, 110, 112, 114, 116, 118, 120, 122, 124, 126, or 128.

In some embodiments, the light chain variable region comprises an amino acid sequence as set forth in SEQ ID NO: 97, and the heavy chain variable region comprises an amino acid sequence as set forth in SEQ ID NO: 98; the light chain variable region comprises an amino acid sequence as set forth in SEQ ID NO: 99, and the heavy chain variable region comprises an amino acid sequence as set forth in SEQ ID NO: 100; the light chain variable region comprises an amino acid sequence as set forth in SEQ ID NO: 101, and the heavy chain variable region comprises an amino acid sequence as set forth in SEQ ID NO: 102; the light chain variable region comprises an amino acid sequence as set forth in SEQ ID NO: 103, and the heavy chain variable region comprises an amino acid sequence as set forth in SEQ ID NO: 104; the light chain variable region comprises an amino acid sequence as set forth in SEQ ID NO: 105, and the heavy chain variable region comprises an amino acid sequence as set forth in SEQ ID NO: 106; the light chain variable region comprises an amino acid sequence as set forth in SEQ ID NO: 107, and the heavy chain variable region comprises an amino acid sequence as set forth in SEQ ID NO: 108; the light chain variable region comprises an amino acid sequence as set forth in SEQ ID NO: 109, and the heavy chain variable region comprises an amino acid sequence as set forth in SEQ ID NO: 110; the light chain variable region comprises an amino acid sequence as set forth in SEQ ID NO: 111, and the heavy chain variable region comprises an amino acid sequence as set forth in SEQ ID NO: 112; the light chain variable region comprises an amino acid sequence as set forth in SEQ ID NO: 113, and the heavy chain variable region comprises an amino acid sequence as set forth in SEQ ID NO: 114; the light chain variable region comprises an amino acid sequence as set forth in SEQ ID NO: 115, and the heavy chain variable region comprises an amino acid sequence as set forth in SEQ ID NO: 116; the light chain variable region comprises an amino acid sequence as set forth in SEQ ID NO: 117, and the heavy chain variable region comprises an amino acid sequence as set forth in SEQ ID NO: 118; the light chain variable region comprises an amino acid sequence as set forth in SEQ ID NO: 119, and the heavy chain variable region comprises an amino acid sequence as set forth in SEQ ID NO: 120; the light chain variable region comprises an amino acid sequence as set forth in SEQ ID NO: 121, and the heavy chain variable region comprises an amino acid sequence as set forth in SEQ ID NO: 122; the light chain variable region comprises an amino acid sequence as set forth in SEQ ID NO: 123, and the heavy chain variable region comprises an amino acid sequence as set forth in SEQ ID NO: 124; the light chain variable region comprises an amino acid sequence as set forth in SEQ ID NO: 125, and the heavy chain variable region comprises an amino acid sequence as set forth in SEQ ID NO: 126; or the light chain variable region comprises an amino acid sequence as set forth in SEQ ID NO: 127, and the heavy chain variable region comprises an amino acid sequence as set forth in SEQ ID NO: 128.

In some embodiments, the antibodies or antigen binding fragments thereof that bind IL-13 comprise a light chain comprising an amino acid sequence that is at least about 90%, at least about 95%, or at least about 99% identical to SEQ ID NO: 129, 131, 133, 135, 137, 139, 141, 143, 145, 147, 149, 151, 153, 155, 157, or 159; and/or a heavy chain comprising an amino acid sequence that is at least about 90%, at least about 95%, or at least about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof that bind IL-13 comprise a light chain comprising an amino acid sequence that is about 90%, about 95%, or about 99% identical to SEQ ID NO: 129, 131, 133, 135, 137, 139, 141, 143, 145, 147, 149, 151, 153, 155, 157, or 159; and/or a heavy chain comprising an amino acid sequence that is about 90%, about 95%, or about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the light chain comprises an amino acid sequence as set forth in SEQ ID NO: 129, 131, 133, 135, 137, 139, 141, 143, 145, 147, 149, 151, 153, 155, 157, or 159; and/or the heavy chain comprises an amino acid sequence as set forth in SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the light chain comprises an amino acid sequence as set forth in SEQ ID NO: 129, and the heavy chain comprises an amino acid sequence as set forth in SEQ ID NO: 130; the light chain comprises an amino acid sequence as set forth in SEQ ID NO: 131, and the heavy chain comprises an amino acid sequence as set forth in SEQ ID NO: 132; the light chain comprises an amino acid sequence as set forth in SEQ ID NO: 133, and the heavy chain comprises an amino acid sequence as set forth in SEQ ID NO: 134; the light chain comprises an amino acid sequence as set forth in SEQ ID NO: 135, and the heavy chain comprises an amino acid sequence as set forth in SEQ ID NO: 136; the light chain comprises an amino acid sequence as set forth in SEQ ID NO: 137, and the heavy chain comprises an amino acid sequence as set forth in SEQ ID NO: 138; the light chain comprises an amino acid sequence as set forth in SEQ ID NO: 139, and the heavy chain comprises an amino acid sequence as set forth in SEQ ID NO: 140; the light chain comprises an amino acid sequence as set forth in SEQ ID NO: 141, and the heavy chain comprises an amino acid sequence as set forth in SEQ ID NO: 142; the light chain comprises an amino acid sequence as set forth in SEQ ID NO: 143, and the heavy chain comprises an amino acid sequence as set forth in SEQ ID NO: 144; the light chain comprises an amino acid sequence as set forth in SEQ ID NO: 145, and the heavy chain comprises an amino acid sequence as set forth in SEQ ID NO: 146; the light chain comprises an amino acid sequence as set forth in SEQ ID NO: 147, and the heavy chain comprises an amino acid sequence as set forth in SEQ ID NO: 148; the light chain comprises an amino acid sequence as set forth in SEQ ID NO: 149, and the heavy chain comprises an amino acid sequence as set forth in SEQ ID NO: 150; the light chain comprises an amino acid sequence as set forth in SEQ ID NO: 151, and the heavy chain comprises an amino acid sequence as set forth in SEQ ID NO: 152; the light chain comprises an amino acid sequence as set forth in SEQ ID NO: 153, and the heavy chain comprises an amino acid sequence as set forth in SEQ ID NO: 154; the light chain comprises an amino acid sequence as set forth in SEQ ID NO: 155, and the heavy chain comprises an amino acid sequence as set forth in SEQ ID NO: 156; the light chain comprises an amino acid sequence as set forth in SEQ ID NO: 157, and the heavy chain comprises an amino acid sequence as set forth in SEQ ID NO: 158; the light chain comprises an amino acid sequence as set forth in SEQ ID NO: 159, and the heavy chain comprises an amino acid sequence as set forth in SEQ ID NO: 160; the light chain comprises an amino acid sequence as set forth in SEQ ID NO: 153, and the heavy chain comprises an amino acid sequence as set forth in SEQ ID NO: 182; the light chain comprises an amino acid sequence as set forth in SEQ ID NO: 147, and the heavy chain comprises an amino acid sequence as set forth in SEQ ID NO: 183; the light chain comprises an amino acid sequence as set forth in SEQ ID NO: 141, and the heavy chain comprises an amino acid sequence as set forth in SEQ ID NO: 184; the light chain comprises an amino acid sequence as set forth in SEQ ID NO: 153, and the heavy chain comprises an amino acid sequence as set forth in SEQ ID NO: 185; the light chain comprises an amino acid sequence as set forth in SEQ ID NO: 147, and the heavy chain comprises an amino acid sequence as set forth in SEQ ID NO: 186; the light chain comprises an amino acid sequence as set forth in SEQ ID NO: 141, and the heavy chain comprises an amino acid sequence as set forth in SEQ ID NO: 187; the light chain comprises an amino acid sequence as set forth in SEQ ID NO: 153, and the heavy chain comprises an amino acid sequence as set forth in SEQ ID NO: 188; the light chain comprises an amino acid sequence as set forth in SEQ ID NO: 147, and the heavy chain comprises an amino acid sequence as set forth in SEQ ID NO: 189; the light chain comprises an amino acid sequence as set forth in SEQ ID NO: 141, and the heavy chain comprises an amino acid sequence as set forth in SEQ ID NO: 190; the light chain comprises an amino acid sequence as set forth in SEQ ID NO: 153, and the heavy chain comprises an amino acid sequence as set forth in SEQ ID NO: 191; the light chain comprises an amino acid sequence as set forth in SEQ ID NO: 147, and the heavy chain comprises an amino acid sequence as set forth in SEQ ID NO: 192; the light chain comprises an amino acid sequence as set forth in SEQ ID NO: 141, and the heavy chain comprises an amino acid sequence as set forth in SEQ ID NO: 193; the light chain comprises an amino acid sequence as set forth in SEQ ID NO: 153, and the heavy chain comprises an amino acid sequence as set forth in SEQ ID NO: 194; the light chain comprises an amino acid sequence as set forth in SEQ ID NO: 147, and the heavy chain comprises an amino acid sequence as set forth in SEQ ID NO: 195; the light chain comprises an amino acid sequence as set forth in SEQ ID NO: 141, and the heavy chain comprises an amino acid sequence as set forth in SEQ ID NO: 196; the light chain comprises an amino acid sequence as set forth in SEQ ID NO: 153, and the heavy chain comprises an amino acid sequence as set forth in SEQ ID NO: 197; the light chain comprises an amino acid sequence as set forth in SEQ ID NO: 147, and the heavy chain comprises an amino acid sequence as set forth in SEQ ID NO: 198; the light chain comprises an amino acid sequence as set forth in SEQ ID NO: 141, and the heavy chain comprises an amino acid sequence as set forth in SEQ ID NO: 199; the light chain comprises an amino acid sequence as set forth in SEQ ID NO: 153, and the heavy chain comprises an amino acid sequence as set forth in SEQ ID NO: 200; the light chain comprises an amino acid sequence as set forth in SEQ ID NO: 147, and the heavy chain comprises an amino acid sequence as set forth in SEQ ID NO: 201; the light chain comprises an amino acid sequence as set forth in SEQ ID NO: 141, and the heavy chain comprises an amino acid sequence as set forth in SEQ ID NO: 202; the light chain comprises an amino acid sequence as set forth in SEQ ID NO: 153, and the heavy chain comprises an amino acid sequence as set forth in SEQ ID NO: 203; the light chain comprises an amino acid sequence as set forth in SEQ ID NO: 147, and the heavy chain comprises an amino acid sequence as set forth in SEQ ID NO: 204; the light chain comprises an amino acid sequence as set forth in SEQ ID NO: 141, and the heavy chain comprises an amino acid sequence as set forth in SEQ ID NO: 205; the light chain comprises an amino acid sequence as set forth in SEQ ID NO: 153, and the heavy chain comprises an amino acid sequence as set forth in SEQ ID NO: 206; the light chain comprises an amino acid sequence as set forth in SEQ ID NO: 147, and the heavy chain comprises an amino acid sequence as set forth in SEQ ID NO: 207; the light chain comprises an amino acid sequence as set forth in SEQ ID NO: 141, and the heavy chain comprises an amino acid sequence as set forth in SEQ ID NO: 208; the light chain comprises an amino acid sequence as set forth in SEQ ID NO: 153, and the heavy chain comprises an amino acid sequence as set forth in SEQ ID NO: 209; the light chain comprises an amino acid sequence as set forth in SEQ ID NO: 147, and the heavy chain comprises an amino acid sequence as set forth in SEQ ID NO: 210; or the light chain comprises an amino acid sequence as set forth in SEQ ID NO: 141, and the heavy chain comprises an amino acid sequence as set forth in SEQ ID NO: 211.

In some embodiments, the antibodies or antigen binding fragments thereof that bind IL-13 are monoclonal antibodies.

In some embodiments, the antibodies or antigen binding fragments thereof that bind IL-13 are human antibodies.

In some embodiments, the antibodies or antigen binding fragments thereof that bind IL-13 are bispecific antibodies.

In some embodiments, the antigen binding fragment thereof is selected from the group consisting of Fab, Fab′, F(ab′)₂, Fd, Fv, scFv, a single-chain antibody, a minibody, and a diabody.

In some embodiments, the antigen binding fragment thereof is an scFv.

In some aspects, described herein are antibodies or antigen binding fragments thereof comprising: i) a light chain variable region comprising SEQ ID NO: 1 (LCDR1). SEQ ID NO: 2 (LCDR2), and SEQ ID NO: 3 (LCDR3), and a heavy chain variable region comprising SEQ ID NO: 4 (HCDR1), SEQ ID NO: 5 (HCDR2), and SEQ ID NO: 6 (HCDR3); ii) a light chain variable region comprising SEQ ID NO: 7 (LCDR1), SEQ ID NO: 8 (LCDR2), and SEQ ID NO: 9 (LCDR3), and a heavy chain variable region comprising SEQ ID NO: 10 (HCDR1), SEQ ID NO: 11 (HCDR2), and SEQ ID NO: 12 (HCDR3): iii) a light chain variable region comprising SEQ ID NO: 13 (LCDR1), SEQ ID NO: 14 (LCDR2), and SEQ ID NO: 15 (LCDR3), and a heavy chain variable region comprising SEQ ID NO: 16 (HCDR1), SEQ ID NO: 17 (HCDR2), and SEQ ID NO: 18 (HCDR3): iv) a light chain variable region comprising SEQ ID NO: 19 (LCDR1), SEQ ID NO: 20 (LCDR2), and SEQ ID NO: 21 (LCDR3), and a heavy chain variable region comprising SEQ ID NO: 22 (HCDR1), SEQ ID NO: 23 (HCDR2), and SEQ ID NO: 24 (HCDR3); v) a light chain variable region comprising SEQ ID NO: 25 (LCDR1), SEQ ID NO: 26 (LCDR2), and SEQ ID NO: 27 (LCDR3), and a heavy chain variable region comprising SEQ ID NO: 28 (HCDR1), SEQ ID NO: 29 (HCDR2), and SEQ ID NO: 30 (HCDR3); vi) a light chain variable region comprising SEQ ID NO: 31 (LCDR1), SEQ ID NO: 32 (LCDR2), and SEQ ID NO: 33 (LCDR3), and a heavy chain variable region comprising SEQ ID NO: 34 (HCDR1), SEQ ID NO: 35 (HCDR2), and SEQ ID NO: 36 (HCDR3); vii) a light chain variable region comprising SEQ ID NO: 37 (LCDR1), SEQ ID NO: 38 (LCDR2), and SEQ ID NO: 39 (LCDR3), and a heavy chain variable region comprising SEQ ID NO: 40 (HCDR1), SEQ ID NO: 41 (HCDR2), and SEQ ID NO: 42 (HCDR3); viii) a light chain variable region comprising SEQ ID NO: 43 (LCDR1), SEQ ID NO: 44 (LCDR2), and SEQ ID NO: 45 (LCDR3), and a heavy chain variable region comprising SEQ ID NO: 46 (HCDR1), SEQ ID NO: 47 (HCDR2), and SEQ ID NO: 48 (HCDR3): ix) a light chain variable region comprising SEQ ID NO: 49 (LCDR1), SEQ ID NO: 50 (LCDR2), and SEQ ID NO: 51 (LCDR3), and a heavy chain variable region comprising SEQ ID NO: 52 (HCDR1), SEQ ID NO: 53 (HCDR2), and SEQ ID NO: 54 (HCDR3); x) a light chain variable region comprising SEQ ID NO: 55 (LCDR1). SEQ ID NO: 56 (LCDR2), and SEQ ID NO: 57 (LCDR3), and a heavy chain variable region comprising SEQ ID NO: 58 (HCDR1), SEQ ID NO: 59 (HCDR2), and SEQ ID NO: 60 (HCDR3); xi) a light chain variable region comprising SEQ ID NO: 61 (LCDR1), SEQ ID NO: 62 (LCDR2), and SEQ ID NO: 63 (LCDR3), and a heavy chain variable region comprising SEQ ID NO: 64 (HCDR1), SEQ ID NO: 65 (HCDR2), and SEQ ID NO: 66 (HCDR3); xii) a light chain variable region comprising SEQ ID NO: 67 (LCDR1), SEQ ID NO: 68 (LCDR2), and SEQ ID NO: 69 (LCDR3), and a heavy chain variable region comprising SEQ ID NO: 70 (HCDR1), SEQ ID NO: 71 (HCDR2), and SEQ ID NO: 72 (HCDR3); xiii) a light chain variable region comprising SEQ ID NO: 73 (LCDR1), SEQ ID NO: 74 (LCDR2), and SEQ ID NO: 75 (LCDR3), and a heavy chain variable region comprising SEQ ID NO: 76 (HCDR1), SEQ ID NO: 77 (HCDR2), and SEQ ID NO: 78 (HCDR3); xiv) a light chain variable region comprising SEQ ID NO: 79 (LCDR1), SEQ ID NO: 80 (LCDR2), and SEQ ID NO: 81 (LCDR3), and a heavy chain variable region comprising SEQ ID NO: 82 (HCDR1), SEQ ID NO: 83 (HCDR2), and SEQ ID NO: 84 (HCDR3); xv) a light chain variable region comprising SEQ ID NO: 85 (LCDR1), SEQ ID NO: 86 (LCDR2), and SEQ ID NO: 87 (LCDR3), and a heavy chain variable region comprising SEQ ID NO: 88 (HCDR1), SEQ ID NO: 89 (HCDR2), and SEQ ID NO: 90 (HCDR3); or xvi) a light chain variable region comprising SEQ ID NO: 91 (LCDR1), SEQ ID NO: 92 (LCDR2), and SEQ ID NO: 93 (LCDR3), and a heavy chain variable region comprising SEQ ID NO: 94 (HCDR1), SEQ ID NO: 95 (HCDR2), and SEQ ID NO: 96 (HCDR3).

In some embodiments, the antibodies or antigen binding fragments thereof for binding IL-13 comprise a light chain variable region comprising an amino acid sequence that is at least about 90%, at least about 95%, at least about 96%, at least about 97%, at least about 98%, or at least about 99% identical to SEQ ID NO: 97, 99, 101, 103, 105, 107, 109, 111, 113, 115, 117, 119, 121, 123, 125, or 127; and/or a heavy chain variable region comprising an amino acid sequence that is at least about 90%, at least about 95%, at least about 96%, at least about 97%, at least about 98%, or at least about 99% identical to SEQ ID NO: 98, 100, 102, 104, 106, 108, 110, 112, 114, 116, 118, 120, 122, 124, 126, or 128.

In some embodiments, the antibodies or antigen binding fragments thereof for binding IL-13 comprise a light chain variable region comprising an amino acid sequence that is about 90%, about 95%, about 96%, about 97%, about 98%, or about 99% identical to SEQ ID NO: 97, 99, 101, 103, 105, 107, 109, 111, 113, 115, 117, 119, 121, 123, 125, or 127; and/or a heavy chain variable region comprising an amino acid sequence that is about 90%, about 95%, about 96%, about 97%, about 98%, or about 99% identical to SEQ ID NO: 98, 100, 102, 104, 106, 108, 110, 112, 114, 116, 118, 120, 122, 124, 126, or 128.

In some embodiments, the antibodies or antigen binding fragments thereof for binding IL-13 comprise a light chain variable region comprising an amino acid sequence as set forth in SEQ ID NO: 97, 99, 101, 103, 105, 107, 109, 111, 113, 115, 117, 119, 121, 123, 125, or 127; and/or a heavy chain variable region comprising an amino acid sequence as set forth in SEQ ID NO: 98, 100, 102, 104, 106, 108, 110, 112, 114, 116, 118, 120, 122, 124, 126, or 128.

In some embodiments, the antibodies or antigen binding fragments thereof for binding IL-13 comprise: i) a light chain variable region comprising an amino acid sequence as set forth in in SEQ ID NO: 97, and a heavy chain variable region comprising an amino acid sequence as set forth in in SEQ ID NO: 98; ii) a light chain variable region comprising an amino acid sequence as set forth in in SEQ ID NO: 99, and a heavy chain variable region comprising an amino acid sequence as set forth in in SEQ ID NO: 100; iii) a light chain variable region comprising an amino acid sequence as set forth in in SEQ ID NO: 101, and a heavy chain variable region comprising an amino acid sequence as set forth in in SEQ ID NO: 102; vi) a light chain variable region comprising an amino acid sequence as set forth in in SEQ ID NO: 103, and a heavy chain variable region comprising an amino acid sequence as set forth in in SEQ ID NO: 104; v) a light chain variable region comprising an amino acid sequence as set forth in in SEQ ID NO: 105, and a heavy chain variable region comprising an amino acid sequence as set forth in in SEQ ID NO: 106; vi) a light chain variable region comprising an amino acid sequence as set forth in in SEQ ID NO: 107, and a heavy chain variable region comprising an amino acid sequence as set forth in in SEQ ID NO: 108; vii) a light chain variable region comprising an amino acid sequence as set forth in in SEQ ID NO: 109, and a heavy chain variable region comprising an amino acid sequence as set forth in in SEQ ID NO: 110; vii) a light chain variable region comprising an amino acid sequence as set forth in in SEQ ID NO: 111, and a heavy chain variable region comprising an amino acid sequence as set forth in in SEQ ID NO: 112; ix) a light chain variable region comprising an amino acid sequence as set forth in in SEQ ID NO: 113, and a heavy chain variable region comprising an amino acid sequence as set forth in in SEQ ID NO: 114; x) a light chain variable region comprising an amino acid sequence as set forth in in SEQ ID NO: 115, and a heavy chain variable region comprising an amino acid sequence as set forth in in SEQ ID NO: 116; xi) a light chain variable region comprising an amino acid sequence as set forth in in SEQ ID NO: 117, and a heavy chain variable region comprising an amino acid sequence as set forth in in SEQ ID NO: 118; xii) a light chain variable region comprising an amino acid sequence as set forth in in SEQ ID NO: 119, and a heavy chain variable region comprising an amino acid sequence as set forth in in SEQ ID NO: 120; xiii) a light chain variable region comprising an amino acid sequence as set forth in in SEQ ID NO: 121, and a heavy chain variable region comprising an amino acid sequence as set forth in in SEQ ID NO: 122; xiv) a light chain variable region comprising an amino acid sequence as set forth in in SEQ ID NO: 123, and a heavy chain variable region comprising an amino acid sequence as set forth in in SEQ ID NO: 124; xv) a light chain variable region comprising an amino acid sequence as set forth in in SEQ ID NO: 125, and a heavy chain variable region comprising an amino acid sequence as set forth in in SEQ ID NO: 126; or xvi) a light chain variable region comprising an amino acid sequence as set forth in in SEQ ID NO: 127, and a heavy chain variable region comprising an amino acid sequence as set forth in in SEQ ID NO: 128.

In some embodiments, the antibodies or antigen binding fragments thereof for binding IL-13 comprise a light chain comprising an amino acid sequence that is at least about 90%, at least about 95%, at least about 96%, at least about 97%, at least about 98%, or at least about 99% identical to SEQ ID NO: 129, 131, 133, 135, 137, 139, 141, 143, 145, 147, 149, 151, 153, 155, 157, or 159; and/or a heavy chain comprising an amino acid sequence that is at least about 90%, at least about 95%, at least about 96%, at least about 97%, at least about 98%, or at least about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof for binding IL-13 comprise a light chain comprising an amino acid sequence that is about 90%, about 95%, about 96%, about 97%, about 98%, or about 99% identical to SEQ ID NO: 129, 131, 133, 135, 137, 139, 141, 143, 145, 147, 149, 151, 153, 155, 157, or 159; and/or a heavy chain comprising an amino acid sequence that is about 90%, about 95%, about 96%, about 97%, about 98%, or about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof for binding IL-13 comprises a light chain comprising an amino acid sequence as set forth in SEQ ID NO: 129, 131, 133, 135, 137, 139, 141, 143, 145, 147, 149, 151, 153, 155, 157, or 159; and/or a heavy chain comprising an amino acid sequence as set forth in SEQ ID NO: 130, 132, 134, 136, 138, 140, 142, 144, 146, 148, 150, 152, 154, 156, 158, 160, 182 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof for binding IL-13 comprise: i) a light chain comprising an amino acid sequence as set forth in SEQ ID NO: 129, and a heavy chain comprising an amino acid sequence as set forth in SEQ ID NO: 130; ii) a light chain comprising an amino acid sequence as set forth in SEQ ID NO: 131, and a heavy chain comprising an amino acid sequence as set forth in SEQ ID NO: 132; iii) a light chain comprising an amino acid sequence as set forth in SEQ ID NO: 133, and a heavy chain comprising an amino acid sequence as set forth in SEQ ID NO: 134; iv) a light chain comprising an amino acid sequence as set forth in SEQ ID NO: 135, and a heavy chain comprising an amino acid sequence as set forth in SEQ ID NO: 136; v) a light chain comprising an amino acid sequence as set forth in SEQ ID NO: 137, and a heavy chain comprising an amino acid sequence as set forth in SEQ ID NO: 138; vi) a light chain comprising an amino acid sequence as set forth in SEQ ID NO: 139, and a heavy chain comprising an amino acid sequence as set forth in SEQ ID NO: 140; vii) a light chain comprising an amino acid sequence as set forth in SEQ ID NO: 141, and a heavy chain comprising an amino acid sequence as set forth in SEQ ID NO: 142; viii) a light chain comprising an amino acid sequence as set forth in SEQ ID NO: 143, and a heavy chain comprising an amino acid sequence as set forth in SEQ ID NO: 144; ix) a light chain comprising an amino acid sequence as set forth in SEQ ID NO: 145, and a heavy chain comprising an amino acid sequence as set forth in SEQ ID NO: 146; x) a light chain comprising an amino acid sequence as set forth in SEQ ID NO: 147, and a heavy chain comprising an amino acid sequence as set forth in SEQ ID NO: 148; xi) a light chain comprising an amino acid sequence as set forth in SEQ ID NO: 149, and a heavy chain comprising an amino acid sequence as set forth in SEQ ID NO: 150; xii) a light chain comprising an amino acid sequence as set forth in SEQ ID NO: 151, and a heavy chain comprising an amino acid sequence as set forth in SEQ ID NO: 152; xiii) a light chain comprising an amino acid sequence as set forth in SEQ ID NO: 153, and a heavy chain comprising an amino acid sequence as set forth in SEQ ID NO: 154; xiv) a light chain comprising an amino acid sequence as set forth in SEQ ID NO: 155, and a heavy chain comprising an amino acid sequence as set forth in SEQ ID NO: 156; xv) a light chain comprising an amino acid sequence as set forth in SEQ ID NO: 157, and a heavy chain comprising an amino acid sequence as set forth in SEQ ID NO: 158; xvi) a light chain comprising an amino acid sequence as set forth in SEQ ID NO: 159, and a heavy chain comprising an amino acid sequence as set forth in SEQ ID NO: 160; xvii) a light chain comprising an amino acid sequence as set forth in SEQ ID NO: 153, and a heavy chain comprising an amino acid sequence as set forth in SEQ ID NO: 182; xviii) a light chain comprising an amino acid sequence as set forth in SEQ ID NO: 147, and a heavy chain comprising an amino acid sequence as set forth in SEQ ID NO: 183; xix) a light chain comprising an amino acid sequence as set forth in SEQ ID NO: 141, and a heavy chain comprising an amino acid sequence as set forth in SEQ ID NO: 184; xx) a light chain comprising an amino acid sequence as set forth in SEQ ID NO: 153, and a heavy chain comprising an amino acid sequence as set forth in SEQ ID NO: 185; xxi) a light chain comprising an amino acid sequence as set forth in SEQ ID NO: 147, and a heavy chain comprising an amino acid sequence as set forth in SEQ ID NO: 186; xxii) a light chain comprising an amino acid sequence as set forth in SEQ ID NO: 141, and a heavy chain comprising an amino acid sequence as set forth in SEQ ID NO: 187; xxiii) a light chain comprising an amino acid sequence as set forth in SEQ ID NO: 153, and a heavy chain comprising an amino acid sequence as set forth in SEQ ID NO: 188; xxiv) a light chain comprising an amino acid sequence as set forth in SEQ ID NO: 147, and a heavy chain comprising an amino acid sequence as set forth in SEQ ID NO: 189; xxv) a light chain comprising an amino acid sequence as set forth in SEQ ID NO: 141, and a heavy chain comprising an amino acid sequence as set forth in SEQ ID NO: 190; xxvi) a light chain comprising an amino acid sequence as set forth in SEQ ID NO: 153, and a heavy chain comprising an amino acid sequence as set forth in SEQ ID NO: 191; xxvii) a light chain comprising an amino acid sequence as set forth in SEQ ID NO: 147, and a heavy chain comprising an amino acid sequence as set forth in SEQ ID NO: 192; xxviii) a light chain comprising an amino acid sequence as set forth in SEQ ID NO: 141, and a heavy chain comprising an amino acid sequence as set forth in SEQ ID NO: 193; xxix) a light chain comprising an amino acid sequence as set forth in SEQ ID NO: 153, and a heavy chain comprising an amino acid sequence as set forth in SEQ ID NO: 194; xxx) a light chain comprising an amino acid sequence as set forth in SEQ ID NO: 147, and a heavy chain comprising an amino acid sequence as set forth in SEQ ID NO: 195; xxxi) a light chain comprising an amino acid sequence as set forth in SEQ ID NO: 141, and a heavy chain comprising an amino acid sequence as set forth in SEQ ID NO: 196; xxxii) a light chain comprising an amino acid sequence as set forth in SEQ ID NO: 153, and a heavy chain comprising an amino acid sequence as set forth in SEQ ID NO: 197; xxxiii) a light chain comprising an amino acid sequence as set forth in SEQ ID NO: 147, and a heavy chain comprising an amino acid sequence as set forth in SEQ ID NO: 198; xxxiv) a light chain comprising an amino acid sequence as set forth in SEQ ID NO: 141, and a heavy chain comprising an amino acid sequence as set forth in SEQ ID NO: 199; xxxv) a light chain comprising an amino acid sequence as set forth in SEQ ID NO: 153, and a heavy chain comprising an amino acid sequence as set forth in SEQ ID NO: 200; xxxvi) a light chain comprising an amino acid sequence as set forth in SEQ ID NO: 147, and a heavy chain comprising an amino acid sequence as set forth in SEQ ID NO: 201; xxxvii) a light chain comprising an amino acid sequence as set forth in SEQ ID NO: 141, and a heavy chain comprising an amino acid sequence as set forth in SEQ ID NO: 202; xxxviii) a light chain comprising an amino acid sequence as set forth in SEQ ID NO: 153, and a heavy chain comprising an amino acid sequence as set forth in SEQ ID NO: 203; xxxix) a light chain comprising an amino acid sequence as set forth in SEQ ID NO: 147, and a heavy chain comprising an amino acid sequence as set forth in SEQ ID NO: 204; xl) a light chain comprising an amino acid sequence as set forth in SEQ ID NO: 141, and a heavy chain comprising an amino acid sequence as set forth in SEQ ID NO: 205; xli) a light chain comprising an amino acid sequence as set forth in SEQ ID NO: 153, and a heavy chain comprising an amino acid sequence as set forth in SEQ ID NO: 206; xlii) a light chain comprising an amino acid sequence as set forth in SEQ ID NO: 147, and a heavy chain comprising an amino acid sequence as set forth in SEQ ID NO: 207; xliii) a light chain comprising an amino acid sequence as set forth in SEQ ID NO: 141, and a heavy chain comprising an amino acid sequence as set forth in SEQ ID NO: 208; xliv) a light chain comprising an amino acid sequence as set forth in SEQ ID NO: 153, and a heavy chain comprising an amino acid sequence as set forth in SEQ ID NO: 209; xlv) a light chain comprising an amino acid sequence as set forth in SEQ ID NO: 147, and a heavy chain comprising an amino acid sequence as set forth in SEQ ID NO: 210; or xlvi) a light chain comprising an amino acid sequence as set forth in SEQ ID NO: 141, and a heavy chain comprising an amino acid sequence as set forth in SEQ ID NO: 211.

In some embodiments, the antibodies or antigen binding fragments thereof for binding IL-13 are monoclonal antibodies.

In some embodiments, the antibodies or antigen binding fragments thereof for binding IL-13 are human antibodies.

In some embodiments, the antibodies or antigen binding fragments thereof for binding IL-13 are bispecific antibodies.

In some embodiments, the antigen binding fragment thereof is selected from the group consisting of: Fab, Fab′, F(ab′)₂, Fd, Fv, scFv, a single-chain antibody, a minibody, and a diabody.

In some embodiments, the antigen binding fragments thereof is an scFv.

In some aspects, provided herein are antibodies or antigen binding fragments thereof for binding IL-13 used as a medicament.

In some aspects, provided herein is a pharmaceutical composition comprising antibodies or antigen binding fragments thereof for binding IL-13 and a pharmaceutically acceptable carrier.

In some aspects, provided herein is a nucleic acid composition comprising one or more nucleic acids encoding antibodies or antigen binding fragments thereof for binding IL-13.

In some aspects, provided herein is an expression vector composition comprising one or more expression vectors comprising a nucleic acid composition as provided herein.

In some aspects, provided herein is a host cell comprising a nucleic acid composition or an expression vector composition as provided herein.

In some aspects, provided herein is a method for making anti-IL-13 antibodies or antigen binding fragments thereof comprising culturing the host cell as described herein under conditions where the anti-IL-13 antibodies or antigen binding fragments thereof are expressed and recovering the anti-IL-13 antibodies or binding fragments thereof.

In some aspects, also provided herein are antibodies or antigen binding fragments thereof for binding IL-13 comprising: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence at least about 90%, at least about 95%, or at least about 99% identical to SEQ ID NO: 97, 99, 101, 103, 105, 107, 109, 111, 113, 115, 117, 119, 121, 123, 125, or 127; and b2) a heavy chain variable region comprising an amino acid sequence at least about 90%, at least about 95%, or at least about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some aspects, also provided herein are antibodies or antigen binding fragments thereof for binding IL-13 comprising: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence about 90%, about 95%, or about 99% identical to SEQ ID NO: 97, 99, 101, 103, 105, 107, 109, 111, 113, 115, 117, 119, 121, 123, 125, or 127; and b2) a heavy chain variable region comprising an amino acid sequence about 90%, about 95%, or about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some embodiments, the antibodies or antigen binding fragments thereof for binding IL-13 comprise a LCDR1 comprising a serine (S) at amino acid position 27D and an arginine (R) at amino acid position 30; a LCDR1 comprising a tyrosine (Y) at amino acid position 27D and a histidine (H) at amino acid position 30; a LCDR1 comprising a tyrosine (Y) at amino acid position 27D and an asparagine (N) at amino acid position 30; or a LCDR1 comprises a serine (S) at amino acid position 27D and a tyrosine (Y) at amino acid position 32, wherein the amino acid positions are based on Kabat numbering.

In some aspects, also provided herein are antibodies or antigen binding fragments thereof for binding IL-13 comprising a light chain variable region and a heavy chain variable region, wherein the light chain variable region is at least about 95%, at least about 96%, at least about 97%, at least about 98%, or at least about 99% identical to SEQ ID NO: 97, 99, 101, and 103; wherein the heavy chain variable region is at least about 95%, at least about 96%, at least about 97%, at least about 98%, or at least about 99% identical to SEQ ID NO: 98, 100, 102, and 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, and wherein the amino acid positions are based on Kabat numbering.

In some aspects, also provided herein are antibodies or antigen binding fragments thereof for binding IL-13 comprising a light chain variable region and a heavy chain variable region, wherein the light chain variable region is about 95%, about 96%, about 97%, about 98%, or about 99% identical to SEQ ID NO: 97, 99, 101, and 103; wherein the heavy chain variable region is about 95%, about 96%, about 97%, about 98%, or about 99% identical to SEQ ID NO: 98, 100, 102, and 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, and wherein the amino acid positions are based on Kabat numbering.

In some embodiments, the antibodies or antigen binding fragments thereof for binding IL-13 are monoclonal antibodies.

In some embodiments, the antibodies or antigen binding fragments thereof for binding IL-13 are human antibodies.

In some embodiments, the antibodies or antigen binding fragments thereof for binding IL-13 are bispecific antibodies.

In some embodiments, the antigen binding fragment thereof is selected from the group consisting of: Fab, Fab′, F(ab′)₂, Fd, Fv, scFv, a single-chain antibody, a minibody, and a diabody.

In some embodiments, the antigen binding fragment thereof is an scFv.

In some aspects, provided herein are antibodies or antigen binding fragments thereof for binding IL-13 used as a medicament.

In some aspects, provided herein is a pharmaceutical composition comprising antibodies or an antigen binding fragments thereof and a pharmaceutically acceptable carrier.

In some aspects, provided herein is a nucleic acid composition comprising one or more nucleic acids encoding antibodies or antigen binding fragments thereof for binding IL-13.

In some aspects, provided herein is an expression vector composition comprising one or more expression vectors comprising a nucleic acid composition as provided herein.

In some aspects, provided herein is a host cell comprising a nucleic acid composition or an expression vector composition as provided herein.

In some aspects, provided herein is a method for making anti-IL-13 antibodies or antigen binding fragments thereof comprising culturing the host cell as described herein under conditions where the anti-IL-13 antibodies or antigen binding fragments thereof are expressed and recovering the anti-IL-13 antibodies or binding fragments thereof.

In some aspects, described herein is a method of treating a disease or disorder in a subject in need thereof, the method comprising administering to the subject a therapeutically effective dose of antibodies or antigen binding fragments thereof for binding IL-13, wherein the antibodies or antigen binding fragments thereof for binding IL-13 comprise: i) a light chain variable region comprising SEQ ID NO: 1 (LCDR1), SEQ ID NO: 2 (LCDR2), and SEQ ID NO: 3 (LCDR3), and a heavy chain variable region comprising SEQ ID NO: 4 (HCDR1), SEQ ID NO: 5 (HCDR2), and SEQ ID NO: 6 (HCDR3); ii) a light chain variable region comprising SEQ ID NO: 7 (LCDR1), SEQ ID NO: 8 (LCDR2), and SEQ ID NO: 9 (LCDR3), and a heavy chain variable region comprising SEQ ID NO: 10 (HCDR1), SEQ ID NO: 11 (HCDR2), and SEQ ID NO: 12 (HCDR3); iii) a light chain variable region comprising SEQ ID NO: 13 (LCDR1), SEQ ID NO: 14 (LCDR2), and SEQ ID NO: 15 (LCDR3), and a heavy chain variable region comprising SEQ ID NO: 16 (HCDR1), SEQ ID NO: 17 (HCDR2), and SEQ ID NO: 18 (HCDR3); iv) a light chain variable region comprising SEQ ID NO: 19 (LCDR1), SEQ ID NO: 20 (LCDR2), and SEQ ID NO: 21 (LCDR3), and a heavy chain variable region comprising SEQ ID NO: 22 (HCDR1), SEQ ID NO: 23 (HCDR2), and SEQ ID NO: 24 (HCDR3); v) a light chain variable region comprising SEQ ID NO: 25 (LCDR1), SEQ ID NO: 26 (LCDR2), and SEQ ID NO: 27 (LCDR3), and a heavy chain variable region comprising SEQ ID NO: 28 (HCDR1), SEQ ID NO: 29 (HCDR2), and SEQ ID NO: 30 (HCDR3); vi) a light chain variable region comprising SEQ ID NO: 31 (LCDR1), SEQ ID NO: 32 (LCDR2), and SEQ ID NO: 33 (LCDR3), and a heavy chain variable region comprising SEQ ID NO: 34 (HCDR1), SEQ ID NO: 35 (HCDR2), and SEQ ID NO: 36 (HCDR3); vii) a light chain variable region comprising SEQ ID NO: 37 (LCDR1), SEQ ID NO: 38 (LCDR2), and SEQ ID NO: 39 (LCDR3), and a heavy chain variable region comprising SEQ ID NO: 40 (HCDR1), SEQ ID NO: 41 (HCDR2), and SEQ ID NO: 42 (HCDR3); viii) a light chain variable region comprising SEQ ID NO: 43 (LCDR1), SEQ ID NO: 44 (LCDR2), and SEQ ID NO: 45 (LCDR3), and a heavy chain variable region comprising SEQ ID NO: 46 (HCDR1), SEQ ID NO: 47 (HCDR2), and SEQ ID NO: 48 (HCDR3); ix) a light chain variable region comprising SEQ ID NO: 49 (LCDR1), SEQ ID NO: 50 (LCDR2), and SEQ ID NO: 51 (LCDR3), and a heavy chain variable region comprising SEQ ID NO: 52 (HCDR1), SEQ ID NO: 53 (HCDR2), and SEQ ID NO: 54 (HCDR3); x) a light chain variable region comprising SEQ ID NO: 55 (LCDR1), SEQ ID NO: 56 (LCDR2), and SEQ ID NO: 57 (LCDR3), and a heavy chain variable region comprising SEQ ID NO: 58 (HCDR1), SEQ ID NO: 59 (HCDR2), and SEQ ID NO: 60 (HCDR3); xi) a light chain variable region comprising SEQ ID NO: 61 (LCDR1), SEQ ID NO: 62 (LCDR2), and SEQ ID NO: 63 (LCDR3), and a heavy chain variable region comprising SEQ ID NO: 64 (HCDR1), SEQ ID NO: 65 (HCDR2), and SEQ ID NO: 66 (HCDR3); xii) a light chain variable region comprising SEQ ID NO: 67 (LCDR1), SEQ ID NO: 68 (LCDR2), and SEQ ID NO: 69 (LCDR3), and a heavy chain variable region comprising SEQ ID NO: 70 (HCDR1), SEQ ID NO: 71 (HCDR2), and SEQ ID NO: 72 (HCDR3); xiii) a light chain variable region comprising SEQ ID NO: 73 (LCDR1), SEQ ID NO: 74 (LCDR2), and SEQ ID NO: 75 (LCDR3), and a heavy chain variable region comprising SEQ ID NO: 76 (HCDR1), SEQ ID NO: 77 (HCDR2), and SEQ ID NO: 78 (HCDR3); xiv) a light chain variable region comprising SEQ ID NO: 79 (LCDR1), SEQ ID NO: 80 (LCDR2), and SEQ ID NO: 81 (LCDR3), and a heavy chain variable region comprising SEQ ID NO: 82 (HCDR1), SEQ ID NO: 83 (HCDR2), and SEQ ID NO: 84 (HCDR3); xv) a light chain variable region comprising SEQ ID NO: 85 (LCDR1), SEQ ID NO: 86 (LCDR2), and SEQ ID NO: 87 (LCDR3), and a heavy chain variable region comprising SEQ ID NO: 88 (HCDR1), SEQ ID NO: 89 (HCDR2), and SEQ ID NO: 90 (HCDR3); or xvi) a light chain variable region comprising SEQ ID NO: 91 (LCDR1), SEQ ID NO: 92 (LCDR2), and SEQ ID NO: 93 (LCDR3), and a heavy chain variable region comprising SEQ ID NO: 94 (HCDR1), SEQ ID NO: 95 (HCDR2), and SEQ ID NO: 96 (HCDR3).

In some embodiments, the disease or disorder is selected from the group consisting of: asthma (including, but not limited to, mild asthma, mild to moderate asthma, moderate asthma, moderate to severe asthma, and/or severe asthma), allergic asthma, allergen-induced airway obstruction due to asthma and/or allergic asthma, atopic dermatitis (including, but not limited to, mild atopic dermatitis, mild to moderate atopic dermatitis, moderate atopic dermatitis, moderate-to-severe, and/or severe atopic dermatitis), eczema, acute urticaria, idiopathic pulmonary fibrosis (IPF), chronic obstructive pulmonary disease (COPD), chronic obstructive airway disease, emphysema, chronic bronchitis, pulmonary fibrosis, systemic sclerosis, scleroderma, cryptogenic fibrosing alveolitis, usual interstitial pneumonitis, idiopathic interstitial pneumonitis, wound, systemic lupus erythematosus (SLE), rheumatoid arthritis (RA), systemic sclerosis (SS), ulcerative colitis (UC), type 1 diabetes (T1D), sjogren's syndrome (SS), prurigo nodularis, nasal polyposis, recurrent urticaria, aspergillosis, bullous pemphigoid, chronic sinusitis, alopecia areata, hay fever, pemphigus, allergic rhinitis, psoriasis, rosacea, allergic drug reactions, allergic reactions, anaphylaxis, acne, food allergies, inflammatory bowel disease (IBS), Crohn's disease, ulcerative colitis, chronic rhinosinusitis with nasal polyps (CRSwNP), eosinophilic esophagitis, eosinophilic colitis, eosinophilic gastritis, eosinophilic cystitis, eosinophilic fasciitis, eosinophilic pustular folliculitis, eosinophilic granulomatosis, eosinophilic polyangiitis, Churg-Strauss syndrome, biliary cancer, brain cancer, breast cancer, colorectal cancer, genitourinary cancer, head and neck cancer, liver cancer, Hodgkin's lymphoma, esophageal cancer, pancreatic cancer, prostate cancer, renal cancer, Kaposi's sarcoma, sinusitis, or chronic urticaria.

In some embodiments, the light chain variable region comprises an amino acid sequence that is at least about 90%, at least about 95%, or at least about 99% identical to SEQ ID NO: 97, 99, 101, 103, 105, 107, 109, 111, 113, 115, 117, 119, 121, 123, 125, or 127; and the heavy chain variable region comprises an amino acid sequence that is at least about 90%, at least about 95%, or at least about 99% identical to SEQ ID NO: 98, 100, 102, 104, 106, 108, 110, 112, 114, 116, 118, 120, 122, 124, 126, or 128.

In some embodiments, the light chain variable region comprises an amino acid sequence that is about 90%, about 95%, or about 99% identical to SEQ ID NO: 97, 99, 101, 103, 105, 107, 109, 111, 113, 115, 117, 119, 121, 123, 125, or 127; and the heavy chain variable region comprises an amino acid sequence that is about 90%, about 95%, or about 99% identical to SEQ ID NO: 98, 100, 102, 104, 106, 108, 110, 112, 114, 116, 118, 120, 122, 124, 126, or 128.

In some embodiments, the light chain variable region comprises an amino acid sequence as set forth in SEQ ID NO: 97, 99, 101, 103, 105, 107, 109, 111, 113, 115, 117, 119, 121, 123, 125, or 127; and/or the heavy chain variable region comprises an amino acid sequence as set forth in SEQ ID NO: 98, 100, 102, 104, 106, 108, 110, 112, 114, 116, 118, 120, 122, 124, 126, or 128.

In some embodiments, the light chain variable region comprises an amino acid sequence as set forth in SEQ ID NO: 97, and the heavy chain variable region comprises an amino acid sequence as set forth in SEQ ID NO: 98; the light chain variable region comprises an amino acid sequence as set forth in SEQ ID NO: 99, and the heavy chain variable region comprises an amino acid sequence as set forth in SEQ ID NO: 100; the light chain variable region comprises an amino acid sequence as set forth in SEQ ID NO: 101, and the heavy chain variable region comprises an amino acid sequence as set forth in SEQ ID NO: 102; the light chain variable region comprises an amino acid sequence as set forth in SEQ ID NO: 103, and the heavy chain variable region comprises an amino acid sequence as set forth in SEQ ID NO: 104; the light chain variable region comprises an amino acid sequence as set forth in SEQ ID NO: 105, and the heavy chain variable region comprises an amino acid sequence as set forth in SEQ ID NO: 106; the light chain variable region comprises an amino acid sequence as set forth in SEQ ID NO: 107, and the heavy chain variable region comprises an amino acid sequence as set forth in SEQ ID NO: 108; the light chain variable region comprises an amino acid sequence as set forth in SEQ ID NO: 109, and the heavy chain variable region comprises an amino acid sequence as set forth in SEQ ID NO: 110; the light chain variable region comprises an amino acid sequence as set forth in SEQ ID NO: 111, and the heavy chain variable region comprises an amino acid sequence as set forth in SEQ ID NO: 112; the light chain variable region comprises an amino acid sequence as set forth in SEQ ID NO: 113, and the heavy chain variable region comprises an amino acid sequence as set forth in SEQ ID NO: 114; the light chain variable region comprises an amino acid sequence as set forth in SEQ ID NO: 115, and the heavy chain variable region comprises an amino acid sequence as set forth in SEQ ID NO: 116; the light chain variable region comprises an amino acid sequence as set forth in SEQ ID NO: 117, and the heavy chain variable region comprises an amino acid sequence as set forth in SEQ ID NO: 118; the light chain variable region comprises an amino acid sequence as set forth in SEQ ID NO: 119, and the heavy chain variable region comprises an amino acid sequence as set forth in SEQ ID NO: 120; the light chain variable region comprises an amino acid sequence as set forth in SEQ ID NO: 121, and the heavy chain variable region comprises an amino acid sequence as set forth in SEQ ID NO: 122; the light chain variable region comprises an amino acid sequence as set forth in SEQ ID NO: 123, and the heavy chain variable region comprises an amino acid sequence as set forth in SEQ ID NO: 124; the light chain variable region comprises an amino acid sequence as set forth in SEQ ID NO: 125, and the heavy chain variable region comprises an amino acid sequence as set forth in SEQ ID NO: 126; or the light chain variable region comprises an amino acid sequence as set forth in SEQ ID NO: 127, and the heavy chain variable region comprises an amino acid sequence as set forth in SEQ ID NO: 128.

In some embodiments, the antibodies or antigen binding fragments thereof for binding IL-13 comprise a light chain comprising an amino acid sequence that is at least about 90%, at least about 95%, or at least about 99% identical to SEQ ID NO: 129, 131, 133, 135, 137, 139, 141, 143, 145, 147, 149, 151, 153, 155, 157, or 159; and/or a heavy chain comprising an amino acid sequence that is at least about 90%, at least about 95%, or at least about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the antibodies or antigen binding fragments thereof for binding IL-13 comprise a light chain comprising an amino acid sequence that is about 90%, about 95%, or about 99% identical to SEQ ID NO: 129, 131, 133, 135, 137, 139, 141, 143, 145, 147, 149, 151, 153, 155, 157, or 159; and/or a heavy chain comprising an amino acid sequence that is about 90%, about 95%, or about 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the light chain comprises an amino acid sequence as set forth in SEQ ID NO: 129, 131, 133, 135, 137, 139, 141, 143, 145, 147, 149, 151, 153, 155, 157, or 159; and/or the heavy chain comprises an amino acid sequence as set forth in SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

In some embodiments, the light chain comprises an amino acid sequence as set forth in SEQ ID NO: 129, and the heavy chain comprises an amino acid sequence as set forth in SEQ ID NO: 130; the light chain comprises an amino acid sequence as set forth in SEQ ID NO: 131, and the heavy chain comprises an amino acid sequence as set forth in SEQ ID NO: 132; the light chain comprises an amino acid sequence as set forth in SEQ ID NO: 133, and the heavy chain comprises an amino acid sequence as set forth in SEQ ID NO: 134; the light chain comprises an amino acid sequence as set forth in SEQ ID NO: 135, and the heavy chain comprises an amino acid sequence as set forth in SEQ ID NO: 136; the light chain comprises an amino acid sequence as set forth in SEQ ID NO: 137, and the heavy chain comprises an amino acid sequence as set forth in SEQ ID NO: 138; the light chain comprises an amino acid sequence as set forth in SEQ ID NO: 139, and the heavy chain comprises an amino acid sequence as set forth in SEQ ID NO: 140; the light chain comprises an amino acid sequence as set forth in SEQ ID NO: 141, and the heavy chain comprises an amino acid sequence as set forth in SEQ ID NO: 142; the light chain comprises an amino acid sequence as set forth in SEQ ID NO: 143, and the heavy chain comprises an amino acid sequence as set forth in SEQ ID NO: 144; the light chain comprises an amino acid sequence as set forth in SEQ ID NO: 145, and the heavy chain comprises an amino acid sequence as set forth in SEQ ID NO: 146; the light chain comprises an amino acid sequence as set forth in SEQ ID NO: 147, and the heavy chain comprises an amino acid sequence as set forth in SEQ ID NO: 148; the light chain comprises an amino acid sequence as set forth in SEQ ID NO: 149, and the heavy chain comprises an amino acid sequence as set forth in SEQ ID NO: 150; the light chain comprises an amino acid sequence as set forth in SEQ ID NO: 151, and the heavy chain comprises an amino acid sequence as set forth in SEQ ID NO: 152; the light chain comprises an amino acid sequence as set forth in SEQ ID NO: 153, and the heavy chain comprises an amino acid sequence as set forth in SEQ ID NO: 154; the light chain comprises an amino acid sequence as set forth in SEQ ID NO: 155, and the heavy chain comprises an amino acid sequence as set forth in SEQ ID NO: 156; the light chain comprises an amino acid sequence as set forth in SEQ ID NO: 157, and the heavy chain comprises an amino acid sequence as set forth in SEQ ID NO: 158; the light chain comprises an amino acid sequence as set forth in SEQ ID NO: 159, and the heavy chain comprises an amino acid sequence as set forth in SEQ ID NO: 160; the light chain comprises an amino acid sequence as set forth in SEQ ID NO: 153, and the heavy chain comprises an amino acid sequence as set forth in SEQ ID NO: 182; the light chain comprises an amino acid sequence as set forth in SEQ ID NO: 147, and the heavy chain comprises an amino acid sequence as set forth in SEQ ID NO: 183; the light chain comprises an amino acid sequence as set forth in SEQ ID NO: 141, and the heavy chain comprises an amino acid sequence as set forth in SEQ ID NO: 184; the light chain comprises an amino acid sequence as set forth in SEQ ID NO: 153, and the heavy chain comprises an amino acid sequence as set forth in SEQ ID NO: 185; the light chain comprises an amino acid sequence as set forth in SEQ ID NO: 147, and the heavy chain comprises an amino acid sequence as set forth in SEQ ID NO: 186; the light chain comprises an amino acid sequence as set forth in SEQ ID NO: 141, and the heavy chain comprises an amino acid sequence as set forth in SEQ ID NO: 187; the light chain comprises an amino acid sequence as set forth in SEQ ID NO: 153, and the heavy chain comprises an amino acid sequence as set forth in SEQ ID NO: 188; the light chain comprises an amino acid sequence as set forth in SEQ ID NO: 147, and the heavy chain comprises an amino acid sequence as set forth in SEQ ID NO: 189; the light chain comprises an amino acid sequence as set forth in SEQ ID NO: 141, and the heavy chain comprises an amino acid sequence as set forth in SEQ ID NO: 190; the light chain comprises an amino acid sequence as set forth in SEQ ID NO: 153, and the heavy chain comprises an amino acid sequence as set forth in SEQ ID NO: 191; the light chain comprises an amino acid sequence as set forth in SEQ ID NO: 147, and the heavy chain comprises an amino acid sequence as set forth in SEQ ID NO: 192; the light chain comprises an amino acid sequence as set forth in SEQ ID NO: 141, and the heavy chain comprises an amino acid sequence as set forth in SEQ ID NO: 193; the light chain comprises an amino acid sequence as set forth in SEQ ID NO: 153, and the heavy chain comprises an amino acid sequence as set forth in SEQ ID NO: 194; the light chain comprises an amino acid sequence as set forth in SEQ ID NO: 147, and the heavy chain comprises an amino acid sequence as set forth in SEQ ID NO: 195; the light chain comprises an amino acid sequence as set forth in SEQ ID NO: 141, and the heavy chain comprises an amino acid sequence as set forth in SEQ ID NO: 196; the light chain comprises an amino acid sequence as set forth in SEQ ID NO: 153, and the heavy chain comprises an amino acid sequence as set forth in SEQ ID NO: 197; the light chain comprises an amino acid sequence as set forth in SEQ ID NO: 147, and the heavy chain comprises an amino acid sequence as set forth in SEQ ID NO: 198; the light chain comprises an amino acid sequence as set forth in SEQ ID NO: 141, and the heavy chain comprises an amino acid sequence as set forth in SEQ ID NO: 199; the light chain comprises an amino acid sequence as set forth in SEQ ID NO: 153, and the heavy chain comprises an amino acid sequence as set forth in SEQ ID NO: 200; the light chain comprises an amino acid sequence as set forth in SEQ ID NO: 147, and the heavy chain comprises an amino acid sequence as set forth in SEQ ID NO: 201; the light chain comprises an amino acid sequence as set forth in SEQ ID NO: 141, and the heavy chain comprises an amino acid sequence as set forth in SEQ ID NO: 202; the light chain comprises an amino acid sequence as set forth in SEQ ID NO: 153, and the heavy chain comprises an amino acid sequence as set forth in SEQ ID NO: 203; the light chain comprises an amino acid sequence as set forth in SEQ ID NO: 147, and the heavy chain comprises an amino acid sequence as set forth in SEQ ID NO: 204; the light chain comprises an amino acid sequence as set forth in SEQ ID NO: 141, and the heavy chain comprises an amino acid sequence as set forth in SEQ ID NO: 205; the light chain comprises an amino acid sequence as set forth in SEQ ID NO: 153, and the heavy chain comprises an amino acid sequence as set forth in SEQ ID NO: 206; the light chain comprises an amino acid sequence as set forth in SEQ ID NO: 147, and the heavy chain comprises an amino acid sequence as set forth in SEQ ID NO: 207; the light chain comprises an amino acid sequence as set forth in SEQ ID NO: 141, and the heavy chain comprises an amino acid sequence as set forth in SEQ ID NO: 208; the light chain comprises an amino acid sequence as set forth in SEQ ID NO: 153, and the heavy chain comprises an amino acid sequence as set forth in SEQ ID NO: 209; the light chain comprises an amino acid sequence as set forth in SEQ ID NO: 147, and the heavy chain comprises an amino acid sequence as set forth in SEQ ID NO: 210; or the light chain comprises an amino acid sequence as set forth in SEQ ID NO: 141, and the heavy chain comprises an amino acid sequence as set forth in SEQ ID NO: 211.

In some embodiments, the antibodies or antigen binding fragments thereof for binding IL-13 are monoclonal antibodies.

In some embodiments, the antibodies or antigen binding fragments thereof for binding IL-13 are human antibodies.

In some embodiments, the antibodies or antigen binding fragments thereof for binding IL-13 are bispecific antibodies.

In some embodiments, the antigen binding fragment thereof is selected from the group consisting of Fab, Fab′, F(ab′)₂, Fd, Fv, scFv, a single-chain antibody, a minibody, and a diabody.

In some embodiments, the antigen binding fragment thereof is an scFv.

In an aspect, provided herein is a computer-implemented method comprising scoring a polypeptide comprising an amino acid sequence with a computationally binding optimized (CBO) model for a functional property relating to modulating the activity of a target molecule, the CBO model: for each amino acid position of the amino acid sequence of the polypeptide, calculating a plurality of energy scores, the energy scores based on the amino acid at a given position in the amino acid sequence, adding each energy score calculated to an array, generating a normalized sum of the energy scores for each position, and generating a score of the polypeptide by summing each energy score calculated, the score representing the functional property of the polypeptide, the functional property relating to the polypeptide modulating the activity of the target molecule.

In some embodiments, the computer-implemented method further comprises calculating a logarithm of each energy score calculated; wherein summing each energy score calculated is performed by summing the logarithms of each energy score calculated; wherein the energy scores are further calculated based on having substituted the amino acid at the given position in the amino acid sequence with each of a plurality of different amino acids.

In some embodiments, the scoring the polypeptide using the CBO model is implementable by the script of Appendix A, and the CBO model is substantially similar to the table of Appendix B.

In some embodiments, the functional property is at least one of: a binding affinity for an interleukin-13 (IL-13) polypeptide characterized by a K_{D} of 10 pM or less (optionally, as measured by KinExA); a binding specificity for the IL-13 polypeptide; a neutralizing activity against the IL-13 polypeptide (optionally, a full-length human IL-13); or an inhibitory activity against IL-13-mediated signaling, or a combination of the foregoing.

In some embodiments, the target molecule is interleukin-13 (IL-13), and the functional property of the relating to the polypeptide modulates the activity of the IL-13.

In an aspect, provided herein is a system comprising: a processor; and a memory with computer code instructions stored thereon, the processor and the memory, with the computer code instructions, being configured to cause the system to: score a polypeptide comprising an amino acid sequence with a CBO model for a functional property relating to modulating the activity of a target molecule, the CBO model: for each amino acid position of the amino acid sequence of the polypeptide, calculating a plurality of energy scores, the energy scores based on the amino acid at a given position in the amino acid sequence, add each energy score calculated to an array, generate a normalized sum of the energy scores for each position, and generate a score of the amino acid sequence by summing each energy score calculated, the score representing a functional property of the polypeptide, the functional property relating to the polypeptide modulating the activity of the target molecules.

In some embodiments, the instructions are further configured to cause the system to calculate a logarithm of each energy score calculated; wherein summing each energy score calculated is performed by summing the logarithms of each energy score calculated; wherein the energy scores are further based on having substituted the amino acid at the given position in the amino acid sequence with each of a plurality of different amino acids.

In some embodiments, scoring the polypeptide using the CBO model is implemented by the script of Appendix A and wherein the CBO model is substantially similar to the table of Appendix B.

In some embodiments, the functional property is at least one of: a binding affinity for an interleukin-13 (IL-13) polypeptide characterized by a K_{D} of 10 pM or less (optionally, as measured by KinExA); a binding specificity for the IL-13 polypeptide; a neutralizing activity against the IL-13 polypeptide (optionally, a full-length human IL-13); or an inhibitory activity against IL-13-mediated signaling, or a combination of the foregoing.

In some embodiments, the target molecule is interleukin-13 (IL-13), and the functional property of the relating to the polypeptide modulates the activity of the IL-13.

In an aspect, provided herein is a polypeptide that specifically binds an interleukin-13 (IL-13): wherein the polypeptide comprises an amino acid sequence that is assigned a score above a predetermined threshold by a computationally binding optimized (CBO) model upon scoring the amino acid sequence of the polypeptide.

In some embodiments, the polypeptide is scorable by the script of Computer Program Listing Appendix A and wherein the CBO model is calculated by a table substantially similar to that of Computer Program Listing Appendix B and wherein the polypeptide is assigned the score that is calculated using the Computer Program Listing Appendix B; wherein the polypeptide is assigned the score by a script substantially similar to Computer Program Listing Appendix A, the script employing the CBO model substantially similar to the table of Computer Program Listing Appendix B, wherein the polypeptide has a logarithmic score of at least about: −1.7, −1.0, −0.5, 0, 0.5, 1, 1.5, 1.8, 2.0, and 3.0.

In some embodiments, the polypeptide has one or more properties selected from: a binding affinity for an interleukin-13 (IL-13) polypeptide characterized by a K_{D} of 10 pM or less (optionally, as measured by KinExA); a binding specificity for the IL-13 polypeptide; a neutralizing activity against the IL-13 polypeptide (optionally, a full-length human IL-13); or an inhibitory activity against IL-13-mediated signaling, or a combination of the foregoing.

In some embodiments, the CBO model outputs a score that is equal to or above a score from the CBO model of one or more of a reference polypeptide that comprises a VL and VH pair selected from: SEQ ID NO: 97 and SEQ ID NO: 98 (AbA); SEQ ID NO: 99 and SEQ ID NO: 100 (AbB); SEQ ID NO: 101 and SEQ ID NO: 102 (AbC); SEQ ID NO: 103 and SEQ ID NO: 104 (AbD); SEQ ID NO: 105 and SEQ ID NO: 106 (AbE); SEQ ID NO: 107 and SEQ ID NO: 108 (AbF); SEQ ID NO: 109 and SEQ ID NO: 110 (AbG); SEQ ID NO: 111 and SEQ ID NO: 112 (AbH); SEQ ID NO: 113 and SEQ ID NO: 114 (AbI); SEQ ID NO: 115 and SEQ ID NO: 116 (AbJ); SEQ ID NO: 117 and SEQ ID NO: 118 (AbK); SEQ ID NO: 119 and SEQ ID NO: 120 (AbL); SEQ ID NO: 121 and SEQ ID NO: 122 (AbM); SEQ ID NO: 123 and SEQ ID NO: 124 (AbN); SEQ ID NO: 125 and SEQ ID NO: 126 (AbO); or SEQ ID NO: 127 and SEQ ID NO: 128 (AbP).

In some embodiments, the polypeptide does not comprise a heavy chain comprising SEQ ID NO: 179 or 181 and a light chain comprising SEQ ID NO: 178 or 180.

In an aspect, provided herein is a polypeptide: wherein the polypeptide comprises an amino acid sequence that is assigned a score above a predetermined threshold by a computationally binding optimized (CBO) model upon scoring the amino acid sequence of the polypeptide.

In an aspect, provided herein is a polypeptide that binds human interleukin-13 (IL-13), wherein the polypeptide is designed by a method comprising: generating a polypeptide sequence with a CBO model; verifying the generated polypeptide sequence using the CBO model by: for each amino acid position of the polypeptide sequence, calculating a plurality of energy scores, the energy scores based on having substituted the amino acid at a given position in the polypeptide sequence with each of a plurality of different amino acids, adding each energy score calculated to an array, generating a normalized sum of the energy scores for each position, calculating a logarithm of each energy score calculated, and generating a score of the polypeptide sequence by summing the logarithms of each energy score calculated, the score representing a functional property of the polypeptide's ability to bind to human IL-13.

### BRIEF DESCRIPTION OF THE DRAWINGS

The foregoing summary, as well as the following detailed description, will be better understood when read in conjunction with the appended figures. For the purpose of illustration, shown in the figures are embodiments. It should be understood, however, that the summary, detailed description, and figures are not limited to the precise arrangements, examples, and instrumentalities shown.

FIGS. 1A-F demonstrate anti-IL-13 antibodies neutralizing the signaling of IL-13 through IL-13Ra1/IL-4Ra expressed on HEK-Blue cells. IL-13Ra1/IL-4Ra HEK-Blue cells express STAT6 and a STAT6 inducible SEAP reporter that is induced by IL-13 binding to IL-13Ra1. Ref1 is a reference molecule and positive control. Data are analyzed by normalizing response to the IgG1 isotype negative control to obtain percent inhibition. IC₅₀ values are calculated by fitting a 4-parameter non-linear regression curve using the average of four technical replicates per antibody concentration.

FIG. 2 demonstrates an anti-IL-13 antibody blocking IL-13 and inhibiting pSTAT6 induced SEAP reporter activity in the HEK-Blue IL-4/IL-13 Assay. Results displayed are expressed in percent inhibition (n=3 technical replicates from one representative biological replicate), as compared to stimulated but unblocked cells (IL-13 alone), as mean+/−SEM.

FIG. 3 demonstrates an anti-IL-13 antibody blocking human IL-13 and prevent it from signaling through the IL-4Ra1/IL-13Ra1 complex and downstream pSTAT6 signal on human monocytes (n=3 technical replicates from Donor 3). Results displayed are expressed in percent inhibition of pSTAT6 signal, as compared to an isotype control, as mean+/−SEM.

FIG. 4 demonstrates an anti-IL-13 antibody blocking cynomolgus IL-13 and prevent it from signaling through the IL-4Ra1/IL-13Ra1 complex and downstream pSTAT6 signal on cynomolgus monocytes (n=3 technical replicates from Donor 1). Results displayed are expressed in percent inhibition of pSTAT6 signal, as compared to an isotype control, as mean+/−SEM.

FIG. 5 is a schematic illustrating an exemplary hIL-13 administration and anti-IL-13 antibody dosing schedule in C57BL/6 mice. Animals in the vehicle control group received PBS intranasally every two days (Days 0-10). All other groups were administered 20 μL of 0.25 mg/mL IL-13 (5 μg/mouse) intranasally over the same period. Antibodies were administered via intraperitoneal injection on Days-1 and 5. On Day 11, serum and bronchoalveolar lavage fluid (BALF) samples were collected for measurement of total IgE and eotaxin/CCL11 levels using ELISA. Cell counts and flow cytometry were performed on BALF samples. Lung tissues were assessed for mucus overproduction and goblet cell metaplasia using Periodic acid-Schiff (PAS) staining.

FIGS. 6A-D demonstrate 6A) total serum IgE levels (ng/ml), 6B) eotaxin/CCL11 levels (pg/mL) in bronchoalveolar lavage fluid (BALF), 6C), total BAL counts, and 6D) eosinophil cell counts in mice treated with 1 mg/kg, 5 mg/kg, or 25 mg/kg of an exemplary anti-IL-13 antibody of the present disclosure (Antibody AbAE), reference antibody (Ref1), or isotype control (Isotype) and dosed with hIL-13 according to the schedule depicted in FIG. 5. ns=not significant, ***p≤0.001, ****p≤0.0001.

FIGS. 7A-B depict representative images of lung sections and inflammatory endpoint histological scores collected from mice dosed according to the schedule depicted in FIG. 5. FIG. 7A shows micrographs of Periodic acid-Schiff (PAS)-stained lung sections collected from mice treated with 1 mg/kg, 5 mg/kg, or 25 mg/kg of an exemplary anti-IL-13 antibody of the present disclosure (Antibody AbAE), reference antibody (Ref1), or isotype control (Isotype) and dosed with hIL-13 according to the schedule depicted in FIG. 5. FIG. 7B shows PAS scores in lung sections collected from mice treated with 1 mg/kg, 5 mg/kg, or 25 mg/kg of an exemplary anti-IL-13 antibody of the present disclosure (Antibody AbAE), reference antibody (Ref1), or isotype control (Isotype) and dosed with hIL-13 according to the schedule depicted in FIG. 5. *p≤0.05, **p≤0.01, ****p≤0.0001.

FIG. 8 shows a heat map with shaded cells to indicate bidirectional, blocking, non-binding interactions (response≤0.1 nm) and unshaded cells to indicate bidirectional, non-blocking, binding interactions (>0.1 nm).

FIG. 9 shows the normalized responses of Antibody AbAB, Ref2, isotype-hIgG1, and isotype-hIgG1-YTE-TM binding to a panel of recombinant human Fcγ receptors at 10,000 nM. Antibody binding responses were normalized to human Fcγ receptor capture levels and reported as normalized binding responses of one experiment.

FIG. 10 shows the normalized responses of human C1q binding to Antibody AbAB, Ref2, isotype-hIgG4, isotype-hIgG1, and isotype-hIgG1-YTE-TM. The dotted line is positioned at 0.1 nm to indicate a non-binding interaction (≤0.1 nm) from a binding interaction (>0.1 nm).

FIG. 11 depicts a multiple sequence alignment of molecules that are near in sequence space (at most 4 mutations) to Antibody AbG but do not bind IL-13 with high affinity. The concatenated CDRs are shown on the top, with any mutations displayed in the alignment below corresponding to the antibody name.

### DETAILED DESCRIPTION

IL-13 is “type 2” cytokine that is upregulated in a variety of inflammatory diseases, such as asthma, urticaria, prurigo nodularis, nasal polyposis, and atopic dermatitis. In these type 2 inflammatory diseases IL-13 is implicated in the down regulation of epithelial derived proteins, such as filaggrin, resulting in loss of epithelial barrier integrity, tissue remodeling and subsequent increase in inflammation, pruritis, skin thickening and fibrosis as disease progresses. Despite development of treatments for IL-13 mediated diseases and disorders such treatments are inefficacious due to the requirement for repeated dosing every 2-4 weeks resulting in a lack of patient compliance.

Provided herein are anti-IL-13 antibodies or antigen binding fragments thereof (e.g., anti-human IL-13 antibodies or antigen binding fragments thereof) that have one or more advantages over conventional IL-13 antibodies including, for example, higher binding affinity, and/or an extended half-life, which may lead to reduced dosing frequency and/or improved patient compliance. Such antibodies or antigen binding fragments thereof may be useful as a monotherapy or in combination with one or more other standard of care therapies for the treatment and/or prevention of an inflammatory disease or disorder.

### Definitions

Definitions of certain terms to be used herein are provided. Unless defined otherwise, all technical and scientific terms used herein have the same meaning as is commonly understood.

The term “Interleukin-13”, or “IL-13” includes human IL-13, including the native-sequence polypeptide, isoforms, chimeric polypeptides, all homologs, fragments, and precursors of IL-13. An exemplary amino acid sequence for human IL-13 is provided in NCBI Reference Sequences: NG_012090.1 (SEQ ID NO: 161).

The term “antibody” herein is used in the broadest sense and encompasses various antibody structures, including but not limited to monoclonal antibodies, polyclonal antibodies, chimeric antibodies, humanized antibodies, human antibodies, and multi-specific antibodies (e.g., bispecific antibodies).

An exemplary antibody such as an IgG comprises two heavy chains (abbreviated herein as HC) and two light chains (abbreviated herein as LC). Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino terminus to carboxy terminus in the following order: FR1, CDR1, FR2, CDR2, FR3, CDR3, and FR4.

The hypervariable region generally encompasses amino acid residues from about amino acid residues 27-32 (LCDR1; “L” denotes light chain), 50-52 (LCDR2) and 89-97 (LCDR3) in the light chain variable region and around about 26-33 (HCDR1; “H” denotes heavy chain), 51-57 (HCDR2), and 93-102 (HCDR3) in the heavy chain variable region; (Kabat et al., NIH. 1:103-108, 324-331; 1991) and/or those residues forming a hypervariable loop (e.g., residues 27-32 (LCDR1), 50-52 (LCDR2) and 89-97 (LCDR3) in the light chain variable region and 26-33 (HCDR1), 51-57 (HCDR2) and 93-102 (HCDR3) in the heavy chain variable region; (Chothia and Lesk, J Mol Biol. 196:901-917; 1987) and/or those residues forming a hypervariable loop (e.g., residues 27-38 (CDR1), 56-65 (CDR2) and 105-117 (CDR3)) in light and heavy chain variable regions (Lefranc, The Immunologist. 7:132-136; 1999; Lefranc et al., Dev Comp Immunol. 27:55-77; 2003).

The term “monoclonal antibody” as used herein refers to an antibody obtained from a population of substantially homogeneous antibodies, e.g., the individual antibodies comprising the population are identical and/or bind the same epitope, except for possible variant antibodies, e.g., containing naturally occurring mutations and/or arising during production of a monoclonal antibody preparation (e.g., variants having a C-terminal lysine deletion). In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used may be made by a variety of techniques, including but not limited to the hybridoma method, recombinant DNA methods, phage display methods, and methods utilizing transgenic animals containing all or part of the human immunoglobulin loci, such methods and other exemplary methods for making monoclonal antibodies being described herein.

The term “chimeric” antibody refers to a recombinant antibody in which a portion of the heavy and/or light chain is identical with or homologous to corresponding sequences in antibodies derived from a particular species, or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. In addition, complementarity determining region grafting may be performed to alter certain properties of the antibody molecule including affinity or specificity. Typically, the variable domains are obtained from an antibody from an experimental animal (the “parental antibody”), such as a rodent, and the constant domain sequences are obtained from human antibodies, so that the resulting chimeric antibody can direct effector functions in a human subject and will be less likely to elicit an adverse immune response than the parental (e.g., mouse) antibody from which it is derived.

The term “humanized antibody” refers to an antibody that has been engineered to comprise one or more human framework regions in the variable region together with non-human (e.g., mouse, rat, or hamster) complementarity-determining regions of the heavy and/or light chain. In some embodiments, a humanized antibody comprises sequences that are entirely human except for the CDR regions. Humanized antibodies are typically less immunogenic to humans, relative to non-humanized antibodies, and thus offer therapeutic benefits in certain situations. Many examples exist of humanized antibodies and suitable techniques for their generation. See for example, Hwang et al., Methods. 36:35, 2005; Queen et al., Proc. Natl. Acad. Sci. USA. 86:10029-10033, 1989; Jones et al., Nature. 321:522-25, 1986, Riechmann et al., Nature. 332:323-27, 1988; Verhoeyen et al., Science. 239:1534-36, 1988; Orlandi et al., Proc. Natl. Acad. Sci. USA. 86:3833-37, 1989; U.S. Pat. Nos. 5,225,539; 5,530,101; 5,585,089; 5,693,761, 5,693,762; 6,180,370; and Selick et al. (WO 90/07861), each of which is incorporated herein by reference in its entirety.

A “human antibody” is an antibody that possesses an amino-acid sequence corresponding to that of an antibody capable of being produced by the human genome and/or has been made using any of the techniques for making human antibodies. This definition of a human antibody specifically excludes a humanized antibody comprising non-human antigen-binding residues. Human antibodies can be produced using various techniques, including methods described in Cole et al., Monoclonal Antibodies and Cancer Therapy. 27:77-96, 1985; Boemer et al., J. Immuno. 147(1):86-95; 1991. See also van Dijk and van de Winkel, Curr. Opin. Pharmacol. 5:368-74; 2001. Human antibodies can be prepared by administering the antigen to a transgenic animal that has been modified to produce such antibodies in response to antigenic challenge, but whose endogenous loci have been disabled, e.g., immunized HuMab mice (see, e.g., Lonberg et al., Nature. 368:856-859, 1994; WO 98/24884; WO 94/25585; WO 92/22645; U.S. Pat. No. 5,569,825 (WO 92/03918); and U.S. patent application Ser. No. 10/031,722 (WO 01/09187) regarding HuMab mice), xenomice (see, e.g., U.S. Pat. Nos. 5,569,825, 6,075,181 and 6,150,584 regarding XENOMOUSE™ technology).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The terms “antigen-binding domain” of an antibody (or simply “binding domain”) of an antibody or similar terms refer to one or more fragments of an antibody that retain the ability to specifically bind to an antigen complex. Examples of binding fragments encompassed within the term “antigen-binding portion” of an antibody include (i) Fab fragments, monovalent fragments consisting of the VL, VH, constant light (CL) and constant heavy (CH) domains; (ii) F(ab′)₂ fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., Nature. 341: 544-546; 1989), which consist of a VH domain; (vi) isolated complementarity determining regions, and (vii) combinations of two or more isolated CDRs which may optionally be joined by a synthetic linker.

The “variable domain” (V domain) or “variable region” of an antibody mediates binding and confers antigen specificity of a particular antibody. However, the variability is not evenly distributed across the 110-amino acid span of the variable domains. Instead, the V regions consist of relatively invariant stretches called framework regions of 15-30 amino acids separated by shorter regions of extreme variability referred to herein as “hypervariable regions” or CDRs that are each 9-12 amino acids long. The exact numbering and placement of the CDRs can be different among different numbering systems. However, a variable heavy and/or variable light sequence includes the associated CDRs. Accordingly, each variable heavy region includes the vhCDRs (e.g., vhCDR1 (also referred to herein as HCDR1), vhCDR2 (also referred to herein as HCDR2), and vhCDR3 (also referred to herein as HCDR3)) and each variable light region includes the vlCDRs (e.g., vlCDR1 (also referred to herein as LCDR1), vlCDR2 (also referred to herein as LCDR2), and vlCDR3 (also referred to herein as LCDR3)).

“Complementarity determining region” or “CDR” as the terms are used herein refer to short polypeptide sequences within the variable region of both heavy and light chain polypeptides that are primarily responsible for mediating specific antigen recognition. There are three CDRs (termed CDR1, CDR2, and CDR3) within each VL and each VH. Unless stated otherwise herein, CDR and framework regions are annotated according to the Kabat numbering scheme (Kabat et al., NIH. 1:103-108, 324-331; 1991).

In some embodiments, the CDRs of an antibody can be determined according to MacCallum et al., J Mol Biol. 262:732-745; 1996, herein incorporated by reference in its entirety or according to the IMGT numbering system as described in Lefranc, The Immunologist. 7:132-136; 1999 and Lefranc et al., Nucleic Acids Res. 27:209-212; 1999, each of which is herein incorporated by reference in its entirety. See also, e.g., Martin et al., Antibody Engineering. 31:422-439; 2001, herein incorporated by reference in its entirety. In some embodiments, the CDRs of an antibody can be determined according to the AbM numbering scheme, which refers to AbM hypervariable regions, which represent a compromise between the Kabat CDRs and Chothia structural loops and are used by Oxford Molecular's AbM antibody modeling software (Oxford Molecular Group, Inc.), herein incorporated by reference in its entirety.

“Framework” or “framework region” or “FR” refers to variable domain residues other than hypervariable region (HVR) residues. The FR of a variable domain generally consists of four FR domains: FR1, FR2, FR3, and FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., NIH. 1:103-108, 324-331; 1991. In some embodiments, for the VL, the subgroup is subgroup kappa I as in Kabat et al., supra. In some embodiments, for the VH, the subgroup is subgroup III as in Kabat et al., supra.

The “hinge region” is generally defined as stretching from 216-238 (EU numbering) or 226-251 (Kabat numbering) of human IgG1. The hinge can be further divided into three distinct regions, the upper, middle (e.g., core), and lower hinge.

The term “Fc region” herein is used to define a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., NIH. 1:103-108, 324-331; 1991.

An “antibody that binds to the same epitope” as a reference antibody (e.g., a reference IL-13 antibody) refers to an antibody that contacts an overlapping set of amino acid residues of the antigen as compared to the reference antibody or blocks binding of the reference antibody to its antigen in a competition assay by at least about 50%, about 50%, at least about 60%, about 60%, at least about 70%, about 70%, at least about 80%, about 80%, at least about 90%, about 90%, or more. The amino acid residues of an antibody that contact an antigen can be determined, for example, by determining the crystal structure of the antibody in complex with the antigen or by performing hydrogen/deuterium exchange. In some embodiments, residues of an antibody that are within 5 Å to the antigen are considered to contact the antigen. In some embodiments, residues of an antibody that are within 4 Å to the antigen are considered to contact the antigen. In some embodiments, residues of an antibody that make a non-covalent interaction with one or more residues of the antigen are considered to contact the antigen, including but not limited to hydrogen bonds, van der Waals interactions, salt bridges, Pi stacking interactions, hydrophobic interactions, or water-mediated hydrogen bonds.

In some embodiments, an antibody that binds to the same epitope as a reference antibody blocks binding of the reference antibody to its antigen in a competition assay by at least about 50%, about 50%, at least about 60%, about 60%, at least about 70%, about 70%, at least about 80%, about 80%, at least about 90%, about 90%, or more, and conversely, the reference antibody blocks binding of the antibody to its antigen in a competition assay by at least about 50%, about 50%, at least about 60%, about 60%, at least about 70%, about 70%, at least about 80%, about 80%, at least about 90%, about 90%, or more.

The term “antigen binding fragment” or “antibody fragment” refers to a molecule other than an intact antibody (e.g., a full-length antibody) that comprises a portion of an intact antibody that binds the antigen to which the intact antibody binds. Examples of antigen binding fragments include but are not limited to Fv, Fab, Fab′, Fab′-SH, F(ab)₂; diabodies; linear antibodies; single-chain antibody molecules (e.g., scFv). Papain digestion of antibodies produces two identical antigen-binding fragments, called “Fab” fragments, and a residual “Fc” fragment, a designation reflecting the ability to crystallize readily. The Fab fragment consists of an entire light (L) chain along with the variable region domain of the heavy (H) chain (VH), and the first constant domain of one heavy chain (CHI). Pepsin treatment of an antibody yields a single large F(ab)₂ fragment which roughly corresponds to two disulfide linked Fab fragments having divalent antigen-binding activity and is still capable of cross-linking antigen. Fab fragments differ from Fab′ fragments by having additional few residues at the carboxy terminus of the CHI domain including one or more cysteines from the antibody hinge region. Fab′-SH is the designation herein for Fab′ in which the cysteine residue(s) of the constant domains bear a free thiol group. F(ab′)₂ antigen binding fragments originally were produced as pairs of Fab′ fragments which have hinge cysteines between them. Other chemical couplings of antigen binding fragments are also known.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antigen binding fragments that comprise the VH and VL antibody domains connected into a single polypeptide chain. The sFv polypeptide may further comprise a linker (e.g., a polypeptide linker) between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “multispecific antibody” is used in the broadest sense and specifically covers an antibody comprising a heavy chain variable domain (VH) and a light chain variable domain (VL), where the VH-VL unit has polyepitopic specificity (e.g., is capable of binding to two different epitopes on one biological molecule or each epitope on a different biological molecule). Such multispecific antibodies include, but are not limited to, full-length antibodies, antibodies having two or more VL and VH domains, bispecific diabodies and triabodies. “Polyepitopic specificity” refers to the ability to specifically bind to two or more different epitopes on the same or different target(s).

“Dual specificity” or “bispecificity” refers to the ability to specifically bind to two different epitopes on the same or different target(s). However, in contrast to bispecific antibodies, dual-specific antibodies have two antigen-binding arms that are identical in amino acid sequence and each Fab arm can recognize two antigens. Dual specificity allows the antibodies to interact with high affinity with two different antigens as a single Fab or IgG molecule. According to some embodiments, the multispecific antibody in an IgG1 form binds to each epitope with an affinity of 5 μM to 0.001 pM, 3 μM to 0.001 pM, 1 μM to 0.001 pM, 0.5 μM to 0.001 pM, or 0.1 μM to 0.001 pM. “Monospecific” refers to the ability to bind only one epitope. Multi-specific antibodies can have structures similar to full immunoglobulin molecules and include Fc regions, for example IgG Fc regions.

As used herein, the term “bispecific antibody” refers to a monoclonal, often human or humanized, antibody that has binding specificities for at least two different antigens. One of the binding specificities can be directed towards IL-13, the other can be for any other antigen, e.g., for a cell-surface protein, receptor, receptor subunit, tissue-specific antigen, virally derived protein, virally encoded envelope protein, bacterially derived protein, or bacterial surface protein, etc.

As used herein, the term “diabody” refers to a bivalent antibody comprising two polypeptide chains, in which each polypeptide chain includes VH and VL domains joined by a linker that is too short (e.g., a linker composed of five amino acids) to allow for intramolecular association of VH and VL domains on the same peptide chain. This configuration forces each domain to pair with a complementary domain on another polypeptide chain so as to form a homodimeric structure. Accordingly, the term “triabody” refers to a trivalent antibody comprising three peptide chains, each of which contains one VH domain and one VL domain joined by a linker that is exceedingly short (e.g., a linker composed of 1-2 amino acids) to permit intramolecular association of VH and VL domains within the same peptide chain.

The term an “isolated antibody” or an “isolated antigen binding fragment” when used to describe the various antibodies or antigen binding fragments thereof provided herein, means an antibody or antigen binding fragment that has been identified and separated and/or recovered from a cell or cell culture from which it was expressed. An isolated antibody or antigen binding fragment may include variants of the antibody or antigen binding fragment having one or more co- or post-translational modifications that arise during production, purification, and/or storage of the antibody or antigen binding fragment. Contaminant components of its natural environment are materials that would typically interfere with diagnostic or therapeutic uses for the polypeptide, and can include enzymes, hormones, and other proteinaceous or non-proteinaceous solutes. In some embodiments, an isolated antibody is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC) approaches. For a review of methods for assessment of antibody purity, see, for example, Flatman et al., J Chromatogr. 848:79-87; 2007. In some embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or silver stain.

With regards to the binding of an antibody to a target molecule, the term “specific binding” or “specifically binds” or is “specific for” a particular polypeptide or protein such as IL-13 or an epitope on a particular polypeptide or protein target such as IL-13 means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target (e.g., inhibited by at least about 10%, about 10%, at least about 20%, about 20%, at least about 30%, about 30%, at least about 40%, about 40%, at least about 50%, about 50%, at least about 60%, about 60%, at least about 70%, about 70%, at least about 80%, about 80%, at least about 90%, about 90%, at least about 100%, or about 10%). The term “specific binding” or “specifically binds to” or is “specific for” a particular polypeptide or an epitope on a particular polypeptide target as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. Affinity and K_{D} values are inversely related. A high affinity for an antigen is measured by a low K_{D} value. In some embodiments, the term “specific binding” refers to binding where a molecule binds to IL-13 or to an IL-13 epitope without substantially binding to any other polypeptide or polypeptide epitope. As used herein, the term “binding” may refer to “specific binding” such that each and every occurrence of the term “binding” may be replaced with the term “specific binding.”

The term “affinity” as used herein, means the strength of the binding of an antibody to an epitope. The affinity of an antibody is given by the equilibrium dissociation constant K_{D}, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant K_{A} is defined by 1/K_{D}. Methods for determining the affinity of monoclonal antibodies (mAbs) can be found in Harlow, et al., Antibodies: A Laboratory Manual, Cold Spring Harbor Laboratory Press, Cold Spring Harbor, N.Y., (1988), Coligan et al., Current Protocols in Immunology, Greene Publishing Assoc, and Wiley Interscience, N.Y., (1992, 1993), and Muller, Meth. Enzymol. 92:589-601; 1983, which are entirely incorporated herein by reference. One standard method for determining the affinity of mAbs is the use of surface plasmon resonance (SPR) screening (such as by analysis with a BIAcore™ SPR analytical device).

An “epitope” indicates the site or sites of interaction between an antibody and its antigen(s). As described by (Janeway, C, Jr., P. Travers, et al., (2001). Immunobiology: the immune system in health and disease. Part II, Section 3-8. New York, Garland Publishing, Inc.): “An antibody generally recognizes only a small region on the surface of a large molecule such as a protein . . . [Certain epitopes] are likely to be composed of amino acids from different parts of the [antigen] polypeptide chain that have been brought together by protein folding. Antigenic determinants of this kind are known as conformational or discontinuous epitopes because the structure recognized is composed of segments of the protein that are discontinuous in the amino acid sequence of the antigen but are brought together in the three-dimensional structure. In contrast, an epitope composed of a single segment of polypeptide chain is termed a continuous or linear epitope” (Janeway, C. Jr., P. Travers, et al., (2001). Immunobiology: the immune system in health and disease. Part II, Section 3-8. New York, Garland Publishing, Inc.). An epitope may be a structural epitope or a functional epitope.

The term “K_{D}”, as used herein, refers to the equilibrium dissociation constant, which is obtained from the ratio of k_{off} to kₒₙ (e.g., k_{off}/kₒₙ) and is expressed as a molar concentration (M). K_{D} values for antibodies can be determined using well-established methods. Methods for determining the K_{D} of an antibody include biolayer interferometry (BLI) analysis, using a Fortebio Octet RED device, surface plasmon resonance, using a biosensor system such as a BIACORE® surface plasmon resonance system, or flow cytometry and Scatchard analysis.

“EC₅₀” with respect to an agent and a particular activity (e.g., binding to a cell, inhibition of enzymatic activity, activation, or inhibition of an immune cell), refers to the efficient concentration of the agent which produces 50% of its maximum response or effect with respect to such activity. “EC₁₀₀” with respect to an agent and a particular activity refers to the efficient concentration of the agent which produces its substantially maximum response with respect to such activity.

“IC₅₀” with respect to an agent and a particular activity (e.g., binding to a cell, inhibition of enzymatic activity, activation, or inhibition of an immune cell), refers to the inhibitory concentration of the agent which inhibits half of its maximum response or effect with respect to such activity. “IC₁₀₀” with respect to an agent and a particular activity refers to the inhibitory concentration of the agent which inhibits its substantially maximum response with respect to such activity.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., 1990).

As used herein, the term “paratope” refers to a set of amino acid residues in an antibody or an antigen-binding fragment thereof that contribute to a binding interaction with an epitope of a target protein. The binding interaction can be a hydrogen bond, a salt bridge, a van der Waal interaction, an ionic bond, or a combination thereof. A binding interaction may be direct, or indirect, e.g., via a coordinated intermediate molecule, such as an ion or water. The residues of a paratope, in some embodiments, comprise only residues that are part of a defined CDR. In other embodiments, the residues of a paratope further comprise one or more residues that are not part of a defined CDR. In some embodiments, a paratope is oriented less than about 5.0 angstroms from an epitope on a target antigen when a polypeptide is bound to the target antigen, e.g., less than about: 4.5, 4.0, 3.5, 3.0, 2.5, 2.4, 2.3, 2.2, 2.1, 2.0, 1.9, 1.8, 1.7, 1.6, 1.5, 1.4, 1.3, 1.2, 1.1, 1.0 or 0.9 angstroms, or about: 0.9-5.0, 0.9-4.8.1.0-5, 1.0-4.5, 1.0-4.0, 1.0-3.5, 1.1-3.5, 1.1-3.0, 1.2-3.0, 1.2-2.5, 1.3-2.5, 1.3-2.4, 1.4-2.4, 1.4-2.3, 1.5-2.3, 1.5-2.2, 1.6-2.2, 1.6-2.1, 1.7-2.1, 1.7-2.0 or 1.8-2.0 angstroms, from the epitope. In some embodiments, less than all of the amino acid residues constituting a paratope (e.g., about 40%, about 50%, about 60%, about 70%, about 80%, about 90%, about 95%, about 96%, about 97%, about 98%, or about 99% of the amino acid residues) in the paratope are oriented less than about 5.0 angstroms from an epitope on a target antigen when a polypeptide is bound to the target antigen.

### Anti-IL-13 Antibodies and Antigen Binding Fragments Thereof

Provided herein are anti-IL-13 antibodies and antigen binding fragments thereof. In some embodiments, the anti-IL-13 antibodies or antigen binding fragments thereof bind to IL-13 including, for example, human IL-13 (SEQ ID NO: 161). As used herein, the term “anti-IL-13 antibodies and antigen binding fragments thereof” is used interchangeably with the terms “polypeptides” or “proteins” such that the term “polypeptides” or “proteins” can replace each and every occurrence of the term “anti-IL-13 antibodies and antigen binding fragments thereof.”

The anti-IL-13 antibody and antibody fragments described herein may comprise heavy and/or light chain CDRs from AbA, AbB, AbC, AbD, AbE, AbF, AbG, AbH, AbI, AbJ, AbK, AbL, AbM, AbN, AbO, or AbP. In some embodiments, the CDRs of an anti-IL-13 antibody or antigen binding fragment thereof can be determined according to the Kabat system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). In other embodiments, the CDRs of an anti-IL-13 antibody or antigen binding fragment thereof can be determined according to the Chothia system, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1):175-82; and U.S. Pat. No. 7,709,226). In yet other embodiments, the CDRs of an anti-IL-13 antibody or antigen binding fragment thereof can be determined according to the ImMunoGeneTics (IMGT) system, which will be referred to herein as the “IMGT CDRs”, for example, as described in Lefranc, M.-P., 1999, The Immunologist, 7:132-136 and Lefranc, M.-P. et al., 1999, Nucleic Acids Res., 27:209-212. In still further embodiments, the CDRs of an anti-IL-13 antibody or antigen binding fragment thereof can be determined according to the AbM system, which will be referred to herein as the “AbM CDRs,” for example as described in MacCallum et al., 1996, J. Mol. Biol., 262:732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001). In other embodiments, the CDRs of an anti-IL-13 antibody or antigen binding fragment thereof can be determined according to the Contact system, which will be referred to herein as the “Contact CDRs” (see, e.g., MacCallum R M et al., 1996, J Mol Biol 5:732-745). The Contact CDRs are based on an analysis of the available complex crystal structures.

In some embodiments, the anti-IL-13 antibodies or antigen binding fragments thereof comprise a VH having a set of CDRs (HCDR1, HCDR2, and HCDR3) provided in Table 1. In some embodiments, the antibodies or antigen binding fragments thereof that bind IL-13 comprise a VH having a set of CDRs (HCDR1, HCDR2, and HCDR3) provided in Table 1. In some embodiments, the anti-IL-13 antibody or antigen binding fragment thereof comprises a VH having a set of CDRs (HCDR1, HCDR2, and HCDR3), provided in Table 1, wherein the anti-IL-13 antibody or antigen binding fragment thereof binds IL-13. For example, the anti-IL-13 antibody or antigen binding fragment thereof may comprise a set of CDRs corresponding to those CDRs in one or more of the anti-IL-13 antibodies provided in Table 1 (e.g., the CDRs of AbA, AbB, AbC. AbD, AbE, AbF, Antibody AbG, AbH, AbI, AbJ, AbK, AbL, AbM, AbN, AbO, or AbP). The anti-IL-13 antibody or antigen binding fragment thereof that binds IL-13 may also comprise a set of CDRs corresponding to those CDRs in one or more of the anti-IL-13 antibodies provided in Table 1 (e.g., the CDRs of AbA, AbB, AbC, AbD, AbE, AbF, AbG, AbH, AbI, AbJ, AbK, AbL, AbM, AbN, AbO, or AbP). For another example, the anti-IL-13 antibody or antigen binding fragment thereof may comprise a set of CDRs corresponding to those CDRs in one or more of the anti-IL-13 antibodies provided in Table 1 (e.g., the CDRs of AbA, AbB, AbC, AbD, AbE, AbF, AbG, AbH, AbI, AbJ, AbK, AbL, AbM, AbN, AbO, or AbP), wherein the anti-IL-13 antibody or antibody fragment thereof binds IL-13.

TABLE 1
CDR Sequences of Anti-IL-13 Antibody Variable Heavy Chains According
to Kabat Numbering

| Anti-IL-13 Ab | HCDR1 | HCDR2 | HCDR3 |
|---|---|---|---|
| AbA | AYSVN | MIWGDGKIVYNSALKS | DGYYPYAMDN |
| | (SEQ ID NO: 4) | (SEQ ID NO: 5) | (SEQ ID NO: 6) |
| AbB | AYSVN | MIWGDGKIVYNSALKS | DGYYPYAMDN |
| | (SEQ ID NO: 10) | (SEQ ID NO: 11) | (SEQ ID NO: 12) |
| AbC | AYSVN | MIWGDGKIVYNSALKS | DGYYPYAMDY |
| | (SEQ ID NO: 16) | (SEQ ID NO: 17) | (SEQ ID NO: 18) |
| AbD | AYSVN | MIWGDGKVVYNSALKS | DGYFPYAMDN |
| | (SEQ ID NO: 22) | (SEQ ID NO: 23) | (SEQ ID NO: 24) |
| AbE | AYSVN | MIWGDGKIVYNSALKS | DGYFPYAMDN |
| | (SEQ ID NO: 28) | (SEQ ID NO: 29) | (SEQ ID NO: 30) |
| AbF | RYSVN | MIWGDAKIVYNSALKS | DGYFPYAMDN |
| | (SEQ ID NO: 34) | (SEQ ID NO: 35) | (SEQ ID NO: 36) |
| AbG | AYSVN | MIWGDGKIVYNSALKS | DGYFPYAMDN |
| | (SEQ ID NO: 40) | (SEQ ID NO: 41) | (SEQ ID NO: 42) |
| AbH | RYSVN | MIWGDGKIVYNSALKS | DGYYPYAMDN |
| | (SEQ ID NO: 46) | (SEQ ID NO: 47) | (SEQ ID NO: 48) |
| AbI | RYSVN | MIWGDGKIVYNSALKS | DGFYPYAMDN |
| | (SEQ ID NO: 52) | (SEQ ID NO: 53) | (SEQ ID NO: 54) |
| AbJ | AYSVN | MIWGDRKKVYNSALKS | DGYFPYAMDL |
| | (SEQ ID NO: 58) | (SEQ ID NO: 59) | (SEQ ID NO: 60) |
| AbK | RYSVN | MIWGDGKIVYNSALKS | DGYYPYAMDN |
| | (SEQ ID NO: 64) | (SEQ ID NO: 65) | (SEQ ID NO: 66) |
| AbL | RYSVN | MIWGDGKIVYNSALKS | DGYYPYAMDN |
| | (SEQ ID NO: 70) | (SEQ ID NO: 71) | (SEQ ID NO: 72) |
| AbM | AYSVN | MIWGDGKIVYNSALKS | DGYFPYAMDN |
| | (SEQ ID NO: 76) | (SEQ ID NO: 77) | (SEQ ID NO: 78) |
| AbN | AYSVN | MIWGDGKIVYNSALKS | DGYYPYAMDN |
| | (SEQ ID NO: 82) | (SEQ ID NO: 83) | (SEQ ID NO: 84) |
| AbO | AYSVN | MIWGDGKIVYNSALKS | DGYFPYAMDV |
| | (SEQ ID NO: 88) | (SEQ ID NO: 88) | (SEQ ID NO: 90) |
| AbP | AYSVN | MIWGDGKVVYNSALKS | DGYFPYAMDH |
| | (SEQ ID NO: 94) | (SEQ ID NO: 95) | (SEQ ID NO: 96) |

In some embodiments, the anti-IL-13 antibodies or antigen binding fragments thereof comprise a VL having a set of CDRs (LCDR1, LCDR2, and LCDR3) as provided in Table 2. In some embodiments, the antibodies or antigen binding fragments thereof that bind IL-13 comprise a VL having a set of CDRs (LCDR1, LCDR2, and LCDR3) as provided in Table 2. In some embodiments, the anti-IL-13 antibody or antigen binding fragment thereof comprises a VL having a set of CDRs (LCDR1, LCDR2, and LCDR3) wherein the anti-IL-13 antibody or antigen binding fragment thereof binds IL-13, as provided in Table 2. For example, the anti-IL-13 antibody or antigen binding fragment thereof may comprise a set of CDRs corresponding to those CDRs in one or more of the anti-IL-13 antibodies provided in Table 2 (e.g., the CDRs of AbA, AbB, AbC, AbD, AbE, AbF, AbG, AbH, AbI, AbJ, AbK, AbL, AbM, AbN, AbO, or AbP). The antibody or antigen binding fragment thereof that binds IL-13 may also comprise a set of CDRs corresponding to those CDRs in one or more of the antibodies provided in Table 2 (e.g., the CDRs of AbA, AbB, AbC, AbD, AbE, AbF, AbG, AbH, AbI, AbJ, AbK, AbL, AbM, AbN, AbO, or AbP). For another example, the anti-IL-13 antibody or antigen binding fragment thereof may comprise a set of CDRs corresponding to those CDRs in one or more of the anti-IL-13 antibodies provided in Table 2 (e.g., the CDRs of AbA, AbB, AbC, AbD, AbE, AbF, AbG, AbH, AbI, AbJ, AbK, AbL, AbM, AbN, AbO, or AbP), wherein the anti-IL-13 antibody or antigen binding fragment thereof binds IL-13.

TABLE 2
CDR Sequences of Anti-IL-13 Antibody Variable Light Chains According
to Kabat Numbering

| Anti-IL-13 Ab | LCDR1 | LCDR2 | LCDR3 |
|---|---|---|---|
| AbA | RASKSVDSYGRSYMH | LASNLES | QQNNEDPRT |
| | (SEQ ID NO: 1) | (SEQ ID NO: 2) | (SEQ ID NO: 3) |
| AbB | RASKSVDYYGHSYMH | LASNLES | QQNNEDPRT |
| | (SEQ ID NO: 7) | (SEQ ID NO: 8) | (SEQ ID NO: 9) |
| AbC | RASKSVDYYGNSYMH | LASNLES | QQNNEDPRT |
| | (SEQ ID NO: 13) | (SEQ ID NO: 14) | (SEQ ID NO: 15) |
| AbD | RASKSVDSYGNSYMH | LASNLES | QQNNEDPRT |
| | (SEQ ID NO: 19) | (SEQ ID NO: 20) | (SEQ ID NO: 21) |
| AbE | RASKSVDSYGHSYMH | WASNLES | QQNNEDPRT |
| | (SEQ ID NO: 25) | (SEQ ID NO: 26) | (SEQ ID NO: 27) |
| AbF | RASESVDSYGNSYMH | LASNLES | QQNNEDPRT |
| | (SEQ ID NO: 31) | (SEQ ID NO: 32) | (SEQ ID NO: 33) |
| AbG | RASKSVDSYGNSYMH | LASNLES | QQNNEDPRT |
| | (SEQ ID NO: 37) | (SEQ ID NO: 38) | (SEQ ID NO: 39) |
| AbH | RASKSVDSYGNSYMH | RASNLES | QQNNEDPRT |
| | (SEQ ID NO: 43) | (SEQ ID NO: 44) | (SEQ ID NO: 45) |
| AbI | RASKSVDSYGNSYMH | LASNLES | QQNNEDPRT |
| | (SEQ ID NO: 49) | (SEQ ID NO: 50) | (SEQ ID NO: 51) |
| AbJ | RASKSVDSYGHSYMH | WASNLES | QQNNEDPRT |
| | (SEQ ID NO: 55) | (SEQ ID NO: 56) | (SEQ ID NO: 57) |
| AbK | RASQSVDSYGNSYMH | LASNLES | QQNNEDPRT |
| | (SEQ ID NO: 61) | (SEQ ID NO: 62) | (SEQ ID NO: 63) |
| AbL | RASKSVDSYGRSYMH | LASNLES | QQNNEDPRT |
| | (SEQ ID NO: 67) | (SEQ ID NO: 68) | (SEQ ID NO: 69) |
| AbM | RASKSVDYYGRSYMH | LASNLES | QQNNEDPRT |
| | (SEQ ID NO: 73) | (SEQ ID NO: 74) | (SEQ ID NO: 75) |
| AbN | RASESVDYYGHSYMH | LASNLES | QQNNEDPRT |
| | (SEQ ID NO: 79) | (SEQ ID NO: 80) | (SEQ ID NO: 81) |
| AbO | RASKSVDYYGHSYMH | LASNLES | QQNNEDPRT |
| | (SEQ ID NO: 85) | (SEQ ID NO: 86) | (SEQ ID NO: 87) |
| AbP | RASKSVDSYGNSYMH | WASNLES | QQNNEDPRT |
| | (SEQ ID NO: 91) | (SEQ ID NO: 92) | (SEQ ID NO: 93) |

In some embodiments, the anti-IL-13 antibody or antigen binding fragment thereof comprises a VH having a set of CDRs (HCDR1, HCDR2, and HCDR3) as provided in Table 1, and a VL having a set of CDRs (LCDR1, LCDR2, and LCDR3) as provided in Table 2.

In some embodiments, the antibody may be a monoclonal, chimeric, bispecific, humanized, or human antibody. In some embodiments, the anti-IL-13 antibody or antigen binding fragment thereof comprises all six of the CDR regions of AbA, AbB, AbC, AbD, AbE, AbF, AbG, AbH, AbI, AbJ, AbK, AbL, AbM, AbN, AbO, or AbP.

In some embodiments, the anti-IL-13 antibody or antigen binding fragment thereof comprises a VH having a set of complementarity-determining regions (CDR1, CDR2, and CDR3) selected from the group consisting of:
(i) CDR1: SEQ ID NO: 4, CDR2: SEQ ID NO: 5, CDR3: SEQ ID NO: 6;
(ii) CDR1: SEQ ID NO: 10, CDR2: SEQ ID NO: 11, CDR3: SEQ ID NO: 12;
(iii) CDR1: SEQ ID NO: 16, CDR2: SEQ ID NO: 17, CDR3: SEQ ID NO: 18;
(iv) CDR1: SEQ ID NO: 22, CDR2: SEQ ID NO: 23, CDR3: SEQ ID NO: 24;
(v) CDR1: SEQ ID NO: 28, CDR2: SEQ ID NO: 29, CDR3: SEQ ID NO: 30;
(vi) CDR1: SEQ ID NO: 34, CDR2: SEQ ID NO: 35, CDR3: SEQ ID NO: 36;
(vii) CDR1: SEQ ID NO: 40, CDR2: SEQ ID NO: 41, CDR3: SEQ ID NO: 42;
(viii) CDR1: SEQ ID NO: 46, CDR2: SEQ ID NO: 47, CDR3: SEQ ID NO: 48;
(ix) CDR1: SEQ ID NO: 52, CDR2: SEQ ID NO: 53, CDR3: SEQ ID NO: 54;
(x) CDR1: SEQ ID NO: 58, CDR2: SEQ ID NO: 59, CDR3: SEQ ID NO: 60;
(xi) CDR1: SEQ ID NO: 64, CDR2: SEQ ID NO: 65, CDR3: SEQ ID NO: 66;
(xii) CDR1: SEQ ID NO: 70, CDR2: SEQ ID NO: 71, CDR3: SEQ ID NO: 72;
(xiii) CDR1: SEQ ID NO: 76, CDR2: SEQ ID NO: 77, CDR3: SEQ ID NO: 78;
(xiv) CDR1: SEQ ID NO: 82, CDR2: SEQ ID NO: 83, CDR3: SEQ ID NO: 84;
(xv) CDR1: SEQ ID NO: 88, CDR2: SEQ ID NO: 89, CDR3: SEQ ID NO: 90; and
(xvi) CDR1: SEQ ID NO: 94, CDR2: SEQ ID NO: 95, CDR3: SEQ ID NO: 96.

In some embodiments, the anti-IL-13 antibody or antigen binding fragment thereof comprises a VL having a set of complementarity-determining regions (CDR1, CDR2, and CDR3) selected from the group consisting of:
(i) CDR1: SEQ ID NO: 1, CDR2: SEQ ID NO: 2, CDR3: SEQ ID NO: 3;
(ii) CDR1: SEQ ID NO: 7, CDR2: SEQ ID NO: 8, CDR3: SEQ ID NO: 9;
(iii) CDR1: SEQ ID NO: 13, CDR2: SEQ ID NO: 14, CDR3: SEQ ID NO: 15;
(iv) CDR1: SEQ ID NO: 19, CDR2: SEQ ID NO: 20, CDR3: SEQ ID NO: 21;
(v) CDR1: SEQ ID NO: 25, CDR2: SEQ ID NO: 26, CDR3: SEQ ID NO: 27;
(vi) CDR1: SEQ ID NO: 31, CDR2: SEQ ID NO: 32, CDR3: SEQ ID NO: 33;
(vii) CDR1: SEQ ID NO: 37, CDR2: SEQ ID NO: 38, CDR3: SEQ ID NO: 39;
(viii) CDR1: SEQ ID NO: 43, CDR2: SEQ ID NO: 44, CDR3: SEQ ID NO: 45;
(ix) CDR1: SEQ ID NO: 49, CDR2: SEQ ID NO: 50, CDR3: SEQ ID NO: 51;
(x) CDR1: SEQ ID NO: 55, CDR2: SEQ ID NO: 56, CDR3: SEQ ID NO: 57;
(xi) CDR1: SEQ ID NO: 61, CDR2: SEQ ID NO: 62, CDR3: SEQ ID NO: 63;
(xii) CDR1: SEQ ID NO: 67, CDR2: SEQ ID NO: 68, CDR3: SEQ ID NO: 69;
(xiii) CDR1: SEQ ID NO: 73, CDR2: SEQ ID NO: 74, CDR3: SEQ ID NO: 75;
(xiv) CDR1: SEQ ID NO: 79, CDR2: SEQ ID NO: 80, CDR3: SEQ ID NO: 81;
(xv) CDR1: SEQ ID NO: 85, CDR2: SEQ ID NO: 86, CDR3: SEQ ID NO: 87; and
(xvi) CDR1: SEQ ID NO: 91, CDR2: SEQ ID NO: 92, CDR3: SEQ ID NO: 93.

In some embodiments, the anti-IL-13 antibody or antigen binding fragment thereof comprises:
(a) a VH having a set of complementarity-determining regions (CDR1, CDR2, and CDR3) selected from the group consisting of:
   (i) CDR1: SEQ ID NO: 4, CDR2: SEQ ID NO: 5, CDR3: SEQ ID NO: 6;
   (ii) CDR1: SEQ ID NO: 10, CDR2: SEQ ID NO: 11, CDR3: SEQ ID NO: 12;
   (iii) CDR1: SEQ ID NO: 16, CDR2: SEQ ID NO: 17, CDR3: SEQ ID NO: 18;
   (iv) CDR1: SEQ ID NO: 22, CDR2: SEQ ID NO: 23, CDR3: SEQ ID NO: 24;
   (v) CDR1: SEQ ID NO: 28, CDR2: SEQ ID NO: 29, CDR3: SEQ ID NO: 30;
   (vi) CDR1: SEQ ID NO: 34, CDR2: SEQ ID NO: 35, CDR3: SEQ ID NO: 36;
   (vii) CDR1: SEQ ID NO: 40, CDR2: SEQ ID NO: 41, CDR3: SEQ ID NO: 42;
   (viii) CDR1: SEQ ID NO: 46, CDR2: SEQ ID NO: 47, CDR3: SEQ ID NO: 48;
   (ix) CDR1: SEQ ID NO: 52, CDR2: SEQ ID NO: 53, CDR3: SEQ ID NO: 54;
   (x) CDR1: SEQ ID NO: 58, CDR2: SEQ ID NO: 59, CDR3: SEQ ID NO: 60;
   (xi) CDR1: SEQ ID NO: 64, CDR2: SEQ ID NO: 65, CDR3: SEQ ID NO: 66;
   (xii) CDR1: SEQ ID NO: 70, CDR2: SEQ ID NO: 71, CDR3: SEQ ID NO: 72;
   (xiii) CDR1: SEQ ID NO: 76, CDR2: SEQ ID NO: 77, CDR3: SEQ ID NO: 78;
   (xiv) CDR1: SEQ ID NO: 82, CDR2: SEQ ID NO: 83, CDR3: SEQ ID NO: 84;
   (xv) CDR1: SEQ ID NO: 88, CDR2: SEQ ID NO: 89, CDR3: SEQ ID NO: 90; and
   (xvi) CDR1: SEQ ID NO: 94, CDR2: SEQ ID NO: 95, CDR3: SEQ ID NO: 96; and
(b) a VL having a set of complementarity-determining regions (CDR1, CDR2, and CDR3) selected from the group consisting of:
   (i) CDR1: SEQ ID NO: 1, CDR2: SEQ ID NO: 2, CDR3: SEQ ID NO: 3;
   (ii) CDR1: SEQ ID NO: 7, CDR2: SEQ ID NO: 8, CDR3: SEQ ID NO: 9;
   (iii) CDR1: SEQ ID NO: 13, CDR2: SEQ ID NO: 14, CDR3: SEQ ID NO: 15;
   (iv) CDR1: SEQ ID NO: 19, CDR2: SEQ ID NO: 20, CDR3: SEQ ID NO: 21;
   (v) CDR1: SEQ ID NO: 25, CDR2: SEQ ID NO: 26, CDR3: SEQ ID NO: 27;
   (vi) CDR1: SEQ ID NO: 31, CDR2: SEQ ID NO: 32, CDR3: SEQ ID NO: 33;
   (vii) CDR1: SEQ ID NO: 37, CDR2: SEQ ID NO: 38, CDR3: SEQ ID NO: 39;
   (viii) CDR1: SEQ ID NO: 43, CDR2: SEQ ID NO: 44, CDR3: SEQ ID NO: 45;
   (ix) CDR1: SEQ ID NO: 49, CDR2: SEQ ID NO: 50, CDR3: SEQ ID NO: 51;
   (x) CDR1: SEQ ID NO: 55, CDR2: SEQ ID NO: 56, CDR3: SEQ ID NO: 57;
   (xi) CDR1: SEQ ID NO: 61, CDR2: SEQ ID NO: 62, CDR3: SEQ ID NO: 63;
   (xii) CDR1: SEQ ID NO: 67, CDR2: SEQ ID NO: 68, CDR3: SEQ ID NO: 69;
   (xiii) CDR1: SEQ ID NO: 73, CDR2: SEQ ID NO: 74, CDR3: SEQ ID NO: 75;
   (xiv) CDR1: SEQ ID NO: 79, CDR2: SEQ ID NO: 80, CDR3: SEQ ID NO: 81;
   (xv) CDR1: SEQ ID NO: 85, CDR2: SEQ ID NO: 86, CDR3: SEQ ID NO: 87; and
   (xvi) CDR1: SEQ ID NO: 91, CDR2: SEQ ID NO: 92, CDR3: SEQ ID NO: 93.

In some embodiments, the anti-IL-13 antibody or antigen binding fragment thereof comprises a VH and a VL having a set of complementarity-determining regions (CDR1, CDR2 and CDR3) selected from the group consisting of:
(i) VH: CDR1: SEQ ID NO: 4, CDR2: SEQ ID NO: 5, CDR3: SEQ ID NO: 6, VL: CDR1: SEQ ID NO: 1, CDR2: SEQ ID NO: 2, CDR3: SEQ ID NO: 3;
(ii) VH: CDR1: SEQ ID NO: 10, CDR2: SEQ ID NO: 11, CDR3: SEQ ID NO: 12, VL: CDR1: SEQ ID NO: 7, CDR2: SEQ ID NO: 8, CDR3: SEQ ID NO: 9;
(iii) VH: CDR1: SEQ ID NO: 16, CDR2: SEQ ID NO: 17, CDR3: SEQ ID NO: 18, VL: CDR1: SEQ ID NO: 13, CDR2: SEQ ID NO: 14, CDR3: SEQ ID NO: 15; and
(iv) VH: CDR1: SEQ ID NO: 22, CDR2: SEQ ID NO: 23, CDR3: SEQ ID NO: 24, VL: CDR1: SEQ ID NO: 19, CDR2: SEQ ID NO: 20, CDR3: SEQ ID NO: 21.
(v) VH: CDR1: SEQ ID NO: 28, CDR2: SEQ ID NO: 29, CDR3: SEQ ID NO: 30, VL: CDR1: SEQ ID NO: 25, CDR2: SEQ ID NO: 26, CDR3: SEQ ID NO: 27;
(vi) VH: CDR1: SEQ ID NO: 34, CDR2: SEQ ID NO: 35, CDR3: SEQ ID NO: 36, VL: CDR1: SEQ ID NO: 31, CDR2: SEQ ID NO: 32, CDR3: SEQ ID NO: 33;
(vii) VH: CDR1: SEQ ID NO: 40, CDR2: SEQ ID NO: 41, CDR3: SEQ ID NO: 42, VL: CDR1: SEQ ID NO: 37, CDR2: SEQ ID NO: 38, CDR3: SEQ ID NO: 39;
(viii) VH: CDR1: SEQ ID NO: 46, CDR2: SEQ ID NO: 47, CDR3: SEQ ID NO: 48, VL: CDR1: SEQ ID NO: 43, CDR2: SEQ ID NO: 44, CDR3: SEQ ID NO: 45;
(ix) VH: CDR1: SEQ ID NO: 52, CDR2: SEQ ID NO: 53, CDR3: SEQ ID NO: 54, VL: CDR1: SEQ ID NO: 49, CDR2: SEQ ID NO: 50, CDR3: SEQ ID NO: 51;
(x) VH: CDR1: SEQ ID NO: 58, CDR2: SEQ ID NO: 59, CDR3: SEQ ID NO: 60, VL: CDR1: SEQ ID NO: 55, CDR2: SEQ ID NO: 56, CDR3: SEQ ID NO: 57;
(xi) VH: CDR1: SEQ ID NO: 64, CDR2: SEQ ID NO: 65, CDR3: SEQ ID NO: 66, VL: CDR1: SEQ ID NO: 61, CDR2: SEQ ID NO: 62, CDR3: SEQ ID NO: 63;
(xii) VH: CDR1: SEQ ID NO: 70, CDR2: SEQ ID NO: 71, CDR3: SEQ ID NO: 72, VL: CDR1: SEQ ID NO: 67, CDR2: SEQ ID NO: 68, CDR3: SEQ ID NO: 69;
(xiii) VH: CDR1: SEQ ID NO: 76, CDR2: SEQ ID NO: 77, CDR3: SEQ ID NO: 78, VL: CDR1: SEQ ID NO: 73, CDR2: SEQ ID NO: 74, CDR3: SEQ ID NO: 75;
(xiv) VH: CDR1: SEQ ID NO: 82, CDR2: SEQ ID NO: 83, CDR3: SEQ ID NO: 84, VL: CDR1: SEQ ID NO: 79, CDR2: SEQ ID NO: 80, CDR3: SEQ ID NO: 81;
(xv) VH: CDR1: SEQ ID NO: 88, CDR2: SEQ ID NO: 89, CDR3: SEQ ID NO: 90, VL: CDR1: SEQ ID NO: 85, CDR2: SEQ ID NO: 86, CDR3: SEQ ID NO: 87; and
(xvi) VH: CDR1: SEQ ID NO: 94, CDR2: SEQ ID NO: 95, CDR3: SEQ ID NO: 96 CDR1: SEQ ID NO: 91, CDR2: SEQ ID NO: 92, CDR3: SEQ ID NO: 93.

In some embodiments, the anti-IL-13 antibody or antigen binding fragment thereof comprises a variable heavy chain sequence selected from the group consisting of: SEQ ID NO: 97, 99, 101, 103, 105, 107, 109, 111, 113, 115, 117, 119, 121, 123, 125, and 127; and/or a variable light chain sequence selected from the group consisting of: SEQ ID NO: 98, 100, 102, 104, 106, 108, 110, 112, 114, 116, 118, 120, 122, 124, 126, and 128.

In some embodiments, the anti-IL-13 antibody or antigen binding fragment thereof comprises a variable heavy chain (VH) and a variable light chain (VL) as provided in Table 3.

TABLE 3
Variable Heavy and Variable Light Chain Sequences of Anti-IL-13 Antibodies

| Anti-IL-13 Ab | VH | VL |
|---|---|---|
| AbA | QVTLRESGPALVKPTQTLTLTCTVSG | DIVMTQSPDSLSVSLGERATINCR |
| | FSLSAYSVNWIRQPPGKALEWLAMI | ASKSVDSYGRSYMHWYQQKPGQ |
| | WGDGKIVYNSALKSRLTISKDTSKNQ | PPKLLIYLASNLESGVPDRFSGSG |
| | VVLTMTNMDPVDTATYYCAGDGYY | SGTDFTLTISSLQAEDVAVYYCQQ |
| | PYAMDNWGQGSLVTVSS (SEQ ID | NNEDPRTFGGGTKVEIK (SEQ ID |
| | NO: 98) | NO: 97) |
| AbB | QVTLRESGPALVKPTQTLTLTCTVSG | DIVMTQSPDSLSVSLGERATINCR |
| | ASLSAYSVNWIRQPPGKALEWLAMI | ASKSVDYYGHSYMHWYQQKPGQ |
| | WGDGKIVYNSALKSRLTISKDTSKNQ | PPKLLIYLASNLESGVPDRFSGSG |
| | VVLTMTNMDPVDTATYYCAGDGYY | SGTDFTLTISSLQAEDVAVYYCQQ |
| | PYAMDNWGQGSLVTVSS (SEQ ID | NNEDPRTFGGGTKVEIK (SEQ ID |
| | NO: 100) | NO: 99) |
| AbC | QVTLRESGPALVKPTQTLTLTCTVSG | DIVMTQSPDSLSVSLGERATINCR |
| | FSLSAYSVNWIRQPPGKALEWLAMI | ASKSVDYYGNSYMHWYQQKPGQ |
| | WGDGKIVYNSALKSRLTISKDTSKNQ | PPKLLIYLASNLESGVPDRFSGSG |
| | VVLTMTNMDPVDTATYYCAGDGYY | SGTDFTLTISSLQAEDVAVYYCQQ |
| | PYAMDYWGQGSLVTVSS (SEQ ID | NNEDPRTFGGGTKVEIK (SEQ ID |
| | NO: 102) | NO: 101) |
| AbD | QVTLRESGPALVKPTQTLTLTCTVSG | DIVMTQSPDSLSVSLGERATINCR |
| | SSLSAYSVNWIRQPPGKALEWLAMI | ASKSVDSYGNSYMHWYQQKPGQ |
| | WGDGKVVYNSALKSRLTISKDTSKN | PPKLLIYLASNLESGVPDRFSGSG |
| | QVVLTMTNMDPVDTATYYCAGDGY | SGTDFTLTISSLQAEDVAVYYCQQ |
| | FPYAMDNWGQGSLVTVSS (SEQ ID | NNEDPRTFGGGTKVEIK (SEQ ID |
| | NO: 104) | NO: 103) |
| AbE | QVTLRESGPALVKPTQTLTLTCTVSG | DIVMTQSPDSLSVSLGERATINCR |
| | ASLSAYSVNWIRQPPGKALEWLAMI | ASKSVDSYGHSYMHWYQQKPGQ |
| | WGDGKIVYNSALKSRLTISKDTSKNQ | PPKLLIYWASNLESGVPDRFSGSG |
| | VVLTMTNMDPVDTATYYCAGDGYFP | SGTDFTLTISSLQAEDVAVYYCQQ |
| | YAMDNWGQGSLVTVSS (SEQ ID | NNEDPRTFGGGTKVEIK (SEQ ID |
| | NO: 106) | NO: 105) |
| AbF | QVTLRESGPALVKPTQTLTLTCTVSG | DIVMTQSPDSLSVSLGERATINCR |
| | ASLSRYSVNWIRQPPGKALEWLAMI | ASESVDSYGNSYMHWYQQKPGQ |
| | WGDAKIVYNSALKSRLTISKDTSKNQ | PPKLLIYLASNLESGVPDRFSGSG |
| | VVLTMTNMDPVDTATYYCAGDGYFP | SGTDFTLTISSLQAEDVAVYYCQQ |
| | YAMDNWGQGSLVTVSS (SEQ ID | NNEDPRTFGGGTKVEIK (SEQ ID |
| | NO: 108) | NO: 107) |
| AbG | QVTLRESGPALVKPTQTLTLTCTVSG | DIVMTQSPDSLSVSLGERATINCR |
| | ASLSAYSVNWIRQPPGKALEWLAMI | ASKSVDSYGNSYMHWYQQKPGQ |
| | WGDGKIVYNSALKSRLTISKDTSKNQ | PPKLLIYLASNLESGVPDRFSGSG |
| | VVLTMTNMDPVDTATYYCAGDGYFP | SGTDFTLTISSLQAEDVAVYYCQQ |
| | YAMDNWGQGSLVTVSS (SEQ ID | NNEDPRTFGGGTKVEIK (SEQ ID |
| | NO: 110) | NO: 109) |
| AbH | QVTLRESGPALVKPTQTLTLTCTVSG | DIVMTQSPDSLSVSLGERATINCR |
| | ASLSRYSVNWIRQPPGKALEWLAMI | ASKSVDSYGNSYMHWYQQKPGQ |
| | WGDGKIVYNSALKSRLTISKDTSKNQ | PPKLLIYRASNLESGVPDRFSGSG |
| | VVLTMTNMDPVDTATYYCAGDGYY | SGTDFTLTISSLQAEDVAVYYCQQ |
| | PYAMDNWGQGSLVTVSS (SEQ ID | NNEDPRTFGGGTKVEIK (SEQ ID |
| | NO: 112) | NO: 111) |
| AbI | QVTLRESGPALVKPTQTLTLTCTVSG | DIVMTQSPDSLSVSLGERATINCR |
| | ASLSRYSVNWIRQPPGKALEWLAMI | ASKSVDSYGNSYMHWYQQKPGQ |
| | WGDGKIVYNSALKSRLTISKDTSKNQ | PPKLLIYLASNLESGVPDRFSGSG |
| | VVLTMTNMDPVDTATYYCAGDGFYP | SGTDFTLTISSLQAEDVAVYYCQQ |
| | YAMDNWGQGSLVTVSS (SEQ ID | NNEDPRTFGGGTKVEIK (SEQ ID |
| | NO: 114) | NO: 113) |
| AbJ | QVTLRESGPALVKPTQTLTLTCTVSG | DIVMTQSPDSLSVSLGERATINCR |
| | ASLSAYSVNWIRQPPGKALEWLAMI | ASKSVDSYGHSYMHWYQQKPGQ |
| | WGDRKKVYNSALKSRLTISKDTSKN | PPKLLIYWASNLESGVPDRFSGSG |
| | QVVLTMTNMDPVDTATYYCAGDGY | SGTDFTLTISSLQAEDVAVYYCQQ |
| | FPYAMDLWGQGSLVTVSS (SEQ ID | NNEDPRTFGGGTKVEIK (SEQ ID |
| | NO: 116) | NO: 115) |
| AbK | QVTLRESGPALVKPTQTLTLTCTVSG | DIVMTQSPDSLSVSLGERATINCR |
| | SSLSRYSVNWIRQPPGKALEWLAMI | ASQSVDSYGNSYMHWYQQKPGQ |
| | WGDGKIVYNSALKSRLTISKDTSKNQ | PPKLLIYLASNLESGVPDRFSGSG |
| | VVLTMTNMDPVDTATYYCAGDGYY | SGTDFTLTISSLQAEDVAVYYCQQ |
| | PYAMDNWGQGSLVTVSS (SEQ ID | NNEDPRTFGGGTKVEIK (SEQ ID |
| | NO: 118) | NO: 117) |
| AbL | QVTLRESGPALVKPTQTLTLTCTVSG | DIVMTQSPDSLSVSLGERATINCR |
| | FSLSRYSVNWIRQPPGKALEWLAMI | ASKSVDSYGRSYMHWYQQKPGQ |
| | WGDGKIVYNSALKSRLTISKDTSKNQ | PPKLLIYLASNLESGVPDRFSGSG |
| | VVLTMTNMDPVDTATYYCAGDGYY | SGTDFTLTISSLQAEDVAVYYCQQ |
| | PYAMDNWGQGSLVTVSS (SEQ ID | NNEDPRTFGGGTKVEIK (SEQ ID |
| | NO: 120) | NO: 119) |
| AbM | QVTLRESGPALVKPTQTLTLTCTVSG | DIVMTQSPDSLSVSLGERATINCR |
| | ASLSAYSVNWIRQPPGKALEWLAMI | ASKSVDYYGRSYMHWYQQKPGQ |
| | WGDGKIVYNSALKSRLTISKDTSKNQ | PPKLLIYLASNLESGVPDRFSGSG |
| | VVLTMTNMDPVDTATYYCAGDGYFP | SGTDFTLTISSLQAEDVAVYYCQQ |
| | YAMDNWGQGSLVTVSS (SEQ ID | NNEDPRTFGGGTKVEIK (SEQ ID |
| | NO: 122) | NO: 121) |
| AbN | QVTLRESGPALVKPTQTLTLTCTVSG | DIVMTQSPDSLSVSLGERATINCR |
| | ASLSAYSVNWIRQPPGKALEWLAMI | ASESVDYYGHSYMHWYQQKPGQ |
| | WGDGKIVYNSALKSRLTISKDTSKNQ | PPKLLIYLASNLESGVPDRFSGSG |
| | VVLTMTNMDPVDTATYYCAGDGYY | SGTDFTLTISSLQAEDVAVYYCQQ |
| | PYAMDNWGQGSLVTVSS (SEQ ID | NNEDPRTFGGGTKVEIK (SEQ ID |
| | NO: 124) | NO: 123) |
| AbO | QVTLRESGPALVKPTQTLTLTCTVSG | DIVMTQSPDSLSVSLGERATINCR |
| | YSLSAYSVNWIRQPPGKALEWLAMI | ASKSVDYYGHSYMHWYQQKPGQ |
| | WGDGKIVYNSALKSRLTISKDTSKNQ | PPKLLIYLASNLESGVPDRFSGSG |
| | VVLTMTNMDPVDTATYYCAGDGYFP | SGTDFTLTISSLQAEDVAVYYCQQ |
| | YAMDVWGQGSLVTVSS (SEQ ID | NNEDPRTFGGGTKVEIK (SEQ ID |
| | NO: 126) | NO: 125) |
| AbP | QVTLRESGPALVKPTQTLTLTCTVSG | DIVMTQSPDSLSVSLGERATINCR |
| | ASLSAYSVNWIRQPPGKALEWLAMI | ASKSVDSYGNSYMHWYQQKPGQ |
| | WGDGKVVYNSALKSRLTISKDTSKN | PPKLLIYWASNLESGVPDRFSGSG |
| | QVVLTMTNMDPVDTATYYCAGDGY | SGTDFTLTISSLQAEDVAVYYCQQ |
| | FPYAMDHWGQGSLVTVSS (SEQ ID | NNEDPRTFGGGTKVEIK (SEQ ID |
| | NO: 128) | NO: 127) |

In some embodiments, the anti-IL-13 antibody or antigen binding fragment thereof comprises a pair of variable heavy chain and variable light chain sequences, selected from the following combinations: a light chain variable region comprising SEQ ID NO: 97 and a heavy chain variable region comprising SEQ ID NO: 98; a light chain variable region comprising SEQ ID NO: 99 and a heavy chain variable region comprising SEQ ID NO: 100; a light chain variable region comprising SEQ ID NO: 101 and a heavy chain variable region comprising SEQ ID NO: 102; a light chain variable region comprising SEQ ID NO: 103 and a heavy chain variable region comprising SEQ ID NO: 104; a light chain variable region comprising SEQ ID NO: 105 and a heavy chain variable region comprising SEQ ID NO: 106; a light chain variable region comprising SEQ ID NO: 107 and a heavy chain variable region comprising SEQ ID NO: 108; a light chain variable region comprising SEQ ID NO: 109 and a heavy chain variable region comprising SEQ ID NO: 110; a light chain variable region comprising SEQ ID NO: 111 and a heavy chain variable region comprising SEQ ID NO: 112; a light chain variable region comprising SEQ ID NO: 113 and a heavy chain variable region comprising SEQ ID NO: 114; a light chain variable region comprising SEQ ID NO: 115 and a heavy chain variable region comprising SEQ ID NO: 116; a light chain variable region comprising SEQ ID NO: 117 and a heavy chain variable region comprising SEQ ID NO: 118; a light chain variable region comprising SEQ ID NO: 119 and a heavy chain variable region comprising SEQ ID NO: 120; a light chain variable region comprising SEQ ID NO: 121 and a heavy chain variable region comprising SEQ ID NO: 122; a light chain variable region comprising SEQ ID NO: 123 and a heavy chain variable region comprising SEQ ID NO: 124; a light chain variable region comprising SEQ ID NO: 125 and a heavy chain variable region comprising SEQ ID NO: 126; and a light chain variable region comprising SEQ ID NO: 127 and a heavy chain variable region comprising SEQ ID NO: 128.

In some embodiments, the anti-IL-13 antibody or antigen binding fragment thereof comprises a pair of variable heavy chain and variable light chain sequences, selected from the following combinations: a variable heavy chain sequence that is at least about 90%, at least about 95%, at least about 96%, at least about 97%, at least about 98%, or at least about 99% identical to SEQ ID NO: 98 and a variable light chain sequence that is at least about 90%, at least about 95%, at least about 96%, at least about 97%, at least about 98%, or at least about 99% identical to SEQ ID NO: 97; a variable heavy chain sequence that is at least about 90%, at least about 95%, at least about 96%, at least about 97%, at least about 98%, or at least about 99% identical to SEQ ID NO: 100 and a variable light chain sequence that is at least about 90%, at least about 95%, at least about 96%, at least about 97%, at least about 98%, or at least about 99% identical to SEQ ID NO: 99; a variable heavy chain sequence that is at least about 90%, at least about 95%, at least about 96%, at least about 97%, at least about 98%, or at least about 99% identical to SEQ ID NO: 102 and a variable light chain sequence that is at least about 90%, at least about 95%, at least about 96%, at least about 97%, at least about 98%, or at least about 99% identical to SEQ ID NO: 101; a variable heavy chain sequence that is at least about 90%, at least about 95%, at least about 96%, at least about 97%, at least about 98%, or at least about 99% identical to SEQ ID NO: 104 and a variable light chain sequence that is at least about 90%, at least about 95%, at least about 96%, at least about 97%, at least about 98%, or at least about 99% identical to SEQ ID NO: 103; a variable heavy chain sequence that is at least about 90%, at least about 95%, at least about 96%, at least about 97%, at least about 98%, or at least about 99% identical to SEQ ID NO: 106 and a variable light chain sequence that is at least about 90%, at least about 95%, at least about 96%, at least about 97%, at least about 98%, or at least about 99% identical to SEQ ID NO: 105; a variable heavy chain sequence that is at least about 90%, at least about 95%, at least about 96%, at least about 97%, at least about 98%, or at least about 99% identical to SEQ ID NO: 108 and a variable light chain sequence that is at least about 90%, at least about 95%, at least about 96%, at least about 97%, at least about 98%, or at least about 99% identical to SEQ ID NO: 107; a variable heavy chain sequence that is at least about 90%, at least about 95%, at least about 96%, at least about 97%, at least about 98%, or at least about 99% identical to SEQ ID NO: 110 and a variable light chain sequence that is at least about 90%, at least about 95%, at least about 96%, at least about 97%, at least about 98%, or at least about 99% identical to SEQ ID NO: 109; a variable heavy chain sequence that is at least about 90%, at least about 95%, at least about 96%, at least about 97%, at least about 98%, or at least about 99% identical to SEQ ID NO: 112 and a variable light chain sequence that is at least about 90%, at least about 95%, at least about 96%, at least about 97%, at least about 98%, or at least about 99% identical to SEQ ID NO: 111; a variable heavy chain sequence that is at least about 90%, at least about 95%, at least about 96%, at least about 97%, at least about 98%, or at least about 99% identical to SEQ ID NO: 114 and a variable light chain sequence that is at least about 90%, at least about 95%, at least about 96%, at least about 97%, at least about 98%, or at least about 99% identical to SEQ ID NO: 113; a variable heavy chain sequence that is at least about 90%, at least about 95%, at least about 96%, at least about 97%, at least about 98%, or at least about 99% identical to SEQ ID NO: 116 and a variable light chain sequence that is at least about 90%, at least about 95%, at least about 96%, at least about 97%, at least about 98%, or at least about 99% identical to SEQ ID NO: 115; a variable heavy chain sequence that is at least about 90%, at least about 95%, at least about 96%, at least about 97%, at least about 98%, or at least about 99% identical to SEQ ID NO: 118 and a variable light chain sequence that is at least about 90%, at least about 95%, at least about 96%, at least about 97%, at least about 98%, or at least about 99% identical to SEQ ID NO: 117; a variable heavy chain sequence that is at least about 90%, at least about 95%, at least about 96%, at least about 97%, at least about 98%, or at least about 99% identical to SEQ ID NO: 120 and a variable light chain sequence that is at least about 90%, at least about 95%, at least about 96%, at least about 97%, at least about 98%, or at least about 99% identical to SEQ ID NO: 119; a variable heavy chain sequence that is at least about 90%, at least about 95%, at least about 96%, at least about 97%, at least about 98%, or at least about 99% identical to SEQ ID NO: 122 and a variable light chain sequence that is at least about 90%, at least about 95%, at least about 96%, at least about 97%, at least about 98%, or at least about 99% identical to SEQ ID NO: 121; a variable heavy chain sequence that is at least about 90%, at least about 95%, at least about 96%, at least about 97%, at least about 98%, or at least about 99% identical to SEQ ID NO: 124 and a variable light chain sequence that is at least about 90%, at least about 95%, at least about 96%, at least about 97%, at least about 98%, or at least about 99% identical to SEQ ID NO: 123; a variable heavy chain sequence that is at least about 90%, at least about 95%, at least about 96%, at least about 97%, at least about 98%, or at least about 99% identical to SEQ ID NO: 126 and a variable light chain sequence that is at least about 90%, at least about 95%, at least about 96%, at least about 97%, at least about 98%, or at least about 99% identical to SEQ ID NO: 125; and a variable heavy chain sequence that is at least about 90%, at least about 95%, at least about 96%, at least about 97%, at least about 98%, or at least about 99% identical to SEQ ID NO: 128 and a variable light chain sequence that is at least about 90%, at least about 95%, at least about 96%, at least about 97%, at least about 98%, or at least about 99% identical to SEQ ID NO: 127.

In some embodiments, the anti-IL-13 antibody or antigen binding fragment thereof comprises a pair of variable heavy chain and variable light chain sequences, selected from the following combinations: a variable heavy chain sequence that is about 90%, about 95%, about 96%, about 97%, about 98%, or about 99% identical to SEQ ID NO: 98 and a variable light chain sequence that is about 90%, about 95%, about 96%, about 97%, about 98%, or about 99% identical to SEQ ID NO: 97; a variable heavy chain sequence that is about 90%, about 95%, about 96%, about 97%, about 98%, or about 99% identical to SEQ ID NO: 100 and a variable light chain sequence that is about 90%, about 95%, about 96%, about 97%, about 98%, or about 99% identical to SEQ ID NO: 99; a variable heavy chain sequence that is about 90%, about 95%, about 96%, about 97%, about 98%, or about 99% identical to SEQ ID NO: 102 and a variable light chain sequence that is about 90%, about 95%, about 96%, about 97%, about 98%, or about 99% identical to SEQ ID NO: 101; a variable heavy chain sequence that is about 90%, about 95%, about 96%, about 97%, about 98%, or about 99% identical to SEQ ID NO: 104 and a variable light chain sequence that is about 90%, about 95%, about 96%, about 97%, about 98%, or about 99% identical to SEQ ID NO: 103; a variable heavy chain sequence that is about 90%, about 95%, about 96%, about 97%, about 98%, or about 99% identical to SEQ ID NO: 106 and a variable light chain sequence that is about 90%, about 95%, about 96%, about 97%, about 98%, or about 99% identical to SEQ ID NO: 105; a variable heavy chain sequence that is about 90%, about 95%, about 96%, about 97%, about 98%, or about 99% identical to SEQ ID NO: 108 and a variable light chain sequence that is about 90%, about 95%, about 96%, about 97%, about 98%, or about 99% identical to SEQ ID NO: 107; a variable heavy chain sequence that is about 90%, about 95%, about 96%, about 97%, about 98%, or about 99% identical to SEQ ID NO: 110 and a variable light chain sequence that is about 90%, about 95%, about 96%, about 97%, about 98%, or about 99% identical to SEQ ID NO: 109; a variable heavy chain sequence that is about 90%, about 95%, about 96%, about 97%, about 98%, or about 99% identical to SEQ ID NO: 112 and a variable light chain sequence that is about 90%, about 95%, about 96%, about 97%, about 98%, or about 99% identical to SEQ ID NO: 111; a variable heavy chain sequence that is about 90%, about 95%, about 96%, about 97%, about 98%, or about 99% identical to SEQ ID NO: 114 and a variable light chain sequence that is about 90%, about 95%, about 96%, about 97%, about 98%, or about 99% identical to SEQ ID NO: 113; a variable heavy chain sequence that is about 90%, about 95%, about 96%, about 97%, about 98%, or about 99% identical to SEQ ID NO: 116 and a variable light chain sequence that is about 90%, about 95%, about 96%, about 97%, about 98%, or about 99% identical to SEQ ID NO: 115; a variable heavy chain sequence that is about 90%, about 95%, about 96%, about 97%, about 98%, or about 99% identical to SEQ ID NO: 118 and a variable light chain sequence that is about 90%, about 95%, about 96%, about 97%, about 98%, or about 99% identical to SEQ ID NO: 117; a variable heavy chain sequence that is about 90%, about 95%, about 96%, about 97%, about 98%, or about 99% identical to SEQ ID NO: 120 and a variable light chain sequence that is about 90%, about 95%, about 96%, about 97%, about 98%, or about 99% identical to SEQ ID NO: 119; a variable heavy chain sequence that is about 90%, about 95%, about 96%, about 97%, about 98%, or about 99% identical to SEQ ID NO: 122 and a variable light chain sequence that is about 90%, about 95%, about 96%, about 97%, about 98%, or about 99% identical to SEQ ID NO: 121; a variable heavy chain sequence that is about 90%, about 95%, about 96%, about 97%, about 98%, or about 99% identical to SEQ ID NO: 124 and a variable light chain sequence that is about 90%, about 95%, about 96%, about 97%, about 98%, or about 99% identical to SEQ ID NO: 123; a variable heavy chain sequence that is about 90%, about 95%, about 96%, about 97%, about 98%, or about 99% identical to SEQ ID NO: 126 and a variable light chain sequence that is about 90%, about 95%, about 96%, about 97%, about 98%, or about 99% identical to SEQ ID NO: 125; and a variable heavy chain sequence that is about 90%, about 95%, about 96%, about 97%, about 98%, or about 99% identical to SEQ ID NO: 128 and a variable light chain sequence that is about 90%, about 95%, about 96%, about 97%, about 98%, or about 99% identical to SEQ ID NO: 127.

In some embodiments, the anti-IL-13 antibody or antigen binding fragment thereof comprises a heavy chain sequence selected from the group consisting of: SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, and 211; and/or a light chain sequence selected from the group consisting of: SEQ ID NO: 129, 131, 133, 135, 137, 139, 141, 143, 145, 147, 149, 151, 153, 155, 157, and 159.

In some embodiments, the anti-IL-13 antibody or antigen binding fragment thereof comprises a heavy chain (HC) and a light chain (LC) as provided in Table 4.

TABLE 4
Heavy and Light Chain Sequences of Anti-IL-13 Antibodies

| Anti-IL-13 Ab | Chain | Sequence |
|---|---|---|
| AbA | LC | DIVMTQSPDSLSVSLGERATINCRASKSVDSYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 129) |
| AbB | LC | DIVMTQSPDSLSVSLGERATINCRASKSVDYYGHSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 131) |
| AbC | LC | DIVMTQSPDSLSVSLGERATINCRASKSVDYYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 133) |
| AbD | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 135) |
| AbE | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 137) |
| AbF | LC | DIVMTQSPDSLSVSLGERATINCRASESVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 139) |
| AbG | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 141) |
| AbH | LC | DIVMTQSPDSLSVSLGERATINCRASKSVDSYGNSYMHWYQQKPG |
| | | QPPKLLIYRASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 143) |
| AbI | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 145) |
| AbJ | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 147) |
| AbK | LC | DIVMTQSPDSLSVSLGERATINCRASQSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 149) |
| AbL | LC | DIVMTQSPDSLSVSLGERATINCRASKSVDSYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 151) |
| AbM | LC | DIVMTQSPDSLSVSLGERATINCRASKSVDYYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 153) |
| AbN | LC | DIVMTQSPDSLSVSLGERATINCRASESVDYYGHSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 155) |
| AbO | LC | DIVMTQSPDSLSVSLGERATINCRASKSVDYYGHSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 157) |
| AbP | LC | DIVMTQSPDSLSVSLGERATINCRASKSVDSYGNSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 159) |
| AbQ | LC | DIVMTQSPDSLSVSLGERATINCRASKSVDYYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 153) |
| AbR | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 147) |
| AbS | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 141) |
| AbT | LC | DIVMTQSPDSLSVSLGERATINCRASKSVDYYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 153) |
| AbU | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 147) |
| AbV | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 141) |
| AbW | LC | DIVMTQSPDSLSVSLGERATINCRASKSVDYYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 153) |
| AbX | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 147) |
| AbY | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 141) |
| AbZ | LC | DIVMTQSPDSLSVSLGERATINCRASKSVDYYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 153) |
| AbAA | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 147) |
| AbAB | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 141) |
| AbAC | LC | DIVMTQSPDSLSVSLGERATINCRASKSVDYYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 153) |
| AbAD | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 147) |
| AbAE | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 141) |
| AbAF | LC | DIVMTQSPDSLSVSLGERATINCRASKSVDYYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 153) |
| AbAG | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 147) |
| AbAH | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 141) |
| AbAI | LC | DIVMTQSPDSLSVSLGERATINCRASKSVDYYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 153) |
| AbAJ | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 147) |
| AbAK | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 141) |
| AbAL | LC | DIVMTQSPDSLSVSLGERATINCRASKSVDYYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 153) |
| AbAM | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 147) |
| AbAN | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 141) |
| AbAO | LC | DIVMTQSPDSLSVSLGERATINCRASKSVDYYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 153) |
| AbAP | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 147) |
| AbAQ | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 141) |
| AbAR | LC | DIVMTQSPDSLSVSLGERATINCRASKSVDYYGRSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 153) |
| AbAS | LC | DIVMTQSPDSLSVSLGERATINCRASKSVDSYGHSYMHWYQQKPG |
| | | QPPKLLIYWASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 147) |
| AbAT | LC | DIVMTQSPDSLSVSLGERATINCRASKSVDSYGNSYMHWYQQKPG |
| | | QPPKLLIYLASNLESGVPDRFSGSGSGTDFTLTISSLQAEDVAVYYC |
| | | QQNNEDPRTFGGGTKVEIKRTVAAPSVFIFPPSDEQLKSGTASVVCL |
| | | LNNFYPREAKVQWKVDNALQSGNSQESVTEQDSKDSTYSLSSTLTL |
| | | SKADYEKHKVYACEVTHQGLSSPVTKSFNRGEC (SEQ ID NO: 141) |
| AbA | HC | QVTLRESGPALVKPTQTLTLTCTVSGFSLSAYSVNWIRQPPGKALE |
| | | WLAMIWGDGKIVYNSALKSRLTISKDTSKNQVVLTMTNMDPVDTATY |
| | | YCAGDGYYPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 130) |
| AbB | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYYPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 132) |
| AbC | HC | QVTLRESGPALVKPTQTLTLTCTVSGFSLSAYSVNWIRQPPGKALE |
| | | WLAMIWGDGKIVYNSALKSRLTISKDTSKNQVVLTMTNMDPVDTATY |
| | | YCAGDGYYPYAMDYWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 134) |
| AbD | HC | QVTLRESGPALVKPTQTLTLTCTVSGSSLSAYSVNWIRQPPGKALE |
| | | WLAMIWGDGKVVYNSALKSRLTISKDTSKNQVVLTMTNMDPVDTAT |
| | | YYCAGDGYFPYAMDNWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPELLGGPSVFLFPPKPKDTLYITREPEVTCVVVDVSHEDPEVKF |
| | | NWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 136) |
| AbE | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 138) |
| AbF | HC | QVTLRESGPALVKPTQTLTLTCTVSGASLSRYSVNWIRQPPGKALE |
| | | WLAMIWGDAKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 140) |
| AbG | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 142) |
| AbH | HC | QVTLRESGPALVKPTQTLTLTCTVSGASLSRYSVNWIRQPPGKALE |
| | | WLAMIWGDGKIVYNSALKSRLTISKDTSKNQVVLTMTNMDPVDTATY |
| | | YCAGDGYYPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 144) |
| AbI | HC | QVTLRESGPALVKPTQTLTLTCTVSGASLSRYSVNWIRQPPGKALE |
| | | WLAMIWGDGKIVYNSALKSRLTISKDTSKNQVVLTMTNMDPVDTATY |
| | | YCAGDGFYPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 146) |
| AbJ | HC | QVTLRESGPALVKPTQTLTLTCTVSGASLSAYSVNWIRQPPGKALE |
| | | WLAMIWGDRKKVYNSALKSRLTISKDTSKNQVVLTMTNMDPVDTAT |
| | | YYCAGDGYFPYAMDLWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPELLGGPSVFLFPPKPKDTLYITREPEVTCVVVDVSHEDPEVKF |
| | | NWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 148) |
| AbK | HC | QVTLRESGPALVKPTQTLTLTCTVSGSSLSRYSVNWIRQPPGKALE |
| | | WLAMIWGDGKIVYNSALKSRLTISKDTSKNQVVLTMTNMDPVDTATY |
| | | YCAGDGYYPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 150) |
| AbL | HC | QVTLRESGPALVKPTQTLTLTCTVSGFSLSRYSVNWIRQPPGKALE |
| | | WLAMIWGDGKIVYNSALKSRLTISKDTSKNQVVLTMTNMDPVDTATY |
| | | YCAGDGYYPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 152) |
| AbM | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 154) |
| AbN | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYYPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 156) |
| AbO | HC | QVTLRESGPALVKPTQTLTLTCTVSGYSLSAYSVNWIRQPPGKALE |
| | | WLAMIWGDGKIVYNSALKSRLTISKDTSKNQVVLTMTNMDPVDTATY |
| | | YCAGDGYFPYAMDVWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 158) |
| AbP | HC | QVTLRESGPALVKPTQTLTLTCTVSGASLSAYSVNWIRQPPGKALE |
| | | WLAMIWGDGKVVYNSALKSRLTISKDTSKNQVVLTMTNMDPVDTAT |
| | | YYCAGDGYFPYAMDHWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPELLGGPSVFLFPPKPKDTLYITREPEVTCVVVDVSHEDPEVKF |
| | | NWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 160) |
| AbQ | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGAPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 182) |
| AbR | HC | QVTLRESGPALVKPTQTLTLTCTVSGASLSAYSVNWIRQPPGKALE |
| | | WLAMIWGDRKKVYNSALKSRLTISKDTSKNQVVLTMTNMDPVDTAT |
| | | YYCAGDGYFPYAMDLWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPEAAGAPSVFLFPPKPKDTLYITREPEVTCVVVDVSHEDPEVKF |
| | | NWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 183) |
| AbS | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGAPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 184) |
| AbT | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALAAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 185) |
| AbU | HC | QVTLRESGPALVKPTQTLTLTCTVSGASLSAYSVNWIRQPPGKALE |
| | | WLAMIWGDRKKVYNSALKSRLTISKDTSKNQVVLTMTNMDPVDTAT |
| | | YYCAGDGYFPYAMDLWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPEAAGGPSVFLFPPKPKDTLYITREPEVTCVVVDVSHEDPEVKF |
| | | NWYVDGVEVHNAKTKPREEQYNSTYRVVSVLTVLHQDWLNGKEYK |
| | | CKVSNKALAAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 186) |
| AbV | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALAAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 187) |
| AbW | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALGAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 188) |
| AbX | HC | QVTLRESGPALVKPTQTLTLTCTVSGASLSAYSVNWIRQPPGKALE |
| | | WLAMIWGDRKKVYNSALKSRLTISKDTSKNQVVLTMTNMDPVDTAT |
| | | YYCAGDGYFPYAMDLWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPEAAGGPSVFLFPPKPKDTLYITREPEVTCVVVDVSHEDPEVKF |
| | | NWYVDGVEVHNAKTKPREEQYNSTYRVVSVLTVLHQDWLNGKEYK |
| | | CKVSNKALGAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 189) |
| AbY | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALGAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 190) |
| AbZ | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEFEGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPASIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 191) |
| AbAA | HC | QVTLRESGPALVKPTQTLTLTCTVSGASLSAYSVNWIRQPPGKALE |
| | | WLAMIWGDRKKVYNSALKSRLTISKDTSKNQVVLTMTNMDPVDTAT |
| | | YYCAGDGYFPYAMDLWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPEFEGGPSVFLFPPKPKDTLYITREPEVTCVVVDVSHEDPEVKF |
| | | NWYVDGVEVHNAKTKPREEQYNSTYRVVSVLTVLHQDWLNGKEYK |
| | | CKVSNKALPASIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 192) |
| AbAB | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEFEGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPASIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 193) |
| AbAC | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEFEGGPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALPASIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 194) |
| AbAD | HC | QVTLRESGPALVKPTQTLTLTCTVSGASLSAYSVNWIRQPPGKALE |
| | | WLAMIWGDRKKVYNSALKSRLTISKDTSKNQVVLTMTNMDPVDTAT |
| | | YYCAGDGYFPYAMDLWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPEFEGGPSVFLFPPKPKDTLMISRTPEVTCVVVDVSHEDPEVKF |
| | | NWYVDGVEVHNAKTKPREEQYNSTYRVVSVLTVLHQDWLNGKEYK |
| | | CKVSNKALPASIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 195) |
| AbAE | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEFEGGPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALPASIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 196) |
| AbAF | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALGAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 197) |
| AbAG | HC | QVTLRESGPALVKPTQTLTLTCTVSGASLSAYSVNWIRQPPGKALE |
| | | WLAMIWGDRKKVYNSALKSRLTISKDTSKNQVVLTMTNMDPVDTAT |
| | | YYCAGDGYFPYAMDLWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPEAAGGPSVFLFPPKPKDTLMISRTPEVTCVVVDVSHEDPEVKF |
| | | NWYVDGVEVHNAKTKPREEQYNSTYRVVSVLTVLHQDWLNGKEYK |
| | | CKVSNKALGAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 198) |
| AbAH | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALGAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 199) |
| AbAI | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALAAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 200) |
| AbAJ | HC | QVTLRESGPALVKPTQTLTLTCTVSGASLSAYSVNWIRQPPGKALE |
| | | WLAMIWGDRKKVYNSALKSRLTISKDTSKNQVVLTMTNMDPVDTAT |
| | | YYCAGDGYFPYAMDLWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPEAAGGPSVFLFPPKPKDTLMISRTPEVTCVVVDVSHEDPEVKF |
| | | NWYVDGVEVHNAKTKPREEQYNSTYRVVSVLTVLHQDWLNGKEYK |
| | | CKVSNKALAAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 201) |
| AbAK | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALAAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 202) |
| AbAL | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGAPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 203) |
| AbAM | HC | QVTLRESGPALVKPTQTLTLTCTVSGASLSAYSVNWIRQPPGKALE |
| | | WLAMIWGDRKKVYNSALKSRLTISKDTSKNQVVLTMTNMDPVDTAT |
| | | YYCAGDGYFPYAMDLWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPEAAGAPSVFLFPPKPKDTLMISRTPEVTCVVVDVSHEDPEVKF |
| | | NWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 204) |
| AbAN | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGAPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 205) |
| AbAO | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 206) |
| AbAP | HC | QVTLRESGPALVKPTQTLTLTCTVSGASLSAYSVNWIRQPPGKALE |
| | | WLAMIWGDRKKVYNSALKSRLTISKDTSKNQVVLTMTNMDPVDTAT |
| | | YYCAGDGYFPYAMDLWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPEAAGGPSVFLFPPKPKDTLMISRTPEVTCVVVDVSHEDPEVKF |
| | | NWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 207) |
| AbAQ | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLMISRTPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 208) |
| AbAR | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 209) |
| AbAS | HC | QVTLRESGPALVKPTQTLTLTCTVSGASLSAYSVNWIRQPPGKALE |
| | | WLAMIWGDRKKVYNSALKSRLTISKDTSKNQVVLTMTNMDPVDTAT |
| | | YYCAGDGYFPYAMDLWGQGSLVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPEAAGGPSVFLFPPKPKDTLYITREPEVTCVVVDVSHEDPEVKF |
| | | NWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 210) |
| AbAT | HC | QVTLRESGPALVKPTQTLTLTCTVSGASLSAYSVNWIRQPPGKALE |
| | | WLAMIWGDGKIVYNSALKSRLTISKDTSKNQVVLTMTNMDPVDTATY |
| | | YCAGDGYFPYAMDNWGQGSLVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPEAAGGPSVFLFPPKPKDTLYITREPEVTCVVVDVSHEDPEVKFN |
| | | WYVDGVEVHNAKTKPREEQYNSTYRVVSVLTVLHQDWLNGKEYKC |
| | | KVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK (SEQ ID NO: 211) |

In some embodiments, the anti-IL-13 antibody or antigen binding fragment thereof comprises a pair of light chain and heavy chain sequences, selected from the following combinations: a) a light chain comprising SEQ ID NO: 129; and a heavy chain comprising SEQ ID NO: 130; b) a light chain comprising SEQ ID NO: 131; and a heavy chain comprising SEQ ID NO: 132; c) a light chain comprising SEQ ID NO: 133; and a heavy chain comprising SEQ ID NO: 134; d) a light chain comprising SEQ ID NO: 135; and a heavy chain comprising SEQ ID NO: 136; e) a light chain comprising SEQ ID NO: 137; and a heavy chain comprising SEQ ID NO: 138; f) a light chain comprising SEQ ID NO: 139; and a heavy chain comprising SEQ ID NO: 140; g) a light chain comprising SEQ ID NO: 141; and a heavy chain comprising SEQ ID NO: 142; h) a light chain comprising SEQ ID NO: 143; and a heavy chain comprising SEQ ID NO: 144; i) a light chain comprising SEQ ID NO: 145; and a heavy chain comprising SEQ ID NO: 146; j) a light chain comprising SEQ ID NO: 147; and a heavy chain comprising SEQ ID NO: 148; k) a light chain comprising SEQ ID NO: 149; and a heavy chain comprising SEQ ID NO: 150; l) a light chain comprising SEQ ID NO: 151; and a heavy chain comprising SEQ ID NO: 152; m) a light chain comprising SEQ ID NO: 153; and a heavy chain comprising SEQ ID NO: 154; n) a light chain comprising SEQ ID NO: 155; and a heavy chain comprising SEQ ID NO: 156; o) a light chain comprising SEQ ID NO: 157; and a heavy chain comprising SEQ ID NO: 158; p) a light chain comprising SEQ ID NO: 159; and a heavy chain comprising SEQ ID NO: 160; q) a light chain comprising SEQ ID NO: 153; and a heavy chain comprising SEQ ID NO: 182; r) a light chain comprising SEQ ID NO: 147; and a heavy chain comprising SEQ ID NO: 183; s) a light chain comprising SEQ ID NO: 141; and a heavy chain comprising SEQ ID NO: 184; t) a light chain comprising SEQ ID NO: 153; and a heavy chain comprising SEQ ID NO: 185; u) a light chain comprising SEQ ID NO: 147; and a heavy chain comprising SEQ ID NO: 186; v) a light chain comprising SEQ ID NO: 141; and a heavy chain comprising SEQ ID NO: 187; w) a light chain comprising SEQ ID NO: 153; and a heavy chain comprising SEQ ID NO: 188; x) a light chain comprising SEQ ID NO: 147; and a heavy chain comprising SEQ ID NO: 189; y) a light chain comprising SEQ ID NO: 141; and a heavy chain comprising SEQ ID NO: 190; z) a light chain comprising SEQ ID NO: 153; and a heavy chain comprising SEQ ID NO: 191; aa) a light chain comprising SEQ ID NO: 147; and a heavy chain comprising SEQ ID NO: 192; ab) a light chain comprising SEQ ID NO: 141; and a heavy chain comprising SEQ ID NO: 193; ac) a light chain comprising SEQ ID NO: 153; and a heavy chain comprising SEQ ID NO: 194; ad) a light chain comprising SEQ ID NO: 147; and a heavy chain comprising SEQ ID NO: 195; ae) a light chain comprising SEQ ID NO: 141; and a heavy chain comprising SEQ ID NO: 196; af) a light chain comprising SEQ ID NO: 153; and a heavy chain comprising SEQ ID NO: 197; ag) a light chain comprising SEQ ID NO: 147; and a heavy chain comprising SEQ ID NO: 198; ah) a light chain comprising SEQ ID NO: 141; and a heavy chain comprising SEQ ID NO: 199; ai) a light chain comprising SEQ ID NO: 153; and a heavy chain comprising SEQ ID NO: 200; aj) a light chain comprising SEQ ID NO: 147; and a heavy chain comprising SEQ ID NO: 201; ak) a light chain comprising SEQ ID NO: 141; and a heavy chain comprising SEQ ID NO: 202; al) a light chain comprising SEQ ID NO: 153; and a heavy chain comprising SEQ ID NO: 203; am) a light chain comprising SEQ ID NO: 147; and a heavy chain comprising SEQ ID NO: 204; an) a light chain comprising SEQ ID NO: 141; and a heavy chain comprising SEQ ID NO: 205; ao) a light chain comprising SEQ ID NO: 153; and a heavy chain comprising SEQ ID NO: 206; ap) a light chain comprising SEQ ID NO: 147; and a heavy chain comprising SEQ ID NO: 207; aq) a light chain comprising SEQ ID NO: 141; and a heavy chain comprising SEQ ID NO: 208; ar) a light chain comprising SEQ ID NO: 153; and a heavy chain comprising SEQ ID NO: 209; as) a light chain comprising SEQ ID NO: 147; and a heavy chain comprising SEQ ID NO: 210; at) or a light chain comprising SEQ ID NO: 141; and a heavy chain comprising SEQ ID NO: 211. The skilled person will further understand that the light and heavy chains may be independently selected, or mixed and matched, to prepare an anti-IL-13 antibody or antigen binding fragment thereof comprising a combination of heavy and light chain that is distinct from the pairings identified above.

In some embodiments, the anti-IL-13 antibody or antigen binding fragment thereof comprises a pair of heavy chain and light chain sequences, selected from the following combinations: a heavy chain sequence that is at least about 90%, at least about 95%, at least about 96%, at least about 97%, at least about 98%, or at least about 99% identical to SEQ ID NO: 130 and a light chain sequence that is at least about 90%, at least about 95%, at least about 96%, at least about 97%, at least about 98%, or at least about 99% identical to SEQ ID NO: 129; a heavy chain sequence that is at least about 90%, at least about 95%, at least about 96%, at least about 97%, at least about 98%, or at least about 99% identical to SEQ ID NO: 132 and a light chain sequence that is at least about 90%, at least about 95%, at least about 96%, at least about 97%, at least about 98%, or at least about 99% identical to SEQ ID NO: 131; a heavy chain sequence that is at least about 90%, at least about 95%, at least about 96%, at least about 97%, at least about 98%, or at least about 99% identical to SEQ ID NO: 134 and a light chain sequence that is at least about 90%, at least about 95%, at least about 96%, at least about 97%, at least about 98%, or at least about 99% identical to SEQ ID NO: 133; a heavy chain sequence that is at least about 90%, at least about 95%, at least about 96%, at least about 97%, at least about 98%, or at least about 99% identical to SEQ ID NO: 136 and a light chain sequence that is at least about 90%, at least about 95%, at least about 96%, at least about 97%, at least about 98%, or at least about 99% identical to SEQ ID NO: 135; a heavy chain sequence that is at least about 90%, at least about 95%, at least about 96%, at least about 97%, at least about 98%, or at least about 99% identical to SEQ ID NO: 138 and a light chain sequence that is at least about 90%, at least about 95%, at least about 96%, at least about 97%, at least about 98%, or at least about 99% identical to SEQ ID NO: 137; a heavy chain sequence that is at least about 90%, at least about 95%, at least about 96%, at least about 97%, at least about 98%, or at least about 99% identical to SEQ ID NO: 140 and a light chain sequence that is at least about 90%, at least about 95%, at least about 96%, at least about 97%, at least about 98%, or at least about 99% identical to SEQ ID NO: 139; a heavy chain sequence that is at least about 90%, at least about 95%, at least about 96%, at least about 97%, at least about 98%, or at least about 99% identical to SEQ ID NO: 142 and a light chain sequence that is at least about 90%, at least about 95%, at least about 96%, at least about 97%, at least about 98%, or at least about 99% identical to SEQ ID NO: 141; a heavy chain sequence that is at least about 90%, at least about 95%, at least about 96%, at least about 97%, at least about 98%, or at least about 99% identical to SEQ ID NO: 144 and a light chain sequence that is at least about 90%, at least about 95%, at least about 96%, at least about 97%, at least about 98%, or at least about 99% identical to SEQ ID NO: 143; a heavy chain sequence that is at least about 90%, at least about 95%, at least about 96%, at least about 97%, at least about 98%, or at least about 99% identical to SEQ ID NO: 146 and a light chain sequence that is at least about 90%, at least about 95%, at least about 96%, at least about 97%, at least about 98%, or at least about 99% identical to SEQ ID NO: 145; a heavy chain sequence that is at least about 90%, at least about 95%, at least about 96%, at least about 97%, at least about 98%, or at least about 99% identical to SEQ ID NO: 148 and a light chain sequence that is at least about 90%, at least about 95%, at least about 96%, at least about 97%, at least about 98%, or at least about 99% identical to SEQ ID NO: 147; a heavy chain sequence that is at least about 90%, at least about 95%, at least about 96%, at least about 97%, at least about 98%, or at least about 99% identical to SEQ ID NO: 150 and a light chain sequence that is at least about 90%, at least about 95%, at least about 96%, at least about 97%, at least about 98%, or at least about 99% identical to SEQ ID NO: 149; a heavy chain sequence that is at least about 90%, at least about 95%, at least about 96%, at least about 97%, at least about 98%, or at least about 99% identical to SEQ ID NO: 152 and a light chain sequence that is at least about 90%, at least about 95%, at least about 96%, at least about 97%, at least about 98%, or at least about 99% identical to SEQ ID NO: 151; a heavy chain sequence that is at least about 90%, at least about 95%, at least about 96%, at least about 97%, at least about 98%, or at least about 99% identical to SEQ ID NO: 154 and a light chain sequence that is at least about 90%, at least about 95%, at least about 96%, at least about 97%, at least about 98%, or at least about 99% identical to SEQ ID NO: 153; a heavy chain sequence that is at least about 90%, at least about 95%, at least about 96%, at least about 97%, at least about 98%, or at least about 99% identical to SEQ ID NO: 156 and a light chain sequence that is at least about 90%, at least about 95%, at least about 96%, at least about 97%, at least about 98%, or at least about 99% identical to SEQ ID NO: 155; a heavy chain sequence that is at least about 90%, at least about 95%, at least about 96%, at least about 97%, at least about 98%, or at least about 99% identical to SEQ ID NO: 158 and a light chain sequence that is at least about 90%, at least about 95%, at least about 96%, at least about 97%, at least about 98%, or at least about 99% identical to SEQ ID NO: 157; a heavy chain sequence that is at least about 90%, at least about 95%, at least about 96%, at least about 97%, at least about 98%, or at least about 99% identical to SEQ ID NO: 160 and a light chain sequence that is at least about 90%, at least about 95%, at least about 96%, at least about 97%, at least about 98%, or at least about 99% identical to SEQ ID NO: 159; a heavy chain sequence that is at least about 90%, at least about 95%, at least about 96%, at least about 97%, at least about 98%, or at least about 99% identical to SEQ ID NO: 182 and a light chain sequence that is at least about 90%, at least about 95%, at least about 96%, at least about 97%, at least about 98%, or at least about 99% identical to SEQ ID NO: 153; a heavy chain sequence that is at least about 90%, at least about 95%, at least about 96%, at least about 97%, at least about 98%, or at least about 99% identical to SEQ ID NO: 183 and a light chain sequence that is at least about 90%, at least about 95%, at least about 96%, at least about 97%, at least about 98%, or at least about 99% identical to SEQ ID NO: 147; a heavy chain sequence that is at least about 90%, at least about 95%, at least about 96%, at least about 97%, at least about 98%, or at least about 99% identical to SEQ ID NO: 184 and a light chain sequence that is at least about 90%, at least about 95%, at least about 96%, at least about 97%, at least about 98%, or at least about 99% identical to SEQ ID NO: 141; a heavy chain sequence that is at least about 90%, at least about 95%, at least about 96%, at least about 97%, at least about 98%, or at least about 99% identical to SEQ ID NO: 185 and a light chain sequence that is at least about 90%, at least about 95%, at least about 96%, at least about 97%, at least about 98%, or at least about 99% identical to SEQ ID NO: 153; a heavy chain sequence that is at least about 90%, at least about 95%, at least about 96%, at least about 97%, at least about 98%, or at least about 99% identical to SEQ ID NO: 186 and a light chain sequence that is at least about 90%, at least about 95%, at least about 96%, at least about 97%, at least about 98%, or at least about 99% identical to SEQ ID NO: 147; a heavy chain sequence that is at least about 90%, at least about 95%, at least about 96%, at least about 97%, at least about 98%, or at least about 99% identical to SEQ ID NO: 187 and a light chain sequence that is at least about 90%, at least about 95%, at least about 96%, at least about 97%, at least about 98%, or at least about 99% identical to SEQ ID NO: 141; a heavy chain sequence that is at least about 90%, at least about 95%, at least about 96%, at least about 97%, at least about 98%, or at least about 99% identical to SEQ ID NO: 188 and a light chain sequence that is at least about 90%, at least about 95%, at least about 96%, at least about 97%, at least about 98%, or at least about 99% identical to SEQ ID NO: 153; a heavy chain sequence that is at least about 90%, at least about 95%, at least about 96%, at least about 97%, at least about 98%, or at least about 99% identical to SEQ ID NO: 189 and a light chain sequence that is at least about 90%, at least about 95%, at least about 96%, at least about 97%, at least about 98%, or at least about 99% identical to SEQ ID NO: 147; a heavy chain sequence that is at least about 90%, at least about 95%, at least about 96%, at least about 97%, at least about 98%, or at least about 99% identical to SEQ ID NO: 190 and a light chain sequence that is at least about 90%, at least about 95%, at least about 96%, at least about 97%, at least about 98%, or at least about 99% identical to SEQ ID NO: 141; a heavy chain sequence that is at least about 90%, at least about 95%, at least about 96%, at least about 97%, at least about 98%, or at least about 99% identical to SEQ ID NO: 191 and a light chain sequence that is at least about 90%, at least about 95%, at least about 96%, at least about 97%, at least about 98%, or at least about 99% identical to SEQ ID NO: 153; a heavy chain sequence that is at least about 90%, at least about 95%, at least about 96%, at least about 97%, at least about 98%, or at least about 99% identical to SEQ ID NO: 192 and a light chain sequence that is at least about 90%, at least about 95%, at least about 96%, at least about 97%, at least about 98%, or at least about 99% identical to SEQ ID NO: 147; a heavy chain sequence that is at least about 90%, at least about 95%, at least about 96%, at least about 97%, at least about 98%, or at least about 99% identical to SEQ ID NO: 193 and a light chain sequence that is at least about 90%, at least about 95%, at least about 96%, at least about 97%, at least about 98%, or at least about 99% identical to SEQ ID NO: 141, a heavy chain sequence that is at least about 90%, at least about 95%, at least about 96%, at least about 97%, at least about 98%, or at least about 99% identical to SEQ ID NO: 194 and a light chain sequence that is at least about 90%, at least about 95%, at least about 96%, at least about 97%, at least about 98%, or at least about 99% identical to SEQ ID NO: 153; a heavy chain sequence that is at least about 90%, at least about 95%, at least about 96%, at least about 97%, at least about 98%, or at least about 99% identical to SEQ ID NO: 195 and a light chain sequence that is at least about 90%, at least about 95%, at least about 96%, at least about 97%, at least about 98%, or at least about 99% identical to SEQ ID NO: 147; a heavy chain sequence that is at least about 90%, at least about 95%, at least about 96%, at least about 97%, at least about 98%, or at least about 99% identical to SEQ ID NO: 196 and a light chain sequence that is at least about 90%, at least about 95%, at least about 96%, at least about 97%, at least about 98%, or at least about 99% identical to SEQ ID NO: 141; a heavy chain sequence that is at least about 90%, at least about 95%, at least about 96%, at least about 97%, at least about 98%, or at least about 99% identical to SEQ ID NO: 197 and a light chain sequence that is at least about 90%, at least about 95%, at least about 96%, at least about 97%, at least about 98%, or at least about 99% identical to SEQ ID NO: 153; a heavy chain sequence that is at least about 90%, at least about 95%, at least about 96%, at least about 97%, at least about 98%, or at least about 99% identical to SEQ ID NO: 198 and a light chain sequence that is at least about 90%, at least about 95%, at least about 96%, at least about 97%, at least about 98%, or at least about 99% identical to SEQ ID NO: 147; a heavy chain sequence that is at least about 90%, at least about 95%, at least about 96%, at least about 97%, at least about 98%, or at least about 99% identical to SEQ ID NO: 199 and a light chain sequence that is at least about 90%, at least about 95%, at least about 96%, at least about 97%, at least about 98%, or at least about 99% identical to SEQ ID NO: 141; a heavy chain sequence that is at least about 90%, at least about 95%, at least about 96%, at least about 97%, at least about 98%, or at least about 99% identical to SEQ ID NO: 200 and a light chain sequence that is at least about 90%, at least about 95%, at least about 96%, at least about 97%, at least about 98%, or at least about 99% identical to SEQ ID NO: 153; a heavy chain sequence that is at least about 90%, at least about 95%, at least about 96%, at least about 97%, at least about 98%, or at least about 99% identical to SEQ ID NO: 201 and a light chain sequence that is at least about 90%, at least about 95%, at least about 96%, at least about 97%, at least about 98%, or at least about 99% identical to SEQ ID NO: 147; a heavy chain sequence that is at least about 90%, at least about 95%, at least about 96%, at least about 97%, at least about 98%, or at least about 99% identical to SEQ ID NO: 202 and a light chain sequence that is at least about 90%, at least about 95%, at least about 96%, at least about 97%, at least about 98%, or at least about 99% identical to SEQ ID NO: 141; a heavy chain sequence that is at least about 90%, at least about 95%, at least about 96%, at least about 97%, at least about 98%, or at least about 99% identical to SEQ ID NO: 203 and a light chain sequence that is at least about 90%, at least about 95%, at least about 96%, at least about 97%, at least about 98%, or at least about 99% identical to SEQ ID NO: 153; a heavy chain sequence that is at least about 90%, at least about 95%, at least about 96%, at least about 97%, at least about 98%, or at least about 99% identical to SEQ ID NO: 204 and a light chain sequence that is at least about 90%, at least about 95%, at least about 96%, at least about 97%, at least about 98%, or at least about 99% identical to SEQ ID NO: 147; a heavy chain sequence that is at least about 90%, at least about 95%, at least about 96%, at least about 97%, at least about 98%, or at least about 99% identical to SEQ ID NO: 205 and a light chain sequence that is at least about 90%, at least about 95%, at least about 96%, at least about 97%, at least about 98%, or at least about 99% identical to SEQ ID NO: 141; a heavy chain sequence that is at least about 90%, at least about 95%, at least about 96%, at least about 97%, at least about 98%, or at least about 99% identical to SEQ ID NO: 206 and a light chain sequence that is at least about 90%, at least about 95%, at least about 96%, at least about 97%, at least about 98%, or at least about 99% identical to SEQ ID NO: 153: a heavy chain sequence that is at least about 90%, at least about 95%, at least about 96%, at least about 97%, at least about 98%, or at least about 99% identical to SEQ ID NO: 207 and a light chain sequence that is at least about 90%, at least about 95%, at least about 96%, at least about 97%, at least about 98%, or at least about 99% identical to SEQ ID NO: 147; a heavy chain sequence that is at least about 90%, at least about 95%, at least about 96%, at least about 97%, at least about 98%, or at least about 99% identical to SEQ ID NO: 208 and a light chain sequence that is at least about 90%, at least about 95%, at least about 96%, at least about 97%, at least about 98%, or at least about 99% identical to SEQ ID NO: 141; a heavy chain sequence that is at least about 90%, at least about 95%, at least about 96%, at least about 97%, at least about 98%, or at least about 99% identical to SEQ ID NO: 209 and a light chain sequence that is at least about 90%, at least about 95%, at least about 96%, at least about 97%, at least about 98%, or at least about 99% identical to SEQ ID NO: 153; a heavy chain sequence that is at least about 90%, at least about 95%, at least about 96%, at least about 97%, at least about 98%, or at least about 99% identical to SEQ ID NO: 210 and a light chain sequence that is at least about 90%, at least about 95%, at least about 96%, at least about 97%, at least about 98%, or at least about 99% identical to SEQ ID NO: 147; and a heavy chain sequence that is at least about 90%, at least about 95%, at least about 96%, at least about 97%, at least about 98%, or at least about 99% identical to SEQ ID NO: 211 and a light chain sequence that is at least about 90%, at least about 95%, at least about 96%, at least about 97%, at least about 98%, or at least about 99% identical to SEQ ID NO: 141.

In some embodiments, the anti-IL-13 antibody or antigen binding fragment thereof comprises a pair of heavy chain and light chain sequences, selected from the following combinations: a heavy chain sequence that is about 90%, about 95%, about 96%, about 97%, about 98%, or about 99% identical to SEQ ID NO: 130 and a light chain sequence that is about 90%, about 95%, about 96%, about 97%, about 98%, or about 99% identical to SEQ ID NO: 129; a heavy chain sequence that is about 90%, about 95%, about 96%, about 97%, about 98%, or about 99% identical to SEQ ID NO: 132 and a light chain sequence that is about 90%, about 95%, about 96%, about 97%, about 98%, or about 99% identical to SEQ ID NO: 131; a heavy chain sequence that is about 90%, about 95%, about 96%, about 97%, about 98%, or about 99% identical to SEQ ID NO: 134 and a light chain sequence that is about 90%, about 95%, about 96%, about 97%, about 98%, or about 99% identical to SEQ ID NO: 133; a heavy chain sequence that is about 90%, about 95%, about 96%, about 97%, about 98%, or about 99% identical to SEQ ID NO: 136 and a light chain sequence that is about 90%, about 95%, about 96%, about 97%, about 98%, or about 99% identical to SEQ ID NO: 135; a heavy chain sequence that is about 90%, about 95%, about 96%, about 97%, about 98%, or about 99% identical to SEQ ID NO: 138 and a light chain sequence that is about 90%, about 95%, about 96%, about 97%, about 98%, or about 99% identical to SEQ ID NO: 137; a heavy chain sequence that is about 90%, about 95%, about 96%, about 97%, about 98%, or about 99% identical to SEQ ID NO: 140 and a light chain sequence that is about 90%, about 95%, about 96%, about 97%, about 98%, or about 99% identical to SEQ ID NO: 139; a heavy chain sequence that is about 90%, about 95%, about 96%, about 97%, about 98%, or about 99% identical to SEQ ID NO: 142 and a light chain sequence that is about 90%, about 95%, about 96%, about 97%, about 98%, or about 99% identical to SEQ ID NO: 141; a heavy chain sequence that is about 90%, about 95%, about 96%, about 97%, about 98%, or about 99% identical to SEQ ID NO: 144 and a light chain sequence that is about 90%, about 95%, about 96%, about 97%, about 98%, or about 99% identical to SEQ ID NO: 143; a heavy chain sequence that is about 90%, about 95%, about 96%, about 97%, about 98%, or about 99% identical to SEQ ID NO: 146 and a light chain sequence that is about 90%, about 95%, about 96%, about 97%, about 98%, or about 99% identical to SEQ ID NO: 145; a heavy chain sequence that is about 90%, about 95%, about 96%, about 97%, about 98%, or about 99% identical to SEQ ID NO: 148 and a light chain sequence that is about 90%, about 95%, about 96%, about 97%, about 98%, or about 99% identical to SEQ ID NO: 147; a heavy chain sequence that is about 90%, about 95%, about 96%, about 97%, about 98%, or about 99% identical to SEQ ID NO: 150 and a light chain sequence that is about 90%, about 95%, about 96%, about 97%, about 98%, or about 99% identical to SEQ ID NO: 149; a heavy chain sequence that is about 90%, about 95%, about 96%, about 97%, about 98%, or about 99% identical to SEQ ID NO: 152 and a light chain sequence that is about 90%, about 95%, about 96%, about 97%, about 98%, or about 99% identical to SEQ ID NO: 151; a heavy chain sequence that is about 90%, about 95%, about 96%, about 97%, about 98%, or about 99% identical to SEQ ID NO: 154 and a light chain sequence that is about 90%, about 95%, about 96%, about 97%, about 98%, or about 99% identical to SEQ ID NO: 153; a heavy chain sequence that is about 90%, about 95%, about 96%, about 97%, about 98%, or about 99% identical to SEQ ID NO: 156 and a light chain sequence that is about 90%, about 95%, about 96%, about 97%, about 98%, or about 99% identical to SEQ ID NO: 155; a heavy chain sequence that is about 90%, about 95%, about 96%, about 97%, about 98%, or about 99% identical to SEQ ID NO: 158 and a light chain sequence that is about 90%, about 95%, about 96%, about 97%, about 98%, or about 99% identical to SEQ ID NO: 157; a heavy chain sequence that is about 90%, about 95%, about 96%, about 97%, about 98%, or about 99% identical to SEQ ID NO: 160 and a light chain sequence that is about 90%, about 95%, about 96%, about 97%, about 98%, or about 99% identical to SEQ ID NO: 159; a heavy chain sequence that is about 90%, about 95%, about 96%, about 97%, about 98%, or about 99% identical to SEQ ID NO: 182 and a light chain sequence that is about 90%, about 95%, about 96%, about 97%, about 98%, or about 99% identical to SEQ ID NO: 153; a heavy chain sequence that is about 90%, about 95%, about 96%, about 97%, about 98%, or about 99% identical to SEQ ID NO: 183 and a light chain sequence that is about 90%, about 95%, about 96%, about 97%, about 98%, or about 99% identical to SEQ ID NO: 147; a heavy chain sequence that is about 90%, about 95%, about 96%, about 97%, about 98%, or about 99% identical to SEQ ID NO: 184 and a light chain sequence that is about 90%, about 95%, about 96%, about 97%, about 98%, or about 99% identical to SEQ ID NO: 141: a heavy chain sequence that is about 90%, about 95%, about 96%, about 97%, about 98%, or about 99% identical to SEQ ID NO: 185 and a light chain sequence that is about 90%, about 95%, about 96%, about 97%, about 98%, or about 99% identical to SEQ ID NO: 153; a heavy chain sequence that is about 90%, about 95%, about 96%, about 97%, about 98%, or about 99% identical to SEQ ID NO: 186 and a light chain sequence that is about 90%, about 95%, about 96%, about 97%, about 98%, or about 99% identical to SEQ ID NO: 147; a heavy chain sequence that is about 90%, about 95%, about 96%, about 97%, about 98%, or about 99% identical to SEQ ID NO: 187 and a light chain sequence that is about 90%, about 95%, about 96%, about 97%, about 98%, or about 99% identical to SEQ ID NO: 141; a heavy chain sequence that is about 90%, about 95%, about 96%, about 97%, about 98%, or about 99% identical to SEQ ID NO: 188 and a light chain sequence that is about 90%, about 95%, about 96%, about 97%, about 98%, or about 99% identical to SEQ ID NO: 153; a heavy chain sequence that is about 90%, about 95%, about 96%, about 97%, about 98%, or about 99% identical to SEQ ID NO: 189 and a light chain sequence that is about 90%, about 95%, about 96%, about 97%, about 98%, or about 99% identical to SEQ ID NO: 147; a heavy chain sequence that is about 90%, about 95%, about 96%, about 97%, about 98%, or about 99% identical to SEQ ID NO: 190 and a light chain sequence that is about 90%, about 95%, about 96%, about 97%, about 98%, or about 99% identical to SEQ ID NO: 141; a heavy chain sequence that is about 90%, about 95%, about 96%, about 97%, about 98%, or about 99% identical to SEQ ID NO: 191 and a light chain sequence that is about 90%, about 95%, about 96%, about 97%, about 98%, or about 99% identical to SEQ ID NO: 153; a heavy chain sequence that is about 90%, about 95%, about 96%, about 97%, about 98%, or about 99% identical to SEQ ID NO: 192 and a light chain sequence that is about 90%, about 95%, about 96%, about 97%, about 98%, or about 99% identical to SEQ ID NO: 147; a heavy chain sequence that is about 90%, about 95%, about 96%, about 97%, about 98%, or about 99% identical to SEQ ID NO: 193 and a light chain sequence that is about 90%, about 95%, about 96%, about 97%, about 98%, or about 99% identical to SEQ ID NO: 141. a heavy chain sequence that is about 90%, about 95%, about 96%, about 97%, about 98%, or about 99% identical to SEQ ID NO: 194 and a light chain sequence that is about 90%, about 95%, about 96%, about 97%, about 98%, or about 99% identical to SEQ ID NO: 153; a heavy chain sequence that is about 90%, about 95%, about 96%, about 97%, about 98%, or about 99% identical to SEQ ID NO: 195 and a light chain sequence that is about 90%, about 95%, about 96%, about 97%, about 98%, or about 99% identical to SEQ ID NO: 147; a heavy chain sequence that is about 90%, about 95%, about 96%, about 97%, about 98%, or about 99% identical to SEQ ID NO: 196 and a light chain sequence that is about 90%, about 95%, about 96%, about 97%, about 98%, or about 99% identical to SEQ ID NO: 141; a heavy chain sequence that is about 90%, about 95%, about 96%, about 97%, about 98%, or about 99% identical to SEQ ID NO: 197 and a light chain sequence that is about 90%, about 95%, about 96%, about 97%, about 98%, or about 99% identical to SEQ ID NO: 153; a heavy chain sequence that is about 90%, about 95%, about 96%, about 97%, about 98%, or about 99% identical to SEQ ID NO: 198 and a light chain sequence that is about 90%, about 95%, about 96%, about 97%, about 98%, or about 99% identical to SEQ ID NO: 147; a heavy chain sequence that is about 90%, about 95%, about 96%, about 97%, about 98%, or about 99% identical to SEQ ID NO: 199 and a light chain sequence that is about 90%, about 95%, about 96%, about 97%, about 98%, or about 99% identical to SEQ ID NO: 141; a heavy chain sequence that is about 90%, about 95%, about 96%, about 97%, about 98%, or about 99% identical to SEQ ID NO: 200 and a light chain sequence that is about 90%, about 95%, about 96%, about 97%, about 98%, or about 99% identical to SEQ ID NO: 153; a heavy chain sequence that is about 90%, about 95%, about 96%, about 97%, about 98%, or about 99% identical to SEQ ID NO: 201 and a light chain sequence that is about 90%, about 95%, about 96%, about 97%, about 98%, or about 99% identical to SEQ ID NO: 147; a heavy chain sequence that is about 90%, about 95%, about 96%, about 97%, about 98%, or about 99% identical to SEQ ID NO: 202 and a light chain sequence that is about 90%, about 95%, about 96%, about 97%, about 98%, or about 99% identical to SEQ ID NO: 141; a heavy chain sequence that is about 90%, about 95%, about 96%, about 97%, about 98%, or about 99% identical to SEQ ID NO: 203 and a light chain sequence that is about 90%, about 95%, about 96%, about 97%, about 98%, or about 99% identical to SEQ ID NO: 153; a heavy chain sequence that is about 90%, about 95%, about 96%, about 97%, about 98%, or about 99% identical to SEQ ID NO: 204 and a light chain sequence that is about 90%, about 95%, about 96%, about 97%, about 98%, or about 99% identical to SEQ ID NO: 147; a heavy chain sequence that is about 90%, about 95%, about 96%, about 97%, about 98%, or about 99% identical to SEQ ID NO: 205 and a light chain sequence that is about 90%, about 95%, about 96%, about 97%, about 98%, or about 99% identical to SEQ ID NO: 141; a heavy chain sequence that is about 90%, about 95%, about 96%, about 97%, about 98%, or about 99% identical to SEQ ID NO: 206 and a light chain sequence that is about 90%, about 95%, about 96%, about 97%, about 98%, or about 99% identical to SEQ ID NO: 153; a heavy chain sequence that is about 90%, about 95%, about 96%, about 97%, about 98%, or about 99% identical to SEQ ID NO: 207 and a light chain sequence that is about 90%, about 95%, about 96%, about 97%, about 98%, or about 99% identical to SEQ ID NO: 147; a heavy chain sequence that is about 90%, about 95%, about 96%, about 97%, about 98%, or about 99% identical to SEQ ID NO: 208 and a light chain sequence that is about 90%, about 95%, about 96%, about 97%, about 98%, or about 99% identical to SEQ ID NO: 141; a heavy chain sequence that is about 90%, about 95%, about 96%, about 97%, about 98%, or about 99% identical to SEQ ID NO: 209 and a light chain sequence that is about 90%, about 95%, about 96%, about 97%, about 98%, or about 99% identical to SEQ ID NO: 153; a heavy chain sequence that is about 90%, about 95%, about 96%, about 97%, about 98%, or about 99% identical to SEQ ID NO: 210 and a light chain sequence that is about 90%, about 95%, about 96%, about 97%, about 98%, or about 99% identical to SEQ ID NO: 147; and a heavy chain sequence that is about 90%, about 95%, about 96%, about 97%, about 98%, or about 99% identical to SEQ ID NO: 211 and a light chain sequence that is about 90%, about 95%, about 96%, about 97%, about 98%, or about 99% identical to SEQ ID NO: 141.

In some aspects, an antibody or antigen binding fragment thereof that binds IL-13 comprises: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence at least about 90%, at least about 95%, or at least about 99% identical to SEQ ID NO: 97, 99, 101, 103, 105, 107, 109, 111, 113, 115, 117, 119, 121, 123, 125, or 127; and b2) a heavy chain variable region comprising an amino acid sequence at least about 90%, at least about 95%, or at least about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some aspects, an antibody or antigen binding fragment thereof that binds IL-13 comprises: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence about 90%, about 95%, or about 99% identical to SEQ ID NO: 97, 99, 101, 103, 105, 107, 109, 111, 113, 115, 117, 119, 121, 123, 125, or 127; and b2) a heavy chain variable region comprising an amino acid sequence about 90%, about 95%, or about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some embodiments, the antibody or antigen binding fragment thereof that binds IL-13 comprises a) LCDR1 comprises a serine (S) at amino acid position 27D and an arginine (R) at amino acid position 30; b) LCDR1 comprises a tyrosine (Y) at amino acid position 27D and a histidine (H) at amino acid position 30; c) LCDR1 comprises a tyrosine (Y) at amino acid position 27D and an asparagine (N) at amino acid position 30; or d) LCDR1 comprises a serine (S) at amino acid position 27D and a tyrosine (Y) at amino acid position 32, wherein the amino acid positions are based on Kabat numbering.

In some aspects, an antibody or antigen binding fragment thereof that binds IL-13 comprises a light chain variable region and a heavy chain variable region, wherein the light chain variable region is at least about 95% identical to SEQ ID NO: 97, 99, 101, or 103; and wherein the heavy chain variable region is at least about 95% identical to SEQ ID NOs: SEQ ID NO: 98, 100, 102, or 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, wherein the amino acid positions are based on Kabat numbering.

In some aspects, an antibody or antigen binding fragment thereof that binds IL-13 comprises a light chain variable region and a heavy chain variable region, wherein the light chain variable region is at least 95% identical to SEQ ID NO: 97, 99, 101, or 103; and wherein the heavy chain variable region is at least 95% identical to SEQ ID NOs: SEQ ID NO: 98, 100, 102, or 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, wherein the amino acid positions are based on Kabat numbering.

In some embodiments, the antibody is a full-length antibody. In some embodiments, the antibody is an antigen binding fragment, for example, an antigen binding fragment selected from the group consisting of: Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, mini-antibodies, and polypeptides that contain at least a portion of an immunoglobulin that is sufficient to confer IL-13-specific binding to the polypeptide.

In some embodiments, a variable region domain of an anti-IL-13 antibody described herein may be covalently attached at a C-terminal amino acid to at least one other antibody domain or a fragment thereof. Thus, for example, a VH domain that is present in the variable region domain may be linked to an immunoglobulin CH1 domain, or a fragment thereof. Similarly, a VL domain may be linked to a Ck domain or a fragment thereof. In this way, for example, the antibody may be a Fab fragment wherein the antigen binding domain contains associated VH and VL domains covalently linked at their C-termini to a CH1 and Ck domain, respectively. The CH1 domain may be extended with further amino acids, for example to provide a hinge region or a portion of a hinge region domain as found in a Fab fragment, or to provide further domains, such as antibody CH2 and CH3 domains.

In some embodiments, the anti-IL-13 antigen binding fragment comprises at least one CDR as described herein. The antigen binding fragment may comprise at least two, three, four, five, or six CDRs as described herein. The antigen binding fragment further may comprise at least one variable region domain of an antibody described herein. The variable region domain may be of any size or amino acid composition and will generally comprise at least one CDR sequence responsible for binding to human IL-13, for example, HCDR1, HCDR2, HCDR3, LCDR1, LCDR2, and/or LCDR3 as described herein, and which is adjacent to or in frame with one or more framework sequences.

In some embodiments, the anti-IL-13 antibody is a monoclonal antibody. In some embodiments, the anti-IL-13 antibody is a human antibody. In some embodiments, the anti-IL-13 antibody is a murine antibody. In some embodiments, the anti-IL-13 antibody is a chimeric antibody, a bispecific antibody, or a humanized antibody.

In some embodiments, the anti-IL-13 antibody or antigen binding fragment thereof comprises one or more conservative modifications.

In some embodiments, the anti-IL-13 antibody or antigen binding fragment thereof specifically binds to IL-13, and comprises the amino acid sequence of the VH domain and/or VL domain in the sequence listing (e.g., SEQ ID NO: 98, 100, 102, 104, 106, 108, 110, 112, 114, 116, 118, 120, 122, 124, 126, or 128 for VH domains; SEQ ID NO: 97, 99, 101, 103, 105, 107, 109, 111, 113, 115, 117, 119, 121, 123, 125, or 127 for VL domains) but having mutations (e.g., one or more amino acid substitutions) in the framework regions. In some embodiments, antibodies that specifically bind to IL-13 comprise the amino acid sequence of the VH domain and/or VL domain or an antigen-binding fragment thereof of an antibody described herein with one or more amino acid residue substitutions in the framework regions of the VH and/or VL domains.

In some embodiments, the anti-IL-13 antibody or antigen binding fragment thereof comprises a variable heavy chain sequence that comprises an amino acid sequence with at least about 95%, at least about 96%, at least about 97%, at least about 98%, or at least about 99%, sequence identity to the amino acid sequence set forth in SEQ ID NO: 98, 100, 102, 104, 106, 108, 110, 112, 114, 116, 118, 120, 122, 124, 126, or 128. In some embodiments, the antibody or fragment thereof retains the binding and/or functional activity of an antibody or fragment thereof that comprises the variable heavy chain sequence of SEQ ID NO: 98, 100, 102, 104, 106, 108, 110, 112, 114, 116, 118, 120, 122, 124, 126, or 128. In some embodiments, the antibody or fragment thereof comprises the variable heavy chain sequence of SEQ ID NO: 98, 100, 102, 104, 106, 108, 110, 112, 114, 116, 118, 120, 122, 124, 126, or 128 and have one or more conservative amino acid substitutions, e.g., 1, 2, 3, 4, 5, 1-2, 1-3, 1-4 or 1-5 conservative amino acid substitutions in the heavy chain variable sequence. In some embodiments, the one or more conservative amino acid substitutions fall within one or more CDRs and/or framework regions in SEQ ID NO: 98, 100, 102, 104, 106, 108, 110, 112, 114, 116, 118, 120, 122, 124, 126, or 128 (based on the numbering system of Kabat). In some embodiments, the anti-IL-13 antibody or antigen binding fragment specifically binds human IL-13 (SEQ ID NO: 161).

In some embodiments, the anti-IL-13 antibody or antigen binding fragment thereof comprises a variable heavy chain sequence that comprises an amino acid sequence with about 95%, about 96%, about 97%, about 98%, or about 99%, sequence identity to the amino acid sequence set forth in SEQ ID NO: 98, 100, 102, 104, 106, 108, 110, 112, 114, 116, 118, 120, 122, 124, 126, or 128. In some embodiments, the antibody or fragment thereof retains the binding and/or functional activity of an antibody or fragment thereof that comprises the variable heavy chain sequence of SEQ ID NO: 98, 100, 102, 104, 106, 108, 110, 112, 114, 116, 118, 120, 122, 124, 126, or 128. In some embodiments, the antibody or fragment thereof comprises the variable heavy chain sequence of SEQ ID NO: 98, 100, 102, 104, 106, 108, 110, 112, 114, 116, 118, 120, 122, 124, 126, or 128 and have one or more conservative amino acid substitutions, e.g., 1, 2, 3, 4, 5, 1-2, 1-3, 1-4 or 1-5 conservative amino acid substitutions in the heavy chain variable sequence. In some embodiments, the one or more conservative amino acid substitutions fall within one or more CDRs and/or framework regions in SEQ ID NO: 98, 100, 102, 104, 106, 108, 110, 112, 114, 116, 118, 120, 122, 124, 126, or 128 (based on the numbering system of Kabat). In some embodiments, the anti-IL-13 antibody or antigen binding fragment specifically binds human IL-13 (SEQ ID NO: 161).

In some embodiments, the anti-IL-13 antibody or antigen binding fragment thereof comprises a variable heavy chain sequence with at least about 95%, at least about 96%, at least about 97%, at least about 98%, or at least about 99% sequence identity to the heavy chain variable region sequence as set forth in SEQ ID NO: 98, 100, 102, 104, 106, 108, 110, 112, 114, 116, 118, 120, 122, 124, 126, or 128, comprises one or more conservative amino acid substitutions in a framework region (based on the numbering system of Kabat), and retains the binding and/or functional activity of an antibody or fragment thereof that comprises a variable heavy chain sequence as set forth in SEQ ID NO: 98, 100, 102, 104, 106, 108, 110, 112, 114, 116, 118, 120, 122, 124, 126, or 128 and a variable light chain sequence as set forth in SEQ ID NOs: 97, 99, 101, 103, 105, 107, 109, 111, 113, 115, 117, 119, 121, 123, 125, or 127. In some embodiments, the anti-IL-13 antibody or antigen binding fragment specifically binds human IL-13 (SEQ ID NO: 161).

In some embodiments, the anti-IL-13 antibody or antigen binding fragment thereof comprises a variable heavy chain sequence with about 95%, about 96%, about 97%, about 98%, or about 99% sequence identity to the heavy chain variable region sequence as set forth in SEQ ID NO: 98, 100, 102, 104, 106, 108, 110, 112, 114, 116, 118, 120, 122, 124, 126, or 128, comprises one or more conservative amino acid substitutions in a framework region (based on the numbering system of Kabat), and retains the binding and/or functional activity of an antibody or fragment thereof that comprises a variable heavy chain sequence as set forth in SEQ ID NO: 98, 100, 102, 104, 106, 108, 110, 112, 114, 116, 118, 120, 122, 124, 126, or 128 and a variable light chain sequence as set forth in SEQ ID NOs: 97, 99, 101, 103, 105, 107, 109, 111, 113, 115, 117, 119, 121, 123, 125, or 127. In some embodiments, the anti-IL-13 antibody or antigen binding fragment specifically binds human IL-13 (SEQ ID NO: 161).

In some embodiments, the anti-IL-13 antibody or antigen binding fragment thereof comprises a variable light chain sequence that comprises an amino acid sequence with at least about 95%, at least about 96%, at least about 97%, at least about 98%, or at least about 99%, sequence identity to the amino acid sequence set forth in SEQ ID NO: 97, 99, 101, 103, 105, 107, 109, 111, 113, 115, 117, 119, 121, 123, 125, or 127. In some embodiments, the antibody or fragment thereof retains the binding and/or functional activity of an antibody or fragment thereof that comprises the variable light chain sequence of SEQ ID NO: 97, 99, 101, 103, 105, 107, 109, 111, 113, 115, 117, 119, 121, 123, 125, or 127. In some embodiments, the antibody or fragment thereof comprises the variable light chain sequence of SEQ ID NO: 97, 99, 101, 103, 105, 107, 109, 111, 113, 115, 117, 119, 121, 123, 125, or 127 and have one or more conservative amino acid substitutions, e.g., 1, 2, 3, 4, 5, 1-2, 1-3, 1-4 or 1-5 conservative amino acid substitutions in the light chain variable sequence. In some embodiments, the one or more conservative amino acid substitutions fall within one or more CDRs and/or framework regions in SEQ ID NO: 97, 99, 101, 103, 105, 107, 109, 111, 113, 115, 117, 119, 121, 123, 125, or 127 (based on the numbering system of Kabat). In some embodiments, the anti-IL-13 antibody or antigen binding fragment specifically binds human IL-13 (SEQ ID NO: 161).

In some embodiments, the anti-IL-13 antibody or antigen binding fragment thereof comprises a variable light chain sequence that comprises an amino acid sequence with about 95%, about 96%, about 97%, about 98%, or about 99%, sequence identity to the amino acid sequence set forth in SEQ ID NO: 97, 99, 101, 103, 105, 107, 109, 111, 113, 115, 117, 119, 121, 123, 125, or 127. In some embodiments, the antibody or fragment thereof retains the binding and/or functional activity of an antibody or fragment thereof that comprises the variable light chain sequence of SEQ ID NO: 97, 99, 101, 103, 105, 107, 109, 111, 113, 115, 117, 119, 121, 123, 125, or 127. In some embodiments, the antibody or fragment thereof comprises the variable light chain sequence of SEQ ID NO: 97, 99, 101, 103, 105, 107, 109, 111, 113, 115, 117, 119, 121, 123, 125, or 127 and have one or more conservative amino acid substitutions, e.g., 1, 2, 3, 4, 5, 1-2, 1-3, 1-4 or 1-5 conservative amino acid substitutions in the light chain variable sequence. In some embodiments, the one or more conservative amino acid substitutions fall within one or more CDRs and/or framework regions in SEQ ID NO: 97, 99, 101, 103, 105, 107, 109, 111, 113, 115, 117, 119, 121, 123, 125, or 127 (based on the numbering system of Kabat). In some embodiments, the anti-IL-13 antibody or antigen binding fragment specifically binds human IL-13 (SEQ ID NO: 161).

In some embodiments, the anti-IL-13 antibody or antigen binding fragment thereof comprises a variable light chain sequence with at least about 95%, at least about 96%, at least about 97%, at least about 98%, or at least about 99% sequence identity to the light chain variable region sequence as set forth in SEQ ID NOs: 97, 99, 101, 103, 105, 107, 109, 111, 113, 115, 117, 119, 121, 123, 125, or 127, comprises one or more conservative amino acid substitutions in a framework region (based on the numbering system of Kabat), and retains the binding and/or functional activity of an antibody or fragment thereof that comprises a variable heavy chain sequence as set forth in SEQ ID NO: 98, 100, 102, 104, 106, 108, 110, 112, 114, 116, 118, 120, 122, 124, 126, or 128, and a variable light chain sequence as set forth in in SEQ ID NO: 97, 99, 101, 103, 105, 107, 109, 111, 113, 115, 117, 119, 121, 123, 125, or 127. In some embodiments, the anti-IL-13 antibody or antigen binding fragment specifically binds human IL-13 (SEQ ID NO: 161).

In some embodiments, the anti-IL-13 antibody or antigen binding fragment thereof comprises a variable light chain sequence with at least about 95%, at least about 96%, at least about 97%, at least about 98%, or at least about 99% sequence identity to the light chain variable region sequence as set forth in SEQ ID NOs: 97, 99, 101, 103, 105, 107, 109, 111, 113, 115, 117, 119, 121, 123, 125, or 127, comprises one or more conservative amino acid substitutions in a framework region (based on the numbering system of Kabat), and retains the binding and/or functional activity of an antibody or fragment thereof that comprises a variable heavy chain sequence as set forth in SEQ ID NO: 98, 100, 102, 104, 106, 108, 110, 112, 114, 116, 118, 120, 122, 124, 126, or 128, and a variable light chain sequence as set forth in in SEQ ID NO: 97, 99, 101, 103, 105, 107, 109, 111, 113, 115, 117, 119, 121, 123, 125, or 127. In some embodiments, the anti-IL-13 antibody or antigen binding fragment specifically binds human IL-13 (SEQ ID NO: 161).

In some embodiments, the anti-IL-13 antibody or antigen binding fragment thereof comprises a variable light chain sequence that comprises an amino acid sequence with at least about 95%, at least about 96%, at least about 97%, at least about 98%, or at least about 99%, sequence identity to the amino acid sequence set forth in SEQ ID NO: 97, 99, 101, 103, 105, 107, 109, 111, 113, 115, 117, 119, 121, 123, 125, or 127 and a variable heavy chain sequence that comprises an amino acid sequence with at least about 95%, at least about 96%, at least about 97%, at least about 98%, or at least about 99%, sequence identity to the amino acid sequence set forth in SEQ ID NO: 98, 100, 102, 104, 106, 108, 110, 112, 114, 116, 118, 120, 122, 124, 126, or 128. In some embodiments, the antibody or fragment thereof retains the binding and/or functional activity of an antibody or fragment thereof that comprises the variable light chain sequence of in SEQ ID NO: 97, 99, 101, 103, 105, 107, 109, 111, 113, 115, 117, 119, 121, 123, 125, or 127 and the variable heavy chain sequence of SEQ ID NO: 98, 100, 102, 104, 106, 108, 110, 112, 114, 116, 118, 120, 122, 124, 126, or 128, respectively. In some embodiments, the antibody or fragment thereof comprises the variable light chain sequence of in SEQ ID NO: 97, 99, 101, 103, 105, 107, 109, 111, 113, 115, 117, 119, 121, 123, 125, or 127 and has one or more conservative amino acid substitutions, e.g., 1, 2, 3, 4, 5, 1-2, 1-3, 1-4 or 1-5 conservative amino acid substitutions in the light chain variable sequence, and comprises the variable heavy chain sequence of SEQ ID NO: 98, 100, 102, 104, 106, 108, 110, 112, 114, 116, 118, 120, 122, 124, 126, or 128, respectively, and has one or more conservative amino acid substitutions, e.g., 1, 2, 3, 4, 5, 1-2, 1-3, 1-4 or 1-5 conservative amino acid substitutions in the heavy chain variable sequence. In some embodiments, the one or more conservative amino acid substitutions fall within one or more CDRs and/or framework regions in SEQ ID NOs: 97-128 (based on the numbering system of Kabat). In some embodiments, the anti-IL-13 antibody or antigen binding fragment specifically binds human IL-13 (SEQ ID NO: 161).

In some embodiments, the anti-IL-13 antibody or antigen binding fragment thereof comprises a variable light chain sequence that comprises an amino acid sequence with at least about 95%, at least about 96%, at least about 97%, at least about 98%, or at least about 99%, sequence identity to the amino acid sequence set forth in SEQ ID NO: 97, 99, 101, 103, 105, 107, 109, 111, 113, 115, 117, 119, 121, 123, 125, or 127 and a variable heavy chain sequence that comprises an amino acid sequence with about 95%, about 96%, about 97%, about 98%, or about 99%, sequence identity to the amino acid sequence set forth in SEQ ID NO: 98, 100, 102, 104, 106, 108, 110, 112, 114, 116, 118, 120, 122, 124, 126, or 128. In some embodiments, the antibody or fragment thereof retains the binding and/or functional activity of an antibody or fragment thereof that comprises the variable light chain sequence of in SEQ ID NO: 97, 99, 101, 103, 105, 107, 109, 111, 113, 115, 117, 119, 121, 123, 125, or 127 and the variable heavy chain sequence of SEQ ID NO: 98, 100, 102, 104, 106, 108, 110, 112, 114, 116, 118, 120, 122, 124, 126, or 128, respectively. In some embodiments, the antibody or fragment thereof comprises the variable light chain sequence of in SEQ ID NO: 97, 99, 101, 103, 105, 107, 109, 111, 113, 115, 117, 119, 121, 123, 125, or 127 and has one or more conservative amino acid substitutions, e.g., 1, 2, 3, 4, 5, 1-2, 1-3, 1-4 or 1-5 conservative amino acid substitutions in the light chain variable sequence, and comprises the variable heavy chain sequence of SEQ ID NO: 98, 100, 102, 104, 106, 108, 110, 112, 114, 116, 118, 120, 122, 124, 126, or 128, respectively, and has one or more conservative amino acid substitutions, e.g., 1, 2, 3, 4, 5, 1-2, 1-3, 1-4 or 1-5 conservative amino acid substitutions in the heavy chain variable sequence. In some embodiments, the one or more conservative amino acid substitutions fall within one or more CDRs and/or framework regions in SEQ ID NOs: 97-128 (based on the numbering system of Kabat). In some embodiments, the anti-IL-13 antibody or antigen binding fragment specifically binds human IL-13 (SEQ ID NO: 161).

In some embodiments, the anti-IL-13 antibody or antigen binding fragment thereof comprises a variable light chain sequence with at least about 95%, at least about 96%, at least about 97%, at least about 98%, or at least about 99% sequence identity to the light chain variable region sequence as set forth in SEQ ID NO: 97, 99, 101, 103, 105, 107, 109, 111, 113, 115, 117, 119, 121, 123, 125, or 127 and a variable heavy chain sequence with at least about 95%, at least about 96%, at least about 97%, at least about 98%, or at least about 99% sequence identity to the heavy chain variable region sequence as set forth in SEQ ID NO: 98, 100, 102, 104, 106, 108, 110, 112, 114, 116, 118, 120, 122, 124, 126, or 128, comprises one or more conservative amino acid substitutions in a framework region (based on the numbering system of Kabat), and retains the binding and/or functional activity of an antibody or fragment thereof that comprises a variable heavy chain sequence as set forth in SEQ ID NO: 98, 100, 102, 104, 106, 108, 110, 112, 114, 116, 118, 120, 122, 124, 126, or 128 and a variable light chain sequence as set forth in SEQ ID NO: 97, 99, 101, 103, 105, 107, 109, 111, 113, 115, 117, 119, 121, 123, 125, or 127, respectively. In some embodiments, the anti-IL-13 antibody or antigen binding fragment specifically binds human IL-13 (SEQ ID NO: 161).

In some embodiments, the anti-IL-13 antibody or antigen binding fragment thereof comprises a variable light chain sequence with at least about 95%, at least about 96%, at least about 97%, at least about 98%, or at least about 99% sequence identity to the light chain variable region sequence as set forth in SEQ ID NO: 97, 99, 101, 103, 105, 107, 109, 111, 113, 115, 117, 119, 121, 123, 125, or 127 and a variable heavy chain sequence with about 95%, about 96%, about 97%, about 98%, or about 99% sequence identity to the heavy chain variable region sequence as set forth in SEQ ID NO: 98, 100, 102, 104, 106, 108, 110, 112, 114, 116, 118, 120, 122, 124, 126, or 128, comprises one or more conservative amino acid substitutions in a framework region (based on the numbering system of Kabat), and retains the binding and/or functional activity of an antibody or fragment thereof that comprises a variable heavy chain sequence as set forth in SEQ ID NO: 98, 100, 102, 104, 106, 108, 110, 112, 114, 116, 118, 120, 122, 124, 126, or 128 and a variable light chain sequence as set forth in SEQ ID NO: 97, 99, 101, 103, 105, 107, 109, 111, 113, 115, 117, 119, 121, 123, 125, or 127, respectively. In some embodiments, the anti-IL-13 antibody or antigen binding fragment specifically binds human IL-13 (SEQ ID NO: 161).

In some embodiments, the anti-IL-13 antibody or antigen binding fragment thereof specifically binds to IL-13, said antibodies or antigen binding fragments thereof comprising the amino acid sequence of one or more of the CDRs provided herein (e.g., SEQ ID NO: 4, SEQ ID NO: 10, SEQ ID NO: 16, SEQ ID NO: 22, SEQ ID NO: 28, SEQ ID NO: 34, SEQ ID NO: 40, SEQ ID NO: 46, SEQ ID NO: 52, SEQ ID NO: 58, SEQ ID NO: 64, SEQ ID NO: 70, SEQ ID NO: 76, SEQ ID NO: 82, SEQ ID NO: 88, or SEQ ID NO: 94 for HCDR1; SEQ ID NO: 5, SEQ ID NO: 11, SEQ ID NO: 17, SEQ ID NO: 23, SEQ ID NO: 29, SEQ ID NO: 35, SEQ ID NO: 41, SEQ ID NO: 47, SEQ ID NO: 53, SEQ ID NO: 59, SEQ ID NO: 65, SEQ ID NO: 71, SEQ ID NO: 77, SEQ ID NO: 83, SEQ ID NO: 89, or SEQ ID NO: 95 for HCDR2; SEQ ID NO: 6, SEQ ID NO: 12, SEQ ID NO: 18, SEQ ID NO: 24, SEQ ID NO: 30, SEQ ID NO: 36, SEQ ID NO: 42, SEQ ID NO: 48, SEQ ID NO: 54, SEQ ID NO: 60, SEQ ID NO: 64, SEQ ID NO: 72, SEQ ID NO: 78, SEQ ID NO: 84, SEQ ID NO: 90, or SEQ ID NO: 96 for HCDR3; SEQ ID NO: 1, SEQ ID NO: 7, SEQ ID NO: 13, SEQ ID NO: 19, SEQ ID NO: 25, SEQ ID NO: 31, SEQ ID NO: 37, SEQ ID NO: 43, SEQ ID NO: 49, SEQ ID NO: 55, SEQ ID NO: 61, SEQ ID NO: 67, SEQ ID NO: 73, SEQ ID NO: 79, SEQ ID NO: 85, or SEQ ID NO: 91 for LCDR1; SEQ ID NO: 2, SEQ ID NO: 8, SEQ ID NO: 14, SEQ ID NO: 20, SEQ ID NO: 26, SEQ ID NO: 32, SEQ ID NO: 38, SEQ ID NO: 44, SEQ ID NO: 50, SEQ ID NO: 56, SEQ ID NO: 62, SEQ ID NO: 68, SEQ ID NO: 74, SEQ ID NO: 80, SEQ ID NO: 86, or SEQ ID NO: 92 for LCDR2; and SEQ ID NO: 3, SEQ ID NO: 9, SEQ ID NO: 15, SEQ ID NO: 21, SEQ ID NO: 27, SEQ ID NO: 33, SEQ ID NO: 39, SEQ ID NO: 45, SEQ ID NO: 51, SEQ ID NO: 57, SEQ ID NO: 63, SEQ ID NO: 69, SEQ ID NO: 75, SEQ ID NO: 81, SEQ ID NO: 87, or SEQ ID NO: 93 for LCDR3) and human framework regions with one or more amino acid substitutions at one, two, three or more of the following residues: (a) rare framework residues that differ between the murine antibody framework (e.g., donor antibody framework) and the human antibody framework (e.g., acceptor antibody framework); (b) Vernier zone residues when differing between donor antibody framework and acceptor antibody framework; (c) interchain packing residues at the VH/VL interface that differ between the donor antibody framework and the acceptor antibody framework; (d) canonical residues which differ between the donor antibody framework and the acceptor antibody framework sequences, including the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the VH domain and the VL domain. In some embodiments, an anti-IL-13 antibody or antigen binding fragment thereof that specifically binds to an IL-13 antigen comprises the human framework regions with one or more amino acid substitutions at one, two, three or more of the above-identified residues is an antagonistic IL-13 antibody or antigen binding fragment.

In some embodiments, the position of one or more CDRs along the VH (e.g., CDR1, CDR2, or CDR3) and/or VL (e.g., CDR1, CDR2, or CDR3) region of an anti-IL-13 antibody or antigen binding fragment thereof may vary by one, two, three, four, five, or six amino acid positions so long as binding to IL-13 (e.g., human IL-13) is maintained (e.g., substantially maintained, for example, at least about 50%, at least about 60%, at least about 70%, at least about 80%, at least about 90%, at least about 95%). For example, in some embodiments, the position defining a CDR of any of Table 1 or 2 may vary by shifting the N-terminal and/or C-terminal boundary of the CDR by one, two, three, four, five, or six amino acids, relative to the current CDR position, so long as binding to IL-13 (e.g., human IL-13) is maintained (e.g., substantially maintained, for example, at least about 50%, at least about 60%, at least about 70%, at least about 80%, at least about 90%, at least about 95%). In other embodiments, the length of one or more CDRs along the VH (e.g., CDR1, CDR2, or CDR3) and/or VL (e.g., CDR1, CDR2, or CDR3) region of an anti-IL-13 antibody or antigen binding fragment thereof described herein may vary (e.g., be shorter or longer) by one, two, three, four, five, or more amino acids, so long as binding to IL-13 (e.g., human IL-13) is maintained (e.g., substantially maintained, for example, at least about 50%, at least about 60%, at least about 70%, at least about 80%, at least about 90%, at least about 95%). For example, in some embodiments, a VH and/or VL CDR1, CDR2, and/or CDR3 described herein may be one, two, three, four, five or more amino acids shorter than one or more of the CDRs described by SEQ ID NOs: 1-96, so long as binding to IL-13 (e.g., human IL-13) is maintained (e.g., substantially maintained, for example, at least about 50%, at least about 60%, at least about 70%, at least about 80%, at least about 90%, at least about 95%). In other embodiments, a VH and/or VL CDR1, CDR2, and/or CDR3 described herein may be one, two, three, four, five or more amino acids longer than one or more of the CDRs described by SEQ ID NOs: 1-96, so long as binding to IL-13 (e.g., human IL-13) is maintained (e.g., substantially maintained, for example, at least about 50%, at least about 60%, at least about 70%, at least about 80%, at least about 90%, at least about 95%). In other embodiments, the amino terminus of a VH and/or VL CDR1, CDR2, and/or CDR3 described herein may be extended by one, two, three, four, five or more amino acids compared to one or more of the CDRs described by SEQ ID NOs: 1-96 so long as binding to IL-13 (e.g., human IL-13) is maintained (e.g., substantially maintained, for example, at least about 50%, at least about 60%, at least about 70%, at least about 80%, at least about 90%, at least about 95%). In other embodiments, the carboxy terminus of a VH and/or VL CDR1, CDR2, and/or CDR3 described herein may be extended by one, two, three, four, five or more amino acids compared to one or more of the CDRs described by SEQ ID NOs: 1-96, so long as binding to IL-13 (e.g., human IL-13) is maintained (e.g., substantially maintained, for example, at least about 50%, at least about 60%, at least about 70%, at least about 80%, at least about 90%, at least about 95%). In other embodiments, the amino terminus of a VH and/or VL CDR1, CDR2, and/or CDR3 described herein may be shortened by one, two, three, four, five or more amino acids compared to one or more of the CDRs described by SEQ ID NOs: 1-96, so long as binding to IL-13 (e.g., human IL-13) is maintained (e.g., substantially maintained, for example, at least about 50%, at least about 60%, at least about 70%, at least about 80%, at least about 90%, at least about 95%). In some embodiments, the carboxy terminus of a VH and/or VL CDR1, CDR2, and/or CDR3 described herein may be shortened by one, two, three, four, five or more amino acids compared to one or more of the CDRs described by SEQ ID NOs: 1-96 so long as binding to IL-13 (e.g., human IL-13) is maintained (e.g., substantially maintained, for example, at least about 50%, at least about 60%, at least about 70%, at least about 80%, at least about 90%, at least about 95%). Any method known in the art can be used to ascertain whether binding to IL-13 (e.g., human IL-13) is maintained, for example, the binding assays and conditions described in the “Examples” section described herein.

In some embodiments, the position of one or more CDRs along the VH (e.g., CDR1, CDR2, or CDR3) and/or VL (e.g., CDR1, CDR2, or CDR3) region of an anti-IL-13 antibody or antigen binding fragment thereof may vary by one, two, three, four, five, or six amino acid positions so long as binding to IL-13 (e.g., human IL-13) is maintained (e.g., substantially maintained, for example, at least about 50%, at least about 60%, at least about 70%, at least about 80%, at least about 90%, at least about 95%). For example, in some embodiments, the position defining a CDR of any of Table 1 or 2 may vary by shifting the N-terminal and/or C-terminal boundary of the CDR by one, two, three, four, five, or six amino acids, relative to the current CDR position, so long as binding to IL-13 (e.g., human IL-13) is maintained (e.g., substantially maintained, for example, at least about 50%, at least about 60%, at least about 70%, at least about 80%, at least about 90%, at least about 95%). In other embodiments, the length of one or more CDRs along the VH (e.g., CDR1, CDR2, or CDR3) and/or VL (e.g., CDR1, CDR2, or CDR3) region of an anti-IL-13 antibody or antigen binding fragment thereof described herein may vary (e.g., be shorter or longer) by one, two, three, four, five, or more amino acids, so long as binding to IL-13 (e.g., human IL-13) is maintained (e.g., substantially maintained, for example, at least about 50%, at least about 60%, at least about 70%, at least about 80%, at least about 90%, at least about 95%). For example, in some embodiments, a VH and/or VL CDR1, CDR2, and/or CDR3 described herein may be one, two, three, four, five or more amino acids shorter than one or more of the CDRs described by SEQ ID NOS: 1-96, so long as binding to IL-13 (e.g., human IL-13) is maintained (e.g., substantially maintained, for example, at least about 50%, at least about 60%, at least about 70%, at least about 80%, at least about 90%, at least about 95%). In other embodiments, a VH and/or VL CDR1, CDR2, and/or CDR3 described herein may be one, two, three, four, five or more amino acids longer than one or more of the CDRs described by SEQ ID NOs: 1-96, so long as binding to IL-13 (e.g., human IL-13) is maintained (e.g., substantially maintained, for example, at least about 50%, at least about 60%, at least about 70%, at least about 80%, at least about 90%, at least about 95%). In other embodiments, the amino terminus of a VH and/or VL CDR1, CDR2, and/or CDR3 described herein may be extended by one, two, three, four, five or more amino acids compared to one or more of the CDRs described by SEQ ID NOs: 1-96 so long as binding to IL-13 (e.g., human IL-13) is maintained (e.g., substantially maintained, for example, at least about 50%, at least about 60%, at least about 70%, at least about 80%, at least about 90%, at least about 95%). In other embodiments, the carboxy terminus of a VH and/or VL CDR1, CDR2, and/or CDR3 described herein may be extended by one, two, three, four, five or more amino acids compared to one or more of the CDRs described by SEQ ID NOs: 1-96, so long as binding to IL-13 (e.g., human IL-13) is maintained (e.g., substantially maintained, for example, at least about 50%, at least about 60%, at least about 70%, at least about 80%, at least about 90%, at least about 95%). In other embodiments, the amino terminus of a VH and/or VL CDR1, CDR2, and/or CDR3 described herein may be shortened by one, two, three, four, five or more amino acids compared to one or more of the CDRs described by SEQ ID NOs: 1-96, so long as binding to IL-13 (e.g., human IL-13) is maintained (e.g., substantially maintained, for example, at least about 50%, at least about 60%, at least about 70%, at least about 80%, at least about 90%, at least about 95%). In some embodiments, the carboxy terminus of a VH and/or VL CDR1, CDR2, and/or CDR3 described herein may be shortened by one, two, three, four, five or more amino acids compared to one or more of the CDRs described by SEQ ID NOs: 1-96 so long as binding to IL-13 (e.g., human IL-13) is maintained (e.g., substantially maintained, for example, at least about 50%, at least about 60%, at least about 70%, at least about 80%, at least about 90%, at least about 95%). Any method known in the art can be used to ascertain whether binding to IL-13 (e.g., human IL-13) is maintained, for example, the binding assays and conditions described in the “Examples” section described herein.

In some embodiments, the anti-IL-13 antibody or antigen binding fragment thereof comprises an IgG (e.g., IgG1, IgG2, IgG3, or IgG4), IgM, IgE, IgD, or IgA Fc region. In some embodiments, the Fc region comprises L234A and L235A substitutions wherein numbering is according to EU numbering. In some embodiments, the Fc region comprises a G237A substitution wherein numbering is according to EU numbering. In some embodiments, the Fc region comprises M252Y, S254T, and T256E substitutions wherein numbering is according to EU numbering. In some embodiments, the Fc region comprises a P329G substitution wherein numbering is according to EU numbering. In some embodiments, the Fc region comprises a P329A substitution wherein numbering is according to EU numbering. In some embodiments, the Fc region comprises L234F, L235E, and P331S substitutions wherein numbering is according to EU numbering. In some embodiments, the Fc region comprises 234A, 235A, 252Y, 254T, and 256E substitutions wherein numbering is according to EU numbering. In some embodiments, the Fc region comprises 234A, 235A, and 237A substitutions wherein numbering is according to EU numbering. In some embodiments, the Fc region comprises 234A, 235A, 237A, 252Y, 254T, and 256E substitutions wherein numbering is according to EU numbering. In some embodiments, the Fc region comprises 234A, 235A, and 329A substitutions wherein numbering is according to EU numbering. In some embodiments, the Fc region comprises 234A, 235A, 329A, 252Y, 254T, and 256E substitutions wherein numbering is according to EU numbering. In some embodiments, the Fc region comprises 234A, 235A, and 329G substitutions wherein numbering is according to EU numbering. In some embodiments, the Fc region comprises 234A, 235A, 329G, 252Y, 254T, and 256E substitutions wherein numbering is according to EU numbering. In some embodiments, the Fc region comprises 234F, 235E, 331S, 252Y, 254T, and 256E substitutions wherein numbering is according to EU numbering. In some embodiments, the anti-IL-13 antibody or antigen binding fragment thereof may be used in the context of a bispecific or multispecific antibody, engineered with at least one binding specificity to IL-13. Such antibodies may be used for any of the purposes herein described. In some embodiments, a bi- or tri-specific antibody may have at least one binding specificity for IL-13, and at least one binding specificity for another ligand or antigen.

In some embodiments, the anti-IL-13 antibody or antigen binding fragment thereof may be used in the context of a bispecific or multispecific antibody, engineered with at least one binding specificity to IL-13. Such antibodies may be used for any of the purposes herein described. In some embodiments, a bi- or tri-specific antibody may have at least one binding specificity for IL-13, and at least one binding specificity for another ligand or antigen.

### Polynucleotides, Vectors, Host Cells, and Methods of Making

In some aspects polynucleotides (e.g., isolated polynucleotides) are provided that encode an anti-IL-13 antibody or antigen binding fragment thereof, vectors, and host cells comprising the polynucleotides, and recombinant techniques for production of the antibody or antigen binding fragment thereof. The isolated polynucleotides can encode any desired form of the anti-IL-13 antibody including, for example, full length monoclonal antibodies, linear antibodies, single-chain antibodies, chimeric antibodies, humanized antibodies, bispecific antibodies, and multi-specific antibodies (e.g., formed from antigen binding fragments). The isolated polynucleotides may also encode diabodies, Fab, Fab′, F(ab)₂, or Fv fragments.

In some embodiments, a polynucleotide encodes the heavy chain variable region comprising a set of complementarity-determining regions (CDR1, CDR2, and CDR3) selected from the group consisting of: (i) CDR1: SEQ ID NO: 4, CDR2: SEQ ID NO: 5, CDR3: SEQ ID NO: 6; (ii) CDR1: SEQ ID NO: 10, CDR2: SEQ ID NO: 11, CDR3: SEQ ID NO: 12; (iii) CDR1: SEQ ID NO: 16, CDR2: SEQ ID NO: 17, CDR3: SEQ ID NO: 18; (iv) CDR1: SEQ ID NO: 22, CDR2: SEQ ID NO: 23, CDR3: SEQ ID NO: 24; (v) CDR1: SEQ ID NO: 28, CDR2: SEQ ID NO: 29, CDR3: SEQ ID NO: 30; (vi) CDR1: SEQ ID NO: 34, CDR2: SEQ ID NO: 35, CDR3: SEQ ID NO: 36; (vii) CDR1: SEQ ID NO: 40, CDR2: SEQ ID NO: 41, CDR3: SEQ ID NO: 42; (viii) CDR1: SEQ ID NO: 46, CDR2: SEQ ID NO: 47, CDR3: SEQ ID NO: 48; (ix) CDR1: SEQ ID NO: 52, CDR2: SEQ ID NO: 53, CDR3: SEQ ID NO: 54; (x) CDR1: SEQ ID NO: 58, CDR2: SEQ ID NO: 59, CDR3: SEQ ID NO: 60; (xi) CDR1: SEQ ID NO: 64, CDR2: SEQ ID NO: 65, CDR3: SEQ ID NO: 66; (xii) CDR1: SEQ ID NO: 70, CDR2: SEQ ID NO: 71, CDR3: SEQ ID NO: 72; (xiii) CDR1: SEQ ID NO: 76, CDR2: SEQ ID NO: 77, CDR3: SEQ ID NO: 78; (xiv) CDR1: SEQ ID NO: 82, CDR2: SEQ ID NO: 83, CDR3: SEQ ID NO: 84; (xv) CDR1: SEQ ID NO: 88, CDR2: SEQ ID NO: 89, CDR3: SEQ ID NO: 90; and (xvi) CDR1: SEQ ID NO: 94, CDR2: SEQ ID NO: 95, CDR3: SEQ ID NO: 96.

In some embodiments, a polynucleotide encodes the light chain variable region comprising a set of complementarity-determining regions (CDR1, CDR2, and CDR3) selected from the group consisting of: (i) CDR1: SEQ ID NO: 1, CDR2: SEQ ID NO: 2, CDR3: SEQ ID NO: 3; (ii) CDR1: SEQ ID NO: 7, CDR2: SEQ ID NO: 8, CDR3: SEQ ID NO: 9; (iii) CDR1: SEQ ID NO: 13, CDR2: SEQ ID NO: 14, CDR3: SEQ ID NO: 15; (iv) CDR1: SEQ ID NO: 19, CDR2: SEQ ID NO: 20, CDR3: SEQ ID NO: 21; (v) CDR1: SEQ ID NO: 25, CDR2: SEQ ID NO: 26, CDR3: SEQ ID NO: 27; (vi) CDR1: SEQ ID NO: 31, CDR2: SEQ ID NO: 32, CDR3: SEQ ID NO: 33; (vii) CDR1: SEQ ID NO: 37, CDR2: SEQ ID NO: 38, CDR3: SEQ ID NO: 39; (viii) CDR1: SEQ ID NO: 43, CDR2: SEQ ID NO: 44, CDR3: SEQ ID NO: 45; (ix) CDR1: SEQ ID NO: 49, CDR2: SEQ ID NO: 50, CDR3: SEQ ID NO: 51; (x) CDR1: SEQ ID NO: 55, CDR2: SEQ ID NO: 56, CDR3: SEQ ID NO: 57; (xi) CDR1: SEQ ID NO: 61, CDR2: SEQ ID NO: 62, CDR3: SEQ ID NO: 63; (xii) CDR1: SEQ ID NO: 67, CDR2: SEQ ID NO: 68, CDR3: SEQ ID NO: 69; (xiii) CDR1: SEQ ID NO: 73, CDR2: SEQ ID NO: 74, CDR3: SEQ ID NO: 75; (xiv) CDR1: SEQ ID NO: 79, CDR2: SEQ ID NO: 80, CDR3: SEQ ID NO: 81; (xv) CDR1: SEQ ID NO: 85, CDR2: SEQ ID NO: 86, CDR3: SEQ ID NO: 87; and (xvi) CDR1: SEQ ID NO: 91, CDR2: SEQ ID NO: 92, CDR3: SEQ ID NO: 93.

In some embodiments, a polynucleotide encodes a) a VH region comprising a set of complementarity-determining regions (CDR1, CDR2, and CDR3) selected from the group consisting of: (i) CDR1: SEQ ID NO: 4, CDR2: SEQ ID NO: 5, CDR3: SEQ ID NO: 6; (ii) CDR1: SEQ ID NO: 10, CDR2: SEQ ID NO: 11, CDR3: SEQ ID NO: 12; (iii) CDR1: SEQ ID NO: 16, CDR2: SEQ ID NO: 17, CDR3: SEQ ID NO: 18; (iv) CDR1: SEQ ID NO: 22, CDR2: SEQ ID NO: 23, CDR3: SEQ ID NO: 24; (v) CDR1: SEQ ID NO: 28, CDR2: SEQ ID NO: 29, CDR3: SEQ ID NO: 30; (vi) CDR1: SEQ ID NO: 34, CDR2: SEQ ID NO: 35, CDR3: SEQ ID NO: 36; (vii) CDR1: SEQ ID NO: 40, CDR2: SEQ ID NO: 41, CDR3: SEQ ID NO: 42; (viii) CDR1: SEQ ID NO: 46, CDR2: SEQ ID NO: 47, CDR3: SEQ ID NO: 48; (ix) CDR1: SEQ ID NO: 52, CDR2: SEQ ID NO: 53, CDR3: SEQ ID NO: 54; (x) CDR1: SEQ ID NO: 58, CDR2: SEQ ID NO: 59, CDR3: SEQ ID NO: 60; (xi) CDR1: SEQ ID NO: 64, CDR2: SEQ ID NO: 65, CDR3: SEQ ID NO: 66; (xii) CDR1: SEQ ID NO: 70, CDR2: SEQ ID NO: 71, CDR3: SEQ ID NO: 72; (xiii) CDR1: SEQ ID NO: 76, CDR2: SEQ ID NO: 77, CDR3: SEQ ID NO: 78; (xiv) CDR1: SEQ ID NO: 82, CDR2: SEQ ID NO: 83, CDR3: SEQ ID NO: 84; (xv) CDR1: SEQ ID NO: 88, CDR2: SEQ ID NO: 89, CDR3: SEQ ID NO: 90; and (xvi) CDR1: SEQ ID NO: 94, CDR2: SEQ ID NO: 95, CDR3: SEQ ID NO: 96; and (b) a VL region comprising a set of complementarity-determining regions (CDR1, CDR2, and CDR3) selected from the group consisting of: (i) CDR1: SEQ ID NO: 1, CDR2: SEQ ID NO: 2, CDR3: SEQ ID NO: 3; (ii) CDR1: SEQ ID NO: 7, CDR2: SEQ ID NO: 8, CDR3: SEQ ID NO: 9; (iii) CDR1: SEQ ID NO: 13, CDR2: SEQ ID NO: 14, CDR3: SEQ ID NO: 15; (iv) CDR1: SEQ ID NO: 19, CDR2: SEQ ID NO: 20, CDR3: SEQ ID NO: 21; (v) CDR1: SEQ ID NO: 25, CDR2: SEQ ID NO: 26, CDR3: SEQ ID NO: 27; (vi) CDR1: SEQ ID NO: 31, CDR2: SEQ ID NO: 32, CDR3: SEQ ID NO: 33; (vii) CDR1: SEQ ID NO: 37, CDR2: SEQ ID NO: 38, CDR3: SEQ ID NO: 39; (viii) CDR1: SEQ ID NO: 43, CDR2: SEQ ID NO: 44, CDR3: SEQ ID NO: 45; (ix) CDR1: SEQ ID NO: 49, CDR2: SEQ ID NO: 50, CDR3: SEQ ID NO: 51; (x) CDR1: SEQ ID NO: 55, CDR2: SEQ ID NO: 56, CDR3: SEQ ID NO: 57; (xi) CDR1: SEQ ID NO: 61, CDR2: SEQ ID NO: 62, CDR3: SEQ ID NO: 63; (xii) CDR1: SEQ ID NO: 67, CDR2: SEQ ID NO: 68, CDR3: SEQ ID NO: 69; (xiii) CDR1: SEQ ID NO: 73, CDR2: SEQ ID NO: 74, CDR3: SEQ ID NO: 75; (xiv) CDR1: SEQ ID NO: 79, CDR2: SEQ ID NO: 80, CDR3: SEQ ID NO: 81; (xv) CDR1: SEQ ID NO: 85, CDR2: SEQ ID NO: 86, CDR3: SEQ ID NO: 87; and (xvi) CDR1: SEQ ID NO: 91, CDR2: SEQ ID NO: 92, CDR3: SEQ ID NO: 93.

In some embodiments, a polynucleotide encodes a heavy chain variable region of an antibody or antigen binding fragment thereof comprising the amino acid sequence of SEQ ID NO: 98, 100, 102, 104, 106, 108, 110, 112, 114, 116, 118, 120, 122, 124, 126, or 128. In some embodiments, a polynucleotide encodes a light chain variable region of an antibody or antigen binding fragment thereof comprising the amino acid sequence of any of SEQ ID NO: 97, 99, 101, 103, 105, 107, 109, 111, 113, 115, 117, 119, 121, 123, 125, or 127.

In some embodiments, the polynucleotide encodes an antibody or antigen binding fragment thereof comprising:
(a) a variable heavy chain sequence comprising SEQ ID NO: 98 and a variable light chain sequence comprising SEQ ID NO: 97;
(b) a variable heavy chain sequence comprising SEQ ID NO: 100 and a variable light chain sequence comprising SEQ ID NO: 99;
(c) a variable heavy chain sequence comprising SEQ ID NO: 102 and a variable light chain sequence comprising SEQ ID NO: 101;
(d) a variable heavy chain sequence comprising SEQ ID NO: 104 and a variable light chain sequence comprising SEQ ID NO: 103;
(e) a variable heavy chain sequence comprising SEQ ID NO: 106 and a variable light chain sequence comprising SEQ ID NO: 105;
(f) a variable heavy chain sequence comprising SEQ ID NO: 108 and a variable light chain sequence comprising SEQ ID NO: 107;
(g) a variable heavy chain sequence comprising SEQ ID NO: 110 and a variable light chain sequence comprising SEQ ID NO: 109;
(h) a variable heavy chain sequence comprising SEQ ID NO: 112 and a variable light chain sequence comprising SEQ ID NO: 111;
(i) a variable heavy chain sequence comprising SEQ ID NO: 114 and a variable light chain sequence comprising SEQ ID NO: 113;
(j) a variable heavy chain sequence comprising SEQ ID NO: 116 and a variable light chain sequence comprising SEQ ID NO: 115;
(k) a variable heavy chain sequence comprising SEQ ID NO: 118 and a variable light chain sequence comprising SEQ ID NO: 117;
(l) a variable heavy chain sequence comprising SEQ ID NO: 120 and a variable light chain sequence comprising SEQ ID NO: 119;
(m) a variable heavy chain sequence comprising SEQ ID NO: 122 and a variable light chain sequence comprising SEQ ID NO: 121;
(n) a variable heavy chain sequence comprising SEQ ID NO: 124 and a variable light chain sequence comprising SEQ ID NO: 123;
(o) a variable heavy chain sequence comprising SEQ ID NO: 126 and a variable light chain sequence comprising SEQ ID NO: 125; or
(p) a variable heavy chain sequence comprising SEQ ID NO: 128 and a variable light chain sequence comprising SEQ ID NO: 127.

In some embodiments, the polynucleotide encodes an antibody or antigen binding fragment thereof comprising:
(a) a variable heavy chain sequence that is at least about 90%, at least about 95%, at least about 96%, at least about 97%, at least about 98%, or at least about 99% identical to SEQ ID NO: 98 and a variable light chain sequence that is at least about 90%, at least about 95%, at least about 96%, at least about 97%, at least about 98%, or at least about 99% identical to SEQ ID NO: 97;
(b) a variable heavy chain sequence that is at least about 90%, at least about 95%, at least about 96%, at least about 97%, at least about 98%, or at least about 99% identical to SEQ ID NO: 100 and a variable light chain sequence that is at least about 90%, at least about 95%, at least about 96%, at least about 97%, at least about 98%, or at least about 99% identical to SEQ ID NO: 99;
(c) a variable heavy chain sequence that is at least about 90%, at least about 95%, at least about 96%, at least about 97%, at least about 98%, or at least about 99% identical to SEQ ID NO: 102 and a variable light chain sequence that is at least about 90%, at least about 95%, at least about 96%, at least about 97%, at least about 98%, or at least about 99% identical to SEQ ID NO: 101;
(d) a variable heavy chain sequence that is at least about 90%, at least about 95%, at least about 96%, at least about 97%, at least about 98%, or at least about 99% identical to SEQ ID NO: 104 and a variable light chain sequence that is at least about 90%, at least about 95%, at least about 96%, at least about 97%, at least about 98%, or at least about 99% identical to SEQ ID NO: 103;
(e) a variable heavy chain sequence that is at least about 90%, at least about 95%, at least about 96%, at least about 97%, at least about 98%, or at least about 99% identical to SEQ ID NO: 106 and a variable light chain sequence that is at least about 90%, at least about 95%, at least about 96%, at least about 97%, at least about 98%, or at least about 99% identical to SEQ ID NO: 105;
(f) a variable heavy chain sequence that is at least about 90%, at least about 95%, at least about 96%, at least about 97%, at least about 98%, or at least about 99% identical to SEQ ID NO: 108 and a variable light chain sequence that is at least about 90%, at least about 95%, at least about 96%, at least about 97%, at least about 98%, or at least about 99% identical to SEQ ID NO: 107;
(g) a variable heavy chain sequence that is at least about 90%, at least about 95%, at least about 96%, at least about 97%, at least about 98%, or at least about 99% identical to SEQ ID NO: 110 and a variable light chain sequence that is at least about 90%, at least about 95%, at least about 96%, at least about 97%, at least about 98%, or at least about 99% identical to SEQ ID NO: 109;
(h) a variable heavy chain sequence that is at least about 90%, at least about 95%, at least about 96%, at least about 97%, at least about 98%, or at least about 99% identical to SEQ ID NO: 112 and a variable light chain sequence that is at least about 90%, at least about 95%, at least about 96%, at least about 97%, at least about 98%, or at least about 99% identical to SEQ ID NO: 111;
(i) a variable heavy chain sequence that is at least about 90%, at least about 95%, at least about 96%, at least about 97%, at least about 98%, or at least about 99% identical to SEQ ID NO: 114 and a variable light chain sequence that is at least about 90%, at least about 95%, at least about 96%, at least about 97%, at least about 98%, or at least about 99% identical to SEQ ID NO: 113;
(j) a variable heavy chain sequence that is at least about 90%, at least about 95%, at least about 96%, at least about 97%, at least about 98%, or at least about 99% identical to SEQ ID NO: 116 and a variable light chain sequence that is at least about 90%, at least about 95%, at least about 96%, at least about 97%, at least about 98%, or at least about 99% identical to SEQ ID NO: 115;
(k) a variable heavy chain sequence that is at least about 90%, at least about 95%, at least about 96%, at least about 97%, at least about 98%, or at least about 99% identical to SEQ ID NO: 118 and a variable light chain sequence that is at least about 90%, at least about 95%, at least about 96%, at least about 97%, at least about 98%, or at least about 99% identical to SEQ ID NO: 117;
(l) a variable heavy chain sequence that is at least about 90%, at least about 95%, at least about 96%, at least about 97%, at least about 98%, or at least about 99% identical to SEQ ID NO: 120 and a variable light chain sequence that is at least about 90%, at least about 95%, at least about 96%, at least about 97%, at least about 98%, or at least about 99% identical to SEQ ID NO: 119;
(m) a variable heavy chain sequence that is at least about 90%, at least about 95%, at least about 96%, at least about 97%, at least about 98%, or at least about 99% identical to SEQ ID NO: 122 and a variable light chain sequence that is at least about 90%, at least about 95%, at least about 96%, at least about 97%, at least about 98%, or at least about 99% identical to SEQ ID NO: 121;
(n) a variable heavy chain sequence that is at least about 90%, at least about 95%, at least about 96%, at least about 97%, at least about 98%, or at least about 99% identical to SEQ ID NO: 124 and a variable light chain sequence that is at least about 90%, at least about 95%, at least about 96%, at least about 97%, at least about 98%, or at least about 99% identical to SEQ ID NO: 123;
(o) a variable heavy chain sequence that is at least about 90%, at least about 95%, at least about 96%, at least about 97%, at least about 98%, or at least about 99% identical to SEQ ID NO: 126 and a variable light chain sequence that is at least about 90%, at least about 95%, at least about 96%, at least about 97%, at least about 98%, or at least about 99% identical to SEQ ID NO: 125; or
(p) a variable heavy chain sequence that is at least about 90%, at least about 95%, at least about 96%, at least about 97%, at least about 98%, or at least about 99% identical to SEQ ID NO: 128 and a variable light chain sequence that is at least about 90%, at least about 95%, at least about 96%, at least about 97%, at least about 98%, or at least about 99% identical to SEQ ID NO: 127.

In some embodiments, the polynucleotide encodes an antibody or antigen binding fragment thereof comprising:
(a) a variable heavy chain sequence that is about 90%, about 95%, about 96%, about 97%, about 98%, or about 99% identical to SEQ ID NO: 98 and a variable light chain sequence that is about 90%, about 95%, about 96%, about 97%, about 98%, or about 99% identical to SEQ ID NO: 97;
(b) a variable heavy chain sequence that is about 90%, about 95%, about 96%, about 97%, about 98%, or about 99% identical to SEQ ID NO: 100 and a variable light chain sequence that is about 90%, about 95%, about 96%, about 97%, about 98%, or about 99% identical to SEQ ID NO: 99;
(c) a variable heavy chain sequence that is about 90%, about 95%, about 96%, about 97%, about 98%, or about 99% identical to SEQ ID NO: 102 and a variable light chain sequence that is about 90%, about 95%, about 96%, about 97%, about 98%, or about 99% identical to SEQ ID NO: 101;
(d) a variable heavy chain sequence that is about 90%, about 95%, about 96%, about 97%, about 98%, or about 99% identical to SEQ ID NO: 104 and a variable light chain sequence that is about 90%, about 95%, about 96%, about 97%, about 98%, or about 99% identical to SEQ ID NO: 103;
(e) a variable heavy chain sequence that is about 90%, about 95%, about 96%, about 97%, about 98%, or about 99% identical to SEQ ID NO: 106 and a variable light chain sequence that is about 90%, about 95%, about 96%, about 97%, about 98%, or about 99% identical to SEQ ID NO: 105;
(f) a variable heavy chain sequence that is about 90%, about 95%, about 96%, about 97%, about 98%, or about 99% identical to SEQ ID NO: 108 and a variable light chain sequence that is about 90%, about 95%, about 96%, about 97%, about 98%, or about 99% identical to SEQ ID NO: 107;
(g) a variable heavy chain sequence that is about 90%, about 95%, about 96%, about 97%, about 98%, or about 99% identical to SEQ ID NO: 110 and a variable light chain sequence that is about 90%, about 95%, about 96%, about 97%, about 98%, or about 99% identical to SEQ ID NO: 109;
(h) a variable heavy chain sequence that is about 90%, about 95%, about 96%, about 97%, about 98%, or about 99% identical to SEQ ID NO: 112 and a variable light chain sequence that is about 90%, about 95%, about 96%, about 97%, about 98%, or about 99% identical to SEQ ID NO: 111;
(i) a variable heavy chain sequence that is about 90%, about 95%, about 96%, about 97%, about 98%, or about 99% identical to SEQ ID NO: 114 and a variable light chain sequence that is about 90%, about 95%, about 96%, about 97%, about 98%, or about 99% identical to SEQ ID NO: 113;
(j) a variable heavy chain sequence that is about 90%, about 95%, about 96%, about 97%, about 98%, or about 99% identical to SEQ ID NO: 116 and a variable light chain sequence that is about 90%, about 95%, about 96%, about 97%, about 98%, or about 99% identical to SEQ ID NO: 115;
(k) a variable heavy chain sequence that is about 90%, about 95%, about 96%, about 97%, about 98%, or about 99% identical to SEQ ID NO: 118 and a variable light chain sequence that is about 90%, about 95%, about 96%, about 97%, about 98%, or about 99% identical to SEQ ID NO: 117;
(l) a variable heavy chain sequence that is about 90%, about 95%, about 96%, about 97%, about 98%, or about 99% identical to SEQ ID NO: 120 and a variable light chain sequence that is about 90%, about 95%, about 96%, about 97%, about 98%, or about 99% identical to SEQ ID NO: 119;
(m) a variable heavy chain sequence that is about 90%, about 95%, about 96%, about 97%, about 98%, or about 99% identical to SEQ ID NO: 122 and a variable light chain sequence that is about 90%, about 95%, about 96%, about 97%, about 98%, or about 99% identical to SEQ ID NO: 121;
(n) a variable heavy chain sequence that is about 90%, about 95%, about 96%, about 97%, about 98%, or about 99% identical to SEQ ID NO: 124 and a variable light chain sequence that is about 90%, about 95%, about 96%, about 97%, about 98%, or about 99% identical to SEQ ID NO: 123;
(o) a variable heavy chain sequence that is about 90%, about 95%, about 96%, about 97%, about 98%, or about 99% identical to SEQ ID NO: 126 and a variable light chain sequence that is about 90%, about 95%, about 96%, about 97%, about 98%, or about 99% identical to SEQ ID NO: 125; or
(p) a variable heavy chain sequence that is about 90%, about 95%, about 96%, about 97%, about 98%, or about 99% identical to SEQ ID NO: 128 and a variable light chain sequence that is about 90%, about 95%, about 96%, about 97%, about 98%, or about 99% identical to SEQ ID NO: 127.

In some embodiments, the polynucleotide encodes an antibody or antigen binding fragment thereof that binds IL-13 wherein: a) the light chain comprises an amino acid sequence as set forth in SEQ ID NO: 129; and the heavy chain comprises an amino acid sequence as set forth in SEQ ID NO: 130; b) the light chain comprises an amino acid sequence as set forth in SEQ ID NO: 131; and the heavy chain comprises an amino acid sequence as set forth in SEQ ID NO: 132; c) the light chain comprises an amino acid sequence as set forth in SEQ ID NO: 133; and the heavy chain comprises an amino acid sequence as set forth in SEQ ID NO: 134; d) the light chain comprises an amino acid sequence as set forth in SEQ ID NO: 135; and the heavy chain comprises an amino acid sequence as set forth in SEQ ID NO: 136; e) the light chain comprises an amino acid sequence as set forth in SEQ ID NO: 137; and the heavy chain comprises an amino acid sequence as set forth in SEQ ID NO: 138; f) the light chain comprises an amino acid sequence as set forth in SEQ ID NO: 139; and the heavy chain comprises an amino acid sequence as set forth in SEQ ID NO: 140; g) the light chain comprises an amino acid sequence as set forth in SEQ ID NO: 141; and the heavy chain comprises an amino acid sequence as set forth in SEQ ID NO: 142; h) the light chain comprises an amino acid sequence as set forth in SEQ ID NO: 143; and the heavy chain comprises an amino acid sequence as set forth in SEQ ID NO: 144; i) the light chain comprises an amino acid sequence as set forth in SEQ ID NO: 145; and the heavy chain comprises an amino acid sequence as set forth in SEQ ID NO: 146; j) the light chain comprises an amino acid sequence as set forth in SEQ ID NO: 147; and the heavy chain comprises an amino acid sequence as set forth in SEQ ID NO: 148; k) the light chain comprises an amino acid sequence as set forth in SEQ ID NO: 149; and the heavy chain comprises an amino acid sequence as set forth in SEQ ID NO: 150; l) the light chain comprises an amino acid sequence as set forth in SEQ ID NO: 151; and the heavy chain comprises an amino acid sequence as set forth in SEQ ID NO: 152; m) the light chain comprises an amino acid sequence as set forth in SEQ ID NO: 153; and the heavy chain comprises an amino acid sequence as set forth in SEQ ID NO: 154; n) the light chain comprises an amino acid sequence as set forth in SEQ ID NO: 155; and the heavy chain comprises an amino acid sequence as set forth in SEQ ID NO: 156; o) the light chain comprises an amino acid sequence as set forth in SEQ ID NO: 157; and the heavy chain comprises an amino acid sequence as set forth in SEQ ID NO: 158; p) the light chain comprises an amino acid sequence as set forth in SEQ ID NO: 159; and the heavy chain comprises an amino acid sequence as set forth in SEQ ID NO: 160; q) the light chain comprises an amino acid sequence as set forth in SEQ ID NO: 153 and the heavy chain comprises an amino acid sequence as set forth in SEQ ID NO: 182; r) the light chain comprises an amino acid sequence as set forth in SEQ ID NO: 147; and the heavy chain comprises an amino acid sequence as set forth in SEQ ID NO: 183; s) the light chain comprises an amino acid sequence as set forth in SEQ ID NO: 141; and the heavy chain comprises an amino acid sequence as set forth in SEQ ID NO: 184; t) the light chain comprises an amino acid sequence as set forth in SEQ ID NO: 153; and the heavy chain comprises an amino acid sequence as set forth in SEQ ID NO: 185; u) the light chain comprises an amino acid sequence as set forth in SEQ ID NO: 147; and the heavy chain comprises an amino acid sequence as set forth in SEQ ID NO: 186; v) the light chain comprises an amino acid sequence as set forth in SEQ ID NO: 141; and the heavy chain comprises an amino acid sequence as set forth in SEQ ID NO: 187; w) the light chain comprises an amino acid sequence as set forth in SEQ ID NO: 153; and the heavy chain comprises an amino acid sequence as set forth in SEQ ID NO: 188; x) the light chain comprises an amino acid sequence as set forth in SEQ ID NO: 147; and the heavy chain comprises an amino acid sequence as set forth in SEQ ID NO: 189; y) the light chain comprises an amino acid sequence as set forth in SEQ ID NO: 141; and the heavy chain comprises an amino acid sequence as set forth in SEQ ID NO: 190; z) the light chain comprises an amino acid sequence as set forth in SEQ ID NO: 153; and the heavy chain comprises an amino acid sequence as set forth in SEQ ID NO: 191; aa) the light chain comprises an amino acid sequence as set forth in SEQ ID NO: 147; and the heavy chain comprises an amino acid sequence as set forth in SEQ ID NO: 192; ab) the light chain comprises an amino acid sequence as set forth in SEQ ID NO: 141; and the heavy chain comprises an amino acid sequence as set forth in SEQ ID NO: 193; ac) the light chain comprises an amino acid sequence as set forth in SEQ ID NO: 153; and the heavy chain comprises an amino acid sequence as set forth in SEQ ID NO: 194; ad) the light chain comprises an amino acid sequence as set forth in SEQ ID NO: 147; and the heavy chain comprises an amino acid sequence as set forth in SEQ ID NO: 195; ae) the light chain comprises an amino acid sequence as set forth in SEQ ID NO: 141; and the heavy chain comprises an amino acid sequence as set forth in SEQ ID NO: 196; af) the light chain comprises an amino acid sequence as set forth in SEQ ID NO: 153; and the heavy chain comprises an amino acid sequence as set forth in SEQ ID NO: 197; ag) the light chain comprises an amino acid sequence as set forth in SEQ ID NO: 147; and the heavy chain comprises an amino acid sequence as set forth in SEQ ID NO: 198; ah) the light chain comprises an amino acid sequence as set forth in SEQ ID NO: 141; and the heavy chain comprises an amino acid sequence as set forth in SEQ ID NO: 199; ai) the light chain comprises an amino acid sequence as set forth in SEQ ID NO: 153; and the heavy chain comprises an amino acid sequence as set forth in SEQ ID NO: 200; aj) the light chain comprises an amino acid sequence as set forth in SEQ ID NO: 147; and the heavy chain comprises an amino acid sequence as set forth in SEQ ID NO: 201; ak) the light chain comprises an amino acid sequence as set forth in SEQ ID NO: 141; and the heavy chain comprises an amino acid sequence as set forth in SEQ ID NO: 202; al) the light chain comprises an amino acid sequence as set forth in SEQ ID NO: 153; and the heavy chain comprises an amino acid sequence as set forth in SEQ ID NO: 203; am) the light chain comprises an amino acid sequence as set forth in SEQ ID NO: 147; and the heavy chain comprises an amino acid sequence as set forth in SEQ ID NO: 204; an) the light chain comprises an amino acid sequence as set forth in SEQ ID NO: 141; and the heavy chain comprises an amino acid sequence as set forth in SEQ ID NO: 205; ao) the light chain comprises an amino acid sequence as set forth in SEQ ID NO: 153; and the heavy chain comprises an amino acid sequence as set forth in SEQ ID NO: 206; ap) the light chain comprises an amino acid sequence as set forth in SEQ ID NO: 147; and the heavy chain comprises an amino acid sequence as set forth in SEQ ID NO: 207; aq) the light chain comprises an amino acid sequence as set forth in SEQ ID NO: 141; and the heavy chain comprises an amino acid sequence as set forth in SEQ ID NO: 208; ar) the light chain comprises an amino acid sequence as set forth in SEQ ID NO: 153; and the heavy chain comprises an amino acid sequence as set forth in SEQ ID NO: 209; as) the light chain comprises an amino acid sequence as set forth in SEQ ID NO: 147; and the heavy chain comprises an amino acid sequence as set forth in SEQ ID NO: 210; or at) the light chain comprises an amino acid sequence as set forth in SEQ ID NO: 141; and the heavy chain comprises an amino acid sequence as set forth in SEQ ID NO: 211.

In some embodiments, the polynucleotide encodes an antibody or antigen binding fragment thereof that binds IL-13 comprising: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence at least about 90%, at least about 95%, or at least about 99% identical to SEQ ID NO: 97, 99, 101, 103, 105, 107, 109, 111, 113, 115, 117, 119, 121, 123, 125, or 127; and b2) a heavy chain variable region comprising an amino acid sequence at least about 90%, at least about 95%, or at least about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some embodiments, the polynucleotide encodes an antibody or antigen binding fragment thereof that binds IL-13 comprising: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence about 90%, about 95%, or about 99% identical to SEQ ID NO: 97, 99, 101, 103, 105, 107, 109, 111, 113, 115, 117, 119, 121, 123, 125, or 127; and b2) a heavy chain variable region comprising an amino acid sequence about 90%, about 95%, or about 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

In some embodiments, the polynucleotide encodes an antibody or antigen binding fragment thereof that binds IL-13, wherein: a) LCDR1 comprises a serine (S) at amino acid position 27D and an arginine (R) at amino acid position 30; b) LCDR1 comprises a tyrosine (Y) at amino acid position 27D and a histidine (H) at amino acid position 30; c) LCDR1 comprises a tyrosine (Y) at amino acid position 27D and an asparagine (N) at amino acid position 30; or d) LCDR1 comprises a serine (S) at amino acid position 27D and a tyrosine (Y) at amino acid position 32, wherein the amino acid positions are based on Kabat numbering.

In some embodiments, the polynucleotide encodes an antibody or antigen binding fragment thereof that binds IL-13 comprising a light chain variable region and a heavy chain variable region, wherein the light chain variable region is at least about 95% identical to SEQ ID NO: 97, 99, 101, or 103; and wherein the heavy chain variable region is at least about 95% identical to SEQ ID NOs: SEQ ID NO: 98, 100, 102, or 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, wherein the amino acid positions are based on Kabat numbering.

In some embodiments, the polynucleotide encodes an antibody or antigen binding fragment thereof that binds IL-13 comprising a light chain variable region and a heavy chain variable region, wherein the light chain variable region is about 95% identical to SEQ ID NO: 97, 99, 101, or 103; and wherein the heavy chain variable region is about 95% identical to SEQ ID NOs: SEQ ID NO: 98, 100, 102, or 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, wherein the amino acid positions are based on Kabat numbering.

The polynucleotides that encode an anti-IL-13 antibody or antigen binding fragment thereof can be fused (e.g., operably linked) to one or more regulatory or control sequence and can be contained in suitable expression vectors or host cell. Each of the polynucleotides encoding the heavy or light chain variable domains can be independently fused to a polynucleotide sequence encoding a constant domain, such as a human constant domain, enabling the production of intact antibodies. Alternatively, polynucleotides, or portions thereof, can be fused together, providing a template for production of a single chain antibody.

For recombinant production, a polynucleotide encoding the antibody may be inserted into a replicable vector for cloning (amplification of the DNA) or for expression. Many suitable vectors for expressing the recombinant antibody are available. The vector components generally include, but are not limited to, one or more of the following: a signal sequence, an origin of replication, one or more marker genes, an enhancer element, a promoter, and a transcription termination sequence.

Expression and cloning vectors contain a nucleic acid sequence that enables the vector to replicate in one or more selected host cells. Generally, in cloning vectors this sequence is one that enables the vector to replicate independently of the host chromosomal DNA and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV, and BPV) are useful for cloning vectors in mammalian cells. Generally, the origin of replication component is not needed for mammalian expression vectors (the SV40 origin may typically be used only because it contains the early promoter).

Expression and cloning vectors may contain a gene that encodes a selectable marker to facilitate identification of expression. Typical selectable marker genes encode proteins that confer resistance to antibiotics or other toxins, e.g., ampicillin, neomycin, methotrexate, or tetracycline, or alternatively, are complement auxotrophic deficiencies, or in other alternatives supply specific nutrients that are not present in complex media, e.g., the gene encoding D-alanine racemase for Bacilli.

The anti-IL-13 antibodies or antigen binding fragments thereof can also be produced as fusion polypeptides, in which the antibody or fragment is fused with a heterologous polypeptide, such as a signal sequence or other polypeptide having a specific cleavage site at the amino terminus of the mature protein or polypeptide. The heterologous signal sequence selected is typically one that is recognized and processed (e.g., cleaved by a signal peptidase) by the host cell. For prokaryotic host cells that do not recognize and process the anti-IL-13 antibody signal sequence, the signal sequence can be substituted by a prokaryotic signal sequence. The signal sequence can be, for example, alkaline phosphatase, penicillinase, lipoprotein, heat-stable enterotoxin II leaders, and the like. For yeast secretion, the native signal sequence can be substituted, for example, with a leader sequence obtained from yeast invertase alpha-factor (including Saccharomyces and Kluyveromyces α-factor leaders), acid phosphatase, C. albicans glucoamylase, or the signal described in U.S. Pat. No. 5,631,144 (WO 90/13646). In mammalian cells, mammalian signal sequences as well as viral secretory leaders, for example, the herpes simplex gD signal, can be used. The DNA for such precursor region is ligated in reading frame to DNA encoding the anti-IL-13 antibody or antigen binding fragment thereof.

Anti-IL-13 antibodies or antigen binding fragments thereof may be made by any method including, for example, by recombinant means. For example, anti-IL-13 antibodies or antigen binding fragment thereof can be made using the expression vectors provided herein including, for example, transfecting a host cell with the expression vectors comprising polynucleotides coding for the anti-IL-13 antibody or antigen binding fragment thereof.

In some embodiments, the ability of the produced antibody to bind to IL-13 and/or other related members of the IL protein family can be assessed using standard binding assays, such as surface plasmon resonance (SPR), ForteBio (BLI), ELISA, Western Blot, Immunofluorescent, or flow cytometric analysis.

### Pharmaceutical Compositions and Methods of Treatment

Also provided herein are compositions including, for example, pharmaceutical compositions that comprise an anti-IL-13 antibody or antigen binding fragment thereof as described herein. Such compositions may be used in the treatment and/or prevention of a subject predisposed to or having a disease or disorder including, for example, an immunological related disease or disorder such as asthma (including, but not limited to, mild asthma, mild to moderate asthma, moderate asthma, moderate to severe asthma, and/or severe asthma), allergic asthma, allergen-induced airway obstruction due to asthma and/or allergic asthma, atopic dermatitis (including, but not limited to, mild atopic dermatitis, mild to moderate atopic dermatitis, moderate atopic dermatitis, moderate-to-severe, and/or severe atopic dermatitis), eczema, acute urticaria, idiopathic pulmonary fibrosis (IPF), chronic obstructive pulmonary disease (COPD), chronic obstructive airway disease, emphysema, chronic bronchitis, pulmonary fibrosis, systemic sclerosis, scleroderma, cryptogenic fibrosing alveolitis, usual interstitial pneumonitis, idiopathic interstitial pneumonitis, wound, systemic lupus erythematosus (SLE), rheumatoid arthritis (RA), systemic sclerosis (SS), ulcerative colitis (UC), type 1 diabetes (T1D), sjogren's syndrome (SS), prurigo nodularis, nasal polyposis, recurrent urticaria, aspergillosis, bullous pemphigoid, chronic sinusitis, alopecia areata, hay fever, pemphigus, allergic rhinitis, psoriasis, rosacea, allergic drug reactions, allergic reactions, anaphylaxis, acne, food allergies, inflammatory bowel disease (IBS), Crohn's disease, ulcerative colitis, chronic rhinosinusitis with nasal polyps (CRSwNP), eosinophilic esophagitis, eosinophilic colitis, eosinophilic gastritis, eosinophilic cystitis, eosinophilic fasciitis, eosinophilic pustular folliculitis, eosinophilic granulomatosis, eosinophilic polyangiitis, Churg-Strauss syndrome, biliary cancer, brain cancer, breast cancer, colorectal cancer, genitourinary cancer, head and neck cancer, liver cancer, Hodgkin's lymphoma, esophageal cancer, pancreatic cancer, prostate cancer, renal cancer, Kaposi's sarcoma, sinusitis, or chronic urticaria.

Also provided herein are methods of treating a disease or disorder (e.g., an immunological-related disease or disorder) in a subject (e.g., a human patient) in need thereof that comprise administering to the subject a therapeutically effective dose of an anti-IL-13 antibody or antigen binding fragment thereof as described herein (e.g., a pharmaceutical composition comprising an anti-IL-13 antibody or antigen binding fragment thereof as described herein), wherein the IL-13 antibody or antigen binding fragment thereof comprises: a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3); b) a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3); c) a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3); d) a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3); e) a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3); f) a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3); g) a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3); h) a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3); i) a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3); j) a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3); k) a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3); l) a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3); m) a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3); n) a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3); o) a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); or p) a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3). In some embodiments, the disease or disorder is an immunological related disease or disorder such as asthma (including, but not limited to, mild asthma, mild to moderate asthma, moderate asthma, moderate to severe asthma, and/or severe asthma), allergic asthma, allergen-induced airway obstruction due to asthma and/or allergic asthma, atopic dermatitis (including, but not limited to, mild atopic dermatitis, mild to moderate atopic dermatitis, moderate atopic dermatitis, moderate-to-severe, and/or severe atopic dermatitis), eczema, acute urticaria, idiopathic pulmonary fibrosis (IPF), chronic obstructive pulmonary disease (COPD), chronic obstructive airway disease, emphysema, chronic bronchitis, pulmonary fibrosis, systemic sclerosis, scleroderma, cryptogenic fibrosing alveolitis, usual interstitial pneumonitis, idiopathic interstitial pneumonitis, wound, systemic lupus erythematosus (SLE), rheumatoid arthritis (RA), systemic sclerosis (SS), ulcerative colitis (UC), type 1 diabetes (T1D), sjogren's syndrome (SS), prurigo nodularis, nasal polyposis, recurrent urticaria, aspergillosis, bullous pemphigoid, chronic sinusitis, alopecia areata, hay fever, pemphigus, allergic rhinitis, psoriasis, rosacea, allergic drug reactions, allergic reactions, anaphylaxis, acne, food allergies, inflammatory bowel disease (IBS), Crohn's disease, ulcerative colitis, chronic rhinosinusitis with nasal polyps (CRSwNP), eosinophilic esophagitis, eosinophilic colitis, eosinophilic gastritis, eosinophilic cystitis, eosinophilic fasciitis, eosinophilic pustular folliculitis, eosinophilic granulomatosis, eosinophilic polyangiitis, Churg-Strauss syndrome, biliary cancer, brain cancer, breast cancer, colorectal cancer, genitourinary cancer, head and neck cancer, liver cancer, Hodgkin's lymphoma, esophageal cancer, pancreatic cancer, prostate cancer, renal cancer, Kaposi's sarcoma, sinusitis, or chronic urticaria.

Also provided herein are methods of treating a disease or disorder (e.g., an immunological-related disease or disorder) in a subject (e.g., a human patient) in need thereof that is not responsive to one or more standard of care treatments (e.g., a corticosteroid treatment) that comprise administering to the subject a therapeutically effective dose of an anti-IL-13 antibody or antigen binding fragment thereof as described herein (e.g., a pharmaceutical composition comprising an anti-IL-13 antibody or antigen binding fragment thereof as described herein), wherein the IL-13 antibody or antigen binding fragment thereof comprises: a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3); b) a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3); c) a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3); d) a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3); e) a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3); f) a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3); g) a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3); h) a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3); i) a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3); j) a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3); k) a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3); l) a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3); m) a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3); n) a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3); o) a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); or p) a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3). In some embodiments, the disease or disorder is an immunological related disease or disorder such as asthma (including, but not limited to, mild asthma, mild to moderate asthma, moderate asthma, moderate to severe asthma, and/or severe asthma), allergic asthma, allergen-induced airway obstruction due to asthma and/or allergic asthma, atopic dermatitis (including, but not limited to, mild atopic dermatitis, mild to moderate atopic dermatitis, moderate atopic dermatitis, moderate-to-severe, and/or severe atopic dermatitis), eczema, acute urticaria, idiopathic pulmonary fibrosis (IPF), chronic obstructive pulmonary disease (COPD), chronic obstructive airway disease, emphysema, chronic bronchitis, pulmonary fibrosis, systemic sclerosis, scleroderma, cryptogenic fibrosing alveolitis, usual interstitial pneumonitis, idiopathic interstitial pneumonitis, wound, systemic lupus erythematosus (SLE), rheumatoid arthritis (RA), systemic sclerosis (SS), ulcerative colitis (UC), type 1 diabetes (T1D), sjogren's syndrome (SS), prurigo nodularis, nasal polyposis, recurrent urticaria, aspergillosis, bullous pemphigoid, chronic sinusitis, alopecia areata, hay fever, pemphigus, allergic rhinitis, psoriasis, rosacea, allergic drug reactions, allergic reactions, anaphylaxis, acne, food allergies, inflammatory bowel disease (IBS), Crohn's disease, ulcerative colitis, chronic rhinosinusitis with nasal polyps (CRSwNP), eosinophilic esophagitis, eosinophilic colitis, eosinophilic gastritis, eosinophilic cystitis, eosinophilic fasciitis, eosinophilic pustular folliculitis, eosinophilic granulomatosis, eosinophilic polyangiitis, Churg-Strauss syndrome, biliary cancer, brain cancer, breast cancer, colorectal cancer, genitourinary cancer, head and neck cancer, liver cancer, Hodgkin's lymphoma, esophageal cancer, pancreatic cancer, prostate cancer, renal cancer, Kaposi's sarcoma, sinusitis, or chronic urticaria.

Also provided is a therapeutically effective dose of an anti-IL-13 antibody or antigen binding fragment thereof as described herein (e.g., a pharmaceutical composition comprising an anti-IL-13 antibody or antigen binding fragment thereof as described herein), for use as a medicament, or for use in a method of treating a disease or disorder (e.g., such as asthma (including, but not limited to, mild asthma, mild to moderate asthma, moderate asthma, moderate to severe asthma, and/or severe asthma), allergic asthma, allergen-induced airway obstruction due to asthma and/or allergic asthma, atopic dermatitis (including, but not limited to, mild atopic dermatitis, mild to moderate atopic dermatitis, moderate atopic dermatitis, moderate-to-severe, and/or severe atopic dermatitis), eczema, acute urticaria, idiopathic pulmonary fibrosis (IPF), chronic obstructive pulmonary disease (COPD), chronic obstructive airway disease, emphysema, chronic bronchitis, pulmonary fibrosis, systemic sclerosis, scleroderma, cryptogenic fibrosing alveolitis, usual interstitial pneumonitis, idiopathic interstitial pneumonitis, wound, systemic lupus erythematosus (SLE), rheumatoid arthritis (RA), systemic sclerosis (SS), ulcerative colitis (UC), type 1 diabetes (T1D), sjogren's syndrome (SS), prurigo nodularis, nasal polyposis, recurrent urticaria, aspergillosis, bullous pemphigoid, chronic sinusitis, alopecia areata, hay fever, pemphigus, allergic rhinitis, psoriasis, rosacea, allergic drug reactions, allergic reactions, anaphylaxis, acne, food allergies, inflammatory bowel disease (IBS), Crohn's disease, ulcerative colitis, chronic rhinosinusitis with nasal polyps (CRSwNP), eosinophilic esophagitis, eosinophilic colitis, eosinophilic gastritis, eosinophilic cystitis, eosinophilic fasciitis, eosinophilic pustular folliculitis, eosinophilic granulomatosis, eosinophilic polyangiitis, Churg-Strauss syndrome, biliary cancer, brain cancer, breast cancer, colorectal cancer, genitourinary cancer, head and neck cancer, liver cancer, Hodgkin's lymphoma, esophageal cancer, pancreatic cancer, prostate cancer, renal cancer, Kaposi's sarcoma, sinusitis, or chronic urticaria), wherein the IL-13 antibody or antigen binding fragment thereof comprises: a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3); b) a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3); c) a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3); d) a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3); e) a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3); f) a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3); g) a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3); h) a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3); i) a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3); j) a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3); k) a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3); l) a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3); m) a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3); n) a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3); o) a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); or p) a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3).

In some embodiments, the anti-IL-13 antibody or antigen binding fragment thereof (e.g., a pharmaceutical composition comprising an anti-IL-13 antibody or antigen binding fragment thereof as described herein) is combined with one or more standard of care therapies selected from: an immunosuppressant, an antihistamine (e.g., diphenhydramine, chlorpheniramine, cetirizine, loratadine, fexofenadine, or doxylamine) a corticosteroid, a calcineurin inhibitor, a phosphodiesterase-4 inhibitor, an antimicrobial, and an antibody that blocks signaling of Interleukin (IL)-4/IL-13, or combinations thereof. In some embodiments, the combination of the anti-IL-13 antibody or antigen binding fragment thereof as described herein and standard of care therapy is administered to a subject in need thereof.

In some embodiments, the anti-IL-13 antibody or antigen binding fragment thereof as described herein (e.g., a pharmaceutical composition comprising an anti-IL-13 antibody or antigen binding fragment thereof as described herein) is combined with a thymic stromal lymphopoietin (TSLP) antagonist (e.g., an antibody to TSLP). In some embodiments the antibody to TSLP is tezepelumab. In some embodiments the antibody to TSLP is GB-0895. In some embodiments, the combination of the anti-IL-13 antibody or antigen binding fragment thereof and TSLP antagonist is administered to a subject in need thereof.

In some embodiments, the anti-IL-13 antibody or antigen binding fragment thereof as described herein (e.g., a pharmaceutical composition comprising an anti-IL-13 antibody or antigen binding fragment thereof as described herein) is combined with a OX40L antagonist (e.g., an antibody to OX40L). In some embodiments, the anti-IL-13 antibody or antigen binding fragment thereof (e.g., a pharmaceutical composition comprising an anti-IL-13 antibody or antigen binding fragment thereof) is combined with a TL1A antagonist (e.g., an antibody to TL1A). In some embodiments, the combination of the anti-IL-13 antibody or antigen binding fragment thereof as described herein and OX40L or TL1A antagonist is administered to a subject in need thereof.

In some embodiments, the anti-IL-13 antibody or antigen binding fragment thereof described herein (e.g., a pharmaceutical composition comprising an anti-IL-13 antibody or antigen binding fragment thereof as described herein) is combined with another (e.g., a different) anti-IL-13 antibody (e.g., lebrikizumab).

The pharmaceutical composition described herein may be administered in an effective amount.

The pharmaceutical composition may be formulated with a pharmaceutically acceptable carrier or diluent as well as any other known adjuvants and excipients in accordance with conventional techniques such as those provided in Remington: The Science and Practice of Pharmacy, 19th Edition, Gennaro, Ed., Mack Publishing Co., Easton, Pa., 1995.

As used herein, “pharmaceutically acceptable carrier” includes any and all solvents, dispersion media, coatings, antibacterial and antifungal agents, isotonic and absorption delaying agents, and the like that are physiologically compatible. The carrier may be suitable for intravenous, intramuscular, subcutaneous, parenteral, spinal, or epidermal administration (e.g., by injection or infusion). Depending on the route of administration, the anti-IL-13 antibody as described herein may be coated in a material to protect the compound from the action of acids and other natural conditions that may inactivate the compound.

Typically, compositions for administration by injection are solutions in sterile isotonic aqueous buffer. Where necessary, the pharmaceutical composition can also include a solubilizing agent and a local anesthetic such as lignocaine to ease pain at the site of the injection. Generally, the ingredients are supplied either separately or mixed in unit dosage form, for example, as a dry lyophilized powder or water free concentrate in a hermetically sealed container such as an ampoule or sachette indicating the quantity of the active agent. Where the pharmaceutical composition is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

Dosage levels of the anti-IL-13 antibody or antigen binding fragment thereof as described herein in the pharmaceutical composition may be varied to obtain an amount of the anti-IL-13 antibody or antigen binding fragment thereof as described herein which is effective to achieve the desired therapeutic response for a particular subject, composition, and mode of administration, without being toxic to the subject. The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

The terms “treatment” and “therapy” and the like, as used herein, are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the anti-IL-13 antibody as described herein, the result should be considered an effective treatment of the underlying disorder regardless of whether all the symptoms of the disorder are alleviated or not.

As used herein, the term “effective amount” means that dose of an anti-IL-13 antibody or antigen binding fragment thereof as described herein that will elicit the biological or medical response of a tissue, system, animal, or human that is being sought, for instance, by a researcher or clinician. Furthermore, the term “therapeutically effective dose” means any dose that, as compared to a corresponding subject who has not received such dose, results in improved treatment, healing, prevention, or amelioration of a disease, disorder, or side effect, or a decrease in the rate of advancement of a disease or disorder. The term also includes within its scope doses effective to enhance normal physiological function. Therapeutically effective amounts and treatment regimens are generally determined empirically and may be dependent on factors, such as the age, weight, and health status of the patient and disease or disorder to be treated. Such factors are within the purview of the attending physician.

The term “subject” for purposes of treatment refers to any animal classified as a mammal, including humans, domesticated and farm animals, and zoo, sports, or pet animals, such as dogs, horses, cats, cows, and the like. Preferably, the mammal is a human.

### Computational Definition and Verification of Sequences

Applicant, through computational design and experimental validation, has facilitated an understanding of structure and function interrelation of anti-IL-13 antibodies and herein provides the skilled artisan the means to use such understanding. For example, the application provides Computer Program Listing Appendices (also referred to herein as Appendix A and Appendix B, respectively) that can be used to both evaluate (score) a given sequence or generate a sequence having a score that, when evaluated, is above a given threshold. These appendices include a Potts model (generalized Ising model) of polypeptides provided by the invention. In some embodiments, an implementation of a Potts model supposes that amino acids interact with their nearest neighbor but is able to determine relationship with distant (e.g., more than one position away, therefore representing features of secondary and tertiary structure) amino acids as well.

The Potts model is a second order sequence model. In some embodiments, the Potts model is energy based. The model is designed to output an energy for a particular configuration. Given a sequence provided to an energy-based model, the model can be trained to produce a quantification of any measure. The Potts model is represented by a table of model weights. The table includes first- and second-order weights. In the first-order weights, the table includes position of a residue, the residue (e.g., amino acid), and a score associated with the residue at that position. In the second-order weights, the table includes two positions, two residues, and one score for the two residues at the respective positions. The scores are self-energies or energy scores.

The Computer Program Listing Appendices are referred to as Appendix A (score.py) and Appendix B (model.etab), which are available online at the respective addresses listed above and incorporated by reference in full into this application. A person having ordinary skill in the art can recognize that Appendix A can be executed in a python environment, compiler, or other equivalent environment that can run python scripts. A person having ordinary skill in the art can recognize that Appendix B can be loaded by the script of Appendix A. When running the score.py file, two sequences (or one combined sequence) can be inputted. In embodiments, the below description further describes the source code and model of Appendices A and Appendix B.

A zero-order model is a set of unordered amino acids. As the model is zero-order, there is no order or structure to the amino acids. In a physical metaphor, it can be thought of as a “bag” of amino acids because the amino acids placed in the bag would jumble around and have no order. Next, a first order model is a matrix which is represented as a quantification (e.g., a peptide mass fingerprinting (PMF) of amino acids) at each residue of a protein sequence. Each row of the matrix is an amino acid sequence, and each column is configured to have a sum of the quantification of each residue in that column be 1. These first order energies can be referred to as an h-tensor, represented below as the “first hash table.” A first order term in the model is said to be at a given position, and the model can assign a number of points for each particular position. With pair terms, the model can compare the scores given any two positions. The model can output an “energy,” which is a number, and the lower the number the better, in embodiments.

A second-order sequence model, or Potts model, represents energy for two residues being found together and energies associated with the first-order effects. The second-order energies of the two residues being found together are considered the j-tensor, while the energies associated with the first-order effects are referred to as the h-tensor. The second order energies, in other words, compare the energies between two positions of the amino acid sequence. The second order sequences can therefore compare the energies between two positions of any amino acid sequence. A person of ordinary skill in the art can recognize that this can be repeated for multiple sequences, thereby rating many similar sequences all residue pairs of similar sequences.

Described herein is an example of the process used by embodiments of this disclosure to determine claimed sequences. First, the process receives a given polypeptide sequence or pair of sequences. The sequence pair, in different embodiments, can be separate polypeptide chains or, in some embodiments, a single polypeptide chain, e.g., an scFv. A person of ordinary skill in the art can further recognize that model of Appendix B, in embodiments, can be used to generate polypeptide sequences or pairs of sequences to test using the script of Appendix A. The script of Appendix A scores the sequence using model weights provided in Appendix B. The script then determines whether the score is above a predetermined threshold (although, a person of ordinary skill in the art can recognize that with different scoring regimes, the score may be below the threshold). If not, the polypeptide sequence or pair of sequences is not verified. If it is, the polypeptide sequence or pair of sequences is verified.

The Potts Model disclosed herein was generated from experimental validation of approximately 300 sequences to identify sequences with advantageous functions. The model coefficients are stored in the ASCII text file (model.etab). These sequences when assayed had favorable binding to human IL-13 as measured by an SPR-based assay.

In some embodiments, a script is employed to determine whether a sequence pair (e.g., VH and VL—although, for clarity, the sequence pair, in different embodiments, can be separate polypeptide chains or, in some embodiments, a single polypeptide chain, e.g., an scFv) is claimed under the model, when scored, is above a threshold. The script is referred to as a computationally binding optimizing (CBO) script. Two hash tables are loaded from an existing file, and the script extracts first- and second-order index/position values, residue values, and corresponding scores for each. For the first-order table, the script extracts one position, one residue, and one score associated with the residue at the position. For the second-order table, the script extracts a first position, a second position, a first residue, a second residue, and a score associated with the first residue at the first position and the second residue at the second position.

The two sequences, the VH sequence and VL sequence, are inputted to the script are then aligned. The alignment confirms that the VH sequence and VL sequence are the same length as reference sequence(s) and that given sections of both sequences are the same as the reference sequence(s). Once confirmed, residues from both of the inputted sequences at specified positions are extracted to form a trimmed and concatenated sequence. The positions used to extract the residues are positions where mutations were applied during training of the model. The positions used to confirm that the given sections of both sequences are the same as the reference sequence(s) are the positions where mutations were not applied during training of the model. Once trimmed to the positions used to train the model, the two input sequences are concatenated together. Then an energy score is calculated for the given concatenated sequence and the two hash tables. The following steps describe some embodiments of calculating the energy score.

First, let \vec{σ} designate the sequence being scored with \vec{σ}ᵢ being its i-th amino acids. Further, let h represent the self-energy matrix (e.g., first-order matrix) of the Potts model, such that hᵢ(a) is the energy associated with amino acid a being at position i. Finally, let J represent the pair-energy matrix (e.g., second-order matrix) of the model, such that Jᵢⱼ(a, b) is the pair energy associated with amino acids a and b being at positions i and j, respectively. With this notation, the total energy of sequence o under the model is:

$$E ( \vec{σ} ) = {\sum }_{i = 1}^{N} {h}_{i} ( {σ}_{i} ) + {\sum }_{i = 1}^{N - 1} {\sum }_{j = i + 1}^{N} {J}_{ij} ( {σ}_{i} , {σ}_{j} )$$

where N is the length of the sequence. Further, \vec{σ}_{i→a} can represent a sequence resulting in replacing the i-th amino acids of sequence \vec{σ} with amino acid a. With this notation, a pseudo-likelihood of sequence \vec{σ} under the model can be calculated as:

$$ρ ( \vec{σ} ) = {{\prod }^{N}}_{i = 1} \frac{{e}^{- E ( \vec{σ} )}}{{\sum }_{a = 1}^{μ} {e}^{- E ( {\vec{σ}}_{i \to a} )}}$$

where μ is the total number of possible amino acids. In some embodiments, μ is 20 but a person of ordinary skill in the art can recognize that the value of μ can vary. Finally, the logarithm of the pseudo-likelihood of \vec{σ} can be expressed as:

$$log [ ρ ( \vec{σ} ) ] = {\sum }_{i = 1}^{N} log [ \frac{{e}^{- E ( \vec{σ} )}}{{\sum }_{a = 1}^{μ} {e}^{- E ( {\vec{σ}}_{i \to a} )}} ] = - {\sum }_{i = 1}^{N} ( E ( \vec{σ} ) + log [ {\sum }_{a = 1}^{μ} {e}^{- E ( {\vec{σ}}_{i \to a} )} ] )$$

The score sequence function of the script of Appendix A calculates and returns the energy according to the Potts model. If the score is greater than the threshold, which in this embodiment is 0, then the sequences can meet the required parameters (e.g., be confirmed/verified by the Potts model) and the function returns true. Otherwise, the function returns false and the sequence pair is not confirmed the model. When the function returns true, this indicates that the sequence pair is suitable for the given purpose (e.g., a purpose related to binding to human IL-13).

A person of ordinary skill in the art can recognize that other thresholds can be employed that represent a logarithmic probability that sequences can meet the required parameters. In some embodiments, an example range of thresholds calculated by the script can include about −1.7, −1.0, −0.5, 0, 0.5, 1, 1.5, 1.8, 2.0, and 3.0. As the thresholds increase, the sequences spaces scored within those thresholds represent smaller sequence spaces that have higher levels of confidence. In some embodiments, the constants may be modified in script or the hash table, such as by multiplying all constants by a constant. In such an event, the threshold may be adjusted, as can be determined by a person of ordinary skill in the art. The threshold can further be calculated in a non-logarithmic manner, and represented as a confidence interval, or in a non-logarithmic range, or represented as another probabilistic threshold.

In some embodiments, the script of Appendix A defines a sequence scoring function (score_sequence_energy). This function calculates a sequence energy for the replacement of a residue (e.g., an amino acid) with each other possible residue a group of positions in the sequence using a Potts model trained on replacing residues at those positions. As described above, the scoring checks each possible residue at each position requested. A person of ordinary skill in the art can recognize that the script can replace residues at each given position (as in the script of Appendix A). In some embodiments, a similar script could add residues into the sequence. Each score is calculated with a score_sequence_energy function and is added to an array of scores. For each position examined, sum of the scores in the array are summed and returned.

In some embodiments, the sequence energy function inputs a sequence and the hash tables determined from the inputted etab file (or another format). The function iterates over all first order combinations, scoring them with a first hash table, and all second order combinations, scoring them with a second hash table. The score_sequence_energy function considers all replacement residues and their first and second order combinations and sums those energies together.

In some embodiments, the etab file is read with a read_etab function. Each line in the file having three items is unpacked and converted into an entry in a first hash table for first order combinations. Each line in the file having five items is unpacked and converted into an entry of a second hash table for second order combinations. The two hash tables are returned.

The above steps and functions describe a “computationally binding optimized” (CBO) sequence, amino acid sequence, or polypeptide sequence. Such a sequence is a sequence that, when inputted into the above computationally binding optimizing script, returns a value of true. A person having ordinary skill in the art can recognize that the script can be implemented in other ways or with other series of steps. A person having ordinary skill in the art can also recognize that other functionally equivalent tables and constants can be used. However, the provided Python script, tables, and constants, when executed by a processor, are configured to output whether a given polypeptide sequence is a CBO sequence. Therefore, any sequence that, when run by the script, causes a Boolean “true” output is considered a CBO sequence. Any sequence that, when run by the script, causes a Boolean “false” output is considered excluded from the group of the CBO sequences. Thereby, in some embodiments, the script of Appendix A itself does not determine CBO sequences but is a tool to confirm whether a given sequence is a CBO sequence or not. When a CBO sequence is an amino acid sequence, the corresponding molecule having the amino acid sequence is referred to as a CBO polypeptide.

In some embodiments, a similar script is able to return the calculated scores of a given or multiple sequences in addition to or separate from a Boolean value representing a threshold.

In some embodiments, a similar script can return one or more generated sequences from the Potts model. To generate sequences, Markov Chain Monte Carlo (MCMC) sampling can be performed on the model. Then, the resulting samples can be reintegrated into the constant template. In some embodiments, a brute force method can be applied using the scoring functions.

The sequences for AbA, AbB, AbC, AbD, AbE, AbF, AbG, AbH, AbI, AbJ, AbK, AbL, AbM, AbN, AbO, and AbP are illustrated in Tables 3. All of the proteins AbA, AbB, AbC, AbD, AbE, AbF, AbG, AbH, AbI, AbJ, AbK, AbL, AbM, AbN, AbO, and AbP were or would be verified by the script because their Potts model scores were above 0.0.

A person of ordinary skill in the art can recognize that equivalent Potts model scores, such as scores having a different scale or being multiplied by negative one, may result in scores being at or above or below a different threshold.

A computer network or similar digital processing environment is described in which embodiments of the present invention may be implemented. Client computer(s)/devices and server computer(s) provide processing, storage, and input/output devices executing application programs and the like. The client computer(s)/devices can also be linked through communications network to other computing devices, including other client devices/processes and server computer(s). The communications network can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

An example internal structure of a computer (e.g., client processor/device or server computers) is described in the computer system as described herein. Each computer contains a system bus, where a bus is a set of hardware lines used for data transfer among the components of a computer or processing system. The system bus is essentially a shared conduit that connects different elements of a computer system (e.g., processor, disk storage, memory, input/output ports, network ports, etc.) that enables the transfer of information between the elements. Attached to the system bus is an I/O device interface for connecting various input and output devices (e.g., keyboard, mouse, displays, printers, speakers, etc.) to the computer. A network interface allows the computer to connect to various other devices attached to a network (e.g., network). Memory provides volatile storage for computer software instructions and data used to implement some embodiments of the present invention (e.g., CBO determination script, Potts model, model weights code detailed above and in Appendices A and B). Disk storage provides non-volatile storage for computer software instructions and data used to implement some embodiments of the present invention. A central processor unit is also attached to the system bus and provides for the execution of computer instructions.

In one embodiment, the processor routines and data are a computer program product, including a non-transitory computer-readable medium (e.g., a removable storage medium such as one or more DVD-ROM's, CD-ROM's, diskettes, tapes, etc.) that provides at least a portion of the software instructions for the invention system. The computer program product can be installed by any suitable software installation procedure, as is well known in the art. In another embodiment, at least a portion of the software instructions may also be downloaded over a cable communication and/or wireless connection. In some embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program.

In another aspect, the disclosure provides a polypeptide that specifically binds an IL-13 (e.g., an hIL-13): wherein a CBO model outputs a score that is above a predetermined threshold upon scoring an amino acid sequence of the polypeptide.

In another aspect, the disclosure provides a CBO polypeptide that specifically binds an IL-13 (e.g., an hIL-13): wherein a CBO model outputs a score that is above a predetermined threshold upon scoring an amino acid sequence representing the CBO polypeptide.

In another aspect, the disclosure provides a polypeptide that specifically binds an IL-13 (e.g., an hIL-13) and receives a score above a predetermined threshold from a CBO model upon scoring an amino acid sequence representing the polypeptide.

In another aspect, the disclosure provides a polypeptide that specifically binds an IL-13 (e.g., an hIL-13): wherein the polypeptide receives a score above a predetermined threshold from a CBO model upon scoring an amino acid sequence representing the polypeptide.

In another aspect, the disclosure provides a computationally binding optimized (CBO) polypeptide, determined by a computationally binding optimized (CBO) model that specifically binds an IL-13 (e.g., an hIL-13).

In some embodiments, a CBO polypeptide comprises a paratope that is substantially similar to the paratope of an antibody comprising an amino acid sequence selected from: SEQ ID NO: 97 and SEQ ID NO: 98 (AbA); SEQ ID NO: 99 and SEQ ID NO: 100 (AbB); SEQ ID NO: 101 and SEQ ID NO: 102 (AbC); SEQ ID NO: 103 and SEQ ID NO: 104 (AbD); SEQ ID NO: 105 and SEQ ID NO: 106 (AbE); SEQ ID NO: 107 and SEQ ID NO: 108 (AbF); SEQ ID NO: 109 and SEQ ID NO: 110 (AbG); SEQ ID NO: 111 and SEQ ID NO: 112 (AbH); SEQ ID NO: 113 and SEQ ID NO: 114 (AbI); SEQ ID NO: 115 and SEQ ID NO: 116 (AbJ); SEQ ID NO: 117 and SEQ ID NO: 118 (AbK); SEQ ID NO: 119 and SEQ ID NO: 120 (AbL); SEQ ID NO: 121 and SEQ ID NO: 122 (AbM); SEQ ID NO: 123 and SEQ ID NO: 124 (AbN); SEQ ID NO: 125 and SEQ ID NO: 126 (AbO); or SEQ ID NO: 127 and SEQ ID NO: 128 (AbP), or a combination of the foregoing.

In some embodiments, the CBO polypeptide comprises a paratope that is substantially similar (e.g., having at least about 90% sequence identity; having 100% sequence identity) to the paratope of a VL/VH combination selected from: SEQ ID NO: 97/SEQ ID NO: 98 (AbA); SEQ ID NO: 99/SEQ ID NO: 100 (AbB); SEQ ID NO: 101/SEQ ID NO: 102 (AbC); SEQ ID NO: 103/SEQ ID NO: 104 (AbD); SEQ ID NO: 105/SEQ ID NO: 106 (AbE); SEQ ID NO: 107/SEQ ID NO: 108 (AbF); SEQ ID NO: 109/SEQ ID NO: 110 (AbG); SEQ ID NO: 111/SEQ ID NO: 112 (AbH); SEQ ID NO: 113/SEQ ID NO: 114 (AbI); SEQ ID NO: 115/SEQ ID NO: 116 (AbJ); SEQ ID NO: 117/SEQ ID NO: 118 (AbK); SEQ ID NO: 119/SEQ ID NO: 120 (AbL); SEQ ID NO: 121/SEQ ID NO: 122 (AbM); SEQ ID NO: 123/SEQ ID NO: 124 (AbN); SEQ ID NO: 125/SEQ ID NO: 126 (AbO); or SEQ ID NO: 127/SEQ ID NO: 128 (AbP).

In some embodiments, the CBO polypeptide comprises a paratope that differs from the paratope of a VL/VH combination selected from: SEQ ID NO: 97/SEQ ID NO: 98 (AbA); SEQ ID NO: 99/SEQ ID NO: 100 (AbB); SEQ ID NO: 101/SEQ ID NO: 102 (AbC); SEQ ID NO: 103/SEQ ID NO: 104 (AbD); SEQ ID NO: 105/SEQ ID NO: 106 (AbE); SEQ ID NO: 107/SEQ ID NO: 108 (AbF); SEQ ID NO: 109/SEQ ID NO: 110 (AbG); SEQ ID NO: 111/SEQ ID NO: 112 (AbH); SEQ ID NO: 113/SEQ ID NO: 114 (AbI); SEQ ID NO: 115/SEQ ID NO: 116 (AbJ); SEQ ID NO: 117/SEQ ID NO: 118 (AbK); SEQ ID NO: 119/SEQ ID NO: 120 (AbL); SEQ ID NO: 121/SEQ ID NO: 122 (AbM); SEQ ID NO: 123/SEQ ID NO: 124 (AbN); SEQ ID NO: 125/SEQ ID NO: 126 (AbO); or SEQ ID NO: 127/SEQ ID NO: 128 (AbP), by substitution (e.g., conservative such as highly conservative substitution) of from 1 to 3 (e.g., 1, 2 or 3) residues.

In particular embodiments, the CBO polypeptide does not comprise a VL and VH pair selected from: SEQ ID NO: 97 and SEQ ID NO: 98 (AbA); SEQ ID NO: 99 and SEQ ID NO: 100 (AbB); SEQ ID NO: 101 and SEQ ID NO: 102 (AbC); SEQ ID NO: 103 and SEQ ID NO: 104 (AbD); SEQ ID NO: 105 and SEQ ID NO: 106 (AbE); SEQ ID NO: 107 and SEQ ID NO: 108 (AbF); SEQ ID NO: 109 and SEQ ID NO: 110 (AbG); SEQ ID NO: 111 and SEQ ID NO: 112 (AbH); SEQ ID NO: 113 and SEQ ID NO: 114 (AbI); SEQ ID NO: 115 and SEQ ID NO: 116 (AbJ); SEQ ID NO: 117 and SEQ ID NO: 118 (AbK); SEQ ID NO: 119 and SEQ ID NO: 120 (AbL); SEQ ID NO: 121 and SEQ ID NO: 122 (AbM); SEQ ID NO: 123 and SEQ ID NO: 124 (AbN); SEQ ID NO: 125 and SEQ ID NO: 126 (AbO); SEQ ID NO: 127 and SEQ ID NO: 128 (AbP), and combinations thereof.

In some embodiments, a CBO polypeptide comprises: a VH amino acid sequence comprising a HCDR1, a HCDR2 and a HCDR3 that are substantially similar to a HCDR1, a HCDR2 and a HCDR3, respectively, of SEQ ID NO: 98, 100, 102, 104, 106, 108, 110, 112, 114, 116, 118, 120, 122, 124, 126, or 128; and a VL amino acid sequence comprising a LCDR1, a LCDR2 and a LCDR3 that are substantially similar to a LCDR1, a LCDR2 and a LCDR3, respectively, of SEQ ID NO: 97, 99, 101, 103, 105, 107, 109, 111, 113, 115, 117, 119, 121, 123, 125, or 127.

In some embodiments, the CBO polypeptide comprises a HCDR1, a HCDR2, a HCDR3, a LCDR1, a LCDR2 and a LCDR3 that substantially preserve one or more functional properties of a HCDR1, a HCDR2, a HCDR3, a LCDR1, a LCDR2 and a LCDR3 of AbA, AbB, AbC, AbD, AbE, AbF, AbG, AbH, AbI, AbJ, AbK, AbL, AbM, AbN, AbO, or AbP.

In some embodiments, the CBO polypeptide comprises a HCDR1, a HCDR2, a HCDR3, a LCDR1, a LCDR2 and a LCDR3 comprising only one or more conservative substitutions (e.g., only one or more highly conservative substitutions), relative a HCDR1, a HCDR2, a HCDR3, a LCDR1, a LCDR2 and a LCDR3 of AbA, AbB, AbC, AbD, AbE, AbF, AbG, AbH, AbI, AbJ, AbK, AbL, AbM, AbN, AbO, or AbP.

In certain embodiments, the CBO polypeptide comprises a HCDR1, a HCDR2, a HCDR3, a LCDR1, a LCDR2 and a LCDR3 comprising up to 1, 2, or 3 conservative substitutions (e.g., up to 1, 2, or 3 highly conservative substitutions), relative a HCDR1, a HCDR2, a HCDR3, a LCDR1, a LCDR2 and a LCDR3 of AbA, AbB, AbC, AbD, AbE, AbF, AbG, AbH, AbI, AbJ, AbK, AbL, AbM, AbN, AbO, or AbP.

In particular embodiments, the CBO polypeptide comprises a HCDR1, a HCDR2, a HCDR3, a LCDR1, a LCDR2 and a LCDR3 having 100% sequence identity to a HCDR1, a HCDR2, a HCDR3, a LCDR1, a LCDR2 and a LCDR3 of AbA, AbB, AbC, AbD, AbE, AbF, AbG, AbH, AbI, AbJ, AbK, AbL, AbM, AbN, AbO, or AbP.

In some embodiments, the CBO polypeptide comprises: a) a HCDR1 comprising at least 1 amino acid substitution relative to the amino acid sequence of SEQ ID NO: 4, SEQ ID NO: 10, SEQ ID NO: 16, SEQ ID NO: 22, SEQ ID NO: 28, SEQ ID NO: 34, SEQ ID NO: 40, SEQ ID NO: 46, SEQ ID NO: 52, SEQ ID NO: 58, SEQ ID NO: 64, SEQ ID NO: 70, SEQ ID NO: 76, SEQ ID NO: 82, SEQ ID NO: 88, or SEQ ID NO: 94 (e.g., at least one amino acid sequence set forth SEQ ID NO: 4, SEQ ID NO: 10, SEQ ID NO: 16, SEQ ID NO: 22, SEQ ID NO: 28, SEQ ID NO: 34, SEQ ID NO: 40, SEQ ID NO: 46, SEQ ID NO: 52, SEQ ID NO: 58, SEQ ID NO: 64, SEQ ID NO: 70, SEQ ID NO: 76, SEQ ID NO: 82, SEQ ID NO: 88, or SEQ ID NO: 94); b) a HCDR2 comprising at least 1 amino acid substitution relative to at least one amino acid sequence set forth in SEQ ID NO: 5, SEQ ID NO: 11, SEQ ID NO: 17, SEQ ID NO: 23, SEQ ID NO: 29, SEQ ID NO: 35, SEQ ID NO: 41, SEQ ID NO: 47, SEQ ID NO: 53, SEQ ID NO: 59, SEQ ID NO: 65, SEQ ID NO: 71, SEQ ID NO: 77, SEQ ID NO: 83, SEQ ID NO: 89, or SEQ ID NO: 95 (e.g., at least one amino acid sequence set forth in SEQ ID NO: 5, SEQ ID NO: 11, SEQ ID NO: 17, SEQ ID NO: 23, SEQ ID NO: 29, SEQ ID NO: 35, SEQ ID NO: 41, SEQ ID NO: 47, SEQ ID NO: 53, SEQ ID NO: 59, SEQ ID NO: 65, SEQ ID NO: 71, SEQ ID NO: 77, SEQ ID NO: 83, SEQ ID NO: 89, or SEQ ID NO: 95); c) a HCDR3 comprising at least 1 amino acid substitution relative to at least one amino acid sequence set forth in SEQ ID NO: 6, SEQ ID NO: 12, SEQ ID NO: 18, SEQ ID NO: 24, SEQ ID NO: 30, SEQ ID NO: 36, SEQ ID NO: 42, SEQ ID NO: 48, SEQ ID NO: 54, SEQ ID NO: 60, SEQ ID NO: 64, SEQ ID NO: 72, SEQ ID NO: 78, SEQ ID NO: 84, SEQ ID NO: 90, or SEQ ID NO: 96 (e.g., at least one amino acid sequence set forth in SEQ ID NO: 6, SEQ ID NO: 12, SEQ ID NO: 18, SEQ ID NO: 24, SEQ ID NO: 30, SEQ ID NO: 36, SEQ ID NO: 42, SEQ ID NO: 48, SEQ ID NO: 54, SEQ ID NO: 60, SEQ ID NO: 64, SEQ ID NO: 72, SEQ ID NO: 78, SEQ ID NO: 84, SEQ ID NO: 90, or SEQ ID NO: 96); d) a LCDR1 comprising at least 1 amino acid substitution relative to the amino acid sequence set forth in SEQ ID NO: 1, SEQ ID NO: 7, SEQ ID NO: 13, SEQ ID NO: 19, SEQ ID NO: 25, SEQ ID NO: 31, SEQ ID NO: 37, SEQ ID NO: 43, SEQ ID NO: 49, SEQ ID NO: 55, SEQ ID NO: 61, SEQ ID NO: 67, SEQ ID NO: 73, SEQ ID NO: 79, SEQ ID NO: 85, or SEQ ID NO: 91 (e.g., at least one amino acid sequence set forth in SEQ ID NO: 1, SEQ ID NO: 7, SEQ ID NO: 13, SEQ ID NO: 19, SEQ ID NO: 25, SEQ ID NO: 31, SEQ ID NO: 37, SEQ ID NO: 43, SEQ ID NO: 49, SEQ ID NO: 55, SEQ ID NO: 61, SEQ ID NO: 67, SEQ ID NO: 73, SEQ ID NO: 79, SEQ ID NO: 85, or SEQ ID NO: 91); e) a LCDR2 comprising at least 1 amino acid substitution relative to the amino acid sequence of SEQ ID NO: 2, SEQ ID NO: 8, SEQ ID NO: 14, SEQ ID NO: 20, SEQ ID NO: 26, SEQ ID NO: 32, SEQ ID NO: 38, SEQ ID NO: 44, SEQ ID NO: 50, SEQ ID NO: 56, SEQ ID NO: 62, SEQ ID NO: 68, SEQ ID NO: 74, SEQ ID NO: 80, SEQ ID NO: 86, or SEQ ID NO: 92 (e.g., at least one amino acid sequence set forth SEQ ID NO: 2, SEQ ID NO: 8, SEQ ID NO: 14, SEQ ID NO: 20, SEQ ID NO: 26, SEQ ID NO: 32, SEQ ID NO: 38, SEQ ID NO: 44, SEQ ID NO: 50, SEQ ID NO: 56, SEQ ID NO: 62, SEQ ID NO: 68, SEQ ID NO: 74, SEQ ID NO: 80, SEQ ID NO: 86, or SEQ ID NO: 92); f) a LCDR3 comprising at least 1 amino acid substitution relative to at least one amino acid sequence set forth in SEQ ID NO: 3, SEQ ID NO: 9, SEQ ID NO: 15, SEQ ID NO: 21, SEQ ID NO: 27, SEQ ID NO: 33, SEQ ID NO: 39, SEQ ID NO: 45, SEQ ID NO: 51, SEQ ID NO: 57, SEQ ID NO: 63, SEQ ID NO: 69, SEQ ID NO: 75, SEQ ID NO: 81, SEQ ID NO: 87, or SEQ ID NO: 93 (e.g., at least one amino acid sequence set forth in SEQ ID NO: 3, SEQ ID NO: 9, SEQ ID NO: 15, SEQ ID NO: 21, SEQ ID NO: 27, SEQ ID NO: 33, SEQ ID NO: 39, SEQ ID NO: 45, SEQ ID NO: 51, SEQ ID NO: 57, SEQ ID NO: 63, SEQ ID NO: 69, SEQ ID NO: 75, SEQ ID NO: 81, SEQ ID NO: 87, or SEQ ID NO: 93); or a combination of the foregoing.

In certain embodiments, a CBO polypeptide comprises: a VH amino acid sequence comprising a HCDR1, a HCDR2 and a HCDR3 that are substantially similar to a HCDR1, a HCDR2 and a HCDR3, respectively, of
(i) CDR1: SEQ ID NO: 4, CDR2: SEQ ID NO: 5, CDR3: SEQ ID NO: 6;
(ii) CDR1: SEQ ID NO: 10, CDR2: SEQ ID NO: 11, CDR3: SEQ ID NO: 12;
(iii) CDR1: SEQ ID NO: 16, CDR2: SEQ ID NO: 17, CDR3: SEQ ID NO: 18;
(iv) CDR1: SEQ ID NO: 22, CDR2: SEQ ID NO: 23, CDR3: SEQ ID NO: 24;
(v) CDR1: SEQ ID NO: 28, CDR2: SEQ ID NO: 29, CDR3: SEQ ID NO: 30;
(vi) CDR1: SEQ ID NO: 34, CDR2: SEQ ID NO: 35, CDR3: SEQ ID NO: 36;
(vii) CDR1: SEQ ID NO: 40, CDR2: SEQ ID NO: 41, CDR3: SEQ ID NO: 42;
(viii) CDR1: SEQ ID NO: 46, CDR2: SEQ ID NO: 47, CDR3: SEQ ID NO: 48;

(ix) CDR1: SEQ ID NO: 52, CDR2: SEQ ID NO: 53, CDR3: SEQ ID NO: 54;
(x) CDR1: SEQ ID NO: 58, CDR2: SEQ ID NO: 59, CDR3: SEQ ID NO: 60;
(xi) CDR1: SEQ ID NO: 64, CDR2: SEQ ID NO: 65, CDR3: SEQ ID NO: 66;
(xii) CDR1: SEQ ID NO: 70, CDR2: SEQ ID NO: 71, CDR3: SEQ ID NO: 72;
(xiii) CDR1: SEQ ID NO: 76, CDR2: SEQ ID NO: 77, CDR3: SEQ ID NO: 78;
(xiv) CDR1: SEQ ID NO: 82, CDR2: SEQ ID NO: 83, CDR3: SEQ ID NO: 84;
(xv) CDR1: SEQ ID NO: 88, CDR2: SEQ ID NO: 89, CDR3: SEQ ID NO: 90; and
(xvi) CDR1: SEQ ID NO: 94, CDR2: SEQ ID NO: 95, CDR3: SEQ ID NO: 96;
   and a VL amino acid sequence comprising a LCDR1, a LCDR2 and a LCDR3 that are substantially similar to a LCDR1, a LCDR2 and a LCDR3, respectively, of
(i) CDR1: SEQ ID NO: 1, CDR2: SEQ ID NO: 2, CDR3: SEQ ID NO: 3;
(ii) CDR1: SEQ ID NO: 7, CDR2: SEQ ID NO: 8, CDR3: SEQ ID NO: 9;
(iii) CDR1: SEQ ID NO: 13, CDR2: SEQ ID NO: 14, CDR3: SEQ ID NO: 15;
(iv) CDR1: SEQ ID NO: 19, CDR2: SEQ ID NO: 20, CDR3: SEQ ID NO: 21;
(v) CDR1: SEQ ID NO: 25, CDR2: SEQ ID NO: 26, CDR3: SEQ ID NO: 27;
(vi) CDR1: SEQ ID NO: 31, CDR2: SEQ ID NO: 32, CDR3: SEQ ID NO: 33;
(vii) CDR1: SEQ ID NO: 37, CDR2: SEQ ID NO: 38, CDR3: SEQ ID NO: 39;
(viii) CDR1: SEQ ID NO: 43, CDR2: SEQ ID NO: 44, CDR3: SEQ ID NO: 45;
(ix) CDR1: SEQ ID NO: 49, CDR2: SEQ ID NO: 50, CDR3: SEQ ID NO: 51;
(x) CDR1: SEQ ID NO: 55, CDR2: SEQ ID NO: 56, CDR3: SEQ ID NO: 57;
(xi) CDR1: SEQ ID NO: 61, CDR2: SEQ ID NO: 62, CDR3: SEQ ID NO: 63;
(xii) CDR1: SEQ ID NO: 67, CDR2: SEQ ID NO: 68, CDR3: SEQ ID NO: 69;
(xiii) CDR1: SEQ ID NO: 73, CDR2: SEQ ID NO: 74, CDR3: SEQ ID NO: 75;
(xiv) CDR1: SEQ ID NO: 79, CDR2: SEQ ID NO: 80, CDR3: SEQ ID NO: 81;
(xv) CDR1: SEQ ID NO: 85, CDR2: SEQ ID NO: 86, CDR3: SEQ ID NO: 87; and
(xvi) CDR1: SEQ ID NO: 91, CDR2: SEQ ID NO: 92, CDR3: SEQ ID NO: 93.

In some embodiments, a CBO polypeptide comprises: a) a VH comprising an amino acid sequence that has at least 55% (e.g., at least about 60%, at least about 65%, at least about 70%, at least about 75%, at least about 80%, at least about 85%, at least about 90%, at least about 95%, at least about 98%, or at least about 99%) sequence identity to SEQ ID NO: 98, 100, 102, 104, 106, 108, 110, 112, 114, 116, 118, 120, 122, 124, 126, or 128, b) a VL comprising an amino acid sequence that has at least 55% (e.g., at least 60, 65, 70, 75, 80, 85, 90, 95, 98, or 99%) sequence identity to SEQ ID NO: 97, 99, 101, 103, 105, 107, 109, 111, 113, 115, 117, 119, 121, 123, 125, or 127; or both a) and b), wherein the CBO polypeptide does not comprise all 6 CDRs of an antibody comprising a VH amino acid sequence of SEQ ID NO: 98, 100, 102, 104, 106, 108, 110, 112, 114, 116, 118, 120, 122, 124, 126, or 128 and a VL amino acid sequence of SEQ ID NO: 97, 99, 101, 103, 105, 107, 109, 111, 113, 115, 117, 119, 121, 123, 125, or 127.

In some embodiments, the CBO polypeptide comprises a VH that has at least about 70% sequence identity to the amino acid sequence of SEQ ID NO: 98, 100, 102, 104, 106, 108, 110, 112, 114, 116, 118, 120, 122, 124, 126, or 128. For example, the VH can have at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 98, 100, 102, 104, 106, 108, 110, 112, 114, 116, 118, 120, 122, 124, 126, or 128. In some embodiments, the VH has at least about 85% or at least about 90% sequence identity to the amino acid sequence of SEQ ID NO: 98, 100, 102, 104, 106, 108, 110, 112, 114, 116, 118, 120, 122, 124, 126, or 128.

In some embodiments, the CBO polypeptide comprises a VH that comprises at least 1 amino acid substitution relative to the amino acid sequence of SEQ ID NO: 98, 100, 102, 104, 106, 108, 110, 112, 114, 116, 118, 120, 122, 124, 126, or 128. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the VH comprises about 1-10 amino acid substitutions, relative to the amino acid sequence of SEQ ID NO: 98, 100, 102, 104, 106, 108, 110, 112, 114, 116, 118, 120, 122, 124, 126, or 128. In certain embodiments, the at least 1 amino acid substitution replaces only a HCDR1, a HCDR2 and/or a HCDR3 residue, of SEQ ID NO: 98, 100, 102, 104, 106, 108, 110, 112, 114, 116, 118, 120, 122, 124, 126, or 128. In particular embodiments, the at least 1 amino acid substitution replaces only a non-CDR residue (e.g., within a framework region), of SEQ ID NO: 98, 100, 102, 104, 106, 108, 110, 112, 114, 116, 118, 120, 122, 124, 126, or 128.

In some embodiments, the CBO polypeptide comprises a VL that has at least about 70% sequence identity to the amino acid sequence of SEQ ID NO: 97, 99, 101, 103, 105, 107, 109, 111, 113, 115, 117, 119, 121, 123, 125, or 127. For example, the VL can be at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 97, 99, 101, 103, 105, 107, 109, 111, 113, 115, 117, 119, 121, 123, 125, or 127. In some embodiments, the VL has at least about 85% or at least about 90% sequence identity to the amino acid sequence of SEQ ID NO: 97, 99, 101, 103, 105, 107, 109, 111, 113, 115, 117, 119, 121, 123, 125, or 127.

In some embodiments, the CBO polypeptide comprises a VL that comprises at least 1 amino acid substitution relative to the amino acid sequence of SEQ ID NO: 97, 99, 101, 103, 105, 107, 109, 111, 113, 115, 117, 119, 121, 123, 125, or 127. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the VL comprises about 1-10 amino acid substitutions, relative to the amino acid sequence of SEQ ID NO: 97, 99, 101, 103, 105, 107, 109, 111, 113, 115, 117, 119, 121, 123, 125, or 127. In certain embodiments, the at least 1 amino acid substitution replaces only a LCDR1, a LCDR2 and/or a LCDR3 residue, of SEQ ID NO: 97, 99, 101, 103, 105, 107, 109, 111, 113, 115, 117, 119, 121, 123, 125, or 127. In particular embodiments, the at least 1 amino acid substitution replaces only a non-CDR residue (e.g., within a framework region), of SEQ ID NO: 97, 99, 101, 103, 105, 107, 109, 111, 113, 115, 117, 119, 121, 123, 125, or 127.

In some embodiments, the CBO polypeptide comprises a VH that has at least about 70% sequence identity to the amino acid sequence of SEQ ID NO: 98, 100, 102, 104, 106, 108, 110, 112, 114, 116, 118, 120, 122, 124, 126, or 128, or a combination of the foregoing. For example, the VH can have at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 98, 100, 102, 104, 106, 108, 110, 112, 114, 116, 118, 120, 122, 124, 126, or 128, or a combination of the foregoing. In some embodiments, the VH has at least about 85% or at least about 90% sequence identity to the amino acid sequence of SEQ ID NO: 98, 100, 102, 104, 106, 108, 110, 112, 114, 116, 118, 120, 122, 124, 126, or 128, or a combination of the foregoing.

In some embodiments, the CBO polypeptide comprises a VH that comprises at least 1 amino acid substitution relative to the amino acid sequence of SEQ ID NO: 98, 100, 102, 104, 106, 108, 110, 112, 114, 116, 118, 120, 122, 124, 126, or 128, or a combination of the foregoing. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the VH comprises about 1-10 amino acid substitutions, relative to the amino acid sequence of SEQ ID NO: 98, 100, 102, 104, 106, 108, 110, 112, 114, 116, 118, 120, 122, 124, 126, or 128, or a combination of the foregoing. In certain embodiments, the at least 1 amino acid substitution replaces only a HCDR1, a HCDR2 and/or a HCDR3 residue, of SEQ ID NO: 98, 100, 102, 104, 106, 108, 110, 112, 114, 116, 118, 120, 122, 124, 126, or 128, or a combination of the foregoing. In particular embodiments, the at least 1 amino acid substitution replaces only a non-CDR residue (e.g., within a framework region), of SEQ ID NO: 98, 100, 102, 104, 106, 108, 110, 112, 114, 116, 118, 120, 122, 124, 126, or 128, or a combination of the foregoing.

In some embodiments, the CBO polypeptide comprises a VL that has at least about 70% sequence identity to the amino acid sequence of SEQ ID NO: 97, 99, 101, 103, 105, 107, 109, 111, 113, 115, 117, 119, 121, 123, 125, or 127, or a combination of the foregoing. For example, the VL can be at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 97, 99, 101, 103, 105, 107, 109, 111, 113, 115, 117, 119, 121, 123, 125, or 127, or a combination of the foregoing. In some embodiments, the VL has at least about 85% or at least about 90% sequence identity to the amino acid sequence of SEQ ID NO: 97, 99, 101, 103, 105, 107, 109, 111, 113, 115, 117, 119, 121, 123, 125, or 127, or a combination of the foregoing.

In some embodiments, the CBO polypeptide comprises a VL that comprises at least 1 amino acid substitution relative to the amino acid sequence of SEQ ID NO: 97, 99, 101, 103, 105, 107, 109, 111, 113, 115, 117, 119, 121, 123, 125, or 127, or a combination of the foregoing. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, the VL comprises about 1-10 amino acid substitutions, relative to the amino acid sequence of SEQ ID NO: 97, 99, 101, 103, 105, 107, 109, 111, 113, 115, 117, 119, 121, 123, 125, or 127, or a combination of the foregoing. In certain embodiments, the at least 1 amino acid substitution replaces only a LCDR1, a LCDR2 and/or a LCDR3 residue, of SEQ ID NO: 97, 99, 101, 103, 105, 107, 109, 111, 113, 115, 117, 119, 121, 123, 125, or 127, or a combination of the foregoing. In particular embodiments, the at least 1 amino acid substitution replaces only a non-CDR residue (e.g., within a framework region), of SEQ ID NO: 97, 99, 101, 103, 105, 107, 109, 111, 113, 115, 117, 119, 121, 123, 125, or 127, or a combination of the foregoing.

In some embodiments, a computer-implemented method includes scoring a polypeptide comprising an amino acid sequence with a computationally binding optimized (CBO) model for a functional property relating to modulating the activity of a target molecule. The CBO model, for each amino acid position of the amino acid sequence of the polypeptide, calculates multiple energy scores, the multiple energy scores based on the amino acid at a given position in the amino acid sequence. The CBO model further adds each energy score calculated to an array. The CBO model further generates a normalized sum of the energy scores for each position. The CBO model further generates a score of the polypeptide by summing each energy score calculated, the score representing a functional property of the polypeptide, the functional property relating to the polypeptide modulating the activity of the target molecule.

In some embodiments, the computer-implemented method includes calculating a logarithm of each energy score calculated.

In some embodiments, summing each energy score calculated is performed by summing the logarithms of each energy score calculated.

In some embodiments, the energy scores are further calculated based on having substituted the amino acid at the given position in the amino acid sequence with each of a plurality of different amino acids.

In some embodiments, the target molecule is a target polypeptide.

In some embodiments, the scoring the polypeptide using the CBO model is implemented by the script of Appendix A.

In some embodiments, the CBO model is substantially similar to the table of Appendix B. A substantially similar table to the Computer Programming Listing Appendix B is a table that scores polypeptides in line with the Computer Programming Listing Appendix B. In some embodiments, such a substantially similar table scores polypeptides so as to maintain their relative rank order. In certain embodiments, a substantially similar table returns scores within about: 30, 25, 20, 15, 10, 9, 8, 7, 6, 5, 4, 3, 2, or 1% of the score provided by the Computer Programming Listing Appendix B. In some embodiments, table substantially similar to the Computer Programming Listing Appendix B comprises substantially the same dimensions (rows and columns, e.g., within about: 1, 2, 3, 4, 5, 6, 7, 8, 9, 10, 12, 13, 14, 15%, or in some embodiments, the identical dimensions), wherein each entry in the table is substantially similar to a corresponding value in the Computer Programming Listing Appendix B, e.g., within about: 30, 25, 20, 15, 10, 9, 8, 7, 6, 5, 4, 3, 2, 1%, or identical.

In some embodiments, scoring the polypeptide can further include extracting positions from the amino acid sequence, the extracted positions being positions where mutations were applied during training of the CBO model. Scoring the polypeptide can further include calculating the multiple energy scores is performed on the extracted positions.

In some embodiments, scoring the polypeptide further includes scoring a pair of amino acid sequences. Scoring the polypeptide can further include aligning a first amino acid sequence and second amino acid sequence of the pair of amino acid sequences such that (a) the first amino acid sequence and second amino acid sequence are the same length and that (b) given sections of the first amino acid sequence and second amino acid sequence are the same.

In some embodiments, the multiple energy scores include a first-order energy score associated with each amino acid being at its position. The multiple energy scores further include, for each pair of amino acid positions of the amino acid sequence, a second-order energy score associated with the pair of amino acid positions. The first-order energy score and second-order energy score can be calculated based on corresponding entries of a trained first- and second-order energy score table of outputted weights of a model trained by a plurality of known amino acid sequences and corresponding properties of the known amino acid sequences.

In some embodiments, the computer-implemented method includes returning, based on the score, a flag indicating whether the sequence has the functional property associated with the polypeptide.

In some embodiments, the functional property is one or more of: a) a binding affinity for an interleukin-13 (IL-13) polypeptide characterized by a K_{D} of 10 pM or less (optionally, as measured by KinExA); a binding specificity for the IL-13 polypeptide; a neutralizing activity against the IL-13 polypeptide (optionally, a full-length human IL-13); or an inhibitory activity against IL-13-mediated signaling, or a combination of the foregoing.

In some embodiments, a system includes a processor and a memory with computer code instructions stored thereon. The processor and the memory, with the computer code instructions, are configured to cause the system to score a polypeptide comprising an amino acid sequence with a CBO model for a functional property relating to modulating the activity of a target molecule. The CBO model, for each amino acid position of the amino acid sequence of the polypeptide, calculates multiple energy scores, the multiple energy scores based on the amino acid at a given position in the amino acid sequence. The CBO model further adds each energy score calculated to an array. The CBO model further generates a normalized sum of the energy scores for each position. The CBO model further generates a score of the polypeptide by summing each energy score calculated, the score representing a functional property of the polypeptide.

In some embodiments, the instructions are further configured to cause the system to calculate a logarithm of each energy score calculated.

In some embodiments, summing each energy score calculated is performed by summing the logarithms of each energy score calculated.

In some embodiments, the energy scores are further calculated based on having substituted the amino acid at the given position in the amino acid sequence with each of a plurality of different amino acids.

In some embodiments, the target molecule is a target polypeptide.

In some embodiments, the scoring the polypeptide using the CBO model is implemented by the script of Appendix A.

In some embodiments, the CBO model is substantially similar to the table of Appendix B. A substantially similar table to the Computer Programming Listing Appendix B is a table that scores polypeptides in line with the Computer Programming Listing Appendix B. In some embodiments, such a substantially similar table scores polypeptides so as to maintain their relative rank order. In certain embodiments, a substantially similar table returns scores within about: 30, 25, 20, 15, 10, 9, 8, 7, 6, 5, 4, 3, 2, 1% of the score provided by the Computer Programming Listing Appendix B. In some embodiments, table substantially similar to the Computer Programming Listing Appendix B comprises substantially the same dimensions (rows and columns, e.g., within about: 1, 2, 3, 4, 5, 6, 7, 8, 9, 10, 12, 13, 14, 15%, in some embodiments, the identical dimensions), wherein each entry in the table is substantially similar to a corresponding value in the Computer Programming Listing Appendix B, e.g., within about: 30, 25, 20, 15, 10, 9, 8, 7, 6, 5, 4, 3, 2, 1%.

In some embodiments, scoring the polypeptide further includes extracting positions from the amino acid sequence. The extracted positions can be positions where mutations were applied during training of the CBO model. Scoring the polypeptide further includes calculating the multiple energy scores is performed on the extracted positions.

In some embodiments, scoring the polypeptide further includes scoring a pair of amino acid sequences. Scoring the polypeptide further includes aligning a first amino acid sequence and second amino acid sequence of the pair of amino acid sequences such that (a) the first amino acid sequence and second amino acid sequence are the same length and that (b) given sections of the first amino acid sequence and second amino acid sequence are the same.

In some embodiments, the multiple energy scores include a first-order energy score associated with each amino acid being at its position. The multiple energy scores further include, for each pair of amino acid positions of the amino acid sequence, a second-order energy score associated with the pair of amino acid positions. The first-order energy score and second-order energy score are calculated by respective entries from a trained first- and second-order energy score table of outputted weights of a model trained by a plurality of known amino acid sequences and corresponding properties of the known amino acid sequences.

In some embodiments, the processor further is configured to cause the system to return, based on the score, a flag indicating whether the sequence has the functional property associated with the polypeptide.

In some embodiments, the functional property is at least one of: a) a binding affinity for an interleukin-13 (IL-13) polypeptide characterized by a K_{D} of 10 pM or less (optionally, as measured by KinExA); a binding specificity for the IL-13 polypeptide; a neutralizing activity against the IL-13 polypeptide (optionally, a full-length human IL-13); or an inhibitory activity against IL-13-mediated signaling, or a combination of the foregoing.

In some embodiments, the score being above the particular threshold represents that the functional property is met for the given polypeptide.

In some embodiments, a polypeptide that specifically binds an interleukin-13 (IL-13) comprises an amino acid sequence that is assigned a score above a predetermined threshold by a computationally binding optimized (CBO) model upon scoring the amino acid sequence of the polypeptide.

In some embodiments, the polypeptide is assigned the score by the CBO model that is calculated by a table substantially similar to that of Computer Program Listing Appendix B.

In some embodiments, the polypeptide is assigned the score that is calculated using the Computer Program Listing Appendix B.

In some embodiments, the polypeptide is assigned the score by a script substantially similar to Computer Program Listing Appendix A, the script employing the CBO model substantially similar to the table of Computer Program Listing Appendix B. In some embodiments, the polypeptide has a logarithmic score above or equivalent to being above at least one of −7.7, −7.0, −6.5, −6.0, −5.5, −5.0, −4.5, −4.0, −3.5, −3.0, 2.9, −2.8, −2.7, −2.6, −2.5, −2.4, −2.3, −2.2, −2.1, −2.0, −1.9, −1.8, −1.7, −1.6, −1.5, −1.4, −1.3, −1.2, −1.1, −1.0, 0.9, −0.8, −0.7, −0.6, −0.5, −0.4, −0.3, −0.2, and −0.1.

In some embodiments, the polypeptide is scorable by the script of Computer Program Listing Appendix A.

In some embodiments, the predetermined threshold is the logarithmic probability that the sequence inputted to the script would occur given the weights of the Potts model.

In some embodiments, a CBO script assigning the score to the polypeptide includes receiving a trained first- and second-order energy score table of outputted weights of a model trained by a plurality of known amino acid sequences and corresponding properties of the known amino acid sequences.

In some embodiments, the polypeptide comprises an immunoglobulin heavy chain variable domain (VH) and an immunoglobulin light chain variable domain (VL).

In some embodiments, the polypeptide has one or more properties selected from: a) a binding affinity for an interleukin-13 (IL-13) polypeptide characterized by a K_{D} of 10 pM or less (optionally, as measured by KinExA); a binding specificity for the IL-13 polypeptide; a neutralizing activity against the IL-13 polypeptide (optionally, a full-length human IL-13); or an inhibitory activity against IL-13-mediated signaling, or a combination of the foregoing.

In some embodiments, the score being above the particular threshold represents that the functional property is met for the given polypeptide.

In some embodiments, the CBO model outputs a score that is equal to or above a score from the CBO model of one or more of a reference polypeptide that comprises a VH and VL pair selected from: SEQ ID NO: 97 and SEQ ID NO: 98 (AbA); SEQ ID NO: 99 and SEQ ID NO: 100 (AbB); SEQ ID NO: 101 and SEQ ID NO: 102 (AbC); SEQ ID NO: 103 and SEQ ID NO: 104 (AbD); SEQ ID NO: 105 and SEQ ID NO: 106 (AbE); SEQ ID NO: 107 and SEQ ID NO: 108 (AbF); SEQ ID NO: 109 and SEQ ID NO: 110 (AbG); SEQ ID NO: 111 and SEQ ID NO: 112 (AbH); SEQ ID NO: 113 and SEQ ID NO: 114 (AbI); SEQ ID NO: 115 and SEQ ID NO: 116 (AbJ); SEQ ID NO: 117 and SEQ ID NO: 118 (AbK); SEQ ID NO: 119 and SEQ ID NO: 120 (AbL); SEQ ID NO: 121 and SEQ ID NO: 122 (AbM); SEQ ID NO: 123 and SEQ ID NO: 124 (AbN); SEQ ID NO: 125 and SEQ ID NO: 126 (AbO); or SEQ ID NO: 127 and SEQ ID NO: 128 (AbP).

In some embodiments, the polypeptide comprises a VH and VL pair selected from: SEQ ID NO: 97 and SEQ ID NO: 98 (AbA); SEQ ID NO: 99 and SEQ ID NO: 100 (AbB); SEQ ID NO: 101 and SEQ ID NO: 102 (AbC); SEQ ID NO: 103 and SEQ ID NO: 104 (AbD); SEQ ID NO: 105 and SEQ ID NO: 106 (AbE); SEQ ID NO: 107 and SEQ ID NO: 108 (AbF); SEQ ID NO: 109 and SEQ ID NO: 110 (AbG); SEQ ID NO: 111 and SEQ ID NO: 112 (AbH); SEQ ID NO: 113 and SEQ ID NO: 114 (AbI); SEQ ID NO: 115 and SEQ ID NO: 116 (AbJ); SEQ ID NO: 117 and SEQ ID NO: 118 (AbK); SEQ ID NO: 119 and SEQ ID NO: 120 (AbL); SEQ ID NO: 121 and SEQ ID NO: 122 (AbM); SEQ ID NO: 123 and SEQ ID NO: 124 (AbN); SEQ ID NO: 125 and SEQ ID NO: 126 (AbO); or SEQ ID NO: 127 and SEQ ID NO: 128 (AbP).

In some embodiments, the polypeptide does not comprise a VH and VL pair selected from: SEQ ID NO: 97 and SEQ ID NO: 98 (AbA); SEQ ID NO: 99 and SEQ ID NO: 100 (AbB); SEQ ID NO: 101 and SEQ ID NO: 102 (AbC); SEQ ID NO: 103 and SEQ ID NO: 104 (AbD); SEQ ID NO: 105 and SEQ ID NO: 106 (AbE); SEQ ID NO: 107 and SEQ ID NO: 108 (AbF); SEQ ID NO: 109 and SEQ ID NO: 110 (AbG); SEQ ID NO: 111 and SEQ ID NO: 112 (AbH); SEQ ID NO: 113 and SEQ ID NO: 114 (AbI); SEQ ID NO: 115 and SEQ ID NO: 116 (AbJ); SEQ ID NO: 117 and SEQ ID NO: 118 (AbK); SEQ ID NO: 119 and SEQ ID NO: 120 (AbL); SEQ ID NO: 121 and SEQ ID NO: 122 (AbM); SEQ ID NO: 123 and SEQ ID NO: 124 (AbN); SEQ ID NO: 125 and SEQ ID NO: 126 (AbO); or SEQ ID NO: 127 and SEQ ID NO: 128 (AbP).

In some embodiments, the polypeptide does not comprise a heavy chain comprising SEQ ID NO: 179 or 18 and a light chain comprising SEQ ID NO: 178 or 180.

In some embodiments, the polypeptide is an antibody or an antigen-binding fragment thereof.

In some embodiments, a polypeptide comprises an amino acid sequence that is assigned a score above a predetermined threshold by a computationally binding optimized (CBO) model upon scoring the amino acid sequence of the polypeptide.

In some embodiments, a polypeptide that binds human interleukin-13 (IL-13) is designed by a method comprising generating a polypeptide sequence with a CBO model. The polypeptide is further designed by verifying the generated polypeptide sequence using the CBO model by, for each amino acid position of the polypeptide sequence, calculating a plurality of energy scores. The energy scores can be based on having substituted the amino acid at a given position in the polypeptide sequence with each of a plurality of different amino acids. The polypeptide is further verified by adding each energy score calculated to an array. The polypeptide is further verified by generating a normalized sum of the energy scores for each position. The polypeptide is further verified by calculating a logarithm of each energy score calculated. The polypeptide is further verified by generating a score of the polypeptide sequence by summing the logarithms of each energy score calculated. The score represents a functional property of the polypeptide's ability to bind to human IL-13. In some embodiments, the polypeptide sequence is verified if the polypeptide scores above a particular threshold.

In some embodiments, the polypeptide is scored at least a score of one of more of −7.7, −7.0, −6.5, −6.0, −5.5, −5.0, −4.5, −4.0, −3.5, −3.0, 2.9, −2.8, −2.7, −2.6, −2.5, −2.4, −2.3, −2.2, −2.1, −2.0, −1.9, −1.8, −1.7, −1.6, −1.5, −1.4, −1.3, −1.2, −1.1, −1.0, 0.9, −0.8, −0.7, −0.6, −0.5, −0.4, −0.3, −0.2, and −0.1 (e.g., to be verified). In some embodiments, the polypeptide is an antibody or an antigen-binding fragment thereof.

In some embodiments, the polypeptide comprises a VH and VL pair comprising an amino acid sequence at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to a VH and VL pair selected from: SEQ ID NO: 97 and SEQ ID NO: 98 (AbA); SEQ ID NO: 99 and SEQ ID NO: 100 (AbB); SEQ ID NO: 101 and SEQ ID NO: 102 (AbC); SEQ ID NO: 103 and SEQ ID NO: 104 (AbD); SEQ ID NO: 105 and SEQ ID NO: 106 (AbE); SEQ ID NO: 107 and SEQ ID NO: 108 (AbF); SEQ ID NO: 109 and SEQ ID NO: 110 (AbG); SEQ ID NO: 111 and SEQ ID NO: 112 (AbH); SEQ ID NO: 113 and SEQ ID NO: 114 (AbI); SEQ ID NO: 115 and SEQ ID NO: 116 (AbJ); SEQ ID NO: 117 and SEQ ID NO: 118 (AbK); SEQ ID NO: 119 and SEQ ID NO: 120 (AbL); SEQ ID NO: 121 and SEQ ID NO: 122 (AbM); SEQ ID NO: 123 and SEQ ID NO: 124 (AbN); SEQ ID NO: 125 and SEQ ID NO: 126 (AbO); or SEQ ID NO: 127 and SEQ ID NO: 128 (AbP).

In some embodiments, the polypeptide comprises a VH and VL pair does not comprise an amino acid sequence 100% identical to a VH and VL pair selected from: SEQ ID NO: 97 and SEQ ID NO: 98 (AbA); SEQ ID NO: 99 and SEQ ID NO: 100 (AbB); SEQ ID NO: 101 and SEQ ID NO: 102 (AbC); SEQ ID NO: 103 and SEQ ID NO: 104 (AbD); SEQ ID NO: 105 and SEQ ID NO: 106 (AbE); SEQ ID NO: 107 and SEQ ID NO: 108 (AbF); SEQ ID NO: 109 and SEQ ID NO: 110 (AbG); SEQ ID NO: 111 and SEQ ID NO: 112 (AbH); SEQ ID NO: 113 and SEQ ID NO: 114 (AbI); SEQ ID NO: 115 and SEQ ID NO: 116 (AbJ); SEQ ID NO: 117 and SEQ ID NO: 118 (AbK); SEQ ID NO: 119 and SEQ ID NO: 120 (AbL); SEQ ID NO: 121 and SEQ ID NO: 122 (AbM); SEQ ID NO: 123 and SEQ ID NO: 124 (AbN); SEQ ID NO: 125 and SEQ ID NO: 126 (AbO); or SEQ ID NO: 127 and SEQ ID NO: 128 (AbP).

### Illustration of Subject Technology as Clauses

Various examples of aspects are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1: An antibody or antigen binding fragment thereof that binds IL-13 comprising: a) a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3); b) a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3); c) a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3); d) a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3); e) a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3); f) a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3); g) a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3); h) a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3); i) a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3); j) a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3); k) a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3); l) a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3); m) a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3); n) a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3); o) a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); or p) a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3).

Clause 2: The antibody or antigen binding fragment thereof that binds IL-13 according to clause 1, wherein the light chain variable region comprises an amino acid sequence that is at least 90%, at least 95%, or at least 99% identical to SEQ ID NO: 97, 99, 101, 103, 105, 107, 109, 111, 113, 115, 117, 119, 121, 123, 125, or 127; and the heavy chain variable region comprises an amino acid sequence that is at least 90%, at least 95%, or at least 99% identical to SEQ ID NO: 98, 100, 102, 104, 106, 108, 110, 112, 114, 116, 118, 120, 122, 124, 126, or 128.

Clause 3: The antibody or antigen binding fragment thereof that binds IL-13 according to clause 1, wherein the light chain variable region comprises an amino acid sequence as set forth in SEQ ID NO: 97, 99, 101, 103, 105, 107, 109, 111, 113, 115, 117, 119, 121, 123, 125, or 127; and/or the heavy chain variable region comprises an amino acid sequence as set forth in SEQ ID NO: 98, 100, 102, 104, 106, 108, 110, 112, 114, 116, 118, 120, 122, 124, 126, or 128.

Clause 4: The antibody or antigen binding fragment thereof that binds IL-13 according to clause 3, wherein: a) the light chain variable region comprises an amino acid sequence as set forth in SEQ ID NO: 97; and the heavy chain variable region comprises an amino acid sequence as set forth in SEQ ID NO: 98; b) the light chain variable region comprises an amino acid sequence as set forth in SEQ ID NO: 99; and the heavy chain variable region comprises an amino acid sequence as set forth in SEQ ID NO: 100; c) the light chain variable region comprises an amino acid sequence as set forth in SEQ ID NO: 101; and the heavy chain variable region comprises an amino acid sequence as set forth in SEQ ID NO: 102; d) the light chain variable region comprises an amino acid sequence as set forth in SEQ ID NO: 103; and the heavy chain variable region comprises an amino acid sequence as set forth in SEQ ID NO: 104; e) the light chain variable region comprises an amino acid sequence as set forth in SEQ ID NO: 105; and the heavy chain variable region comprises an amino acid sequence as set forth in SEQ ID NO: 106; f) the light chain variable region comprises an amino acid sequence as set forth in SEQ ID NO: 107; and the heavy chain variable region comprises an amino acid sequence as set forth in SEQ ID NO: 108; g) the light chain variable region comprises an amino acid sequence as set forth in SEQ ID NO: 109; and the heavy chain variable region comprises an amino acid sequence as set forth in SEQ ID NO: 110; h) the light chain variable region comprises an amino acid sequence as set forth in SEQ ID NO: 111; and the heavy chain variable region comprises an amino acid sequence as set forth in SEQ ID NO: 112; i) the light chain variable region comprises an amino acid sequence as set forth in SEQ ID NO: 113; and the heavy chain variable region comprises an amino acid sequence as set forth in SEQ ID NO: 114; j) the light chain variable region comprises an amino acid sequence as set forth in SEQ ID NO: 115; and the heavy chain variable region comprises an amino acid sequence as set forth in SEQ ID NO: 116; k) the light chain variable region comprises an amino acid sequence as set forth in SEQ ID NO: 117; and the heavy chain variable region comprises an amino acid sequence as set forth in SEQ ID NO: 118; l) the light chain variable region comprises an amino acid sequence as set forth in SEQ ID NO: 119; and the heavy chain variable region comprises an amino acid sequence as set forth in SEQ ID NO: 120; m) the light chain variable region comprises an amino acid sequence as set forth in SEQ ID NO: 121; and the heavy chain variable region comprises an amino acid sequence as set forth in SEQ ID NO: 122; n) the light chain variable region comprises an amino acid sequence as set forth in SEQ ID NO: 123; and the heavy chain variable region comprises an amino acid sequence as set forth in SEQ ID NO: 124; o) the light chain variable region comprises an amino acid sequence as set forth in SEQ ID NO: 125; and the heavy chain variable region comprises an amino acid sequence as set forth in SEQ ID NO: 126; or p) the light chain variable region comprises an amino acid sequence as set forth in SEQ ID NO: 127; and the heavy chain variable region comprises an amino acid sequence as set forth in SEQ ID NO: 128.

Clause 5: The antibody or antigen binding fragment thereof that binds IL-13 according to clause 1, wherein the antibody comprises a light chain comprising an amino acid sequence that is at least 90%, at least 95%, or at least 99% identical to SEQ ID NO: 129, 131, 133, 135, 137, 139, 141, 143, 145, 147, 149, 151, 153, 155, 157, or 159; and/or a heavy chain comprising an amino acid sequence that is at least 90%, at least 95%, or at least 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

Clause 6: The antibody or antigen binding fragment thereof that binds IL-13 according to clause 5, wherein the light chain comprises an amino acid sequence as set forth in SEQ ID NO: 129, 131, 133, 135, 137, 139, 141, 143, 145, 147, 149, 151, 153, 155, 157, or 159; and/or the heavy chain comprises an amino acid sequence as set forth in SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

Clause 7: The antibody or antigen binding fragment thereof that binds IL-13 according to clause 6, wherein: a) the light chain comprises an amino acid sequence as set forth in SEQ ID NO: 129; and the heavy chain comprises an amino acid sequence as set forth in SEQ ID NO: 130; b) the light chain comprises an amino acid sequence as set forth in SEQ ID NO: 131; and the heavy chain comprises an amino acid sequence as set forth in SEQ ID NO: 132; c) the light chain comprises an amino acid sequence as set forth in SEQ ID NO: 133; and the heavy chain comprises an amino acid sequence as set forth in SEQ ID NO: 134; d) the light chain comprises an amino acid sequence as set forth in SEQ ID NO: 135; and the heavy chain comprises an amino acid sequence as set forth in SEQ ID NO: 136; e) the light chain comprises an amino acid sequence as set forth in SEQ ID NO: 137; and the heavy chain comprises an amino acid sequence as set forth in SEQ ID NO: 138; f) the light chain comprises an amino acid sequence as set forth in SEQ ID NO: 139; and the heavy chain comprises an amino acid sequence as set forth in SEQ ID NO: 140; g) the light chain comprises an amino acid sequence as set forth in SEQ ID NO: 141; and the heavy chain comprises an amino acid sequence as set forth in SEQ ID NO: 142; h) the light chain comprises an amino acid sequence as set forth in SEQ ID NO: 143; and the heavy chain comprises an amino acid sequence as set forth in SEQ ID NO: 144; i) the light chain comprises an amino acid sequence as set forth in SEQ ID NO: 145; and the heavy chain comprises an amino acid sequence as set forth in SEQ ID NO: 146; j) the light chain comprises an amino acid sequence as set forth in SEQ ID NO: 147; and the heavy chain comprises an amino acid sequence as set forth in SEQ ID NO: 148; k) the light chain comprises an amino acid sequence as set forth in SEQ ID NO: 149; and the heavy chain comprises an amino acid sequence as set forth in SEQ ID NO: 150; l) the light chain comprises an amino acid sequence as set forth in SEQ ID NO: 151; and the heavy chain comprises an amino acid sequence as set forth in SEQ ID NO: 152; m) the light chain comprises an amino acid sequence as set forth in SEQ ID NO: 153; and the heavy chain comprises an amino acid sequence as set forth in SEQ ID NO: 154; n) the light chain comprises an amino acid sequence as set forth in SEQ ID NO: 155; and the heavy chain comprises an amino acid sequence as set forth in SEQ ID NO: 156; o) the light chain comprises an amino acid sequence as set forth in SEQ ID NO: 157; and the heavy chain comprises an amino acid sequence as set forth in SEQ ID NO: 158; p) the light chain comprises an amino acid sequence as set forth in SEQ ID NO: 159; and the heavy chain comprises an amino acid sequence as set forth in SEQ ID NO: 160; q) the light chain comprises an amino acid sequence as set forth in SEQ ID NO: 153; and the heavy chain comprises an amino acid sequence as set forth in SEQ ID NO: 182; r) the light chain comprises an amino acid sequence as set forth in SEQ ID NO: 147; and the heavy chain comprises an amino acid sequence as set forth in SEQ ID NO: 183; s) the light chain comprises an amino acid sequence as set forth in SEQ ID NO: 141; and the heavy chain comprises an amino acid sequence as set forth in SEQ ID NO: 184; t) the light chain comprises an amino acid sequence as set forth in SEQ ID NO: 153; and the heavy chain comprises an amino acid sequence as set forth in SEQ ID NO: 185; u) the light chain comprises an amino acid sequence as set forth in SEQ ID NO: 147; and the heavy chain comprises an amino acid sequence as set forth in SEQ ID NO: 186; v) the light chain comprises an amino acid sequence as set forth in SEQ ID NO: 141; and the heavy chain comprises an amino acid sequence as set forth in SEQ ID NO: 187; w) the light chain comprises an amino acid sequence as set forth in SEQ ID NO: 153; and the heavy chain comprises an amino acid sequence as set forth in SEQ ID NO: 188; x) the light chain comprises an amino acid sequence as set forth in SEQ ID NO: 147; and the heavy chain comprises an amino acid sequence as set forth in SEQ ID NO: 189; y) the light chain comprises an amino acid sequence as set forth in SEQ ID NO: 141; and the heavy chain comprises an amino acid sequence as set forth in SEQ ID NO: 190; z) the light chain comprises an amino acid sequence as set forth in SEQ ID NO: 153; and the heavy chain comprises an amino acid sequence as set forth in SEQ ID NO: 191; aa) the light chain comprises an amino acid sequence as set forth in SEQ ID NO: 147; and the heavy chain comprises an amino acid sequence as set forth in SEQ ID NO: 192; ab) the light chain comprises an amino acid sequence as set forth in SEQ ID NO: 141; and the heavy chain comprises an amino acid sequence as set forth in SEQ ID NO: 193; ac) the light chain comprises an amino acid sequence as set forth in SEQ ID NO: 153; and the heavy chain comprises an amino acid sequence as set forth in SEQ ID NO: 194; ad) the light chain comprises an amino acid sequence as set forth in SEQ ID NO: 147; and the heavy chain comprises an amino acid sequence as set forth in SEQ ID NO: 195; ae) the light chain comprises an amino acid sequence as set forth in SEQ ID NO: 141; and the heavy chain comprises an amino acid sequence as set forth in SEQ ID NO: 196; af) the light chain comprises an amino acid sequence as set forth in SEQ ID NO: 153; and the heavy chain comprises an amino acid sequence as set forth in SEQ ID NO: 197; ag) the light chain comprises an amino acid sequence as set forth in SEQ ID NO: 147; and the heavy chain comprises an amino acid sequence as set forth in SEQ ID NO: 198; ah) the light chain comprises an amino acid sequence as set forth in SEQ ID NO: 141; and the heavy chain comprises an amino acid sequence as set forth in SEQ ID NO: 199; ai) the light chain comprises an amino acid sequence as set forth in SEQ ID NO: 153; and the heavy chain comprises an amino acid sequence as set forth in SEQ ID NO: 200; aj) the light chain comprises an amino acid sequence as set forth in SEQ ID NO: 147; and the heavy chain comprises an amino acid sequence as set forth in SEQ ID NO: 201; ak) the light chain comprises an amino acid sequence as set forth in SEQ ID NO: 141; and the heavy chain comprises an amino acid sequence as set forth in SEQ ID NO: 202; al) the light chain comprises an amino acid sequence as set forth in SEQ ID NO: 153; and the heavy chain comprises an amino acid sequence as set forth in SEQ ID NO: 203; am) the light chain comprises an amino acid sequence as set forth in SEQ ID NO: 147; and the heavy chain comprises an amino acid sequence as set forth in SEQ ID NO: 204; an) the light chain comprises an amino acid sequence as set forth in SEQ ID NO: 141; and the heavy chain comprises an amino acid sequence as set forth in SEQ ID NO: 205; ao) the light chain comprises an amino acid sequence as set forth in SEQ ID NO: 153; and the heavy chain comprises an amino acid sequence as set forth in SEQ ID NO: 206; ap) the light chain comprises an amino acid sequence as set forth in SEQ ID NO: 147; and the heavy chain comprises an amino acid sequence as set forth in SEQ ID NO: 207; aq) the light chain comprises an amino acid sequence as set forth in SEQ ID NO: 141; and the heavy chain comprises an amino acid sequence as set forth in SEQ ID NO: 208; ar) the light chain comprises an amino acid sequence as set forth in SEQ ID NO: 153; and the heavy chain comprises an amino acid sequence as set forth in SEQ ID NO: 209; as) the light chain comprises an amino acid sequence as set forth in SEQ ID NO: 147; and the heavy chain comprises an amino acid sequence as set forth in SEQ ID NO: 210; or at) the light chain comprises an amino acid sequence as set forth in SEQ ID NO: 141; and the heavy chain comprises an amino acid sequence as set forth in SEQ ID NO: 211.

Clause 8: The antibody or antigen binding fragment thereof that binds IL-13 according to clause 1, wherein the antibody is a monoclonal antibody.

Clause 9: The antibody or antigen binding fragment thereof that binds IL-13 according to clause 1, wherein the antibody is a human antibody.

Clause 10: The antibody or antigen binding fragment thereof that binds IL-13 according to clause 1, wherein the antibody is a bispecific antibody.

Clause 11: The antibody or antigen binding fragment thereof that binds IL-13 according to clause 1, wherein the binding fragment thereof is selected from the group consisting of Fab, Fab′, F(ab′)₂, Fd, Fv, scFv, a single-chain antibody, a minibody, and a diabody.

Clause 12: The antibody or antigen binding fragment thereof according to clause 11, wherein the binding fragment thereof is an scFv.

Clause 13: The antibody or antigen binding fragment thereof that binds IL-13 according to any one of clauses 1 to 12 for use as a medicament.

Clause 14: A pharmaceutical composition comprising an antibody or antigen binding fragment thereof that binds IL-13 according to any one of clauses 1 to 12 and a pharmaceutically acceptable carrier.

Clause 15: A nucleic acid composition comprising one or more nucleic acids encoding the light chain variable region and heavy chain variable region of clause 1.

Clause 16: An expression vector composition comprising one or more expression vectors comprising the nucleic acid composition of clause 15.

Clause 17: A host cell comprising the nucleic acid composition of clause 15 or the expression vector composition of clause 16.

Clause 18: A method of making the antibody or antigen binding fragment thereof that binds IL-13 according to clause 1 comprising culturing the host cell of clause 17 under conditions where the antibody or binding fragment thereof that binds IL-13 is expressed and recovering the antibody or binding fragment thereof that binds IL-13.

Clause 19: An antibody or antigen binding fragment thereof that binds IL-13 comprising: a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3); a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3); a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3); a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3); a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3); a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3); a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3); a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3); a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3); a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3); a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3); a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3); a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3); a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3); a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3); or b1) a light chain variable region comprising an amino acid sequence at least 90%, at least 95%, or at least 99% identical to SEQ ID NO: 97, 99, 101, 103, 105, 107, 109, 111, 113, 115, 117, 119, 121, 123, 125, or 127; and b2) a heavy chain variable region comprising an amino acid sequence at least 90%, at least 95%, or at least 99% identical to SEQ ID NO: 98, 100, 102, 104, 106, 108, 110, 112, 114, 116, 118, 120, 122, 124, 126, or 128, wherein the LCDR1 does not include SEQ ID NO: 162 (RASKSVDSYGNSFMH).

Clause 20: An antibody or antigen binding fragment thereof that binds IL-13 according to clause 19, wherein: LCDR1 comprises a serine (S) at amino acid position 27D and an arginine (R) at amino acid position 30; LCDR1 comprises a tyrosine (Y) at amino acid position 27D and a histidine (H) at amino acid position 30; LCDR1 comprises a tyrosine (Y) at amino acid position 27D and an asparagine (N) at amino acid position 30; or LCDR1 comprises a serine (S) at amino acid position 27D and a tyrosine (Y) at amino acid position 32, wherein the amino acid positions are based on Kabat numbering.

Clause 21: An antibody or antigen binding fragment thereof that binds IL-13 comprising a light chain variable region and a heavy chain variable region, wherein the light chain variable region is at least 95% identical to SEQ ID NO: 97, 99, 101, or 103; and wherein the heavy chain variable region is at least 95% identical to SEQ ID NOs: SEQ ID NO: 98, 100, 102, or 104, provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, wherein the amino acid positions are based on Kabat numbering.

Clause 22: The antibody or antigen binding fragment thereof that binds IL-13 according to clause 19 or 21, wherein the antibody is a monoclonal antibody.

Clause 23: The antibody or antigen binding fragment thereof that binds IL-13 according to clause 19 or 21, wherein the antibody is a human antibody.

Clause 24: The antibody or antigen binding fragment thereof that binds IL-13 according to clause 19 or 21, wherein the antibody is a bispecific antibody.

Clause 25: The antibody or antigen binding fragment thereof that binds IL-13 according to clause 19 or 21, wherein the binding fragment thereof is selected from the group consisting of Fab, Fab′, F(ab′)₂, Fd, Fv, scFv, a single-chain antibody, a minibody, and a diabody.

Clause 26: The antibody or antigen binding fragment thereof that binds IL-13 according to clause 25, wherein the binding fragment thereof is an scFv.

Clause 27: The antibody or antigen binding fragment thereof that binds IL-13 according to any one of clauses 19 to 26 for use as a medicament.

Clause 28: A pharmaceutical composition comprising an antibody or antigen binding fragment thereof that binds IL-13 according to any one of clauses 19 to 26 and a pharmaceutically acceptable carrier.

Clause 29: A nucleic acid composition comprising one or more nucleic acids encoding the light chain variable region and heavy chain variable region of clause 19 or 21.

Clause 30: An expression vector composition comprising one or more expression vectors comprising the nucleic acid composition of clause 29.

Clause 31: A host cell comprising the nucleic acid composition of clause 29 or the expression vector composition of clause 30.

Clause 32: A method of making the antibody or antigen binding fragment thereof that binds IL-13 according to clause 19 or 21 comprising culturing the host cell of clause 31 under conditions where the antibody or binding fragment thereof that binds IL-13 is expressed and recovering the antibody or binding fragment thereof that binds IL-13.

Clause 33: A method of treating a disease or disorder in a subject in need thereof, the method comprising administering to the subject a therapeutically effective dose of an antibody or antigen binding fragment thereof that binds IL-13, wherein the antibody or antigen binding fragment thereof that binds IL-13 comprises: a) a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3); b) a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3); c) a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3); d) a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3); e) a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3); f) a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3); g) a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3); h) a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3); i) a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3); j) a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3); k) a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3); l) a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3); m) a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3); n) a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3); o) a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); or p) a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3).

Clause 34: The method of treating a disease or disorder of clause 33, wherein the disease or disorder is selected from the group consisting of: asthma (including, but not limited to, mild asthma, mild to moderate asthma, moderate asthma, moderate to severe asthma, and/or severe asthma), allergic asthma, allergen-induced airway obstruction due to asthma and/or allergic asthma, atopic dermatitis (including, but not limited to, mild atopic dermatitis, mild to moderate atopic dermatitis, moderate atopic dermatitis, moderate-to-severe, and/or severe atopic dermatitis), eczema, acute urticaria, idiopathic pulmonary fibrosis (IPF), chronic obstructive pulmonary disease (COPD), chronic obstructive airway disease, emphysema, chronic bronchitis, pulmonary fibrosis, systemic sclerosis, scleroderma, cryptogenic fibrosing alveolitis, usual interstitial pneumonitis, idiopathic interstitial pneumonitis, wound, systemic lupus erythematosus (SLE), rheumatoid arthritis (RA), systemic sclerosis (SS), ulcerative colitis (UC), type 1 diabetes (T1D), sjogren's syndrome (SS), prurigo nodularis, nasal polyposis, recurrent urticaria, aspergillosis, bullous pemphigoid, chronic sinusitis, alopecia areata, hay fever, pemphigus, allergic rhinitis, psoriasis, rosacea, allergic drug reactions, allergic reactions, anaphylaxis, acne, food allergies, inflammatory bowel disease (IBS), Crohn's disease, ulcerative colitis, chronic rhinosinusitis with nasal polyps (CRSwNP), eosinophilic esophagitis, eosinophilic colitis, eosinophilic gastritis, eosinophilic cystitis, eosinophilic fasciitis, eosinophilic pustular folliculitis, eosinophilic granulomatosis, eosinophilic polyangiitis, Churg-Strauss syndrome, biliary cancer, brain cancer, breast cancer, colorectal cancer, genitourinary cancer, head and neck cancer, liver cancer, Hodgkin's lymphoma, esophageal cancer, pancreatic cancer, prostate cancer, renal cancer, Kaposi's sarcoma, sinusitis, or chronic urticaria.

Clause 35: The method of clause 33, wherein the light chain variable region comprises an amino acid sequence that is at least 90%, at least 95%, or at least 99% identical to SEQ ID NO: 97, 99, 101, 103, 105, 107, 109, 111, 113, 115, 117, 119, 121, 123, 125, or 127; and the heavy chain variable region comprises an amino acid sequence that is at least 90%, at least 95%, or at least 99% identical to SEQ ID NO: 98, 100, 102, 104, 106, 108, 110, 112, 114, 116, 118, 120, 122, 124, 126, or 128.

Clause 36: The method of clause 33, wherein the light chain variable region comprises an amino acid sequence as set forth in SEQ ID NO: 97, 99, 101, 103, 105, 107, 109, 111, 113, 115, 117, 119, 121, 123, 125, or 127; and/or the heavy chain variable region comprises an amino acid sequence as set forth in SEQ ID NO: 98, 100, 102, 104, 106, 108, 110, 112, 114, 116, 118, 120, 122, 124, 126, or 128.

Clause 37: The method of clause 36, wherein: a) the light chain variable region comprises an amino acid sequence as set forth in SEQ ID NO: 97; and the heavy chain variable region comprises an amino acid sequence as set forth in SEQ ID NO: 98; b) the light chain variable region comprises an amino acid sequence as set forth in SEQ ID NO: 99; and the heavy chain variable region comprises an amino acid sequence as set forth in SEQ ID NO: 100; c) the light chain variable region comprises an amino acid sequence as set forth in SEQ ID NO: 101; and the heavy chain variable region comprises an amino acid sequence as set forth in SEQ ID NO: 102; d) the light chain variable region comprises an amino acid sequence as set forth in SEQ ID NO: 103; and the heavy chain variable region comprises an amino acid sequence as set forth in SEQ ID NO: 104; e) the light chain variable region comprises an amino acid sequence as set forth in SEQ ID NO: 105; and the heavy chain variable region comprises an amino acid sequence as set forth in SEQ ID NO: 106; f) the light chain variable region comprises an amino acid sequence as set forth in SEQ ID NO: 107; and the heavy chain variable region comprises an amino acid sequence as set forth in SEQ ID NO: 108; g) the light chain variable region comprises an amino acid sequence as set forth in SEQ ID NO: 109; and the heavy chain variable region comprises an amino acid sequence as set forth in SEQ ID NO: 110; h) the light chain variable region comprises an amino acid sequence as set forth in SEQ ID NO: 111; and the heavy chain variable region comprises an amino acid sequence as set forth in SEQ ID NO: 112; i) the light chain variable region comprises an amino acid sequence as set forth in SEQ ID NO: 113; and the heavy chain variable region comprises an amino acid sequence as set forth in SEQ ID NO: 114; j) the light chain variable region comprises an amino acid sequence as set forth in SEQ ID NO: 115; and the heavy chain variable region comprises an amino acid sequence as set forth in SEQ ID NO: 116; k) the light chain variable region comprises an amino acid sequence as set forth in SEQ ID NO: 117; and the heavy chain variable region comprises an amino acid sequence as set forth in SEQ ID NO: 118; l) the light chain variable region comprises an amino acid sequence as set forth in SEQ ID NO: 119; and the heavy chain variable region comprises an amino acid sequence as set forth in SEQ ID NO: 120; m) the light chain variable region comprises an amino acid sequence as set forth in SEQ ID NO: 121; and the heavy chain variable region comprises an amino acid sequence as set forth in SEQ ID NO: 122; n) the light chain variable region comprises an amino acid sequence as set forth in SEQ ID NO: 123; and the heavy chain variable region comprises an amino acid sequence as set forth in SEQ ID NO: 124; o) the light chain variable region comprises an amino acid sequence as set forth in SEQ ID NO: 125; and the heavy chain variable region comprises an amino acid sequence as set forth in SEQ ID NO: 126; or p) the light chain variable region comprises an amino acid sequence as set forth in SEQ ID NO: 127; and the heavy chain variable region comprises an amino acid sequence as set forth in SEQ ID NO: 128.

Clause 38: The method of clause 33, wherein the antibody comprises a light chain comprising an amino acid sequence that is at least 90%, at least 95%, or at least 99% identical to SEQ ID NO: 129, 131, 133, 135, 137, 139, 141, 143, 145, 147, 149, 151, 153, 155, 157, or 159; and/or a heavy chain comprising an amino acid sequence that is at least 90%, at least 95%, or at least 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

Clause 39: The method of clause 38, wherein the light chain comprises an amino acid sequence as set forth in SEQ ID NO: 129, 131, 133, 135, 137, 139, 141, 143, 145, 147, 149, 151, 153, 155, 157, or 159; and/or the heavy chain comprises an amino acid sequence as set forth in SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

Clause 40: The method of clause 39, wherein: a) the light chain comprises an amino acid sequence as set forth in SEQ ID NO: 129; and the heavy chain comprises an amino acid sequence as set forth in SEQ ID NO: 130; b) the light chain comprises an amino acid sequence as set forth in SEQ ID NO: 131; and the heavy chain comprises an amino acid sequence as set forth in SEQ ID NO: 132; c) the light chain comprises an amino acid sequence as set forth in SEQ ID NO: 133; and the heavy chain comprises an amino acid sequence as set forth in SEQ ID NO: 134; d) the light chain comprises an amino acid sequence as set forth in SEQ ID NO: 135; and the heavy chain comprises an amino acid sequence as set forth in SEQ ID NO: 136; e) the light chain comprises an amino acid sequence as set forth in SEQ ID NO: 137; and the heavy chain comprises an amino acid sequence as set forth in SEQ ID NO: 138; f) the light chain comprises an amino acid sequence as set forth in SEQ ID NO: 139; and the heavy chain comprises an amino acid sequence as set forth in SEQ ID NO: 140; g) the light chain comprises an amino acid sequence as set forth in SEQ ID NO: 141; and the heavy chain comprises an amino acid sequence as set forth in SEQ ID NO: 142; h) the light chain comprises an amino acid sequence as set forth in SEQ ID NO: 143; and the heavy chain comprises an amino acid sequence as set forth in SEQ ID NO: 144; i) the light chain comprises an amino acid sequence as set forth in SEQ ID NO: 145; and the heavy chain comprises an amino acid sequence as set forth in SEQ ID NO: 146; j) the light chain comprises an amino acid sequence as set forth in SEQ ID NO: 147; and the heavy chain comprises an amino acid sequence as set forth in SEQ ID NO: 148; k) the light chain comprises an amino acid sequence as set forth in SEQ ID NO: 149; and the heavy chain comprises an amino acid sequence as set forth in SEQ ID NO: 150; l) the light chain comprises an amino acid sequence as set forth in SEQ ID NO: 151; and the heavy chain comprises an amino acid sequence as set forth in SEQ ID NO: 152; m) the light chain comprises an amino acid sequence as set forth in SEQ ID NO: 153; and the heavy chain comprises an amino acid sequence as set forth in SEQ ID NO: 154; n) the light chain comprises an amino acid sequence as set forth in SEQ ID NO: 155; and the heavy chain comprises an amino acid sequence as set forth in SEQ ID NO: 156; o) the light chain comprises an amino acid sequence as set forth in SEQ ID NO: 157; and the heavy chain comprises an amino acid sequence as set forth in SEQ ID NO: 158; p) the light chain comprises an amino acid sequence as set forth in SEQ ID NO: 159; and the heavy chain comprises an amino acid sequence as set forth in SEQ ID NO: 160; q) the light chain comprises an amino acid sequence as set forth in SEQ ID NO: 153; and the heavy chain comprises an amino acid sequence as set forth in SEQ ID NO: 182; r) the light chain comprises an amino acid sequence as set forth in SEQ ID NO: 147; and the heavy chain comprises an amino acid sequence as set forth in SEQ ID NO: 183; s) the light chain comprises an amino acid sequence as set forth in SEQ ID NO: 141; and the heavy chain comprises an amino acid sequence as set forth in SEQ ID NO: 184; t) the light chain comprises an amino acid sequence as set forth in SEQ ID NO: 153; and the heavy chain comprises an amino acid sequence as set forth in SEQ ID NO: 185; u) the light chain comprises an amino acid sequence as set forth in SEQ ID NO: 147; and the heavy chain comprises an amino acid sequence as set forth in SEQ ID NO: 186; v) the light chain comprises an amino acid sequence as set forth in SEQ ID NO: 141; and the heavy chain comprises an amino acid sequence as set forth in SEQ ID NO: 187; w) the light chain comprises an amino acid sequence as set forth in SEQ ID NO: 153; and the heavy chain comprises an amino acid sequence as set forth in SEQ ID NO: 188; x) the light chain comprises an amino acid sequence as set forth in SEQ ID NO: 147; and the heavy chain comprises an amino acid sequence as set forth in SEQ ID NO: 189; y) the light chain comprises an amino acid sequence as set forth in SEQ ID NO: 141; and the heavy chain comprises an amino acid sequence as set forth in SEQ ID NO: 190; z) the light chain comprises an amino acid sequence as set forth in SEQ ID NO: 153; and the heavy chain comprises an amino acid sequence as set forth in SEQ ID NO: 191; aa) the light chain comprises an amino acid sequence as set forth in SEQ ID NO: 147; and the heavy chain comprises an amino acid sequence as set forth in SEQ ID NO: 192; ab) the light chain comprises an amino acid sequence as set forth in SEQ ID NO: 141; and the heavy chain comprises an amino acid sequence as set forth in SEQ ID NO: 193; ac) the light chain comprises an amino acid sequence as set forth in SEQ ID NO: 153; and the heavy chain comprises an amino acid sequence as set forth in SEQ ID NO: 194; ad) the light chain comprises an amino acid sequence as set forth in SEQ ID NO: 147; and the heavy chain comprises an amino acid sequence as set forth in SEQ ID NO: 195; ae) the light chain comprises an amino acid sequence as set forth in SEQ ID NO: 141; and the heavy chain comprises an amino acid sequence as set forth in SEQ ID NO: 196; af) the light chain comprises an amino acid sequence as set forth in SEQ ID NO: 153; and the heavy chain comprises an amino acid sequence as set forth in SEQ ID NO: 197; ag) the light chain comprises an amino acid sequence as set forth in SEQ ID NO: 147; and the heavy chain comprises an amino acid sequence as set forth in SEQ ID NO: 198; ah) the light chain comprises an amino acid sequence as set forth in SEQ ID NO: 141; and the heavy chain comprises an amino acid sequence as set forth in SEQ ID NO: 199; ai) the light chain comprises an amino acid sequence as set forth in SEQ ID NO: 153; and the heavy chain comprises an amino acid sequence as set forth in SEQ ID NO: 200; aj) the light chain comprises an amino acid sequence as set forth in SEQ ID NO: 147; and the heavy chain comprises an amino acid sequence as set forth in SEQ ID NO: 201; ak) the light chain comprises an amino acid sequence as set forth in SEQ ID NO: 141; and the heavy chain comprises an amino acid sequence as set forth in SEQ ID NO: 202; al) the light chain comprises an amino acid sequence as set forth in SEQ ID NO: 153; and the heavy chain comprises an amino acid sequence as set forth in SEQ ID NO: 203; am) the light chain comprises an amino acid sequence as set forth in SEQ ID NO: 147; and the heavy chain comprises an amino acid sequence as set forth in SEQ ID NO: 204; an) the light chain comprises an amino acid sequence as set forth in SEQ ID NO: 141; and the heavy chain comprises an amino acid sequence as set forth in SEQ ID NO: 205; ao) the light chain comprises an amino acid sequence as set forth in SEQ ID NO: 153; and the heavy chain comprises an amino acid sequence as set forth in SEQ ID NO: 206; ap) the light chain comprises an amino acid sequence as set forth in SEQ ID NO: 147; and the heavy chain comprises an amino acid sequence as set forth in SEQ ID NO: 207; aq) the light chain comprises an amino acid sequence as set forth in SEQ ID NO: 141; and the heavy chain comprises an amino acid sequence as set forth in SEQ ID NO: 208; ar) the light chain comprises an amino acid sequence as set forth in SEQ ID NO: 153; and the heavy chain comprises an amino acid sequence as set forth in SEQ ID NO: 209; as) the light chain comprises an amino acid sequence as set forth in SEQ ID NO: 147; and the heavy chain comprises an amino acid sequence as set forth in SEQ ID NO: 210; or at) the light chain comprises an amino acid sequence as set forth in SEQ ID NO: 141; and the heavy chain comprises an amino acid sequence as set forth in SEQ ID NO: 211.

Clause 41: The method of any of clauses 33-40, wherein the antibody is a monoclonal antibody.

Clause 42: The method of any of clauses 33-40, wherein the antibody is a human antibody.

Clause 43: The method of any of clauses 33-40, wherein the antibody is a bispecific antibody.

Clause 44: The method of any of clauses 33-40, wherein the binding fragment thereof is selected from the group consisting of Fab, Fab′, F(ab′)₂, Fd, Fv, scFv, a single-chain antibody, a minibody, and a diabody.

Clause 45: The method of clause 44, wherein the binding fragment thereof is an scFv.

Clause 46: A computer-implemented method comprising: scoring a polypeptide comprising an amino acid sequence with a computationally binding optimized (CBO) model for a functional property relating to modulating the activity of a target molecule, the CBO model: for each amino acid position of the amino acid sequence of the polypeptide, calculating a plurality of energy scores, the energy scores based on the amino acid at a given position in the amino acid sequence, adding each energy score calculated to an array, generating a normalized sum of the energy scores for each position, and generating a score of the polypeptide by summing each energy score calculated, the score representing the functional property of the polypeptide, the functional property relating to the polypeptide modulating the activity of the target molecule.

Clause 47: The computer-implemented method of clause 46, further comprising: calculating a logarithm of each energy score calculated; wherein summing each energy score calculated is performed by summing the logarithms of each energy score calculated; wherein the energy scores are further calculated based on having substituted the amino acid at the given position in the amino acid sequence with each of a plurality of different amino acids.

Clause 48: The computer-implemented method of any of clauses 46-47, wherein scoring the polypeptide using the CBO model is implementable by the script of Appendix A, and the CBO model is substantially similar to the table of Appendix B.

Clause 49: The method of any of clauses 46-48, wherein the functional property is at least one of: a binding affinity for an interleukin-13 (IL-13) polypeptide characterized by a K_{D} of 10 pM or less (optionally, as measured by KinExA); a binding specificity for the IL-13 polypeptide; a neutralizing activity against the IL-13 polypeptide (optionally, a full-length human IL-13); or an inhibitory activity against IL-13-mediated signaling, or a combination of the foregoing.

Clause 50: The method of clause 49, wherein the target molecule is interleukin-13 (IL-13), and the functional property of the relating to the polypeptide modulates the activity of the IL-13.

Clause 51: A system comprising: a processor; and a memory with computer code instructions stored thereon, the processor and the memory, with the computer code instructions, being configured to cause the system to: score a polypeptide comprising an amino acid sequence with a CBO model for a functional property relating to modulating the activity of a target molecule, the CBO model: for each amino acid position of the amino acid sequence of the polypeptide, calculating a plurality of energy scores, the energy scores based on the amino acid at a given position in the amino acid sequence, add each energy score calculated to an array, generate a normalized sum of the energy scores for each position, and generate a score of the amino acid sequence by summing each energy score calculated, the score representing a functional property of the polypeptide, the functional property relating to the polypeptide modulating the activity of the target molecules.

Clause 52: The system of clause 51, wherein the instructions are further configured to cause the system to calculate a logarithm of each energy score calculated; wherein summing each energy score calculated is performed by summing the logarithms of each energy score calculated; wherein the energy scores are further based on having substituted the amino acid at the given position in the amino acid sequence with each of a plurality of different amino acids.

Clause 53: The system of any of clauses 51-52, wherein scoring the polypeptide using the CBO model is implemented by the script of Appendix A and wherein the CBO model is substantially similar to the table of Appendix B.

Clause 54: The system of any of clauses 51-53 wherein the functional property is at least one of: a binding affinity for an interleukin-13 (IL-13) polypeptide characterized by a K_{D} of 10 pM or less (optionally, as measured by KinExA); a binding specificity for the IL-13 polypeptide; a neutralizing activity against the IL-13 polypeptide (optionally, a full-length human IL-13); or an inhibitory activity against IL-13-mediated signaling, or a combination of the foregoing.

Clause 55: The method of clause 54, wherein the target molecule is interleukin-13 (IL-13), and the functional property of the relating to the polypeptide modulates the activity of the IL-13.

Clause 56: A polypeptide that specifically binds an interleukin-13 (IL-13): wherein the polypeptide comprises an amino acid sequence that is assigned a score above a predetermined threshold by a computationally binding optimized (CBO) model upon scoring the amino acid sequence of the polypeptide.

Clause 57: The polypeptide of clause 56, wherein the polypeptide is scorable by the script of Computer Program Listing Appendix A and wherein the CBO model is calculated by a table substantially similar to that of Computer Program Listing Appendix B and wherein the polypeptide is assigned the score that is calculated using the Computer Program Listing Appendix B; wherein the polypeptide is assigned the score by a script substantially similar to Computer Program Listing Appendix A, the script employing the CBO model substantially similar to the table of Computer Program Listing Appendix B, wherein the polypeptide has a logarithmic score of at least about: −1.7, −1.0, −0.5, 0, 0.5, 1, 1.5, 1.8, 2.0, and 3.0.

Clause 58: The polypeptide of any of clauses 56-57, wherein the polypeptide has one or more properties selected from: a binding affinity for an interleukin-13 (IL-13) polypeptide characterized by a K_{D} of 10 pM or less (optionally, as measured by KinExA); a binding specificity for the IL-13 polypeptide; a neutralizing activity against the IL-13 polypeptide (optionally, a full-length human IL-13); or an inhibitory activity against IL-13-mediated signaling, or a combination of the foregoing.

Clause 59: The polypeptide of any of clauses 56-58, wherein the CBO model outputs a score that is equal to or above a score from the CBO model of one or more of a reference polypeptide that comprises a VL and VH pair selected from: SEQ ID NO: 97 and SEQ ID NO: 98 (AbA); SEQ ID NO: 99 and SEQ ID NO: 100 (AbB); SEQ ID NO: 101 and SEQ ID NO: 102 (AbC); SEQ ID NO: 103 and SEQ ID NO: 104 (AbD); SEQ ID NO: 105 and SEQ ID NO: 106 (AbE); SEQ ID NO: 107 and SEQ ID NO: 108 (AbF); SEQ ID NO: 109 and SEQ ID NO: 110 (AbG); SEQ ID NO: 111 and SEQ ID NO: 112 (AbH); SEQ ID NO: 113 and SEQ ID NO: 114 (AbI); SEQ ID NO: 115 and SEQ ID NO: 116 (AbJ); SEQ ID NO: 117 and SEQ ID NO: 118 (AbK); SEQ ID NO: 119 and SEQ ID NO: 120 (AbL); SEQ ID NO: 121 and SEQ ID NO: 122 (AbM); SEQ ID NO: 123 and SEQ ID NO: 124 (AbN); SEQ ID NO: 125 and SEQ ID NO: 126 (AbO); or SEQ ID NO: 127 and SEQ ID NO: 128 (AbP).

Clause 60: The polypeptide of any of clauses 56-59, wherein the polypeptide does not comprise a heavy chain comprising SEQ ID NO: 179 or 181 and a light chain comprising SEQ ID NO: 178 or 180.

Clause 61: A polypeptide: wherein the polypeptide comprises an amino acid sequence that is assigned a score above a predetermined threshold by a computationally binding optimized (CBO) model upon scoring the amino acid sequence of the polypeptide.

Clause 62: A polypeptide that binds human interleukin-13 (IL-13), wherein the polypeptide is designed by a method comprising: generating a polypeptide sequence with a CBO model; verifying the generated polypeptide sequence using the CBO model by: for each amino acid position of the polypeptide sequence, calculating a plurality of energy scores, the energy scores based on having substituted the amino acid at a given position in the polypeptide sequence with each of a plurality of different amino acids, adding each energy score calculated to an array, generating a normalized sum of the energy scores for each position, calculating a logarithm of each energy score calculated, and generating a score of the polypeptide sequence by summing the logarithms of each energy score calculated, the score representing a functional property of the polypeptide's ability to bind to human IL-13.

### EXAMPLES

The examples herein are provided to illustrate advantages and benefits described herein and to further assist a person of ordinary skill in the art with making and using the IL-13 antibody or antigen binding fragment thereof described herein. The examples herein are also presented in order to more fully illustrate the aspects described herein. The examples should in no way be construed as limiting the scope described herein, as defined by the appended claims. The examples can include or incorporate any of the variations, aspects, or embodiments described herein described above. The variations, aspects or embodiments described above may also further each include or incorporate the variations of any or all other variations, aspects or embodiments described herein.

### Example 1: Kinetics of IL-13 Antibodies

The binding kinetics of a panel of IL-13 antibodies was assessed by Surface Plasmon Resonance (SPR) using a Biacore 8K+ instrument (Cytiva, Cat. No. 2873569).

Briefly, a goat anti-human polyclonal antibody surface was prepared via amine-coupling onto a carboxymethylated dextran (CM4) (Cytiva, Cat. No. 29104989) sensor chip to attain approximately 5,000 response units (RU). Each antibody was prepared at 20 μg/mL and captured for 30 seconds at a flow rate of 10 μL per minute to achieve a capture level of approximately 100 RU. Next, human IL-13 was prepared in HBS-EP+, pH 7.6 running buffer (10 mM HEPES pH 7.4, 150 mM NaCl, 3 mM EDTA, and 0.05% v/v surfactant P20) and injected over the captured antibody surface at 5 concentrations in a 2-fold serial dilution series from 0.6 nM to 10 nM. Each analyte concentration was then injected in a separate cycle for 300 seconds at a flow rate of 30 μL per minute and the complex was allowed to dissociate for 5,400 seconds. The surface was regenerated by injecting two pulses of 1.5% phosphoric acid for 30 seconds at a flow rate of 30 μL per minute in between each cycle. The assay was performed at 37° C. with technical replicates at a collection rate of 1 Hz. Kinetic parameters for the concentration series were obtained by double referencing and globally fitting the data to a 1:1 binding model using the Biacore Insight Evaluation software (Cytiva, version 5.0.18.22102). For binding interactions reaching this recommendation during the 5,400-second dissociation phase, the kinetic and affinity values were reported, and the results were expressed as average±standard deviation of one independent experiment with technical replicates, as shown in Table 5. Table 5 represents the binding and affinity values obtained through Surface Plasmon Resonance (SPR) of reference and variant antibodies in the panel. The kinetic (kₐ and k_{d}) and affinity (K_{D}) values are expressed as average±standard deviation of one independent experiment with technical replicate. For binding interactions that did not attain at least a 5% decrease in the binding response for the highest antigen concentration assessed during the dissociation phase, the affinity values were estimated to be less than 15 pM.

TABLE 5
Binding and affinity values of reference anti-IL-13
antibodies and anti-IL-13 variant antibodies.

| Antibody | Description | Type | kₐ (10⁵ M⁻¹s⁻¹) | k_{d} (10⁻⁶ s⁻¹) | K_{D} (pM) |
|---|---|---|---|---|---|
| Ref2 | reference | hIgG4 | 6.95 ± 3.75 | 32.23 ± 11.88 | 51.17 ± 20.90 |
| Ref1 | reference | hIgG1-YTE | 5.94 ± 1.67 | 26.38 ± 2.64 | 48.41 ± 17.36 |
| AbC | variant | hIgG1-YTE | 10.92 ± 0.39 | 29.79 ± 1.79 | 27.33 ± 2.60 |
| AbB | variant | hIgG1-YTE | 5.52 ± 1.71 | 10.18 ± 3.19 | 21.52 ± 13.38 |
| AbA | variant | hIgG1-YTE | 8.47 ± 5.03 | 11.14 ± 2.66 | 16.24 ± 8.41 |
| AbD | variant | hIgG1-YTE | not reported | not reported | <15 |
| AbE | variant | hIgG1-YTE | not reported | not reported | <15 |
| AbF | variant | hIgG1-YTE | not reported | not reported | <15 |
| AbG | variant | hIgG1-YTE | not reported | not reported | <15 |
| AbH | variant | hIgG1-YTE | not reported | not reported | <15 |
| AbI | variant | hIgG1-YTE | not reported | not reported | <15 |
| AbJ | variant | hIgG1-YTE | not reported | not reported | <15 |
| AbK | variant | hIgG1-YTE | not reported | not reported | <15 |
| AbL | variant | hIgG1-YTE | not reported | not reported | <15 |
| AbM | variant | hIgG1-YTE | not reported | not reported | <15 |
| AbN | variant | hIgG1-YTE | not reported | not reported | <15 |
| AbO | variant | hIgG1-YTE | not reported | not reported | <15 |
| AbP | variant | hIgG1-YTE | not reported | not reported | <15 |

### Example 2: Characterization of Anti-IL-13 Antibody Blocking Activity

The ability of an antibody to block IL-13 from signaling through IL-13Ra1/IL-4Ra was quantitatively assessed using the HEK-Blue colorimetric assay. Briefly, the assay employs IL-13Ra1/IL-4Ra HEK-Blue cells (an engineered Human Embryonic Kidney (HEK) cell line (Invivogen, Cat. No. hkb-il413) that endogenously express the IL-13Ra1/IL-4Ra receptor complex) and are transfected with STAT6 and a STAT6 inducible secreted alkaline phosphatase (SEAP) reporter gene. STAT6 is downstream of the IL-13Ra1/IL-4Ra receptor complex and IL-13 binding leads to STAT6 activation and production of SEAP. Notably, stimulation of IL-13Ra1/IL-4Ra HEK-Blue cells with IL-13 can be blocked with an anti-IL-13 antibody, which prevents expression of SEAP. When SEAP interacts with QUANTI-Blue solution (Invivogen, Cat. No. QBLB 44-02/QBLB 44-05), the color of the QUANTI-Blue solution changes from pink to purple/blue in which the intensity reflects the activity level of SEAP. This assay can thus be used to quantify the ability of an antibody to block IL-13 signaling.

For this assay, HEK-Blue cells were grown to 80% confluency in media consisting of DMEM+Glutamax (Gibco, Cat. No. 105666-616), 10% FBS, 1% Penicillin-Streptomycin (Gibco, Cat. No. 15140-122), and 100 μg/mL Normocin (Invivogen, Cat. No. NC9273499). Cells were then removed from flasks using 0.25% trypsin-EDTA (Gibco, Cat. No. 25200-056), washed in media, and replated in a 384-well flat-bottom plate at 12,500 cells/25 μL media. An anti-IL-13 antibody is prepared in a 2-fold serial dilution in cell culture media for 11 points starting at 3 μg/mL. Next, 50 μL of the diluted antibody was pre-complexed with 50 μL of IL-13 cytokine in media in a 96-well plate. The final concentration of IL-13 in assay was 50 ng/ml. The antibody and cytokine were pre-complexed for 30 minutes at 37° C. Subsequently, 25 μL of antibody/cytokine complex was stamped onto plated cells and incubated overnight at 37° C.

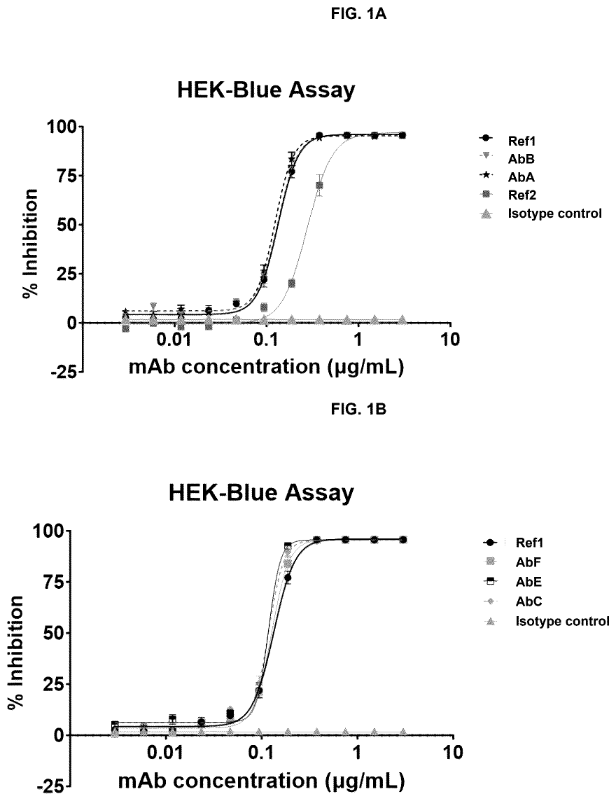

The next day, 5 μl of cell supernatant was extracted from each well and added to 85 μl of QUANTI-Blue solution in a separate 384-well flat-bottom plate. Next, this mixture was allowed to develop protected from the dark for 40 minutes at room temperature. After developing, the absorbance was measured on the Envision plate reader (Perkin Elmer) at 635 nm. Raw data were then analyzed by normalizing response to the IgG1 isotype negative control to obtain percent inhibition. The results for the antibody panel were visualized in FIGS. 1A-F. IC₅₀ values were calculated by fitting a 4-parameter non-linear regression curve using an average of three technical replicates per antibody concentration as reported in Table 6.

TABLE 6
Normalized IC₅₀ values obtained for reference anti-
IL-13 antibodies and variant anti-IL-13 antibodies.

| Antibody | IC₅₀ (μg/mL) |
|---|---|
| Ref1 | 0.143 |
| Ref2 | 0.277 |
| AbA | 0.123 |
| AbB | 0.132 |
| AbC | 0.118 |
| AbD | 0.161 |
| AbE | 0.118 |
| AbF | 0.126 |
| AbG | 0.174 |
| AbH | 0.107 |
| AbI | 0.223 |
| AbJ | 0.167 |
| AbK | 0.172 |
| AbL | 0.320 |
| AbM | 0.136 |
| AbN | 0.140 |
| AbO | 0.165 |
| AbP | 0.152 |

### Example 3. Characterization of an Anti-IL-13 Antibody Blocking Activity

### HEK-Blue Assay

A HEK-Blue IL-4/IL-13 assay, when executed over three days, provided a robust assessment of the functional blockade of IL-13 by Antibody AbAB. For this assay, HEK-Blue cells were grown, harvested, and seeded into 384-well plates at a seeding density of 1,560 cells per well. The plates were incubated at 37° C. for 24 hours. On Day 2, the anti-IL-13 antibodies were then prepared in a 2-fold serial dilution in cell culture media for 11 total dilutions. Next, the diluted antibody was pre-complexed with 3.7 nM of IL-13 cytokine in media at room temperature for 30 minutes, followed by transfer to cell plates and incubation at 37° C. for 24 hours. On Day 3, the cell culture supernatant was then mixed with QUANTI-Blue solution and incubated for six hours at room temperature, and the absorbance was measured on a plate reader at 635 nm.

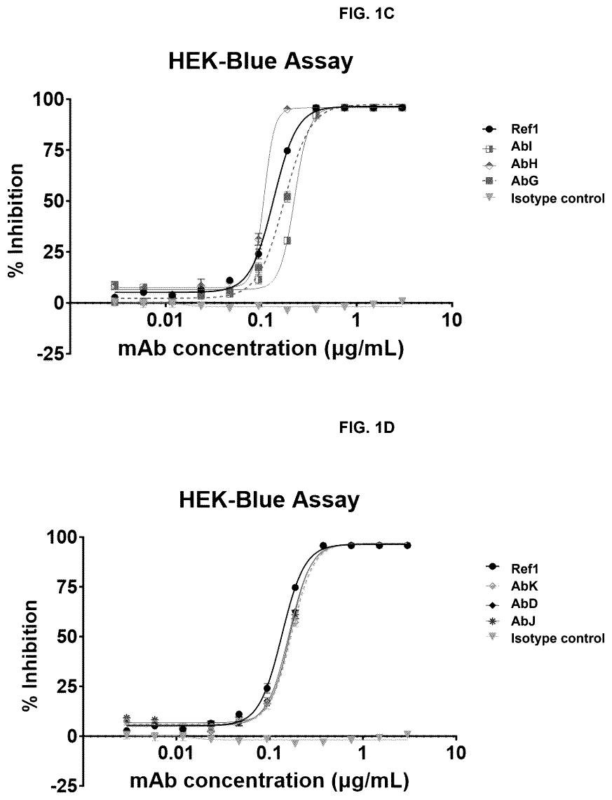

Raw data were then subjected to baseline correction using the signal from wells containing no cytokine or antibody. The SEAP activity in wells treated with IL-13 alone (no antibody) was set as 100% activation for normalization. The percentage of inhibition for antibody treatment was then calculated as (1−(absorbance of well containing antibody/absorbance of well containing IL-13 alone))×100. The results are shown in Table 7 and presented relative to an isotype control antibody in FIG. 2.

TABLE 7
IC₅₀ value obtained for variant anti-IL-13 antibody.

| Identifier | Mean IC₅₀ (nM) | SD | % CV |
|---|---|---|---|
| AbAB | 0.113 | 0.0300 | 26.4% |

pSTAT6 Assay in Human and Cynomolgus CD14⁺ Monocytes

The ability of an antibody to inhibit IL-13 from signaling through IL-13Ra1/IL-4Ra and activating STAT6 in CD14+ monocytes was quantitatively assessed using a Phospho-STAT6 (pSTAT6) assay. The phosphorylation of STAT6 is measured by flow cytometry after treatment with precomplexed anti-IL-13 antibodies and IL-13 cytokine. This assay can thus be used to quantify the ability of an antibody to block IL-13 signaling.

Briefly, human Leukopaks (StemCell Technologies, Cat. No. 70500.1; BioIVT, Cat. No. HUMANLX100) or cynomolgus whole blood (HumanCells BioSciences, Cat. No. M2-010-80) were diluted 1:1 with 1×PBS supplemented with 5 mM EDTA, and leukocytes were isolated by Ficoll-Paque density gradient centrifugation at 700×g for 30 minutes. The cells were washed twice at 300×g for 3 minutes, resuspended in ACK lysis buffer (Gibco, Cat. No. 2975020) for 5 minutes and washed again before the cells were counted and cryopreserved in cryovials in liquid nitrogen. The cells were then thawed and transferred to 15 mL falcon tubes with 10 mL of media (RPMI 1640x+Glutamax (Gibco, Cat. No. 61879-036), 10% FBS (Gibco, Cat. No. A56705-01), and 1% Penicillin-Streptomycin (Gibco, Cat. No. 15140-122)). Cells were washed twice at 1,200 RPM for 3 minutes, counted, and then stained for viability and surface markers CD3 (T cell population) and CD14 (monocyte population). After staining, cells were then resuspended in media (100,000 cells/100 μL for human cells; 150,000/100 μL cells for cynomolgus cells), plated onto a 96-well flat-bottom plate, and incubated for 1 hour at 37° C.

Anti-IL-13 antibodies were serially diluted (starting concentration of 33.3 ug/mL for both species, 2-fold), then pre-complexed with IL-13 cytokine at 1 μg/mL for human samples and 0.5 μg/mL for cynomolgus samples with a 30-minute incubation at 37° C. Then, 100 μL of the complex were added to human or cynomolgus cells, which were further incubated for 30 minutes (human cells) or 1 hour (cynomolgus cells). Cells were then transferred into 96-well U-bottom plates. To stop the STAT6 signal, 2% PFA (Electron Microscopy Sciences, Cat. No. 15710) was added to cells, the mixture was incubated for 10 minutes at room temperature, and the cells were then spun down at 1,200 RPM for 3 minutes. Cells were washed twice in FACS buffer. Cells were resuspended in 150 μL of 100% cold methanol, transferred into a 0.5 mL assay block, and stored at −80° C. overnight. The next day, the blocks were removed from the freezer and cells were transferred into a 96-well U-bottom plate. Plates were centrifuged at 1,200 RPM for 3 minutes and the methanol supernatant was removed. Cells were then washed once in FACS, spinning at 1,200 RPM for 3 minutes. Cells were then stained for intracellular pSTAT6 for 30 minutes in FACS buffer, washed once with FACS buffer, and resuspended in 200 μL of FACS buffer for analysis using a flow cytometer.

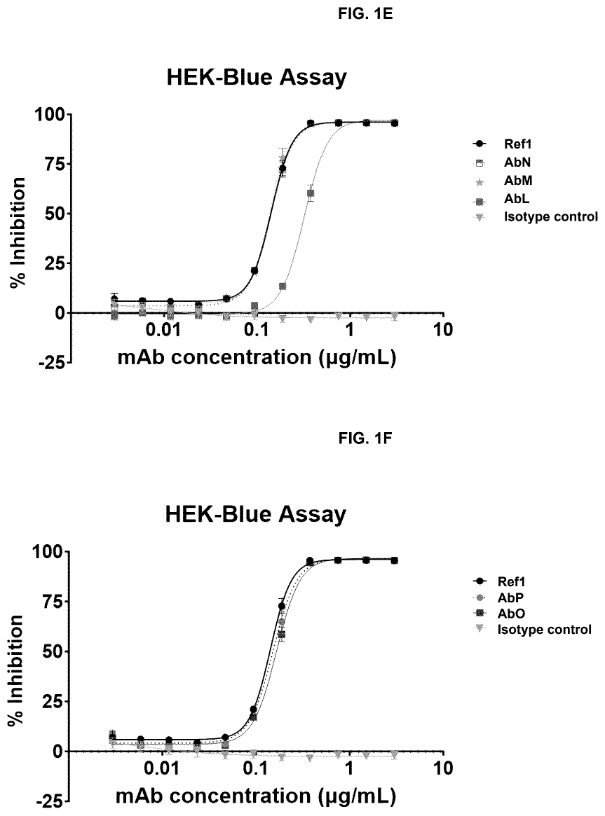

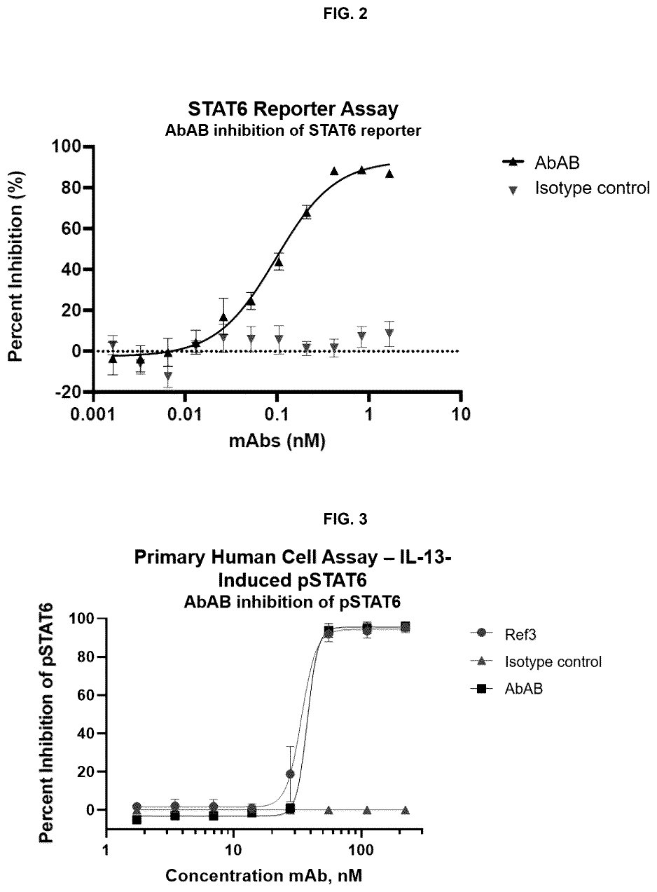

Data from three donors for each human or cynomolgus cells were analyzed with FlowJo software (BD Biosciences; version 10.8.1 for humans and 10.8.2 for cynomolgus), gating the live CD14⁺ monocytes, then gating the pSTAT6 positive percentage of these monocytes. The data were normalized to an isotype control to obtain percentage inhibition, and the percentage pSTAT6⁺ monocytes were plotted against antibody concentration. Percentage inhibition of pSTAT6 for each antibody concentration is shown for representative human Donor 3 (FIG. 3) and representative cynomolgus Donor 1 (FIG. 4). IC₅₀ values were calculated by fitting a 4-parameter non-linear regression curve to the average of three technical replicates for each antibody concentration per donor. Table 8 shows the results for three human donors and Table 9 shows the results for three cynomolgus donors.

TABLE 8
IC₅₀ values obtained in Phospho-STAT6 assay for
anti-IL-13 antibodies from three human donors.

| | Mean | Mean | Mean | | | | |
|---|---|---|---|---|---|---|---|
| | IC₅₀ (nM) | IC₅₀ (nM) | IC₅₀ (nM) | | Standard | Upper | Lower |
| | Donor 1 | Donor 2 | Donor 3 | | Error of | 95% CI | 95% CI |
| Identifier | n = 3 | n = 3 | n = 3 | SD | Mean | of Mean | of Mean |
| Ref3 | 32.18 | 31.32 | 33.89 | 1.31 | 0.755 | 35.71 | 29.24 |
| AbAB | 34.9 | 33.04 | 37.67 | 2.33 | 1.345 | 40.991 | 29.416 |
| isotype | NA | NA | NA | NA | NA | NA | NA |
| IgG1- | | | | | | | |
| YTE-TM | | | | | | | |

TABLE 9
IC₅₀ values obtained in Phospho-STAT6 assay for variant
anti-IL-13 antibody from three cynomolgus donors.

| | Mean | Mean | Mean | | | | |
|---|---|---|---|---|---|---|---|
| | IC₅₀ (nM) | IC₅₀ (nM) | IC₅₀ (nM) | | Standard | Upper | Lower |
| | Donor 1 | Donor 2 | Donor 3 | | Error of | 95% CI | 95% CI |
| Identifier | n = 3 | n = 3 | n = 3 | SD | Mean | of Mean | of Mean |
| AbAB | 18.79 | 15.11 | 18.06 | 1.95 | 1.12 | 12.48 | 22.16 |

### Example 4: Kinetics of Anti-IL-13 Antibodies Measured by SPR and KinExA

The binding kinetics of a subset of anti-IL-13 antibodies was further assessed by Surface Plasmon Resonance (SPR) using a Biacore 8K+instrument.

Briefly, an anti-human Fc antibody capture sensor surface was prepared via amine-coupling onto a carboxymethylated dextran (CM4) sensor chip to attain approximately 5,000 response units (RU) of immobilized anti-human Fc antibody. Each antibody (AbAB, AbAE, Ref1, and Ref2) was prepared in HBS-EP+, pH 7.6 (10 mM HEPES pH 7.4, 150 mM NaCl, 3 mM EDTA, and 0.05% v/v surfactant P20) at 20 mg/mL and captured for 30 seconds at a flow rate of 1 μL per minute to achieve a capture level of approximately 100 RU.

Next, human IL-13 was injected over the captured antibody surface at 5 concentrations in a 2-fold serial dilution series from 0.6 nM to 10 nM. Each analyte concentration was injected in a separate cycle for 300 seconds at a flow rate of 40 μL per minute and the complex was allowed to dissociate for 5,400 seconds using HBS-EP+ as the running buffer. The surface was regenerated by injecting two pulses of 1.5% phosphoric acid for 30 seconds at a flow rate of 40 μL per minute in between each cycle. The assay was performed at 37° C. with technical replicates. Kinetic parameters for the concentration series were obtained by double referencing and globally fitting the data to a 1:1 binding kinetics model using the Biacore Insight Evaluation software. Table 10 represents the binding and affinity values obtained through Surface Plasmon Resonance (SPR) of reference and variant antibodies in the subset of anti-IL-13 antibodies. The kinetic (kₐ and k_{d}) and affinity (K_{D}) values are expressed as average±standard deviation of one independent experiment with technical replicates.

TABLE 10
Kinetic and affinity values of reference anti-IL-13
antibodies and a subset of anti-IL-13 antibodies.

| Antibody | Type | kₐ (10⁵ M⁻¹s⁻¹) | k_{d} (10⁻⁶ s⁻¹) | K_{D} (pM) |
|---|---|---|---|---|
| Ref3 | hIgG1-YTE- | 5.01 ± 0.39 | 25.18 ± 2.72 | 50.66 ± 9.37 |
| | TM | | | |
| Ref1 | hIgG1-YTE | 4.83 ± 0.42 | 26.57 ± 1.40 | 55.13 ± 1.85 |
| AbAB | hIgG1-YTE- | 6.76 ± 0.15 | 4.85 ± 3.47 | 7.22 ± 5.29 |
| | TM | | | |
| AbG | hIgG1-YTE | 6.39 ± 1.11 | 7.68 ± 2.52 | 12.56 ± 6.13 |

The SPR data demonstrated that Antibody AbAB binds to human IL-13 with higher affinity than Ref3. In addition, the affinity values for the antibody containing the same variable regions expressed as human IgG1 with YTE and TM mutations or YTE mutations in the Fc region were within 2-fold. These differences are considered within the range of variability of the methodology. This data suggested that the TM mutations in the Fc region do not impact the binding affinity to IL-13.

Since the surface-linked binding kinetics were approaching the limitations of SPR sensitivity, KinExA, a solution-based approach, was used to overcome these limitations. Briefly, glass beads coated with Ref1 antibody were used to capture a portion of free antigen from an equilibrated sample of antigen and antibody. The captured antigen was detected with a fluorescently labeled anti-HIS-IgG. The fluorescent signal was converted to a voltage signal that is directly proportional to the amount of free antigen in the equilibrated sample. The experiments were performed at 25° C. with either human IL-13 or cynomolgus IL-13. The kinetic and affinity result are presented in Table 11.

TABLE 11
Kinetics and affinity of anti-IL-13 antibodies to human and cynomolgus IL-13.

| Antibody | Type | Antigen | kₐ (10⁶ M⁻¹s⁻¹) | k_{d} (10⁻⁶ s⁻¹) | K_{D} (pM) |
|---|---|---|---|---|---|
| Ref2 | hIgG4 | Human IL-13 | 1.07 | 9.76 | 9.09 |
| Ref1 | hIgG1-YTE | Human IL-13 | 1.53 | 9.37 | 6.13 |
| AbG | hIgG1-YTE | Human IL-13 | 2.06 | 0.48 | 0.23 |
| Ref1 | hIgG1-YTE | Cynomolgus IL-13 | 2.09 | 1.09 | 0.52 |
| AbG | hIgG1-YTE | Cynomolgus IL-13 | 2.46 | 1.77 | 0.72 |

The KinExA data demonstrated that Antibody AbG binds to human IL-13 with a 40-fold higher affinity than Ref2 with K_{D} values of 0.23 pM and 9.09 pM, respectively (Table 11). In addition, the affinity values for the antibody containing the same variable regions expressed as human IgG1 with YTE mutations or human IgG4 (e.g., Ref 1 vs. Ref2) are within 2-fold. These differences are considered within the range of variability of the methodology. These data suggested that the isotype and YTE mutations in the Fc region do not impact the binding affinity to IL-13. In addition, Antibody AbG binds to cynomolgus IL-13 with a similar affinity as Ref1 with K_{D} values of 0.72 pM and 0.52 pM, respectively. In summary, Antibody AbG and Antibody AbAB binds to human IL-13 with higher affinity to human IL-13 and to cynomolgus IL-13 with similar affinity compared to reference molecule Ref1.

### Example 5: Kinetics and Affinity of Anti-IL-13 Antibodies Measured by SPR to Mouse and Rat IL-13

The binding kinetics of a subset of anti-IL-13 antibodies was evaluated by Surface Plasmon Resonance (SPR) using a Biacore 8K+instrument to determine if rodent is an acceptable toxicology species.

Briefly, the kinetics of Antibody AbG and Antibody AbAB binding to recombinant mouse and rat IL-13 was assessed using an anti-human IgG Fc prepared via amine-coupling onto a CM4 sensor chip to attain approximately 3,000 response units (RU) of immobilized anti-human Fc antibody. Antibody AbAB, Antibody AbG, Ref2, Ref3, isotype-hIgG4, isotype-hIgG1-YTE-TM, or tralokinumab-hIgG4 were injected for 30 seconds at a flow rate of 10 μL per minute over the surface to obtain a capture level of approximately 150-225 RU. Human, mouse, or rat IL-13 were injected over the surface using an 8-point, 3-fold serial dilution. For determination of binding kinetics, a maximum concentration of 100, 1000, or 1000 nM was used for human, rat, and mouse IL-13 antigen, respectively. Determination of no binding was defined as a response below 5 RU at a maximum concentration of 1000 nM. Each concentration of antigen was injected in a separate cycle for 300 seconds at a flow rate of 30 μL per minute, and the complex was allowed to dissociate for 900 seconds using HBS-EP+ as the running buffer. The surface was regenerated by injecting two pulses of 1.5% phosphoric acid for 30 seconds at a flow rate of 30 μL per minute in between each cycle. The assay was performed at 25° C. with three technical replicates. Kinetic parameters for the concentration series were obtained by double referencing and globally fitting the data to a 1:1 binding model with mass transport limitation using the Biacore Insight Evaluation software. The results are expressed as an average±standard deviation of one independent experiment with three technical replicates (Table 12). No Binding denotes no detectable binding whereas Not Reported denotes detectable binding with no values reported.

TABLE 12
Kinetics and affinity values of anti-IL-13 antibodies to rat, mouse, and human IL-13.

| Antibody | Antigen | kₐ (10⁵ M⁻¹s⁻¹) | k_{d} (10⁻⁶ s⁻¹) | K_{D} (pM) |
|---|---|---|---|---|
| AbAB | Rat IL-13 | 0.36 ± 0.02 | 0.81 ± 0.04 | 22.80 ± 1.31 |
| AbG | Rat IL-13 | 0.33 ± 0.03 | 0.83 ± 0.04 | 25.58 ± 2.17 |
| Ref2 | Rat IL-13 | 0.18 ± 0.00 | 2.16 ± 0.04 | 118.86 ± 4.79 |
| Ref3 | Rat IL-13 | 0.18 ± 0.00 | 1.96 ± 0.1 | 110.71 ± 7.94 |
| isotype-hIgG4 | Rat IL-13 | No Binding | No Binding | No Binding |
| isotype-hIgG1-YTE-TM | Rat IL-13 | No Binding | No Binding | No Binding |
| tralokinumab-hIgG4 | Rat IL-13 | Not Reported | Not Reported | Not Reported |
| AbAB | Mouse IL-13 | No Binding | No Binding | No Binding |
| AbG | Mouse IL-13 | No Binding | No Binding | No Binding |
| Ref2 | Mouse IL-13 | No Binding | No Binding | No Binding |
| Ref3 | Mouse IL-13 | No Binding | No Binding | No Binding |
| isotype-hIgG4 | Mouse IL-13 | No Binding | No Binding | No Binding |
| isotype-hIgG1-YTE-TM | Mouse IL-13 | No Binding | No Binding | No Binding |
| tralokinumab-hIgG4 | Mouse IL-13 | Not Reported | Not Reported | Not Reported |
| AbAB | Human IL-13 | Not Reported | Not Reported | Not Reported |
| AbG | Human IL-13 | Not Reported | Not Reported | Not Reported |
| Ref2 | Human IL-13 | Not Reported | Not Reported | Not Reported |
| Ref3 | Human IL-13 | Not Reported | Not Reported | Not Reported |
| isotype-hIgG4 | Human IL-13 | No Binding | No Binding | No Binding |
| isotype-hIgG1-YTE-TM | Human IL-13 | No Binding | No Binding | No Binding |
| tralokinumab-hIgG4 | Human IL-13 | Not Reported | Not Reported | Not Reported |

The results in Table 12 demonstrated that Antibody AbAB binds to rat IL-13 with higher affinity than Ref2 with K_{D} values of 22.80 nM and 118.86 nM for Antibody AbAB and Ref2, respectively. No binding of Antibody AbAB or Ref2 to mouse IL-13 was detected at a maximum concentration of 1000 nM. Proper folding of recombinant mouse IL-13 was confirmed by binding of positive control antibody tralokinumab. Human IL-13 was included as a positive control antigen with no kinetic parameters reported in this study. The specificity of these results was confirmed by the lack of binding of the isotype controls to all IL-13 antigens assessed.

In summary, Antibody AbAB binds to rat IL-13 with an affinity several orders of magnitude weaker compared to human IL-13 (see, e.g., Table 10). Antibody AbAB does not display sufficient binding to mouse or rat IL-13 to enable rodent as a viable toxicology species.

### Example 6. Primary In Vivo Pharmacodynamic Assessments

The pharmacodynamics of an exemplary anti-IL-13 antibody were characterized using in vivo experiments in a murine model of asthma. Specifically, the in vivo efficacies of Antibody AbAE and Ref1 were assessed in a hIL-13-induced asthma model in C57BL/6 mice. The in vivo model was designed to test the proof of concept, demonstrating the efficacy of AbAE in reducing asthma biomarkers and airway inflammation. The study was not designed nor powered for statistical comparison between AbAE and Ref1.

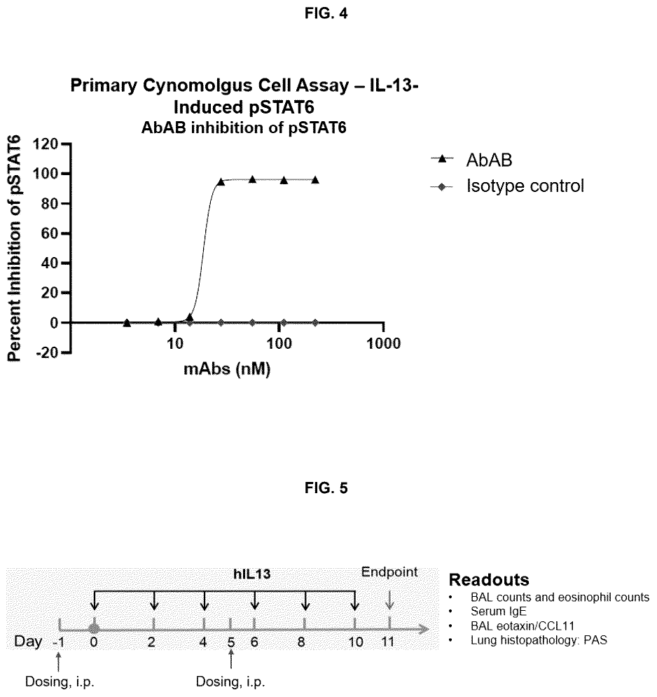

To establish the asthma model, mice were administered 20 μL of 0.25 mg/mL human IL-13 (5 μg/mouse) intranasally every two days (Days 0-10). Mice in the vehicle control group received 20 μL PBS intranasally over the same period. To evaluate drug efficacy, saline and isotype control (25 mg/kg) were administered to the negative and positive control groups, respectively. Antibody AbAE was administered via intraperitoneal injection on the day before dosing (Day −1) and on Day 5, at doses of 1 mg/kg, 5 mg/kg, or 25 mg/kg. Ref1 was administered via intraperitoneal injection on the day before dosing (Day −1) and on Day 5, at doses of 25 mg/kg (FIG. 5). The general condition and body weight of the mice were monitored on a daily basis throughout the duration of the experiment. On day 11, blood, bronchoalveolar lavage fluid (BALF), and lung tissue samples were collected for IgE and chemokine assays, flow cytometry analysis, and histopathology analysis.

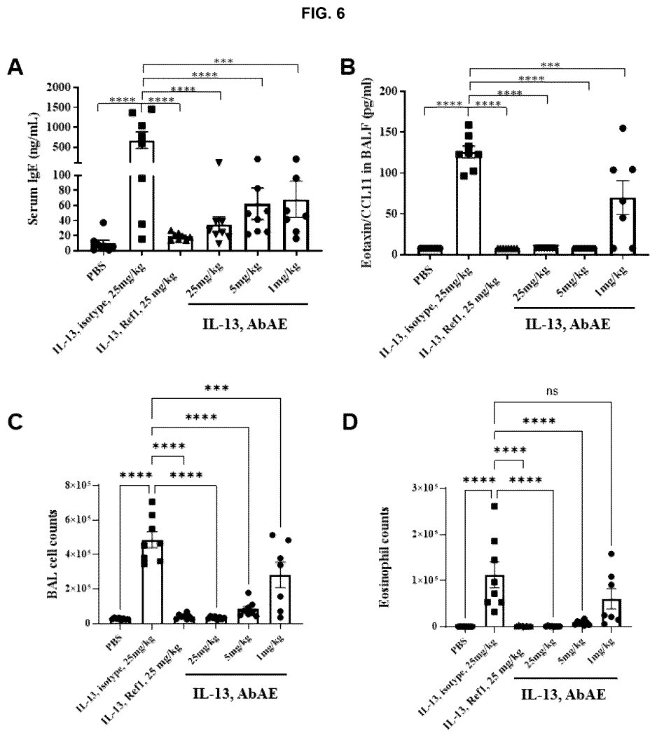

The levels of total serum IgE (FIG. 6A) and BALF eotaxin/CCL11 (FIG. 6B) were elevated in the hIL-13-induced, isotype-treated group as compared to those in the vehicle control group and were significantly reduced with treatment of Antibody AbAE and Ref1 at all doses. Similarly, the total BAL counts (FIG. 6C) and eosinophil counts (FIG. 6D) were elevated in the BALF samples from the hIL-13-induced group as compared to those in the vehicle control group. The levels of total BAL counts were significantly reduced at all doses. The levels of eosinophil counts were significantly reduced at 5 mg/kg and 25 mg/kg doses of Antibody AbAE. Similar findings were observed in mice treated with Ref1.

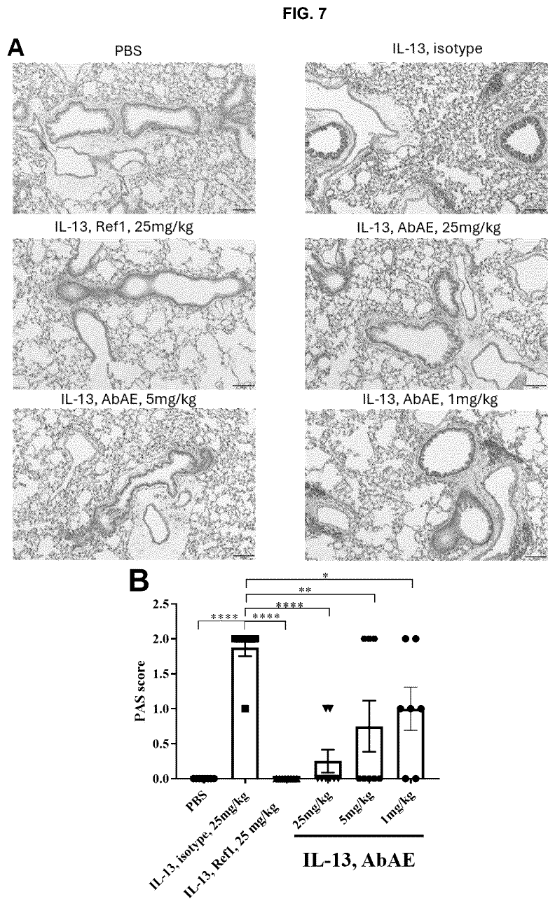

The levels of mucus production evaluated by Periodic acid-Schiff (PAS) staining were elevated in lung tissue samples collected from the hIL-13-induced group as compared to those in the vehicle group (FIG. 7A-B). The levels of mucus production in lung tissue samples collected from mice treated with Antibody AbAE were significantly reduced at all doses. Similar findings were observed in mice treated with Ref1.

Collectively, these data suggest that Antibody AbAE can reduce the concentration of key biomarkers associated with asthma to levels comparable to Ref1.

### Example 7: Epitope Binning of Anti-IL-13 Antibodies Biolayer Interferometry

To determine whether Antibody AbG and Ref1 compete for binding on human IL-13, biolayer interferometry (BLI) was used to measure binding interactions in real-time. Briefly, to evaluate the competition of Antibody AbG and Ref1 for binding to human IL-13, the antibodies were assessed in a pairwise manner using an in-tandem epitope binning approach on an Octet RH96 (Sartorius, Cat. No. FB-70434). All samples were diluted to their final concentration in 1× Kinetics Buffer (Sartorius, Cat. No. 18-1105). HIS-tagged human IL-13 was captured using anti-penta-HIS biosensors in 16-channel mode. All biosensors were hydrated for at least 10 minutes in 1× Kinetics Buffer before use. Measurements were performed in 384 tilted-well microplates with at least 80 mL at a 4 mm sensor offset with a plate temperature of 30° C. with shaking at 1,000 RPM for all steps. The sensors were placed into wells containing 1× Kinetics Buffer for 30 seconds to establish a baseline prior to capturing human IL-13. Next, sensors were placed in wells containing 5 μg/mL of human IL-13 for 120 seconds to achieve a loading response of 0.25-0.35 nm. A baseline was recorded by placing sensors into wells containing 1× Kinetics Buffer for 30 seconds to establish a baseline prior to association with a saturating antibody. The sensors were placed into 200 nM of saturating antibody for 300 seconds. The sensors were then placed in 1× Kinetics Buffer for 30 seconds to establish a baseline prior to association with a panel of competing antibodies. Next, the sensors were placed into 200 nM of competing antibody for 300 seconds. The order of antibodies (e.g., saturating or competing) was varied to address every pairwise permutation for a bidirectional competition assessment for Antibody AbG and Ref1 with tralokinumab-hIgG1-YTE serving as a non-competing antibody control and isotype-hIgG1-YTE as a negative isotype control.

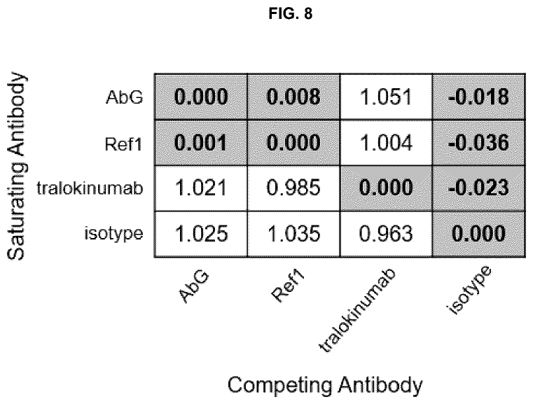

A blank sensor was used as a reference sensor and was subtracted from the data to correct for baseline drift. A buffer well was used as a reference sample and was subtracted from the data to correct for bulk effects. The data were analyzed using the epitope binning feature of the Octet Analysis Studio software (Sartorius, version 13.0.3.52). The curated binning results were displayed in terms of a heat map with shaded cells to indicate blocking interactions and unshaded cells to indicate non-blocking interactions, according to user-defined threshold settings (FIG. 8). A response of ≤0.1 nm was categorized as a non-binding interaction while a response >0.1 nm was categorized as a binding interaction.

The BLI epitope binning data displayed in FIG. 8 revealed that Antibody AbG and Ref1 compete for binding to human IL-13. There is no competition for binding to human IL-13 between Antibody AbG and tralokinumab-hIgG1-YTE or between Ref1 and tralokinumab-hIgG1-YTE. Isotype-hIgG1-YTE is a negative control that does not bind human IL-13 and does not compete with Antibody AbG, Ref1, or tralokinumab-hIgG1-YTE for binding to human IL-13. These data showed that Antibody AbG and Ref1 share a distinct epitope bin while tralokinumab-hIgG1-YTE and isotype-hIgG1-YTE represent distinct, non-overlapping epitope bins.

### Example 8: Affinity and Binding of Anti-IL-13 Antibodies to FcRn, Fcγ, and C1q

To assess the binding of Antibody AbAB to human FcRn, SPR was used, and to assess binding to a panel of human Fcγ receptors and human C1q, BLI was used.

Briefly, the kinetics of Antibody AbAB binding to FcRn was assessed using an anti-human Fab antibody prepared via amine-coupling onto a CM4 sensor chip to attain approximately 2,500 response units (RU) of immobilized anti-human Fab antibody. All antibodies and antigens were diluted to their final concentration in 1×PBS+0.05% Tween 20, pH 6.0 buffer. Antibody AbAB and Ref2 along with isotype controls isotype-hIgG1-YTE-TM, isotype-hIgG4, and isotype-IgG1 antibodies were injected for 30 seconds at a flow rate of 10 μL per minute over the surface to obtain a capture level of approximately 60-100 RU. HIS-tagged human FcRn antigen was injected over the surface using a 10-point, 2-fold serial dilution from 5,000 nM to 9.8 nM. Each antigen concentration was injected in a separate cycle for 180 seconds at a flow rate of 30 μL per minute and the complex was allowed to dissociate for 300 seconds using 1×PBS+0.05% Tween 20, pH 6.0 as the running buffer. The surface was regenerated by injecting two pulses of 10 mM glycine, pH 2.1 for 30 seconds at a flow rate of 30 μL per minute in between each cycle. The assay was performed at 25° C. with two technical replicates. The steady-state affinity parameters for the concentration series were obtained by double referencing and fitting the data to 1:1 steady-state affinity model using the Biacore Insight Evaluation software. The affinity of human FcRn binding to Antibody AbAB and assay controls are shown in Table 13 with affinity (K_{D}) values are expressed as average±standard deviation of one independent experiment with two technical replicates.

TABLE 13
Affinity of Human FcRn Binding to anti-IL-13 antibodies by SPR.

| Antibody | Antigen | K_{D} (nM) |
|---|---|---|
| AbAB | Human FcRn | 129.42 ± 8.89 |
| Ref2 | Human FcRn | 734.19 ± 7.50 |
| isotype-hIgG4 | Human FcRn | 1194.58 ± 34.23 |
| isotype-hIgG1 | Human FcRn | 1161.92 ± 13.16 |
| isotype-hIgG1-YTE | Human FcRn | 155.23 ± 9.45 |
| isotype-hIgG1-YTE-TM | Human FcRn | 155.75 ± 5.38 |

The SPR data demonstrated that Antibody AbAB binds to human FcRn at pH 6 with a 6-fold higher affinity than Ref2 with K_{D} values of 129.42 nM and 734.19 nM, respectively. In addition, the isotype controls containing the YTE mutations in the Fc region (isotype-hIgG1-YTE and isotype-hIgG1-YTE-TM) were within 2-fold of Antibody AbAB whereas the isotype controls without the YTE mutations in the Fc region (isotype-hIgG4 and isotype-hIgG1) were within 2-fold of Ref2. These differences are considered within the range of variability of the methodology. These data confirmed that the YTE mutations in the Fc region result in higher affinity to human FcRn at pH 6.

To evaluate the binding of Antibody AbAB to a panel of human Fcγ receptors, BLI measurements were performed using an Octet RH96. All samples were diluted to their final concentration in 1× Kinetics Buffer. Biotinylated human FcγRI, FcγRIIa-H167, FcγRIIa-R167, FcγRIIb, FcγRIIIa-V176, FcγRIIIa-F176, FcγRIIIb-NA1, and FcγRIIIb-NA2 were captured using streptavidin (SA) biosensors in 16-channel mode. All biosensors were hydrated for at least 10 minutes in 1× Kinetics Buffer before use. Measurements were performed in 384 tilted-well microplates with at least 85 mL at a 4 mm sensor offset with a plate temperature of 30° C. with shaking at 1,000 RPM for all steps. Sensors were placed into wells containing 1× Kinetics Buffer for 30 seconds to establish a baseline prior to capturing the panel of Fcγ receptors. To capture the panel of Fcγ receptors on the streptavidin sensor surface, a column of 8 sensors were placed in wells containing 1-2.4 ug/ml of human FcγRI, FcγRIIa-H167, FcγRIIa-R167, FcγRIIb, FcγRIIIa-V176, FcγRIIIa-F176, FcγRIIIb-NA1, or FcγRIIIb-NA2 for 300 seconds to achieve a minimum loading response of 1.75 nm. In parallel, a column of 8 sensors were placed in wells containing 1× Kinetics Buffer for 300 seconds to serve as reference sensors. A second baseline was recorded by placing sensors into wells containing 1× Kinetics Buffer for 30 seconds prior to a 300-second association of Antibody AbAB, Ref2, and isotype controls isotype-hIgG1-YTE-TM, isotype-hIgG1, and isotype-hIgG4 at 10,000 nM and a 300-second dissociation.

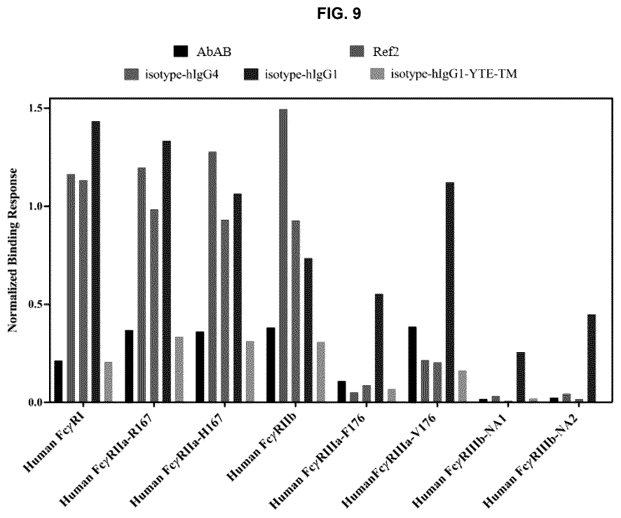

A blank sensor was used as a reference sensor and was subtracted from the data to correct for baseline drift. A buffer well was used as a reference sample and was subtracted from the data to correct for bulk effects. The raw data was aligned to the y-axis by aligning the data at the end of the baseline step, inter-step correction was used to align the start of the dissociation to the end of the association phase, and Savitzky-Golay filtering was applied. Capture level and binding response report points were obtained. The antibody binding responses were normalized to the human Fcγ receptor capture levels using GraphPad Prism and are reported as normalized binding responses, shown in FIG. 9.

The data in FIG. 9 demonstrate a reduced normalized binding response to human FcγRI, FcγRIIa-H167, FcγRIIa-R167, and FcγRIIb for Antibody AbAB compared to Ref2. In addition, the data showed a similar low normalized binding response to human FcγRIIIa-V176 and FcγRIIIa-F176 for Antibody AbAB and Ref2. No binding of Antibody AbAB or Ref2 to human FcγRIIIb-NA1 or FcγRIIIb-NA2 was detected at 10,000 nM. These results were comparable to what was detected for the isotype controls isotype-hIgG1-YTE-TM and isotype-hIgG4. Isotype control isotype-hIgG1 served as the positive control for all human Fcγ receptors evaluated.

Next, to evaluate the binding of Antibody AbAB to human C1q, BLI measurements were performed using an Octet RH96. All samples were diluted to their final concentration in 1× Kinetics Buffer. Antibody AbAB, Ref2, and isotype controls isotype-hIgG1-YTE-TM, isotype-hIgG1, and isotype-hIgG4 were captured using anti-human Fab biosensors in 16-channel mode. All biosensors were hydrated for at least 10 minutes in 1× Kinetics Buffer before use. The measurements were performed in 384 tilted-well microplates with at least 85 μL at a 4 mm sensor offset with a plate temperature of 30° C. and a shaker speed of 1,000 RPM for all steps. The sensors were placed into wells containing 1× Kinetics Buffer for 60 seconds to establish a baseline prior to antibody capture. To capture the panel of antibodies on the anti-human Fab sensor surface, a column of 8 sensors were placed in wells containing 20 μg/mL of Antibody AbAB, Ref2, and isotype controls isotype-hIgG1-YTE-TM, isotype-hIgG1, or isotype-hIgG4 for 180 seconds to achieve a minimum loading response of 0.6 nm. In parallel, a column of 8 sensors were placed in wells containing 1× Kinetics Buffer for 180 seconds to serve as reference sensors. A second baseline was recorded by placing sensors into wells containing 1× Kinetic Buffer prior to the association phase. The sensors were then placed into wells containing a 7-point, 2-fold serial dilution from 250 nM to 3.9 nM of human C1q for 300 seconds before dissociating in 1× Kinetics Buffer for 300 seconds.

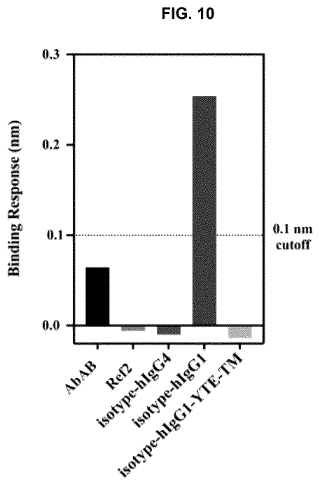

A blank sensor was used as a reference sensor and was subtracted from the data to correct for baseline drift. A buffer well was used as a reference sample and was subtracted from the data to correct for bulk effects. The raw data was aligned to the y-axis by aligning the data at the end of the baseline step, inter-step correction was used to align the start of the dissociation to the end of the association phase, and Savitzky-Golay filtering was applied. Report points of the capture level and binding response of human C1q at 250 nM were obtained. A response of ≤0.1 nm was categorized as a non-binding interaction while a response >0.1 nm was categorized as a binding interaction. As shown in FIG. 10, no detectable binding of human C1q to either Antibody AbAB or Ref2 at 250 nM was observed.

In summary, Antibody AbAB binds to human FcRn at pH 6 with higher affinity compared to Ref2. Antibody AbAB binds to human FcγRI, FcγRIIa-H167, FcγRIIa-R167, and FcγRIIb with reduced responses relative to Ref2. Antibody AbAB and Ref2 bind to human FcγRIIIa-V176 and FcγRIIIa-F176 with similar responses. No binding of Antibody AbAB and Ref2 to human FcγRIIIb-NA1, FcγRIIIb-NA2, or human C1q was detected.

### Example 9: Binding of Computationally Designed Proteins

Using the computer code disclosed herein, antibodies similar to Antibody AbG were computationally designed and then experimentally tested for binding affinity to IL-13 by SPR. Briefly, to evaluate the affinities of a panel of IL-13 antibodies (corresponding to SEQ ID NOs: 212-237) binding to human IL-13 at 25° C., a non-regenerative capture kinetics approach was performed using an LSA instrument (Carterra). A goat anti-human polyclonal antibody lawn was prepared via amine-coupling onto a polycarboxylate hydrogel (HC30M) sensor chip using the single flow cell (SFC) to attain approximately 5,000 response units (RU). Each antibody was prepared at 8 μg/mL and captured onto regions of interest (ROI) for 15 minutes using four serial dockings of the 96-channel printhead to create a 384-antibody array. Human IL-13 was prepared in HBSTE+BSA (10 mM HEPES pH 7.4, 150 mM NaCl, 3 mM EDTA, 0.01% Tween20, 0.5 mg/mL BSA) running buffer and injected over the captured antibody array surface at 8 concentrations in a 3-fold serial dilution series from 0.05 nM to 100 nM. Each concentration was injected sequentially from low to high concentration in a single cycle using the SFC with a 5 minute association phase and a 30 minute dissociation phase. The surface was regenerated by injecting two pulses of 0.85% phosphoric acid for 15 seconds between each cycle. The assay was performed at 25° C. Kinetic parameters for the concentration series were obtained by double referencing and globally fitting the data to a 1:1 binding model with the 5% k_{d} option selected using the Kinetics analysis software (Carterra). The affinities of human IL-13 binding to a panel of IL-13 antibodies are reported and the results are expressed as average±standard deviation when appropriate in Table 14.

TABLE 14
Binding and affinity values of reference anti-IL-13
antibodies and anti-IL-13 variant antibodies.

| Antibody | Description | Type | kₐ (10⁵ M⁻¹s⁻¹) | k_{d} (10⁻⁶ s⁻¹) | K_{D} (nM) |
|---|---|---|---|---|---|
| NB-13 | reference | hIgG4-S228P-YTE | not reported | not reported | <0.10 |
| Ref1 | reference | hIgG1-YTE | not reported | not reported | <0.10 |
| AbG | lead | hIgG1-YTE | not reported | not reported | <0.10 |
| NB-1 | variant | hIgG4-S228P-YTE | 1.17 ± 0.14 | 6.01 ± 0.76 | 5.21 ± 1.29 |
| NB-2 | variant | hIgG4-S228P-YTE | 0.93 | 6.18 | 6.67 |
| NB-3 | variant | hIgG4-S228P-YTE | 2.90 ± 0.36 | 4.70 ± 0.65 | 1.89 ± 0.24 |
| NB-4 | variant | hIgG4-S228P-YTE | 0.74 ± 0.11 | 9.87 ± 1.00 | 13.58 ± 2.53 |
| NB-5 | variant | hIgG4-S228P-YTE | 3.03 ± 0.31 | 12.73 ± 1.77 | 4.24 ± 0.87 |
| NB-6 | variant | hIgG4-S228P-YTE | 2.58 ± 0.15 | 8.55 ± 2.28 | 3.34 ± 1.01 |
| NB-7 | variant | hIgG4-S228P-YTE | 1.00 ± 0.14 | 8.27 ± 0.69 | 8.35 ± 1.34 |
| NB-8 | variant | hIgG4-S228P-YTE | 2.59 ± 0.30 | 6.74 ± 2.19 | 2.56 ± 0.70 |
| NB-9 | variant | hIgG4-S228P-YTE | 3.05 ± 0.04 | 7.34 ± 0.37 | 2.41 ± 0.12 |
| NB-10 | variant | hIgG4-S228P-YTE | 0.62 ± 0.10 | 8.05 ± 1.99 | 13.59 ± 4.94 |
| NB-11 | variant | hIgG4-S228P-YTE | 1.39 ± 0.03 | 1.35 ± 0.44 | 0.97 ± 0.31 |
| NB-12 | variant | hIgG4-S228P-YTE | 1.17 ± 0.08 | 7.11 ± 0.15 | 6.09 ± 0.51 |

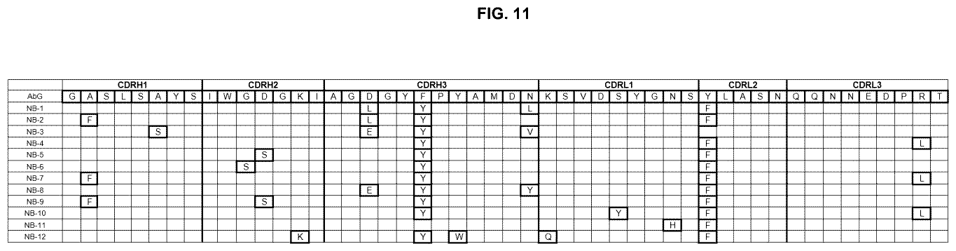

Next, all designed antibodies were scored for their mutational distance to Antibody AbG. The CDRs of antibodies corresponding to SEQ ID NOs: 212-237 with a mutational Hamming distance of 4 or less (e.g., at most 4 substitutions relative to Antibody AbG) are visualized in a multiple sequence alignment view as shown in FIG. 11. Surprisingly, some computationally designed antibodies with similar CDRs to Antibody AbG (e.g., four mutations or less) display notably weaker binding affinity relative to Antibody AbG (Table 14).

Specific embodiments provided herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments so claimed are inherently or expressly described and enabled herein.

In cases where numerical values are indicated herein, the skilled person will understand that the technical effect of the feature in question is ensured within an interval of accuracy, which typically encompasses a deviation of the numerical value given of ±10% or of ±5%. At the very least, and not as an attempt to limit the application of the doctrine of equivalents to the scope of the claims, each numerical parameter should at least be construed considering the number of reported significant digits and by applying ordinary rounding techniques.

Unless otherwise indicated, all numbers expressing quantities of ingredients, properties such as molecular weight and median size, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained.

The terms “a,” “an,” “the” and similar referents used in the context of the description herein (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the specification and does not pose a limitation on the scope of the claims. No language in the specification should be construed as indicating any non-claimed element essential to the practice of the description.

Unless specifically stated or obvious from context, as used herein, the term “or” is understood to be inclusive and covers both “or” and “and.”

Groupings of alternative elements or embodiments provided herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments are described herein, including the best mode known for carrying out methods provided herein. Of course, variations on these described embodiments will become apparent upon reading the foregoing description. One can be expected to employ such variations as appropriate and can be practiced other than as specifically described herein. Accordingly, this description includes all modifications and equivalents of the subject matter recited in the claims appended hereto as permitted by applicable law. Moreover, any combination of the above-described elements in all possible variations thereof is encompassed by the description unless otherwise indicated herein or otherwise clearly contradicted by context.

It is to be understood that the embodiments provided herein are illustrative of the principles of the description herein. Other modifications that can be employed are within the scope of the description. Thus, by way of example, but not of limitation, alternative configurations can be utilized in accordance with the teachings herein. Accordingly, the presented information is not limited to that precisely as shown and described.

While the present description has been described and illustrated herein by references to various specific materials, procedures, and examples, it is understood that the description is not restricted to the particular combinations of materials and procedures selected for that purpose. Numerous variations of such details can be implied as will be appreciated. It is intended that the specification and examples be considered as exemplary only, with the true scope and spirit of the specification being indicated by the following claims. All references, patents, and patent applications referred to in this application are herein incorporated by reference in their entirety.

Unless defined otherwise, all technical and scientific terms used herein have the same meaning as commonly understood. Although other probes, compositions, methods, and kits similar, or equivalent, to those described herein can be used in the practice described herein, the materials and methods are described herein. It is to be understood that the terminology used herein is for the purpose of describing embodiments only and is not intended to be limiting.

Unless specifically stated or obvious from context, as used herein, the term “about” is understood as within a range of normal tolerance, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

A stated range is understood to be any value between and at the limits of the stated range. As examples, a range between 1 and 5 includes 1, 2, 3, 4, and 5; a range between 1 and 10 includes 1, 2, 3, 4, 5, 6, 7, 8, 9, and 10; and a range between 1 and 100 includes 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100.

Any aspect or embodiment described herein can be combined with any other aspect or embodiment as described herein.

What is claimed is:

1. An anti-IL-13 antibody or antigen binding fragment thereof comprising:
a. a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3);
b. a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3);
c. a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3);
d. a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3);
e. a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3);
f. a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3);
g. a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3);
h. a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3);
i. a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3);
j. a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3);
k. a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3);
l. a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3);
m. a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3);
n. a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3);
o. a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); or
p. a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3).

2. The anti-IL-13 antibody or antigen binding fragment thereof according to claim 1, wherein the light chain variable region comprises an amino acid sequence that is at least 90%, at least 95%, or at least 99% identical to SEQ ID NO: 97, 99, 101, 103, 105, 107, 109, 111, 113, 115, 117, 119, 121, 123, 125, or 127; and the heavy chain variable region comprises an amino acid sequence that is at least 90%, at least 95%, or at least 99% identical to SEQ ID NO: 98, 100, 102, 104, 106, 108, 110, 112, 114, 116, 118, 120, 122, 124, 126, or 128.

3. The anti-IL-13 antibody or antigen binding fragment thereof according to claim 1, wherein the light chain variable region comprises an amino acid sequence as set forth in SEQ ID NO: 97, 99, 101, 103, 105, 107, 109, 111, 113, 115, 117, 119, 121, 123, 125, or 127; and/or the heavy chain variable region comprises an amino acid sequence as set forth in SEQ ID NO: 98, 100, 102, 104, 106, 108, 110, 112, 114, 116, 118, 120, 122, 124, 126, or 128.

4. The anti-IL-13 antibody or antigen binding fragment thereof according to claim 3, wherein:
a. the light chain variable region comprises an amino acid sequence as set forth in SEQ ID NO: 97; and the heavy chain variable region comprises an amino acid sequence as set forth in SEQ ID NO: 98;
b. the light chain variable region comprises an amino acid sequence as set forth in SEQ ID NO: 99; and the heavy chain variable region comprises an amino acid sequence as set forth in SEQ ID NO: 100;
c. the light chain variable region comprises an amino acid sequence as set forth in SEQ ID NO: 101; and the heavy chain variable region comprises an amino acid sequence as set forth in SEQ ID NO: 102;
d. the light chain variable region comprises an amino acid sequence as set forth in SEQ ID NO: 103; and the heavy chain variable region comprises an amino acid sequence as set forth in SEQ ID NO: 104;
e. the light chain variable region comprises an amino acid sequence as set forth in SEQ ID NO: 105; and the heavy chain variable region comprises an amino acid sequence as set forth in SEQ ID NO: 106;
f. the light chain variable region comprises an amino acid sequence as set forth in SEQ ID NO: 107; and the heavy chain variable region comprises an amino acid sequence as set forth in SEQ ID NO: 108;
g. the light chain variable region comprises an amino acid sequence as set forth in SEQ ID NO: 109; and the heavy chain variable region comprises an amino acid sequence as set forth in SEQ ID NO: 110;
h. the light chain variable region comprises an amino acid sequence as set forth in SEQ ID NO: 111; and the heavy chain variable region comprises an amino acid sequence as set forth in SEQ ID NO: 112;
i. the light chain variable region comprises an amino acid sequence as set forth in SEQ ID NO: 113; and the heavy chain variable region comprises an amino acid sequence as set forth in SEQ ID NO: 114;
j. the light chain variable region comprises an amino acid sequence as set forth in SEQ ID NO: 115; and the heavy chain variable region comprises an amino acid sequence as set forth in SEQ ID NO: 116;
k. the light chain variable region comprises an amino acid sequence as set forth in SEQ ID NO: 117; and the heavy chain variable region comprises an amino acid sequence as set forth in SEQ ID NO: 118;
l. the light chain variable region comprises an amino acid sequence as set forth in SEQ ID NO: 119; and the heavy chain variable region comprises an amino acid sequence as set forth in SEQ ID NO: 120;
m. the light chain variable region comprises an amino acid sequence as set forth in SEQ ID NO: 121; and the heavy chain variable region comprises an amino acid sequence as set forth in SEQ ID NO: 122
n. the light chain variable region comprises an amino acid sequence as set forth in SEQ ID NO: 123; and the heavy chain variable region comprises an amino acid sequence as set forth in SEQ ID NO: 124;
o. the light chain variable region comprises an amino acid sequence as set forth in SEQ ID NO: 125; and the heavy chain variable region comprises an amino acid sequence as set forth in SEQ ID NO: 126; or
p. the light chain variable region comprises an amino acid sequence as set forth in SEQ ID NO: 127; and the heavy chain variable region comprises an amino acid sequence as set forth in SEQ ID NO: 128.

5. The anti-IL-13 antibody or antigen binding fragment thereof according to claim 1, wherein the antibody comprises a light chain comprising an amino acid sequence that is at least 90%, at least 95%, or at least 99% identical to SEQ ID NO: 129, 131, 133, 135, 137, 139, 141, 143, 145, 147, 149, 151, 153, 155, 157, or 159; and/or a heavy chain comprising an amino acid sequence that is at least 90%, at least 95%, or at least 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

6. The anti-IL-13 antibody or antigen binding fragment thereof according to claim 5, wherein the light chain comprises an amino acid sequence as set forth in SEQ ID NO: 129, 131, 133, 135, 137, 139, 141, 143, 145, 147, 149, 151, 153, 155, 157, or 159; and/or the heavy chain comprises an amino acid sequence as set forth in SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

7. The anti-IL-13 antibody or antigen binding fragment thereof according to claim 6, wherein:
a. the light chain comprises an amino acid sequence as set forth in SEQ ID NO: 129;
and the heavy chain comprises an amino acid sequence as set forth in SEQ ID NO: 130;
b. the light chain comprises an amino acid sequence as set forth in SEQ ID NO: 131; and the heavy chain comprises an amino acid sequence as set forth in SEQ ID NO: 132;
c. the light chain comprises an amino acid sequence as set forth in SEQ ID NO: 133; and the heavy chain comprises an amino acid sequence as set forth in SEQ ID NO: 134;
d. the light chain comprises an amino acid sequence as set forth in SEQ ID NO: 135; and the heavy chain comprises an amino acid sequence as set forth in SEQ ID NO: 136;
e. the light chain comprises an amino acid sequence as set forth in SEQ ID NO: 137; and the heavy chain comprises an amino acid sequence as set forth in SEQ ID NO: 138;
f. the light chain comprises an amino acid sequence as set forth in SEQ ID NO: 139; and the heavy chain comprises an amino acid sequence as set forth in SEQ ID NO: 140;
g. the light chain comprises an amino acid sequence as set forth in SEQ ID NO: 141; and the heavy chain comprises an amino acid sequence as set forth in SEQ ID NO: 142;
h. the light chain comprises an amino acid sequence as set forth in SEQ ID NO: 143; and the heavy chain comprises an amino acid sequence as set forth in SEQ ID NO: 144;
i. the light chain comprises an amino acid sequence as set forth in SEQ ID NO: 145; and the heavy chain comprises an amino acid sequence as set forth in SEQ ID NO: 146;
j. the light chain comprises an amino acid sequence as set forth in SEQ ID NO: 147; and the heavy chain comprises an amino acid sequence as set forth in SEQ ID NO: 148;
k. the light chain comprises an amino acid sequence as set forth in SEQ ID NO: 149; and the heavy chain comprises an amino acid sequence as set forth in SEQ ID NO: 150;
l. the light chain comprises an amino acid sequence as set forth in SEQ ID NO: 151; and the heavy chain comprises an amino acid sequence as set forth in SEQ ID NO: 152;
m. the light chain comprises an amino acid sequence as set forth in SEQ ID NO: 153; and the heavy chain comprises an amino acid sequence as set forth in SEQ ID NO: 154;
n. the light chain comprises an amino acid sequence as set forth in SEQ ID NO: 155; and the heavy chain comprises an amino acid sequence as set forth in SEQ ID NO: 156;
o. the light chain comprises an amino acid sequence as set forth in SEQ ID NO: 157; and the heavy chain comprises an amino acid sequence as set forth in SEQ ID NO: 158;
p. the light chain comprises an amino acid sequence as set forth in SEQ ID NO: 159; and the heavy chain comprises an amino acid sequence as set forth in SEQ ID NO: 160;
q. the light chain comprises an amino acid sequence as set forth in SEQ ID NO: 153; and the heavy chain comprises an amino acid sequence as set forth in SEQ ID NO: 182;
r. the light chain comprises an amino acid sequence as set forth in SEQ ID NO: 147; and the heavy chain comprises an amino acid sequence as set forth in SEQ ID NO: 183;
s. the light chain comprises an amino acid sequence as set forth in SEQ ID NO: 141; and the heavy chain comprises an amino acid sequence as set forth in SEQ ID NO: 184;
t. the light chain comprises an amino acid sequence as set forth in SEQ ID NO: 153; and the heavy chain comprises an amino acid sequence as set forth in SEQ ID NO: 185;
u. the light chain comprises an amino acid sequence as set forth in SEQ ID NO: 147; and the heavy chain comprises an amino acid sequence as set forth in SEQ ID NO: 186;
v. the light chain comprises an amino acid sequence as set forth in SEQ ID NO: 141; and the heavy chain comprises an amino acid sequence as set forth in SEQ ID NO: 187;
w. the light chain comprises an amino acid sequence as set forth in SEQ ID NO: 153; and the heavy chain comprises an amino acid sequence as set forth in SEQ ID NO: 188;
x. the light chain comprises an amino acid sequence as set forth in SEQ ID NO: 147; and the heavy chain comprises an amino acid sequence as set forth in SEQ ID NO: 189;
y. the light chain comprises an amino acid sequence as set forth in SEQ ID NO: 141; and the heavy chain comprises an amino acid sequence as set forth in SEQ ID NO: 190;
z. the light chain comprises an amino acid sequence as set forth in SEQ ID NO: 153; and the heavy chain comprises an amino acid sequence as set forth in SEQ ID NO: 191;
aa. the light chain comprises an amino acid sequence as set forth in SEQ ID NO: 147; and the heavy chain comprises an amino acid sequence as set forth in SEQ ID NO: 192;
bb. the light chain comprises an amino acid sequence as set forth in SEQ ID NO: 141; and the heavy chain comprises an amino acid sequence as set forth in SEQ ID NO: 193;
cc. the light chain comprises an amino acid sequence as set forth in SEQ ID NO: 153; and the heavy chain comprises an amino acid sequence as set forth in SEQ ID NO: 194;
dd. the light chain comprises an amino acid sequence as set forth in SEQ ID NO: 147; and the heavy chain comprises an amino acid sequence as set forth in SEQ ID NO: 195;
ee. the light chain comprises an amino acid sequence as set forth in SEQ ID NO: 141; and the heavy chain comprises an amino acid sequence as set forth in SEQ ID NO: 196;
ff. the light chain comprises an amino acid sequence as set forth in SEQ ID NO: 153; and the heavy chain comprises an amino acid sequence as set forth in SEQ ID NO: 197;
gg. the light chain comprises an amino acid sequence as set forth in SEQ ID NO: 147; and the heavy chain comprises an amino acid sequence as set forth in SEQ ID NO: 198;
hh. the light chain comprises an amino acid sequence as set forth in SEQ ID NO: 141; and the heavy chain comprises an amino acid sequence as set forth in SEQ ID NO: 199;
ii. the light chain comprises an amino acid sequence as set forth in SEQ ID NO: 153; and the heavy chain comprises an amino acid sequence as set forth in SEQ ID NO: 200;
jj. the light chain comprises an amino acid sequence as set forth in SEQ ID NO: 147; and the heavy chain comprises an amino acid sequence as set forth in SEQ ID NO: 201;
kk. the light chain comprises an amino acid sequence as set forth in SEQ ID NO: 141; and the heavy chain comprises an amino acid sequence as set forth in SEQ ID NO: 202;
ll. the light chain comprises an amino acid sequence as set forth in SEQ ID NO: 153; and the heavy chain comprises an amino acid sequence as set forth in SEQ ID NO: 203;
mm. the light chain comprises an amino acid sequence as set forth in SEQ ID NO: 147; and the heavy chain comprises an amino acid sequence as set forth in SEQ ID NO: 204;
nn. the light chain comprises an amino acid sequence as set forth in SEQ ID NO: 141; and the heavy chain comprises an amino acid sequence as set forth in SEQ ID NO: 205;
oo. the light chain comprises an amino acid sequence as set forth in SEQ ID NO: 153; and the heavy chain comprises an amino acid sequence as set forth in SEQ ID NO: 206;
pp. the light chain comprises an amino acid sequence as set forth in SEQ ID NO: 147; and the heavy chain comprises an amino acid sequence as set forth in SEQ ID NO: 207;
qq. the light chain comprises an amino acid sequence as set forth in SEQ ID NO: 141; and the heavy chain comprises an amino acid sequence as set forth in SEQ ID NO: 208;
rr. the light chain comprises an amino acid sequence as set forth in SEQ ID NO: 153; and the heavy chain comprises an amino acid sequence as set forth in SEQ ID NO: 209;
ss. the light chain comprises an amino acid sequence as set forth in SEQ ID NO: 147; and the heavy chain comprises an amino acid sequence as set forth in SEQ ID NO: 210; or
tt. the light chain comprises an amino acid sequence as set forth in SEQ ID NO: 141; and the heavy chain comprises an amino acid sequence as set forth in SEQ ID NO: 211.

8. The anti-IL-13 antibody or antigen binding fragment thereof according to claim 1, wherein the antibody is a monoclonal antibody.

9. The anti-IL-13 antibody or antigen binding fragment thereof according to claim 1, wherein the antibody is a human antibody.

10. The anti-IL-13 antibody or antigen binding fragment thereof according to claim 1, wherein the antibody is a bispecific antibody.

11. The anti-IL-13 antibody or antigen binding fragment thereof according to claim 1, wherein the antigen binding fragment thereof is selected from the group consisting of Fab, Fab′, F(ab′)₂, Fd, Fv, scFv, a single-chain antibody, a minibody, and a diabody.

12. The anti-IL-13 antibody or antigen binding fragment thereof according to claim 11, wherein the binding fragment thereof is an scFv.

13. A pharmaceutical composition comprising:
an anti-IL-13 antibody or antigen binding fragment comprising:
a. a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3);
b. a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3);
c. a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3);
d. a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3);
e. a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3);
f. a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3);
g. a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3);
h. a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3);
i. a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3);
j. a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3);
k. a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3);
l. a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3);
m. a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3);
n. a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3);
o. a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); or
p. a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3), and
a pharmaceutically acceptable carrier.

14. A nucleic acid composition comprising one or more nucleic acids encoding:
a. a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3);
b. a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3);
c. a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3);
d. a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3);
e. a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3);
f. a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3);
g. a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3);
h. a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3);
i. a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3);
j. a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3);
k. a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3);
l. a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3);
m. a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3);
n. a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3);
o. a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); or
p. a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3).

15. An expression vector composition comprising one or more expression vectors, wherein the one or more expression vectors comprise one or more nucleic acids encoding a light chain variable region and a heavy chain variable region pair selected from the group consisting of:
a. a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3);
b. a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3);
c. a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3);
d. a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3);
e. a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3);
f. a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3);
g. a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3);
h. a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3);
i. a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3);
j. a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3);
k. a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3);
l. a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3);
m. a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3);
n. a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3);
o. a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); and
p. a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3).

16. A host cell comprising a nucleic acid composition comprising one or more nucleic acids encoding:
a. a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3);
b. a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3);
c. a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3);
d. a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3);
e. a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3);
f. a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3);
g. a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3);
h. a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3);
i. a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3);
j. a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3);
k. a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3);
l. a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3);
m. a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3);
n. a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3);
o. a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); or
p. a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3).

17. A host cell comprising an expression vector composition comprising one or more expression vectors, wherein the one or more expression vectors comprise one or more nucleic acids encoding a light chain variable region and a heavy chain variable region pair selected from the group consisting of:
a. a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3);
b. a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3);
c. a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3);
d. a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3);
e. a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3);
f. a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3);
g. a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3);
h. a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3);
i. a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3);
j. a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3);
k. a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3);
l. a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3);
m. a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3);
n. a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3);
o. a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); and
p. a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3).

18. A method of making an anti-IL-13 antibody or antigen binding fragment thereof comprising: culturing a host cell under conditions where an antibody or binding fragment thereof that binds IL-13 is expressed, and recovering the antibody or binding fragment thereof that binds IL-13, wherein the anti-IL-13 antibody or antigen binding fragment thereof comprises:
a. a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3);
b. a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3);
c. a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3);
d. a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3);
e. a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3);
f. a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3);
g. a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3);
h. a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3);
i. a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3);
j. a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3);
k. a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3);
l. a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3);
m. a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3);
n. a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3);
o. a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); or
p. a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO: 96 (HCDR3).

19. An anti-IL-13 antibody or antigen binding fragment thereof comprising:
a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3);
a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3);
a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3);
a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3);
a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3);
a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3);
a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3);
a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3);
a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3);
a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3);
a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3);
a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3);
a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3);
a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3);
a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or
a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3);
or
b1) a light chain variable region comprising an amino acid sequence at least 90%, at least 95%, or at least 99% identical to SEQ ID NO: 97, 99, 101, 103, 105, 107, 109, 111, 113, 115, 117, 119, 121, 123, 125, or 127; and
b2) a heavy chain variable region comprising an amino acid sequence at least 90%, at least 95%, or at least 99% identical to SEQ ID NO: 98, 100, 102, 104, 106, 108, 110, 112, 114, 116, 118, 120, 122, 124, 126, or 128,
wherein the LCDR1 does not include SEQ ID NO: 162 (RASKSVDSYGNSFMH).

20. The anti-IL-13 antibody or antigen binding fragment thereof according to claim 19, wherein:
a. LCDR1 comprises a serine (S) at amino acid position 27D and an arginine (R) at amino acid position 30;
b. LCDR1 comprises a tyrosine (Y) at amino acid position 27D and a histidine (H) at amino acid position 30;
c. LCDR1 comprises a tyrosine (Y) at amino acid position 27D and an asparagine (N) at amino acid position 30; or
d. LCDR1 comprises a serine (S) at amino acid position 27D and a tyrosine (Y) at amino acid position 32,
wherein the amino acid positions are based on Kabat numbering.

21. An anti-IL-13 antibody or antigen binding fragment thereof comprising a light chain variable region and a heavy chain variable region,
wherein the light chain variable region is at least 95% identical to SEQ ID NO: 97, 99, 101, or 103; and
wherein the heavy chain variable region is at least 95% identical to SEQ ID NOs: SEQ ID NO: 98, 100, 102, or 104,
provided that when serine (S) is present at amino acid position 27D then arginine (R) is present at amino acid position 30 or tyrosine (Y) is present at amino acid position 32, and when asparagine (N) is present at amino acid position 30 then tyrosine (Y) is present at amino acid position 27D, wherein the amino acid positions are based on Kabat numbering.

22. The anti-IL-13 antibody or antigen binding fragment thereof according to claim 19, wherein the antibody is a monoclonal antibody.

23. The anti-IL-13 antibody or antigen binding fragment thereof according to claim 19, wherein the antibody is a human antibody.

24. The anti-IL-13 antibody or antigen binding fragment thereof according to claim 19, wherein the antibody is a bispecific antibody.

25. The anti-IL-13 antibody or antigen binding fragment thereof according to claim 19, wherein the binding fragment thereof is selected from the group consisting of Fab, Fab′, F(ab′)₂, Fd, Fv, scFv, a single-chain antibody, a minibody, and a diabody.

26. The anti-IL-13 antibody or antigen binding fragment thereof according to claim 25, wherein the binding fragment thereof is an scFv.

27. The anti-IL-13 antibody or antigen binding fragment thereof according to claim 19 for use as a medicament.

28. A pharmaceutical composition comprising:
an anti-IL-13 antibody or antigen binding fragment thereof comprising:
a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3);
a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3);
a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3);
a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3);
a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3);
a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3);
a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3);
a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3);
a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3);
a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3);
a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3);
a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3);
a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3);
a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3);
a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or
a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3);
or
b1) a light chain variable region comprising an amino acid sequence at least 90%, at least 95%, or at least 99% identical to SEQ ID NO: 97, 99, 101, 103, 105, 107, 109, 111, 113, 115, 117, 119, 121, 123, 125, or 127; and
b2) a heavy chain variable region comprising an amino acid sequence at least 90%, at least 95%, or at least 99% identical to SEQ ID NO: 98, 100, 102, 104, 106, 108, 110, 112, 114, 116, 118, 120, 122, 124, 126, or 128,
wherein the LCDR1 does not include SEQ ID NO: 162 (RASKSVDSYGNSFMH); and
a pharmaceutically acceptable carrier.

29. A nucleic acid composition comprising one or more nucleic acids encoding:
a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3);
a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3);
a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3);
a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3);
a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3);
a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3);
a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3);
a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3);
a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3);
a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3);
a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3);
a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3);
a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3);
a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3);
a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or
a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3);
or
b1) a light chain variable region comprising an amino acid sequence at least 90%, at least 95%, or at least 99% identical to SEQ ID NO: 97, 99, 101, 103, 105, 107, 109, 111, 113, 115, 117, 119, 121, 123, 125, or 127; and
b2) a heavy chain variable region comprising an amino acid sequence at least 90%, at least 95%, or at least 99% identical to SEQ ID NO: 98, 100, 102, 104, 106, 108, 110, 112, 114, 116, 118, 120, 122, 124, 126, or 128,
wherein the LCDR1 does not include SEQ ID NO: 162 (RASKSVDSYGNSFMH).

30. An expression vector composition comprising one or more expression vectors, wherein the one or more expression vectors comprise one or more nucleic acids encoding a light chain variable region and a heavy chain variable region pair selected from the group consisting of:
a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3);
a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3);
a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3);
a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3);
a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3);
a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3);
a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3);
a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3);
a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3);
a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3);
a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3);
a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3);
a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3);
a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3);
a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or
a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3);
or
b1) a light chain variable region comprising an amino acid sequence at least 90%, at least 95%, or at least 99% identical to SEQ ID NO: 97, 99, 101, 103, 105, 107, 109, 111, 113, 115, 117, 119, 121, 123, 125, or 127; and
b2) a heavy chain variable region comprising an amino acid sequence at least 90%, at least 95%, or at least 99% identical to SEQ ID NO: 98, 100, 102, 104, 106, 108, 110, 112, 114, 116, 118, 120, 122, 124, 126, or 128,
wherein the LCDR1 does not include SEQ ID NO: 162 (RASKSVDSYGNSFMH).

31. A host cell comprising a nucleic acid composition comprising one or more nucleic acids encoding:
a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3);
a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3);
a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3);
a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3);
a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3);
a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3);
a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3);
a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3);
a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3);
a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3);
a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3);
a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3);
a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3);
a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3);
a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or
a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3);
or
b1) a light chain variable region comprising an amino acid sequence at least 90%, at least 95%, or at least 99% identical to SEQ ID NO: 97, 99, 101, 103, 105, 107, 109, 111, 113, 115, 117, 119, 121, 123, 125, or 127; and
b2) a heavy chain variable region comprising an amino acid sequence at least 90%, at least 95%, or at least 99% identical to SEQ ID NO: 98, 100, 102, 104, 106, 108, 110, 112, 114, 116, 118, 120, 122, 124, 126, or 128,
wherein the LCDR1 does not include SEQ ID NO: 162 (RASKSVDSYGNSFMH).

32. A host cell comprising an expression vector composition comprising one or more expression vectors, wherein the one or more expression vectors comprise one or more nucleic acids encoding a light chain variable region and a heavy chain variable region pair selected from the group consisting of:
a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3);
a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3);
a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3);
a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3);
a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3);
a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3);
a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3);
a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3);
a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3);
a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3);
a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3);
a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3);
a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3);
a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3);
a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or
a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3);
or
b1) a light chain variable region comprising an amino acid sequence at least 90%, at least 95%, or at least 99% identical to SEQ ID NO: 97, 99, 101, 103, 105, 107, 109, 111, 113, 115, 117, 119, 121, 123, 125, or 127; and
b2) a heavy chain variable region comprising an amino acid sequence at least 90%, at least 95%, or at least 99% identical to SEQ ID NO: 98, 100, 102, 104, 106, 108, 110, 112, 114, 116, 118, 120, 122, 124, 126, or 128,
wherein the LCDR1 does not include SEQ ID NO: 162 (RASKSVDSYGNSFMH).

33. A method of making the anti-IL-13 antibody or antigen binding fragment thereof comprising:
culturing a host cell under conditions where an antibody or binding fragment thereof that binds IL-13 is expressed, and recovering the antibody or binding fragment thereof that binds IL-13, wherein the anti-IL-13 antibody or antigen binding fragment thereof comprises:
a1) light chain CDRs comprising amino acid sequences that differ by no more than 1, 2, or 3 amino acids from SEQ ID NO: 1 (LCDR1), SEQ ID NO: 2 (LCDR2), and SEQ ID NO: 3 (LCDR3); and heavy chain CDRs comprising amino acid sequences that differ by no more than 1, 2, or 3 amino acids from SEQ ID NO: 4 (HCDR1), SEQ ID NO: 5 (HCDR2), and SEQ ID NO: 6 (HCDR3);
a2) light chain CDRs comprising amino acid sequences that differ by no more than 1, 2, or 3 amino acids from SEQ ID NO: 7 (LCDR1), SEQ ID NO: 8 (LCDR2), and SEQ ID NO: 9 (LCDR3); and heavy chain CDRs comprising amino acid sequences that differ by no more than 1, 2, or 3 amino acids from SEQ ID NO: 10 (HCDR1), SEQ ID NO: 11 (HCDR2), and SEQ ID NO: 12 (HCDR3);
a3) light chain CDRs comprising amino acid sequences that differ by no more than 1, 2, or 3 amino acids from SEQ ID NO: 13 (LCDR1), SEQ ID NO: 14 (LCDR2), and SEQ ID NO: 15 (LCDR3); and heavy chain CDRs comprising amino acid sequences that differ by no more than 1, 2, or 3 amino acids from SEQ ID NO: 16 (HCDR1), SEQ ID NO: 17 (HCDR2), and SEQ ID NO: 18 (HCDR3);
a4) light chain CDRs comprising amino acid sequences that differ by no more than 1, 2, or 3 amino acids from SEQ ID NO: 19 (LCDR1), SEQ ID NO: 20 (LCDR2), and SEQ ID NO: 21 (LCDR3); and heavy chain CDRs comprising amino acid sequences that differ by no more than 1, 2, or 3 amino acids from SEQ ID NO: 22 (HCDR1), SEQ ID NO: 23 (HCDR2), and SEQ ID NO: 24 (HCDR3);
a5) light chain CDRs comprising amino acid sequences that differ by no more than 1, 2, or 3 amino acids from SEQ ID NO: 25 (LCDR1), SEQ ID NO: 26 (LCDR2), and SEQ ID NO: 27 (LCDR3); and heavy chain CDRs comprising amino acid sequences that differ by no more than 1, 2, or 3 amino acids from SEQ ID NO: 28 (HCDR1), SEQ ID NO: 29 (HCDR2), and SEQ ID NO: 30 (HCDR3);
a6) light chain CDRs comprising amino acid sequences that differ by no more than 1, 2, or 3 amino acids from SEQ ID NO: 31 (LCDR1), SEQ ID NO: 32 (LCDR2), and SEQ ID NO: 33 (LCDR3); and heavy chain CDRs comprising amino acid sequences that differ by no more than 1, 2, or 3 amino acids from SEQ ID NO: 34 (HCDR1), SEQ ID NO: 35 (HCDR2), and SEQ ID NO: 36 (HCDR3);
a7) light chain CDRs comprising amino acid sequences that differ by no more than 1, 2, or 3 amino acids from SEQ ID NO: 37 (LCDR1), SEQ ID NO: 38 (LCDR2), and SEQ ID NO: 39 (LCDR3); and heavy chain CDRs comprising amino acid sequences that differ by no more than 1, 2, or 3 amino acids from SEQ ID NO: 40 (HCDR1), SEQ ID NO: 41 (HCDR2), and SEQ ID NO: 42 (HCDR3);
a8) light chain CDRs comprising amino acid sequences that differ by no more than 1, 2, or 3 amino acids from SEQ ID NO: 43 (LCDR1), SEQ ID NO: 44 (LCDR2), and SEQ ID NO: 45 (LCDR3); and heavy chain CDRs comprising amino acid sequences that differ by no more than 1, 2, or 3 amino acids from SEQ ID NO: 46 (HCDR1), SEQ ID NO: 47 (HCDR2), and SEQ ID NO: 48 (HCDR3);
a9) light chain CDRs comprising amino acid sequences that differ by no more than 1, 2, or 3 amino acids from SEQ ID NO: 49 (LCDR1), SEQ ID NO: 50 (LCDR2), and SEQ ID NO: 51 (LCDR3); and heavy chain CDRs comprising amino acid sequences that differ by no more than 1, 2, or 3 amino acids from SEQ ID NO: 52 (HCDR1), SEQ ID NO: 53 (HCDR2), and SEQ ID NO: 54 (HCDR3);
a10) light chain CDRs comprising amino acid sequences that differ by no more than 1, 2, or 3 amino acids from SEQ ID NO: 55 (LCDR1), SEQ ID NO: 56 (LCDR2), and SEQ ID NO: 57 (LCDR3); and heavy chain CDRs comprising amino acid sequences that differ by no more than 1, 2, or 3 amino acids from SEQ ID NO: 58 (HCDR1), SEQ ID NO: 59 (HCDR2), and SEQ ID NO: 60 (HCDR3);
a11) light chain CDRs comprising amino acid sequences that differ by no more than 1, 2, or 3 amino acids from SEQ ID NO: 61 (LCDR1), SEQ ID NO: 62 (LCDR2), and SEQ ID NO: 63 (LCDR3); and heavy chain CDRs comprising amino acid sequences that differ by no more than 1, 2, or 3 amino acids from SEQ ID NO: 64 (HCDR1), SEQ ID NO: 65 (HCDR2), and SEQ ID NO: 66 (HCDR3);
a12) light chain CDRs comprising amino acid sequences that differ by no more than 1, 2, or 3 amino acids from SEQ ID NO: 67 (LCDR1), SEQ ID NO: 68 (LCDR2), and SEQ ID NO: 69 (LCDR3); and heavy chain CDRs comprising amino acid sequences that differ by no more than 1, 2, or 3 amino acids from SEQ ID NO: 70 (HCDR1), SEQ ID NO: 71 (HCDR2), and SEQ ID NO: 72 (HCDR3);
a13) light chain CDRs comprising amino acid sequences that differ by no more than 1, 2, or 3 amino acids from SEQ ID NO: 73 (LCDR1), SEQ ID NO: 74 (LCDR2), and SEQ ID NO: 75 (LCDR3); and heavy chain CDRs comprising amino acid sequences that differ by no more than 1, 2, or 3 amino acids from SEQ ID NO: 76 (HCDR1), SEQ ID NO: 77 (HCDR2), and SEQ ID NO: 78 (HCDR3);
a14) light chain CDRs comprising amino acid sequences that differ by no more than 1, 2, or 3 amino acids from SEQ ID NO: 79 (LCDR1), SEQ ID NO: 80 (LCDR2), and SEQ ID NO: 81 (LCDR3); and heavy chain CDRs comprising amino acid sequences that differ by no more than 1, 2, or 3 amino acids from SEQ ID NO: 82 (HCDR1), SEQ ID NO: 83 (HCDR2), and SEQ ID NO: 84 (HCDR3);
a15) light chain CDRs comprising amino acid sequences that differ by no more than 1, 2, or 3 amino acids from SEQ ID NO: 85 (LCDR1), SEQ ID NO: 86 (LCDR2), and SEQ ID NO: 87 (LCDR3); and heavy chain CDRs comprising amino acid sequences that differ by no more than 1, 2, or 3 amino acids from SEQ ID NO: 88 (HCDR1), SEQ ID NO: 89 (HCDR2), and SEQ ID NO: 90 (HCDR3); or
a16) light chain CDRs comprising amino acid sequences that differ by no more than 1, 2, or 3 amino acids from SEQ ID NO: 91 (LCDR1), SEQ ID NO: 92 (LCDR2), and SEQ ID NO: 93 (LCDR3); and heavy chain CDRs comprising amino acid sequences that differ by no more than 1, 2, or 3 amino acids from SEQ ID NO: 94 (HCDR1), SEQ ID NO: 95 (HCDR2), and SEQ ID NO: 96 (HCDR3);
or
b1) a light chain variable region comprising an amino acid sequence at least 90%, at least 95%, or at least 99% identical to SEQ ID NO: 97, 99, 101, 103, 105, 107, 109, 111, 113, 115, 117, 119, 121, 123, 125, or 127; and
b2) a heavy chain variable region comprising an amino acid sequence at least 90%, at least 95%, or at least 99% identical to SEQ ID NO: 98, 100, 102, 104, 106, 108, 110, 112, 114, 116, 118, 120, 122, 124, 126, or 128.

34. A method of treating a disease or disorder in a subject in need thereof, the method comprising administering to the subject a therapeutically effective amount of an anti-IL-13 antibody or antigen binding fragment thereof,
wherein the IL-13 antibody or antigen binding fragment thereof comprises:
a. a light chain variable region comprising SEQ ID NO: 1 (LCDR1), SEQ ID NO: 2 (LCDR2), and SEQ ID NO: 3 (LCDR3); and a heavy chain variable region comprising SEQ ID NO: 4 (HCDR1), SEQ ID NO: 5 (HCDR2), and SEQ ID NO: 6 (HCDR3);
b. a light chain variable region comprising SEQ ID NO: 7 (LCDR1), SEQ ID NO: 8 (LCDR2), and SEQ ID NO: 9 (LCDR3); and a heavy chain variable region comprising SEQ ID NO: 10 (HCDR1), SEQ ID NO: 11 (HCDR2), and SEQ ID NO: 12 (HCDR3);
c. a light chain variable region comprising SEQ ID NO: 13 (LCDR1), SEQ ID NO: 14 (LCDR2), and SEQ ID NO: 15 (LCDR3); and a heavy chain variable region comprising SEQ ID NO: 16 (HCDR1), SEQ ID NO: 17 (HCDR2), and SEQ ID NO: 18 (HCDR3);
d. a light chain variable region comprising SEQ ID NO: 19 (LCDR1), SEQ ID NO: 20 (LCDR2), and SEQ ID NO: 21 (LCDR3); and a heavy chain variable region comprising SEQ ID NO: 22 (HCDR1), SEQ ID NO: 23 (HCDR2), and SEQ ID NO: 24 (HCDR3);
e. a light chain variable region comprising SEQ ID NO: 25 (LCDR1), SEQ ID NO: 26 (LCDR2), and SEQ ID NO: 27 (LCDR3); and a heavy chain variable region comprising SEQ ID NO: 28 (HCDR1), SEQ ID NO: 29 (HCDR2), and SEQ ID NO: 30 (HCDR3);
f. a light chain variable region comprising SEQ ID NO: 31 (LCDR1), SEQ ID NO: 32 (LCDR2), and SEQ ID NO: 33 (LCDR3); and a heavy chain variable region comprising SEQ ID NO: 34 (HCDR1), SEQ ID NO: 35 (HCDR2), and SEQ ID NO: 36 (HCDR3);
g. a light chain variable region comprising SEQ ID NO: 37 (LCDR1), SEQ ID NO: 38 (LCDR2), and SEQ ID NO: 39 (LCDR3); and a heavy chain variable region comprising SEQ ID NO: 40 (HCDR1), SEQ ID NO: 41 (HCDR2), and SEQ ID NO: 42 (HCDR3);
h. a light chain variable region comprising SEQ ID NO: 43 (LCDR1), SEQ ID NO: 44 (LCDR2), and SEQ ID NO: 45 (LCDR3); and a heavy chain variable region comprising SEQ ID NO: 46 (HCDR1), SEQ ID NO: 47 (HCDR2), and SEQ ID NO: 48 (HCDR3);
i. a light chain variable region comprising SEQ ID NO: 49 (LCDR1), SEQ ID NO: 50 (LCDR2), and SEQ ID NO: 51 (LCDR3); and a heavy chain variable region comprising SEQ ID NO: 52 (HCDR1), SEQ ID NO: 53 (HCDR2), and SEQ ID NO: 54 (HCDR3);
j. a light chain variable region comprising SEQ ID NO: 55 (LCDR1), SEQ ID NO: 56 (LCDR2), and SEQ ID NO: 57 (LCDR3); and a heavy chain variable region comprising SEQ ID NO: 58 (HCDR1), SEQ ID NO: 59 (HCDR2), and SEQ ID NO: 60 (HCDR3);
k. a light chain variable region comprising SEQ ID NO: 61 (LCDR1), SEQ ID NO: 62 (LCDR2), and SEQ ID NO: 63 (LCDR3); and a heavy chain variable region comprising SEQ ID NO: 64 (HCDR1), SEQ ID NO: 65 (HCDR2), and SEQ ID NO: 66 (HCDR3);
l. a light chain variable region comprising SEQ ID NO: 67 (LCDR1), SEQ ID NO: 68 (LCDR2), and SEQ ID NO: 69 (LCDR3); and a heavy chain variable region comprising SEQ ID NO: 70 (HCDR1), SEQ ID NO: 71 (HCDR2), and SEQ ID NO: 72 (HCDR3);
m. a light chain variable region comprising SEQ ID NO: 73 (LCDR1), SEQ ID NO: 74 (LCDR2), and SEQ ID NO: 75 (LCDR3); and a heavy chain variable region comprising SEQ ID NO: 76 (HCDR1), SEQ ID NO: 77 (HCDR2), and SEQ ID NO: 78 (HCDR3);
n. a light chain variable region comprising SEQ ID NO: 79 (LCDR1), SEQ ID NO: 80 (LCDR2), and SEQ ID NO: 81 (LCDR3); and a heavy chain variable region comprising SEQ ID NO: 82 (HCDR1), SEQ ID NO: 83 (HCDR2), and SEQ ID NO: 84 (HCDR3);
o. a light chain variable region comprising SEQ ID NO: 85 (LCDR1), SEQ ID NO: 86 (LCDR2), and SEQ ID NO: 87 (LCDR3); and a heavy chain variable region comprising SEQ ID NO: 88 (HCDR1), SEQ ID NO: 89 (HCDR2), and SEQ ID NO: 90 (HCDR3); or
p. a light chain variable region comprising SEQ ID NO: 91 (LCDR1), SEQ ID NO: 92 (LCDR2), and SEQ ID NO: 93 (LCDR3); and a heavy chain variable region comprising SEQ ID NO: 94 (HCDR1), SEQ ID NO: 95 (HCDR2), and SEQ ID NO:
96 (HCDR3).

35. The method of claim 34, wherein the disease or disorder is selected from the group consisting of: asthma, allergic asthma, allergen-induced airway obstruction due to asthma and/or allergic asthma, atopic dermatitis (including, but not limited to, mild atopic dermatitis, mild to moderate atopic dermatitis, moderate atopic dermatitis, moderate-to-severe, and/or severe atopic dermatitis), eczema, acute urticaria, idiopathic pulmonary fibrosis (IPF), chronic obstructive pulmonary disease (COPD), chronic obstructive airway disease, emphysema, chronic bronchitis, pulmonary fibrosis, systemic sclerosis, scleroderma, cryptogenic fibrosing alveolitis, usual interstitial pneumonitis, idiopathic interstitial pneumonitis, wound, systemic lupus erythematosus (SLE), rheumatoid arthritis (RA), systemic sclerosis (SS), ulcerative colitis (UC), type 1 diabetes (T1D), sjogren's syndrome (SS), prurigo nodularis, nasal polyposis, recurrent urticaria, aspergillosis, bullous pemphigoid, chronic sinusitis, alopecia areata, hay fever, pemphigus, allergic rhinitis, psoriasis, rosacea, allergic drug reactions, allergic reactions, anaphylaxis, acne, food allergies, inflammatory bowel disease (IBS), Crohn's disease, ulcerative colitis, chronic rhinosinusitis with nasal polyps (CRSwNP), eosinophilic esophagitis, eosinophilic colitis, eosinophilic gastritis, eosinophilic cystitis, eosinophilic fasciitis, eosinophilic pustular folliculitis, eosinophilic granulomatosis, eosinophilic polyangiitis, Churg-Strauss syndrome, biliary cancer, brain cancer, breast cancer, colorectal cancer, genitourinary cancer, head and neck cancer, liver cancer, Hodgkin's lymphoma, esophageal cancer, pancreatic cancer, prostate cancer, renal cancer, Kaposi's sarcoma, sinusitis, or chronic urticaria.

36. The method of claim 34, wherein the light chain variable region comprises an amino acid sequence that is at least 90%, at least 95%, or at least 99% identical to in SEQ ID NO: 97, 99, 101, 103, 105, 107, 109, 111, 113, 115, 117, 119, 121, 123, 125, or 127; and the heavy chain variable region comprises an amino acid sequence that is at least 90%, at least 95%, or at least 99% identical to SEQ ID NO: 98, 100, 102, 104, 106, 108, 110, 112, 114, 116, 118, 120, 122, 124, 126, or 128.

37. The method of claim 34, wherein the light chain variable region comprises an amino acid sequence as set forth in SEQ ID NO: 97, 99, 101, 103, 105, 107, 109, 111, 113, 115, 117, 119, 121, 123, 125, or 127; and/or the heavy chain variable region comprises an amino acid sequence as set forth in SEQ ID NO: 98, 100, 102, 104, 106, 108, 110, 112, 114, 116, 118, 120, 122, 124, 126, or 128.

38. The method of claim 37, wherein:
a. the light chain variable region comprises an amino acid sequence as set forth in SEQ ID NO: 97; and the heavy chain variable region comprises an amino acid sequence as set forth in SEQ ID NO: 98;
b. the light chain variable region comprises an amino acid sequence as set forth in SEQ ID NO: 99; and the heavy chain variable region comprises an amino acid sequence as set forth in SEQ ID NO: 100;
c. the light chain variable region comprises an amino acid sequence as set forth in SEQ ID NO: 101; and the heavy chain variable region comprises an amino acid sequence as set forth in SEQ ID NO: 102;
d. the light chain variable region comprises an amino acid sequence as set forth in SEQ ID NO: 103; and the heavy chain variable region comprises an amino acid sequence as set forth in SEQ ID NO: 104;
e. the light chain variable region comprises an amino acid sequence as set forth in SEQ ID NO: 105; and the heavy chain variable region comprises an amino acid sequence as set forth in SEQ ID NO: 106;
f. the light chain variable region comprises an amino acid sequence as set forth in SEQ ID NO: 107; and the heavy chain variable region comprises an amino acid sequence as set forth in SEQ ID NO: 108;
g. the light chain variable region comprises an amino acid sequence as set forth in SEQ ID NO: 109; and the heavy chain variable region comprises an amino acid sequence as set forth in SEQ ID NO: 110;
h. the light chain variable region comprises an amino acid sequence as set forth in SEQ ID NO: 111; and the heavy chain variable region comprises an amino acid sequence as set forth in SEQ ID NO: 112;
i. the light chain variable region comprises an amino acid sequence as set forth in SEQ ID NO: 113; and the heavy chain variable region comprises an amino acid sequence as set forth in SEQ ID NO: 114;
j. the light chain variable region comprises an amino acid sequence as set forth in SEQ ID NO: 115; and the heavy chain variable region comprises an amino acid sequence as set forth in SEQ ID NO: 116;
k. the light chain variable region comprises an amino acid sequence as set forth in SEQ ID NO: 117; and the heavy chain variable region comprises an amino acid sequence as set forth in SEQ ID NO: 118;
l. the light chain variable region comprises an amino acid sequence as set forth in SEQ ID NO: 119; and the heavy chain variable region comprises an amino acid sequence as set forth in SEQ ID NO: 120;
m. the light chain variable region comprises an amino acid sequence as set forth in SEQ ID NO: 121; and the heavy chain variable region comprises an amino acid sequence as set forth in SEQ ID NO: 122
n. the light chain variable region comprises an amino acid sequence as set forth in SEQ ID NO: 123; and the heavy chain variable region comprises an amino acid sequence as set forth in SEQ ID NO: 124;
o. the light chain variable region comprises an amino acid sequence as set forth in SEQ ID NO: 125; and the heavy chain variable region comprises an amino acid sequence as set forth in SEQ ID NO: 126; or
p. the light chain variable region comprises an amino acid sequence as set forth in SEQ ID NO: 127; and the heavy chain variable region comprises an amino acid sequence as set forth in SEQ ID NO: 128.

39. The method of claim 34, wherein the antibody comprises a light chain comprising an amino acid sequence that is at least 90%, at least 95%, or at least 99% identical to SEQ ID NO: 129, 131, 133, 135, 137, 139, 141, 143, 145, 147, 149, 151, 153, 155, 157, or 159; and/or a heavy chain comprising an amino acid sequence that is at least 90%, at least 95%, or at least 99% identical to SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

40. The method of claim 39, wherein the light chain comprises an amino acid sequence as set forth in SEQ ID NO: 129, 131, 133, 135, 137, 139, 141, 143, 145, 147, 149, 151, 153, 155, 157, or 159; and/or the heavy chain comprises an amino acid sequence as set forth in SEQ ID NO: 130, 132, 134, 136, 138, 140, 142, 144, 146, 148, 150, 152, 154, 156, 158, 160, 182, 183, 184, 185, 186, 187, 188, 189, 190, 191, 192, 193, 194, 195, 196, 197, 198, 199, 200, 201, 202, 203, 204, 205, 206, 207, 208, 209, 210, or 211.

41. The method of claim 40, wherein:
a. the light chain comprises an amino acid sequence as set forth in SEQ ID NO: 129; and the heavy chain comprises an amino acid sequence as set forth in SEQ ID NO: 130;
b. the light chain comprises an amino acid sequence as set forth in SEQ ID NO: 131; and the heavy chain comprises an amino acid sequence as set forth in SEQ ID NO: 132;
c. the light chain comprises an amino acid sequence as set forth in SEQ ID NO: 133; and the heavy chain comprises an amino acid sequence as set forth in SEQ ID NO: 134;
d. the light chain comprises an amino acid sequence as set forth in SEQ ID NO: 135; and the heavy chain comprises an amino acid sequence as set forth in SEQ ID NO: 136;
e. the light chain comprises an amino acid sequence as set forth in SEQ ID NO: 137; and the heavy chain comprises an amino acid sequence as set forth in SEQ ID NO: 138;
f. the light chain comprises an amino acid sequence as set forth in SEQ ID NO: 139; and the heavy chain comprises an amino acid sequence as set forth in SEQ ID NO: 140;
g. the light chain comprises an amino acid sequence as set forth in SEQ ID NO: 141; and the heavy chain comprises an amino acid sequence as set forth in SEQ ID NO: 142;
h. the light chain comprises an amino acid sequence as set forth in SEQ ID NO: 143; and the heavy chain comprises an amino acid sequence as set forth in SEQ ID NO: 144;
i. the light chain comprises an amino acid sequence as set forth in SEQ ID NO: 145; and the heavy chain comprises an amino acid sequence as set forth in SEQ ID NO: 146;
j. the light chain comprises an amino acid sequence as set forth in SEQ ID NO: 147; and the heavy chain comprises an amino acid sequence as set forth in SEQ ID NO: 148;
k. the light chain comprises an amino acid sequence as set forth in SEQ ID NO: 149; and the heavy chain comprises an amino acid sequence as set forth in SEQ ID NO: 150;
l. the light chain comprises an amino acid sequence as set forth in SEQ ID NO: 151; and the heavy chain comprises an amino acid sequence as set forth in SEQ ID NO: 152;
m. the light chain comprises an amino acid sequence as set forth in SEQ ID NO: 153; and the heavy chain comprises an amino acid sequence as set forth in SEQ ID NO: 154;
n. the light chain comprises an amino acid sequence as set forth in SEQ ID NO: 155; and the heavy chain comprises an amino acid sequence as set forth in SEQ ID NO: 156;
o. the light chain comprises an amino acid sequence as set forth in SEQ ID NO: 157; and the heavy chain comprises an amino acid sequence as set forth in SEQ ID NO: 158;
p. the light chain comprises an amino acid sequence as set forth in SEQ ID NO: 159; and the heavy chain comprises an amino acid sequence as set forth in SEQ ID NO: 160;
q. the light chain comprises an amino acid sequence as set forth in SEQ ID NO: 153; and the heavy chain comprises an amino acid sequence as set forth in SEQ ID NO: 182;
r. the light chain comprises an amino acid sequence as set forth in SEQ ID NO: 147; and the heavy chain comprises an amino acid sequence as set forth in SEQ ID NO: 183;
s. the light chain comprises an amino acid sequence as set forth in SEQ ID NO: 141; and the heavy chain comprises an amino acid sequence as set forth in SEQ ID NO: 184;
t. the light chain comprises an amino acid sequence as set forth in SEQ ID NO: 153; and the heavy chain comprises an amino acid sequence as set forth in SEQ ID NO: 185;
u. the light chain comprises an amino acid sequence as set forth in SEQ ID NO: 147; and the heavy chain comprises an amino acid sequence as set forth in SEQ ID NO: 186;
v. the light chain comprises an amino acid sequence as set forth in SEQ ID NO: 141; and the heavy chain comprises an amino acid sequence as set forth in SEQ ID NO: 187;
w. the light chain comprises an amino acid sequence as set forth in SEQ ID NO: 153; and the heavy chain comprises an amino acid sequence as set forth in SEQ ID NO: 188;
x. the light chain comprises an amino acid sequence as set forth in SEQ ID NO: 147; and the heavy chain comprises an amino acid sequence as set forth in SEQ ID NO: 189;
y. the light chain comprises an amino acid sequence as set forth in SEQ ID NO: 141; and the heavy chain comprises an amino acid sequence as set forth in SEQ ID NO: 190;
z. the light chain comprises an amino acid sequence as set forth in SEQ ID NO: 153; and the heavy chain comprises an amino acid sequence as set forth in SEQ ID NO: 191;
aa. the light chain comprises an amino acid sequence as set forth in SEQ ID NO: 147; and the heavy chain comprises an amino acid sequence as set forth in SEQ ID NO: 192;
bb. the light chain comprises an amino acid sequence as set forth in SEQ ID NO: 141; and the heavy chain comprises an amino acid sequence as set forth in SEQ ID NO: 193;
cc. the light chain comprises an amino acid sequence as set forth in SEQ ID NO: 153; and the heavy chain comprises an amino acid sequence as set forth in SEQ ID NO: 194;
dd. the light chain comprises an amino acid sequence as set forth in SEQ ID NO: 147; and the heavy chain comprises an amino acid sequence as set forth in SEQ ID NO: 195;
ee. the light chain comprises an amino acid sequence as set forth in SEQ ID NO: 141; and the heavy chain comprises an amino acid sequence as set forth in SEQ ID NO: 196;
ff. the light chain comprises an amino acid sequence as set forth in SEQ ID NO: 153; and the heavy chain comprises an amino acid sequence as set forth in SEQ ID NO: 197;
gg. the light chain comprises an amino acid sequence as set forth in SEQ ID NO: 147; and the heavy chain comprises an amino acid sequence as set forth in SEQ ID NO: 198;
hh. the light chain comprises an amino acid sequence as set forth in SEQ ID NO: 141; and the heavy chain comprises an amino acid sequence as set forth in SEQ ID NO: 199;
ii. the light chain comprises an amino acid sequence as set forth in SEQ ID NO: 153; and the heavy chain comprises an amino acid sequence as set forth in SEQ ID NO: 200;
jj. the light chain comprises an amino acid sequence as set forth in SEQ ID NO: 147; and the heavy chain comprises an amino acid sequence as set forth in SEQ ID NO: 201;
kk. the light chain comprises an amino acid sequence as set forth in SEQ ID NO: 141; and the heavy chain comprises an amino acid sequence as set forth in SEQ ID NO: 202;
ll. the light chain comprises an amino acid sequence as set forth in SEQ ID NO: 153; and the heavy chain comprises an amino acid sequence as set forth in SEQ ID NO: 203;
mm. the light chain comprises an amino acid sequence as set forth in SEQ ID NO: 147; and the heavy chain comprises an amino acid sequence as set forth in SEQ ID NO: 204;
nn. the light chain comprises an amino acid sequence as set forth in SEQ ID NO: 141; and the heavy chain comprises an amino acid sequence as set forth in SEQ ID NO: 205;
oo. the light chain comprises an amino acid sequence as set forth in SEQ ID NO: 153; and the heavy chain comprises an amino acid sequence as set forth in SEQ ID NO: 206;
pp. the light chain comprises an amino acid sequence as set forth in SEQ ID NO: 147; and the heavy chain comprises an amino acid sequence as set forth in SEQ ID NO: 207;
qq. the light chain comprises an amino acid sequence as set forth in SEQ ID NO: 141; and the heavy chain comprises an amino acid sequence as set forth in SEQ ID NO: 208;
rr. the light chain comprises an amino acid sequence as set forth in SEQ ID NO: 153; and the heavy chain comprises an amino acid sequence as set forth in SEQ ID NO: 209;
ss. the light chain comprises an amino acid sequence as set forth in SEQ ID NO: 147; and the heavy chain comprises an amino acid sequence as set forth in SEQ ID NO: 210; or
tt. the light chain comprises an amino acid sequence as set forth in SEQ ID NO: 141; and the heavy chain comprises an amino acid sequence as set forth in SEQ ID NO: 211.

42. The method of claim 34, wherein the antibody is a monoclonal antibody.

43. The method of claim 34, wherein the antibody is a human antibody.

44. The method of claim 34, wherein the antibody is a bispecific antibody.

45. The method of claim 34, wherein the binding fragment thereof is selected from the group consisting of Fab, Fab′, F(ab′)₂, Fd, Fv, scFv, a single-chain antibody, a minibody, and a diabody.

46. The method of claim 45, wherein the binding fragment thereof is an scFv.

47. A computer-implemented method comprising:
scoring a polypeptide comprising an amino acid sequence with a computationally binding optimized (CBO) model for a functional property relating to modulating activity of a target molecule, the CBO model:
for each amino acid position of the amino acid sequence of the polypeptide, calculating a plurality of energy scores, the energy scores based on the amino acid at a given position in the amino acid sequence,
adding each energy score calculated to an array,
generating a normalized sum of the energy scores for each position, and generating a score of the polypeptide by summing each energy score calculated, the score representing the functional property of the polypeptide, the functional property relating to the polypeptide modulating activity of the target molecule.

48. The computer-implemented method of claim 47, further comprising:
calculating a logarithm of each energy score calculated;
wherein summing each energy score calculated is performed by summing the logarithms of each energy score calculated;
wherein the energy scores are further calculated based on having substituted the amino acid at the given position in the amino acid sequence with each of a plurality of different amino acids.

49. The computer-implemented method of claim 47, wherein scoring the polypeptide using the CBO model is implementable by a script of Appendix A, and the CBO model is substantially similar to a table of Appendix B.

50. The method of claim 47, wherein the functional property is at least one of:
a binding affinity for an interleukin-13 (IL-13) polypeptide characterized by a K_{D} of 10 pM or less (optionally, as measured by KinExA);
a binding specificity for the IL-13 polypeptide;
a neutralizing activity against the IL-13 polypeptide (optionally, a full-length human IL-13); or
an inhibitory activity against IL-13-mediated signaling, or a combination thereof.

51. The method of claim 50, wherein the target molecule is interleukin-13 (IL-13), and the functional property of the relating to the polypeptide modulates the activity of the IL-13.

52. A system comprising:
a processor; and
a memory with computer code instructions stored thereon, the processor and the memory, with the computer code instructions, being configured to cause the system to:
score a polypeptide comprising an amino acid sequence with a CBO model for a functional property relating to modulating activity of a target molecule, the CBO model:
for each amino acid position of the amino acid sequence of the polypeptide, calculating a plurality of energy scores, the energy scores based on the amino acid at a given position in the amino acid sequence,
add each energy score calculated to an array,
generate a normalized sum of the energy scores for each position, and
generate a score of the amino acid sequence by summing each energy score calculated, the score representing a functional property of the polypeptide, the functional property relating to the polypeptide modulating activity of the target molecules.

53. The system of claim 52, wherein the instructions are further configured to cause the system to calculate a logarithm of each energy score calculated;
wherein summing each energy score calculated is performed by summing the logarithms of each energy score calculated;
wherein the energy scores are further based on having substituted the amino acid at the given position in the amino acid sequence with each of a plurality of different amino acids.

54. The system of claim 52, wherein scoring the polypeptide using the CBO model is implemented by a script of Appendix A and wherein the CBO model is substantially similar to a table of Appendix B.

55. The system of claim 52 wherein the functional property is at least one of:
a binding affinity for an interleukin-13 (IL-13) polypeptide characterized by a K_{D} of 10 pM or less (optionally, as measured by KinExA);
a binding specificity for the IL-13 polypeptide;
a neutralizing activity against the IL-13 polypeptide (optionally, a full-length human IL-13); or
an inhibitory activity against IL-13-mediated signaling,
or a combination thereof.

56. The system of claim 55, wherein the target molecule is interleukin-13 (IL-13), and the functional property of the relating to the polypeptide modulates the activity of the IL-13.

57. A polypeptide that specifically binds an interleukin-13 (IL-13):
wherein the polypeptide comprises an amino acid sequence that is assigned a score above a predetermined threshold by a computationally binding optimized (CBO) model upon scoring the amino acid sequence of the polypeptide.

58. The polypeptide of claim 57, wherein the polypeptide is scorable by a script of Computer Program Listing Appendix A and wherein the CBO model is calculated by a table substantially similar to that of Computer Program Listing Appendix B and wherein the polypeptide is assigned the score that is calculated using the Computer Program Listing Appendix B;
wherein the polypeptide is assigned the score by a script substantially similar to Computer Program Listing Appendix A, the script employing the CBO model substantially similar to the table of Computer Program Listing Appendix B, wherein the polypeptide has a logarithmic score of at least about: −1.7, −1.0, −0.5, 0, 0.5, 1, 1.5, 1.8, 2.0, and 3.0.

59. The polypeptide of claim 57, wherein the polypeptide has one or more properties selected from:
a binding affinity for an interleukin-13 (IL-13) polypeptide characterized by a K_{D} of 10 pM or less (optionally, as measured by KinExA);
a binding specificity for the IL-13 polypeptide;
a neutralizing activity against the IL-13 polypeptide (optionally, a full-length human IL-13); or
an inhibitory activity against IL-13-mediated signaling,
or a combination thereof.

60. The polypeptide of claim 57, wherein the CBO model outputs a score that is equal to or above a score from the CBO model of one or more of a reference polypeptide that comprises a VL and VH pair selected from:
SEQ ID NO: 97 and SEQ ID NO: 98 (AbA);
SEQ ID NO: 99 and SEQ ID NO: 100 (AbB);
SEQ ID NO: 101 and SEQ ID NO: 102 (AbC);
SEQ ID NO: 103 and SEQ ID NO: 104 (AbD);
SEQ ID NO: 105 and SEQ ID NO: 106 (AbE);
SEQ ID NO: 107 and SEQ ID NO: 108 (AbF);
SEQ ID NO: 109 and SEQ ID NO: 110 (AbG);
SEQ ID NO: 111 and SEQ ID NO: 112 (AbH);
SEQ ID NO: 113 and SEQ ID NO: 114 (AbI);
SEQ ID NO: 115 and SEQ ID NO: 116 (AbJ);
SEQ ID NO: 117 and SEQ ID NO: 118 (AbK);
SEQ ID NO: 119 and SEQ ID NO: 120 (AbL);
SEQ ID NO: 121 and SEQ ID NO: 122 (AbM);
SEQ ID NO: 123 and SEQ ID NO: 124 (AbN);
SEQ ID NO: 125 and SEQ ID NO: 126 (AbO); or
SEQ ID NO: 127 and SEQ ID NO: 128 (AbP).

61. The polypeptide of claim 57, wherein the polypeptide does not comprise a heavy chain comprising SEQ ID NO: 179 or 181 and a light chain comprising SEQ ID NO: 178 or 180.

62. A polypeptide: wherein the polypeptide comprises an amino acid sequence that is assigned a score above a predetermined threshold by a computationally binding optimized (CBO) model upon scoring the amino acid sequence of the polypeptide.

63. A polypeptide that binds human interleukin-13 (IL-13), wherein the polypeptide is designed by a method comprising:
generating a polypeptide sequence with a CBO model;
verifying the generated polypeptide sequence using the CBO model by:
for each amino acid position of the polypeptide sequence, calculating a plurality of energy scores, the energy scores based on having substituted the amino acid at a given position in the polypeptide sequence with each of a plurality of different amino acids,
adding each energy score calculated to an array,
generating a normalized sum of the energy scores for each position,
calculating a logarithm of each energy score calculated, and
generating a score of the polypeptide sequence by summing the logarithms of each energy score calculated, the score representing a functional property of the polypeptide's ability to bind to human IL-13.


SEQUENCE LISTING (239 sequences)

SEQ ID NO: 1 - protein, 15 aa, synthetic construct
  source 1..15: note = AbA LCDR1 Kabat
RASKSVDSYGRSYMH

SEQ ID NO: 2 - protein, 7 aa, synthetic construct
  source 1..7: note = AbA LCDR2 Kabat
LASNLES

SEQ ID NO: 3 - protein, 9 aa, synthetic construct
  source 1..9: note = AbA LCDR3 Kabat
QQNNEDPRT

SEQ ID NO: 4 - protein, 5 aa, synthetic construct
  source 1..5: note = AbA HCDR1 Kabat
AYSVN

SEQ ID NO: 5 - protein, 16 aa, synthetic construct
  source 1..16: note = AbA HCDR2 Kabat
MIWGDGKIVYNSALKS

SEQ ID NO: 6 - protein, 10 aa, synthetic construct
  source 1..10: note = AbA HCDR3 Kabat
DGYYPYAMDN

SEQ ID NO: 7 - protein, 15 aa, synthetic construct
  source 1..15: note = AbB LCDR1 Kabat
RASKSVDYYGHSYMH

SEQ ID NO: 8 - protein, 7 aa, synthetic construct
  source 1..7: note = AbB LCDR2 Kabat
LASNLES

SEQ ID NO: 9 - protein, 9 aa, synthetic construct
  source 1..9: note = AbB LCDR3 Kabat
QQNNEDPRT

SEQ ID NO: 10 - protein, 5 aa, synthetic construct
  source 1..5: note = AbB HCDR1 Kabat
AYSVN

SEQ ID NO: 11 - protein, 16 aa, synthetic construct
  source 1..16: note = AbB HCDR2 Kabat
MIWGDGKIVYNSALKS

SEQ ID NO: 12 - protein, 10 aa, synthetic construct
  source 1..10: note = AbB HCDR3 Kabat
DGYYPYAMDN

SEQ ID NO: 13 - protein, 15 aa, synthetic construct
  source 1..15: note = AbC LCDR1 Kabat
RASKSVDYYGNSYMH

SEQ ID NO: 14 - protein, 7 aa, synthetic construct
  source 1..7: note = AbC LCDR2 Kabat
LASNLES

SEQ ID NO: 15 - protein, 9 aa, synthetic construct
  source 1..9: note = AbC LCDR3 Kabat
QQNNEDPRT

SEQ ID NO: 16 - protein, 5 aa, synthetic construct
  source 1..5: note = AbC HCDR1 Kabat
AYSVN

SEQ ID NO: 17 - protein, 16 aa, synthetic construct
  source 1..16: note = AbC HCDR2 Kabat
MIWGDGKIVYNSALKS

SEQ ID NO: 18 - protein, 10 aa, synthetic construct
  source 1..10: note = AbC HCDR3 Kabat
DGYYPYAMDY

SEQ ID NO: 19 - protein, 15 aa, synthetic construct
  source 1..15: note = AbD LCDR1 Kabat
RASKSVDSYGNSYMH

SEQ ID NO: 20 - protein, 7 aa, synthetic construct
  source 1..7: note = AbD LCDR2 Kabat
LASNLES

SEQ ID NO: 21 - protein, 9 aa, synthetic construct
  source 1..9: note = AbD LCDR3 Kabat
QQNNEDPRT

SEQ ID NO: 22 - protein, 5 aa, synthetic construct
  source 1..5: note = AbD HCDR1 Kabat
AYSVN

SEQ ID NO: 23 - protein, 16 aa, synthetic construct
  source 1..16: note = AbD HCDR2 Kabat
MIWGDGKVVYNSALKS

SEQ ID NO: 24 - protein, 10 aa, synthetic construct
  source 1..10: note = AbD HCDR3 Kabat
DGYFPYAMDN

SEQ ID NO: 25 - protein, 15 aa, synthetic construct
  source 1..15: note = AbE LCDR1 Kabat
RASKSVDSYGHSYMH

SEQ ID NO: 26 - protein, 7 aa, synthetic construct
  source 1..7: note = AbE LCDR2 Kabat
WASNLES

SEQ ID NO: 27 - protein, 9 aa, synthetic construct
  source 1..9: note = AbE LCDR3 Kabat
QQNNEDPRT

SEQ ID NO: 28 - protein, 5 aa, synthetic construct
  source 1..5: note = AbE HCDR1 Kabat
AYSVN

SEQ ID NO: 29 - protein, 16 aa, synthetic construct
  source 1..16: note = AbE HCDR2 Kabat
MIWGDGKIVYNSALKS

SEQ ID NO: 30 - protein, 10 aa, synthetic construct
  source 1..10: note = AbE HCDR3 Kabat
DGYFPYAMDN

SEQ ID NO: 31 - protein, 15 aa, synthetic construct
  source 1..15: note = AbF LCDR1 Kabat
RASESVDSYGNSYMH

SEQ ID NO: 32 - protein, 7 aa, synthetic construct
  source 1..7: note = AbF LCDR2 Kabat
LASNLES

SEQ ID NO: 33 - protein, 9 aa, synthetic construct
  source 1..9: note = AbF LCDR3 Kabat
QQNNEDPRT

SEQ ID NO: 34 - protein, 5 aa, synthetic construct
  source 1..5: note = AbF HCDR1 Kabat
RYSVN

SEQ ID NO: 35 - protein, 16 aa, synthetic construct
  source 1..16: note = AbF HCDR2 Kabat
MIWGDAKIVYNSALKS

SEQ ID NO: 36 - protein, 10 aa, synthetic construct
  source 1..10: note = AbF HCDR3 Kabat
DGYFPYAMDN

SEQ ID NO: 37 - protein, 15 aa, synthetic construct
  source 1..15: note = AbG LCDR1 Kabat
RASKSVDSYGNSYMH

SEQ ID NO: 38 - protein, 7 aa, synthetic construct
  source 1..7: note = AbG LCDR2 Kabat
LASNLES

SEQ ID NO: 39 - protein, 9 aa, synthetic construct
  source 1..9: note = AbG LCDR3 Kabat
QQNNEDPRT

SEQ ID NO: 40 - protein, 5 aa, synthetic construct
  source 1..5: note = AbG HCDR1 Kabat
AYSVN

SEQ ID NO: 41 - protein, 16 aa, synthetic construct
  source 1..16: note = AbG HCDR2 Kabat
MIWGDGKIVYNSALKS

SEQ ID NO: 42 - protein, 10 aa, synthetic construct
  source 1..10: note = AbG HCDR3 Kabat
DGYFPYAMDN

SEQ ID NO: 43 - protein, 15 aa, synthetic construct
  source 1..15: note = AbH LCDR1 Kabat
RASKSVDSYGNSYMH

SEQ ID NO: 44 - protein, 7 aa, synthetic construct
  source 1..7: note = AbH LCDR2 Kabat
RASNLES

SEQ ID NO: 45 - protein, 9 aa, synthetic construct
  source 1..9: note = AbH LCDR3 Kabat
QQNNEDPRT

SEQ ID NO: 46 - protein, 5 aa, synthetic construct
  source 1..5: note = AbH HCDR1 Kabat
RYSVN

SEQ ID NO: 47 - protein, 16 aa, synthetic construct
  source 1..16: note = AbH HCDR2 Kabat
MIWGDGKIVYNSALKS

SEQ ID NO: 48 - protein, 10 aa, synthetic construct
  source 1..10: note = AbH HCDR3 Kabat
DGYYPYAMDN

SEQ ID NO: 49 - protein, 15 aa, synthetic construct
  source 1..15: note = AbI LCDR1 Kabat
RASKSVDSYGNSYMH

SEQ ID NO: 50 - protein, 7 aa, synthetic construct
  source 1..7: note = AbI LCDR2 Kabat
LASNLES

SEQ ID NO: 51 - protein, 9 aa, synthetic construct
  source 1..9: note = AbI LCDR3 Kabat
QQNNEDPRT

SEQ ID NO: 52 - protein, 5 aa, synthetic construct
  source 1..5: note = AbI HCDR1 Kabat
RYSVN

SEQ ID NO: 53 - protein, 16 aa, synthetic construct
  source 1..16: note = AbI HCDR2 Kabat
MIWGDGKIVYNSALKS

SEQ ID NO: 54 - protein, 10 aa, synthetic construct
  source 1..10: note = AbI HCDR3 Kabat
DGFYPYAMDN

SEQ ID NO: 55 - protein, 15 aa, synthetic construct
  source 1..15: note = AbJ LCDR1 Kabat
RASKSVDSYGHSYMH

SEQ ID NO: 56 - protein, 7 aa, synthetic construct
  source 1..7: note = AbJ LCDR2 Kabat
WASNLES

SEQ ID NO: 57 - protein, 9 aa, synthetic construct
  source 1..9: note = AbJ LCDR3 Kabat
QQNNEDPRT

SEQ ID NO: 58 - protein, 5 aa, synthetic construct
  source 1..5: note = AbJ HCDR1 Kabat
AYSVN

SEQ ID NO: 59 - protein, 16 aa, synthetic construct
  source 1..16: note = AbJ HCDR2 Kabat
MIWGDRKKVYNSALKS

SEQ ID NO: 60 - protein, 10 aa, synthetic construct
  source 1..10: note = AbJ HCDR3 Kabat
DGYFPYAMDL

SEQ ID NO: 61 - protein, 15 aa, synthetic construct
  source 1..15: note = AbK LCDR1 Kabat
RASQSVDSYGNSYMH

SEQ ID NO: 62 - protein, 7 aa, synthetic construct
  source 1..7: note = AbK LCDR2 Kabat
LASNLES

SEQ ID NO: 63 - protein, 9 aa, synthetic construct
  source 1..9: note = AbK LCDR3 Kabat
QQNNEDPRT

SEQ ID NO: 64 - protein, 5 aa, synthetic construct
  source 1..5: note = AbK HCDR1 Kabat
RYSVN

SEQ ID NO: 65 - protein, 16 aa, synthetic construct
  source 1..16: note = AbK HCDR2 Kabat
MIWGDGKIVYNSALKS

SEQ ID NO: 66 - protein, 10 aa, synthetic construct
  source 1..10: note = AbK HCDR3 Kabat
DGYYPYAMDN

SEQ ID NO: 67 - protein, 15 aa, synthetic construct
  source 1..15: note = AbL LCDR1 Kabat
RASKSVDSYGRSYMH

SEQ ID NO: 68 - protein, 7 aa, synthetic construct
  source 1..7: note = AbL LCDR2 Kabat
LASNLES

SEQ ID NO: 69 - protein, 9 aa, synthetic construct
  source 1..9: note = AbL LCDR3 Kabat
QQNNEDPRT

SEQ ID NO: 70 - protein, 5 aa, synthetic construct
  source 1..5: note = AbL HCDR1 Kabat
RYSVN

SEQ ID NO: 71 - protein, 16 aa, synthetic construct
  source 1..16: note = AbL HCDR2 Kabat
MIWGDGKIVYNSALKS

SEQ ID NO: 72 - protein, 10 aa, synthetic construct
  source 1..10: note = AbL HCDR3 Kabat
DGYYPYAMDN

SEQ ID NO: 73 - protein, 15 aa, synthetic construct
  source 1..15: note = AbM LCDR1 Kabat
RASKSVDYYGRSYMH

SEQ ID NO: 74 - protein, 7 aa, synthetic construct
  source 1..7: note = AbM LCDR2 Kabat
LASNLES

SEQ ID NO: 75 - protein, 9 aa, synthetic construct
  source 1..9: note = AbM LCDR3 Kabat
QQNNEDPRT

SEQ ID NO: 76 - protein, 5 aa, synthetic construct
  source 1..5: note = AbM HCDR1 Kabat
AYSVN

SEQ ID NO: 77 - protein, 16 aa, synthetic construct
  source 1..16: note = AbM HCDR2 Kabat
MIWGDGKIVYNSALKS

SEQ ID NO: 78 - protein, 10 aa, synthetic construct
  source 1..10: note = AbM HCDR3 Kabat
DGYFPYAMDN

SEQ ID NO: 79 - protein, 15 aa, synthetic construct
  source 1..15: note = AbN LCDR1 Kabat
RASESVDYYGHSYMH

SEQ ID NO: 80 - protein, 7 aa, synthetic construct
  source 1..7: note = AbN LCDR2 Kabat
LASNLES

SEQ ID NO: 81 - protein, 9 aa, synthetic construct
  source 1..9: note = AbN LCDR3 Kabat
QQNNEDPRT

SEQ ID NO: 82 - protein, 5 aa, synthetic construct
  source 1..5: note = AbN HCDR1 Kabat
AYSVN

SEQ ID NO: 83 - protein, 16 aa, synthetic construct
  source 1..16: note = AbN HCDR2 Kabat
MIWGDGKIVYNSALKS

SEQ ID NO: 84 - protein, 10 aa, synthetic construct
  source 1..10: note = AbN HCDR3 Kabat
DGYYPYAMDN

SEQ ID NO: 85 - protein, 15 aa, synthetic construct
  source 1..15: note = AbO LCDR1 Kabat
RASKSVDYYGHSYMH

SEQ ID NO: 86 - protein, 7 aa, synthetic construct
  source 1..7: note = AbO LCDR2 Kabat
LASNLES

SEQ ID NO: 87 - protein, 9 aa, synthetic construct
  source 1..9: note = AbO LCDR3 Kabat
QQNNEDPRT

SEQ ID NO: 88 - protein, 5 aa, synthetic construct
  source 1..5: note = AbO HCDR1 Kabat
AYSVN

SEQ ID NO: 89 - protein, 16 aa, synthetic construct
  source 1..16: note = AbO HCDR2 Kabat
MIWGDGKIVYNSALKS

SEQ ID NO: 90 - protein, 10 aa, synthetic construct
  source 1..10: note = AbO HCDR3 Kabat
DGYFPYAMDV

SEQ ID NO: 91 - protein, 15 aa, synthetic construct
  source 1..15: note = AbP LCDR1 Kabat
RASKSVDSYGNSYMH

SEQ ID NO: 92 - protein, 7 aa, synthetic construct
  source 1..7: note = AbP LCDR2 Kabat
WASNLES

SEQ ID NO: 93 - protein, 9 aa, synthetic construct
  source 1..9: note = AbP LCDR3 Kabat
QQNNEDPRT

SEQ ID NO: 94 - protein, 5 aa, synthetic construct
  source 1..5: note = AbP HCDR1 Kabat
AYSVN

SEQ ID NO: 95 - protein, 16 aa, synthetic construct
  source 1..16: note = AbP HCDR2 Kabat
MIWGDGKVVYNSALKS

SEQ ID NO: 96 - protein, 10 aa, synthetic construct
  source 1..10: note = AbP HCDR3 Kabat
DGYFPYAMDH

SEQ ID NO: 97 - protein, 111 aa, synthetic construct
  source 1..111: note = AbA variable light chain
DIVMTQSPDSLSVSLGERATINCRASKSVDSYGRSYMHWYQQKPGQPPKLLIYLASNLESGVPDRFSGSGSGTDFTLTISSLQAEDVAVYYCQQNNEDPRTFGGGTKVEIK

SEQ ID NO: 98 - protein, 118 aa, synthetic construct [sequence omitted]

SEQ ID NO: 99 - protein, 111 aa, synthetic construct
  source 1..111: note = AbB variable light chain
DIVMTQSPDSLSVSLGERATINCRASKSVDYYGHSYMHWYQQKPGQPPKLLIYLASNLESGVPDRFSGSGSGTDFTLTISSLQAEDVAVYYCQQNNEDPRTFGGGTKVEIK

SEQ ID NO: 100 - protein, 118 aa, synthetic construct
  source 1..118: note = AbB variable heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 101 - protein, 111 aa, synthetic construct
  source 1..111: note = AbC variable light chain
DIVMTQSPDSLSVSLGERATINCRASKSVDYYGNSYMHWYQQKPGQPPKLLIYLASNLESGVPDRFSGSGSGTDFTLTISSLQAEDVAVYYCQQNNEDPRTFGGGTKVEIK

SEQ ID NO: 102 - protein, 118 aa, synthetic construct
  source 1..118: note = AbC variable heavy chain
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDYWGQGSLVTVSS

SEQ ID NO: 103 - protein, 111 aa, synthetic construct
  source 1..111: note = AbD variable light chain
DIVMTQSPDSLSVSLGERATINCRASKSVDSYGNSYMHWYQQKPGQPPKLLIYLASNLESGVPDRFSGSGSGTDFTLTISSLQAEDVAVYYCQQNNEDPRTFGGGTKVEIK

SEQ ID NO: 104 - protein, 118 aa, synthetic construct
  source 1..118: note = AbD variable heavy chain
QVTLRESGPALVKPTQTLTLTCTVSGSSLSAYSVNWIRQPPGKALEWLAMIWGDGKVVYNSALKSRLTISKDTSKNQVVLTMTNMDPVDTATYYCAGDGYFPYAMDNWGQGSLVTVSS

SEQ ID NO: 105 - protein, 111 aa, synthetic construct
  source 1..111: note = AbE variable light chain
DIVMTQSPDSLSVSLGERATINCRASKSVDSYGHSYMHWYQQKPGQPPKLLIYWASNLESGVPDRFSGSGSGTDFTLTISSLQAEDVAVYYCQQNNEDPRTFGGGTKVEIK

SEQ ID NO: 106 - protein, 118 aa, synthetic construct
  source 1..118: note = AbE variable heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSS

SEQ ID NO: 107 - protein, 111 aa, synthetic construct
  source 1..111: note = AbF variable light chain
DIVMTQSPDSLSVSLGERATINCRASESVDSYGNSYMHWYQQKPGQPPKLLIYLASNLESGVPDRFSGSGSGTDFTLTISSLQAEDVAVYYCQQNNEDPRTFGGGTKVEIK

SEQ ID NO: 108 - protein, 118 aa, synthetic construct
  source 1..118: note = AbF variable heavy chain
QVTLRESGPALVKPTQTLTLTCTVSGASLSRYSVNWIRQPPGKALEWLAMIWGDAKIVYNSALKSRLTISKDTSKNQVVLTMTNMDPVDTATYYCAGDGYFPYAMDNWGQGSLVTVSS

SEQ ID NO: 109 - protein, 111 aa, synthetic construct; identical to SEQ ID NO: 103
  source 1..111: note = AbG variable light chain

SEQ ID NO: 110 - protein, 118 aa, synthetic construct; identical to SEQ ID NO: 106
  source 1..118: note = AbG variable heavy chain

SEQ ID NO: 111 - protein, 111 aa, synthetic construct
  source 1..111: note = AbH variable light chain
DIVMTQSPDSLSVSLGERATINCRASKSVDSYGNSYMHWYQQKPGQPPKLLIYRASNLESGVPDRFSGSGSGTDFTLTISSLQAEDVAVYYCQQNNEDPRTFGGGTKVEIK

SEQ ID NO: 112 - protein, 118 aa, synthetic construct
  source 1..118: note = AbH variable heavy chain
QVTLRESGPALVKPTQTLTLTCTVSGASLSRYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 113 - protein, 111 aa, synthetic construct; identical to SEQ ID NO: 103
  source 1..111: note = AbI variable light chain

SEQ ID NO: 114 - protein, 118 aa, synthetic construct
  source 1..118: note = AbI variable heavy chain
QVTLRESGPALVKPTQTLTLTCTVSGASLSRYSVNWIRQPPGKALEWLAMIWGDGKIVYNSALKSRLTISKDTSKNQVVLTMTNMDPVDTATYYCAGDGFYPYAMDNWGQGSLVTVSS

SEQ ID NO: 115 - protein, 111 aa, synthetic construct; identical to SEQ ID NO: 105
  source 1..111: note = AbJ variable light chain

SEQ ID NO: 116 - protein, 118 aa, synthetic construct
  source 1..118: note = AbJ variable heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSS

SEQ ID NO: 117 - protein, 111 aa, synthetic construct
  source 1..111: note = AbK variable light chain
DIVMTQSPDSLSVSLGERATINCRASQSVDSYGNSYMHWYQQKPGQPPKLLIYLASNLESGVPDRFSGSGSGTDFTLTISSLQAEDVAVYYCQQNNEDPRTFGGGTKVEIK

SEQ ID NO: 118 - protein, 118 aa, synthetic construct
  source 1..118: note = AbK variable heavy chain
QVTLRESGPALVKPTQTLTLTCTVSGSSLSRYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 119 - protein, 111 aa, synthetic construct; identical to SEQ ID NO: 97
  source 1..111: note = AbL variable light chain

SEQ ID NO: 120 - protein, 118 aa, synthetic construct
  source 1..118: note = AbL variable heavy chain
QVTLRESGPALVKPTQTLTLTCTVSGFSLSRYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 121 - protein, 111 aa, synthetic construct
  source 1..111: note = AbM variable light chain
DIVMTQSPDSLSVSLGERATINCRASKSVDYYGRSYMHWYQQKPGQPPKLLIYLASNLESGVPDRFSGSGSGTDFTLTISSLQAEDVAVYYCQQNNEDPRTFGGGTKVEIK

SEQ ID NO: 122 - protein, 118 aa, synthetic construct; identical to SEQ ID NO: 106
  source 1..118: note = AbM variable heavy chain

SEQ ID NO: 123 - protein, 111 aa, synthetic construct
  source 1..111: note = AbN variable light chain
DIVMTQSPDSLSVSLGERATINCRASESVDYYGHSYMHWYQQKPGQPPKLLIYLASNLESGVPDRFSGSGSGTDFTLTISSLQAEDVAVYYCQQNNEDPRTFGGGTKVEIK

SEQ ID NO: 124 - protein, 118 aa, synthetic construct; identical to SEQ ID NO: 100
  source 1..118: note = AbN variable heavy chain

SEQ ID NO: 125 - protein, 111 aa, synthetic construct; identical to SEQ ID NO: 99
  source 1..111: note = AbO variable light chain

SEQ ID NO: 126 - protein, 118 aa, synthetic construct
  source 1..118: note = AbO variable heavy chain
QVTLRESGPALVKPTQTLTLTCTVSGYSLSAYSVNWIRQPPGKALEWLAMIWGDGKIVYNSALKSRLTISKDTSKNQVVLTMTNMDPVDTATYYCAGDGYFPYAMDVWGQGSLVTVSS

SEQ ID NO: 127 - protein, 111 aa, synthetic construct
  source 1..111: note = AbP variable light chain
DIVMTQSPDSLSVSLGERATINCRASKSVDSYGNSYMHWYQQKPGQPPKLLIYWASNLESGVPDRFSGSGSGTDFTLTISSLQAEDVAVYYCQQNNEDPRTFGGGTKVEIK

SEQ ID NO: 128 - protein, 118 aa, synthetic construct
  source 1..118: note = AbP variable heavy chain
QVTLRESGPALVKPTQTLTLTCTVSGASLSAYSVNWIRQPPGKALEWLAMIWGDGKVVYNSALKSRLTISKDTSKNQVVLTMTNMDPVDTATYYCAGDGYFPYAMDHWGQGSLVTVSS

SEQ ID NO: 129 - protein, 218 aa, synthetic construct
  source 1..218: note = AbA light chain
DIVMTQSPDSLSVSLGERATINCRASKSVDSYGRSYMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 448 aa, synthetic construct
  source 1..448: note = AbA heavy chain
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 218 aa, synthetic construct
  source 1..218: note = AbB light chain
DIVMTQSPDSLSVSLGERATINCRASKSVDYYGHSYMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 448 aa, synthetic construct
  source 1..448: note = AbB heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 218 aa, synthetic construct
  source 1..218: note = AbC light chain
DIVMTQSPDSLSVSLGERATINCRASKSVDYYGNSYMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 448 aa, synthetic construct
  source 1..448: note = AbC heavy chain
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 218 aa, synthetic construct
  source 1..218: note = AbD light chain
DIVMTQSPDSLSVSLGERATINCRASKSVDSYGNSYMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 448 aa, synthetic construct
  source 1..448: note = AbD heavy chain
QVTLRESGPALVKPTQTLTLTCTVSGSSLSAYSVNWIRQPPGKALEWLAMIWGDGKVVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 218 aa, synthetic construct
  source 1..218: note = AbE light chain
DIVMTQSPDSLSVSLGERATINCRASKSVDSYGHSYMHWYQQKPGQPPKLLIYWASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 448 aa, synthetic construct
  source 1..448: note = AbE heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 218 aa, synthetic construct
  source 1..218: note = AbF light chain
DIVMTQSPDSLSVSLGERATINCRASESVDSYGNSYMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 448 aa, synthetic construct
  source 1..448: note = AbF heavy chain
QVTLRESGPALVKPTQTLTLTCTVSGASLSRYSVNWIRQPPGKALEWLAMIWGDAKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 218 aa, synthetic construct; identical to SEQ ID NO: 135
  source 1..218: note = AbG light chain

SEQ ID NO: 142 - protein, 448 aa, synthetic construct; identical to SEQ ID NO: 138
  source 1..448: note = AbG heavy chain

SEQ ID NO: 143 - protein, 218 aa, synthetic construct
  source 1..218: note = AbH light chain
DIVMTQSPDSLSVSLGERATINCRASKSVDSYGNSYMHWYQQKPGQPPKLLIYRASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 448 aa, synthetic construct
  source 1..448: note = AbH heavy chain
QVTLRESGPALVKPTQTLTLTCTVSGASLSRYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 218 aa, synthetic construct; identical to SEQ ID NO: 135
  source 1..218: note = AbI light chain

SEQ ID NO: 146 - protein, 448 aa, synthetic construct
  source 1..448: note = AbI heavy chain
QVTLRESGPALVKPTQTLTLTCTVSGASLSRYSVNWIRQPPGKALEWLAMIWGDGKIVYNSALKSRLTISKDTSKNQVVLTMTNMDPVDTATYYCAGDGFYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 218 aa, synthetic construct; identical to SEQ ID NO: 137
  source 1..218: note = AbJ light chain

SEQ ID NO: 148 - protein, 448 aa, synthetic construct
  source 1..448: note = AbJ heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 218 aa, synthetic construct
  source 1..218: note = AbK light chain
DIVMTQSPDSLSVSLGERATINCRASQSVDSYGNSYMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 448 aa, synthetic construct
  source 1..448: note = AbK heavy chain
QVTLRESGPALVKPTQTLTLTCTVSGSSLSRYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 218 aa, synthetic construct; identical to SEQ ID NO: 129
  source 1..218: note = AbL light chain

SEQ ID NO: 152 - protein, 448 aa, synthetic construct
  source 1..448: note = AbL heavy chain
QVTLRESGPALVKPTQTLTLTCTVSGFSLSRYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 218 aa, synthetic construct
  source 1..218: note = AbM light chain
DIVMTQSPDSLSVSLGERATINCRASKSVDYYGRSYMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 448 aa, synthetic construct; identical to SEQ ID NO: 138
  source 1..448: note = AbM heavy chain

SEQ ID NO: 155 - protein, 218 aa, synthetic construct
  source 1..218: note = AbN light chain
DIVMTQSPDSLSVSLGERATINCRASESVDYYGHSYMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 448 aa, synthetic construct; identical to SEQ ID NO: 132
  source 1..448: note = AbN heavy chain

SEQ ID NO: 157 - protein, 218 aa, synthetic construct; identical to SEQ ID NO: 131
  source 1..218: note = AbO light chain

SEQ ID NO: 158 - protein, 448 aa, synthetic construct
  source 1..448: note = AbO heavy chain
QVTLRESGPALVKPTQTLTLTCTVSGYSLSAYSVNWIRQPPGKALEWLAMIWGDGKIVYNSALKSRLTISKDTSKNQVVLTMTNMDPVDTATYYCAGDGYFPYAMDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 218 aa, synthetic construct
  source 1..218: note = AbP light chain
DIVMTQSPDSLSVSLGERATINCRASKSVDSYGNSYMHWYQQKPGQPPKLLIYWASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 448 aa, synthetic construct
  source 1..448: note = AbP heavy chain
QVTLRESGPALVKPTQTLTLTCTVSGASLSAYSVNWIRQPPGKALEWLAMIWGDGKVVYNSALKSRLTISKDTSKNQVVLTMTNMDPVDTATYYCAGDGYFPYAMDHWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 146 aa, Homo sapiens [sequence omitted]

SEQ ID NO: 162 - protein, 15 aa, synthetic construct
  source 1..15: note = Ref1 LCDR1 Kabat
RASKSVDSYGNSFMH

SEQ ID NO: 163 - protein, 7 aa, synthetic construct
  source 1..7: note = Ref1 LCDR2 Kabat
LASNLES

SEQ ID NO: 164 - protein, 9 aa, synthetic construct
  source 1..9: note = Ref1 LCDR3 Kabat
QQNNEDPRT

SEQ ID NO: 165 - protein, 5 aa, synthetic construct
  source 1..5: note = Ref1 HCDR1 Kabat
AYSVN

SEQ ID NO: 166 - protein, 16 aa, synthetic construct
  source 1..16: note = Ref1 HCDR2 Kabat
MIWGDGKIVYNSALKS

SEQ ID NO: 167 - protein, 10 aa, synthetic construct
  source 1..10: note = Ref1 HCDR3 Kabat
DGYYPYAMDN

SEQ ID NO: 168 - protein, 15 aa, synthetic construct
  source 1..15: note = Ref2 LCDR1 Kabat
RASKSVDSYGNSFMH

SEQ ID NO: 169 - protein, 7 aa, synthetic construct
  source 1..7: note = Ref2 LCDR2 Kabat
LASNLES

SEQ ID NO: 170 - protein, 9 aa, synthetic construct
  source 1..9: note = Ref2 LCDR3 Kabat
QQNNEDPRT

SEQ ID NO: 171 - protein, 5 aa, synthetic construct
  source 1..5: note = Ref2 HCDR1 Kabat
AYSVN

SEQ ID NO: 172 - protein, 16 aa, synthetic construct
  source 1..16: note = Ref2 HCDR2 Kabat
MIWGDGKIVYNSALKS

SEQ ID NO: 173 - protein, 10 aa, synthetic construct
  source 1..10: note = Ref2 HCDR3 Kabat
DGYYPYAMDN

SEQ ID NO: 174 - protein, 111 aa, synthetic construct [sequence omitted]

SEQ ID NO: 175 - protein, 118 aa, synthetic construct [sequence omitted]

SEQ ID NO: 176 - protein, 111 aa, synthetic construct [sequence omitted]

SEQ ID NO: 177 - protein, 118 aa, synthetic construct [sequence omitted]

SEQ ID NO: 178 - protein, 218 aa, synthetic construct
  source 1..218: note = Ref1 light chain
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 448 aa, synthetic construct; identical to SEQ ID NO: 130
  source 1..448: note = Ref1 heavy chain

SEQ ID NO: 180 - protein, 218 aa, synthetic construct; identical to SEQ ID NO: 178
  source 1..218: note = Ref2 light chain

SEQ ID NO: 181 - protein, 445 aa, synthetic construct
  source 1..445: note = Ref2 heavy chain
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 182 - protein, 448 aa, synthetic construct
  source 1..448: note = Heavy chain constant domain: hG1-REM-LALA-G237A-YTE; note = AbQ heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 448 aa, synthetic construct
  source 1..448: note = Heavy chain constant domain: hG1-REM-LALA-G237A-YTE; note = AbR heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 448 aa, synthetic construct; identical to SEQ ID NO: 182
  source 1..448: note = Heavy chain constant domain: hG1-REM-LALA-G237A-YTE; note = AbS heavy chain

SEQ ID NO: 185 - protein, 448 aa, synthetic construct
  source 1..448: note = Heavy chain constant domain: hG1-REM-LALA-PA-YTE; note = AbT heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 448 aa, synthetic construct
  source 1..448: note = Heavy chain constant domain: hG1-REM-LALA-PA-YTE; note = AbU heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 448 aa, synthetic construct; identical to SEQ ID NO: 185
  source 1..448: note = Heavy chain constant domain: hG1-REM-LALA-PA-YTE; note = AbV heavy chain

SEQ ID NO: 188 - protein, 448 aa, synthetic construct
  source 1..448: note = Heavy chain constant domain: hG1-REM-LALA-PG-YTE; note = AbW heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 448 aa, synthetic construct
  source 1..448: note = Heavy chain constant domain: hG1-REM-LALA-PG-YTE; note = AbX heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 448 aa, synthetic construct; identical to SEQ ID NO: 188
  source 1..448: note = Heavy chain constant domain: hG1-REM-LALA-PG-YTE; note = AbY heavy chain

SEQ ID NO: 191 - protein, 448 aa, synthetic construct
  source 1..448: note = Heavy chain constant domain: hg1-REM YTE TM; note = AbZ heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 448 aa, synthetic construct
  source 1..448: note = Heavy chain constant domain: hg1-REM YTE TM; note = AbAA heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 448 aa, synthetic construct; identical to SEQ ID NO: 191
  source 1..448: note = Heavy chain constant domain: hg1-REM YTE TM; note = AbAB heavy chain

SEQ ID NO: 194 - protein, 448 aa, synthetic construct
  source 1..448: note = Heavy chain constant domain: hG1-REM TM (L234F/L235E/P331S); note = AbAC heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 448 aa, synthetic construct
  source 1..448: note = Heavy chain constant domain: hG1-REM TM (L234F/L235E/P331S); note = AbAD heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 448 aa, synthetic construct; identical to SEQ ID NO: 194
  source 1..448: note = Heavy chain constant domain: hG1-REM TM (L234F/L235E/P331S); note = AbAE heavy chain

SEQ ID NO: 197 - protein, 448 aa, synthetic construct
  source 1..448: note = Heavy chain constant domain: hG1-REM-LALA-PG; note = AbAF heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 448 aa, synthetic construct
  source 1..448: note = Heavy chain constant domain: hG1-REM-LALA-PG; note = AbAG heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 448 aa, synthetic construct; identical to SEQ ID NO: 197
  source 1..448: note = Heavy chain constant domain: hG1-REM-LALA-PG; note = AbAH heavy chain

SEQ ID NO: 200 - protein, 448 aa, synthetic construct
  source 1..448: note = Heavy chain constant domain: hG1-REM-LALA-PA; note = AbAI heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 448 aa, synthetic construct
  source 1..448: note = Heavy chain constant domain: hG1-REM-LALA-PA; note = AbAJ heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 448 aa, synthetic construct; identical to SEQ ID NO: 200
  source 1..448: note = Heavy chain constant domain: hG1-REM-LALA-PA; note = AbAK heavy chain

SEQ ID NO: 203 - protein, 448 aa, synthetic construct
  source 1..448: note = Heavy chain constant domain: LALA-GA_hG1-REM; note = AbAL heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 448 aa, synthetic construct
  source 1..448: note = Heavy chain constant domain: LALA-GA_hG1-REM; note = AbAM heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 448 aa, synthetic construct; identical to SEQ ID NO: 203
  source 1..448: note = Heavy chain constant domain: LALA-GA_hG1-REM; note = AbAN heavy chain

SEQ ID NO: 206 - protein, 448 aa, synthetic construct
  source 1..448: note = Heavy chain constant domain: hG1 (WT-REM)-LALA; note = AbAO heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 448 aa, synthetic construct
  source 1..448: note = Heavy chain constant domain: hG1 (WT-REM)-LALA; note = AbAP heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 448 aa, synthetic construct; identical to SEQ ID NO: 206
  source 1..448: note = Heavy chain constant domain: hG1 (WT-REM)-LALA; note = AbAQ heavy chain

SEQ ID NO: 209 - protein, 448 aa, synthetic construct
  source 1..448: note = Heavy chain constant domain: hG1 (WT-REM)-LALA-YTE; note = AbAR heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYFPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 448 aa, synthetic construct
  source 1..448: note = Heavy chain constant domain: hG1 (WT-REM)-LALA-YTE; note = AbAS heavy chain
QVTLRESGPALVKPTQTLTLTCTVSGASLSAYSVNWIRQPPGKALEWLAMIWGDRKKVYNSALKSRLTISKDTSKNQVVLTMTNMDPVDTATYYCAGDGYFPYAMDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 448 aa, synthetic construct; identical to SEQ ID NO: 209
  source 1..448: note = Heavy chain constant domain: hG1 (WT-REM)-LALA-YTE; note = AbAT heavy chain

SEQ ID NO: 212 - protein, 445 aa, synthetic construct
  source 1..445: note = NB-13 heavy chain
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 213 - protein, 445 aa, synthetic construct
  source 1..445: note = NB-1 heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGLGYYPYAMDLWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 214 - protein, 445 aa, synthetic construct
  source 1..445: note = NB-2 heavy chain
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGLGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 215 - protein, 445 aa, synthetic construct
  source 1..445: note = NB-3 heavy chain
QVTLRESGPALVKPTQTLTLTCTVSGASLSSYSVNWIRQPPGKALEWLAMIWGDGKIVYNSALKSRLTISKDTSKNQVVLTMTNMDPVDTATYYCAGEGYYPYAMDVWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 216 - protein, 445 aa, synthetic construct
  source 1..445: note = NB-4 heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 217 - protein, 445 aa, synthetic construct
  source 1..445: note = NB-5 heavy chain
QVTLRESGPALVKPTQTLTLTCTVSGASLSAYSVNWIRQPPGKALEWLAMIWGSGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 218 - protein, 445 aa, synthetic construct
  source 1..445: note = NB-6 heavy chain
QVTLRESGPALVKPTQTLTLTCTVSGASLSAYSVNWIRQPPGKALEWLAMISGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 219 - protein, 445 aa, synthetic construct; identical to SEQ ID NO: 212
  source 1..445: note = NB-7 heavy chain

SEQ ID NO: 220 - protein, 445 aa, synthetic construct
  source 1..445: note = NB-8 heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGEGYYPYAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 221 - protein, 445 aa, synthetic construct
  source 1..445: note = NB-9 heavy chain
QVTLRESGPALVKPTQTLTLTCTVSGFSLSAYSVNWIRQPPGKALEWLAMIWGSGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 222 - protein, 445 aa, synthetic construct; identical to SEQ ID NO: 216
  source 1..445: note = NB-10 heavy chain

SEQ ID NO: 223 - protein, 445 aa, synthetic construct
  source 1..445: note = NB-11 heavy chain
QVTLRESGPALVKPTQTLTLTCTVSGASLSAYSVNWIRQPPGKALEWLAMIWGDGTIVYNSALKSRLTISKDTSKNQVVLTMTNMDPVDTATYYCAGDGYFPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 224 - protein, 445 aa, synthetic construct
  source 1..445: note = NB-12 heavy chain
QVTLRESGPALVKPTQTLTLTCTVSGASLSAYSVNWIRQPPGKALEWLAMIWGDGKIVYNSALKSRLTISKDTSKNQVVLTMTNMDPVDTATYYCAGDGYYPWAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 225 - protein, 218 aa, synthetic construct; identical to SEQ ID NO: 178
  source 1..218: note = NB-13 light chain

SEQ ID NO: 226 - protein, 218 aa, synthetic construct; identical to SEQ ID NO: 178
  source 1..218: note = NB-1 light chain

SEQ ID NO: 227 - protein, 218 aa, synthetic construct; identical to SEQ ID NO: 178
  source 1..218: note = NB-2 light chain

SEQ ID NO: 228 - protein, 218 aa, synthetic construct; identical to SEQ ID NO: 135
  source 1..218: note = NB-3 light chain

SEQ ID NO: 229 - protein, 218 aa, synthetic construct
  source 1..218: note = NB-4 light chain
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 218 aa, synthetic construct; identical to SEQ ID NO: 178
  source 1..218: note = NB-5 light chain

SEQ ID NO: 231 - protein, 218 aa, synthetic construct; identical to SEQ ID NO: 178
  source 1..218: note = NB-6 light chain

SEQ ID NO: 232 - protein, 218 aa, synthetic construct; identical to SEQ ID NO: 229
  source 1..218: note = NB-7 light chain

SEQ ID NO: 233 - protein, 218 aa, synthetic construct; identical to SEQ ID NO: 178
  source 1..218: note = NB-8 light chain

SEQ ID NO: 234 - protein, 218 aa, synthetic construct; identical to SEQ ID NO: 178
  source 1..218: note = NB-9 light chain

SEQ ID NO: 235 - protein, 218 aa, synthetic construct
  source 1..218: note = NB-10 light chain
DIVMTQSPDSLSVSLGERATINCRASKSVDYYGNSFMHWYQQKPGQPPKLLIYLASNLESGVPDRFSGSGSGTDFTLTISSLQAEDVAVYYCQQNNEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 218 aa, synthetic construct
  source 1..218: note = NB-11 light chain
DIVMTQSPDSLSVSLGERATINCRASKSVDSYGHSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 218 aa, synthetic construct
  source 1..218: note = NB-12 light chain
DIVMTQSPDSLSVSLGERATINCRASQSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 448 aa, synthetic construct
  source 1..448: note = ref3 heavy chain
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 218 aa, synthetic construct; identical to SEQ ID NO: 178
  source 1..218: note = ref3 light chain